(19) United States
(12) Patent Application Publication
(10) Pub. No.: US 2020/0337311 A1
(43) Pub. Date: Oct. 29, 2020

(54) FLUORALKENYL COMPOUNDS, PROCESS FOR PREPARATION AND USE THEREOF

(71) Applicant: PI INDUSTRIES LTD., Udaipur Rajasthan (IN)

(72) Inventors: Sathishkumar Murugan, Tamilnadu (IN); Renugadevi Gurusamy, Chennai- Tamilnadu (IN); Gopalkrushna Tulshidas Waghule, Ahmednagar, Maharashtra (IN); Suresh Ashamoni, Telangana (IN); Kishor Singh Rathod, Jodhpur, Rajasthan (IN); Vikram Singh Jhala, Udaipur, Rajasthan (IN); Uzma Khan, Aligarh (IN); Deepak Lahanya Ebhad, Palghar, Maharashtra (IN); Anil Kumar Verma, Solan, Himachal Pradesh (IN); Ruchi Garg, Varanasi, UP (IN); Hagalavadi M. Venkatesha, Bengaluru, Karnataka (IN); Alexander G.M. KLAUSENER, Pulheim (DE)

(73) Assignee: PI INDUSTRIES LTD., Udaipur Rajasthan (IN)

(21) Appl. No.: 16/955,809

(22) PCT Filed: Dec. 17, 2018
(86) PCT No.: PCT/IB2018/060164

§ 371 Date: Jun. 19, 2020

(30) Foreign Application Priority Data
Dec. 20, 2017 (IN) 201711045940

(51) Int. Cl. A01N 43/78; A01C 1/06; A01G 7/00; C07D 277/36; C07D 263/46; A01N 43/76; C07D 417/04; A01N 43/82; C07D 233/84; A01N 43/50

(52) U.S. Cl. CPC A01N 43/78; A01C 1/06; A01G 7/00; C07D 277/36; A01N 43/50; A01N 43/76; C07D 417/04; A01N 43/82; C07D 233/84; C07D 263/46

(57) ABSTRACT

The present invention disclosed fluoralkenyl compounds of general formula (I);

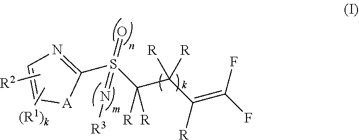

wherein; R, R¹, R², R³, A and integers n, m and k are as defined in description. The present invention further discloses use of the compounds of general formula (I) to protect crops by controlling or preventing against undesired phytopathogenic microorganisms such as nematodes and phytopathogenic fungi.

### FIELD OF THE INVENTION

The present invention relates to novel fluoralkenyl compounds and their use as crop protection agents.

### BACKGROUND OF THE INVENTION

WO200102378, U.S. Pat. No. 3,518,172 describes trifluorobutenyl compounds which have nematicidal activity. JP500037/1988 (WO 86/07590) describes polyhaloalkene compounds which have nematicidal activity. WO95/24403 describes that 4,4-difluorobutenyl compounds have nematicidal activity. JP176141/1997 mentions thiazole compounds having insecticidal and acaricidal activity. WO2017002100, WO2004095930, WO2004095929, DE10254876, WO2004005268, WO2003049541, WO2003029231, WO2002006259, WO2002006257, WO2002006256, WO2001066529, US354979, U.S. Pat. Nos. 3,891,662, 3,780,050, 3,700,668, 3,697,536, 3,692,912, 3,666,818, 3,654,293 also describe polyhaloalkene compounds which have nematicidal activity. WO94/06782 discloses benzthiazoles and benzoxazoles having nematicidal, insecticidal, acaricidal and fungicidal properties. WO94/06777 discloses pyrimidine derivatives having nematicidal, insecticidal, acaricidal and fungicidal properties.

The control of damages to crops caused by phytopathogenic microorganisms and pests is extremely important in achieving high crop efficiency. For instance, plant disease damage to ornamental, vegetable, field, cereal and fruit crops can cause significant reduction in productivity and thereby result in increased cost to the consumer. Many products are commercially available to control such damages. The need continues for new compounds which are more effective, less costly, less toxic, environmentally safer and/or have different modes of action.

The effectiveness of the diflurobutenes, trifluorobutenes and other compounds described in the prior art is good, but are not fully satisfactory in various cases. Therefore, it is always of high interest in agriculture to find novel pesticidal compounds in order to avoid and/or control the development of microorganisms such as fungal or bacterial pathogens or pests being resistant to known active ingredients. It is therefore of high interest to use novel compounds being more active than those already known, with the aim of decreasing the amounts of active compound to be used, whilst at the same time maintaining an effectiveness at least equivalent to the already known compounds.

The present invention describes compounds of formula (I) which possess the above mentioned effects or advantages. Such compounds of formula (I), namely fluoralkenyl compounds wherein the heterocyclic ring is substituted according to the invention, show unexpected and significantly higher activity against undesired microorganisms such as fungal or bacterial pathogens or against pests such as nematodes or insects.

### SUMMARY OF THE INVENTION

Accordingly, the present invention provides a fluoralkenyl compound of the general formula (I),

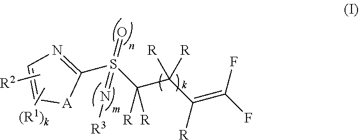

Wherein; R, R¹, R², R³, A and integers n, m and k are as defined in detailed description and their use for controlling or preventing agricultural crops and/or horticultural crops against nematodes and phytopathogenic fungi.

### DESCRIPTION

### Definitions

The terminologies used in the present disclosure are for illustrative purpose only and in no manner limit the scope of the present invention disclosed in the present disclosure.

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of excludes any element, step or ingredient not specified. If in the claim, such would close the claim to the inclusion of materials other than those recited except for impurities ordinarily associated therewith. When the phrase” consisting of appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Further, unless expressly stated to the contrary, “or” refers to an inclusive “or” and not to an exclusive “or”. For example, a condition A “or” B is satisfied by any one of the following: A is true (or present) and B is false (or not present), A is false (or not present) and B is true (or present), and both A and B are true (or present).

Also, the indefinite articles “a” and “an” preceding an element or component of the present invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “pesticide” in each case also always comprises the term “crop protection agent”.

The term “invertebrate pest” includes arthropods, gastropods and nematodes of economic importance as pests. The term “arthropod” includes insects, mites, spiders, scorpions, centipedes, millipedes, pill bugs and symphylans. The term “gastropod” includes snails, slugs and other Stylommatophora. The term “nematode” refers to a living organism of the Phylum Nematoda. The term “helminths” includes roundworms, heartworms, phytophagous nematodes (Nematoda), flukes (Trematoda), acanthocephala and tapeworms (Cestoda).

The term “undesired microorganisms” or “phytopathogenic microorganisms” such as fungal or bacterial pathogens includes Plasmodiophoromycetes, Oomycetes, Chytridiomycetes, Zygomycetes, Ascomycetes, Basidiomycetes and Deuteromycetes and Pseudomonadaceae, Rhizobiaceae, Enterobacteriaceae, Corynebacteriaceae and Streptomycetaceae respectively.

In the context of this disclosure “invertebrate pest control” means inhibition of invertebrate pest development (including mortality, feeding reduction, and/or mating disruption), and related expressions are defined analogously.

The term “agronomic” refers to the production of field crops such as for food and fiber and includes the growth of corn, soybeans and other legumes, rice, cereal (e.g., wheat, oats, barley, rye, rice, maize), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (berries, cherries) and other specialty crops (e.g., canola, sunflower, olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

Nonagronomic applications include protecting an animal from an invertebrate parasitic pest by administering a parasiticidally effective (i.e. biologically effective) amount of a compound of the present invention, typically in the form of a composition formulated for veterinary use, to the animal to be protected. As referred to in the present disclosure and claims, the terms “parasiticidal” and “parasiticidally” refers to observable effects on an invertebrate parasite pest to provide protection of an animal from the pest. Parasiticidal effects typically relate to diminishing the occurrence or activity of the target invertebrate parasitic pest. Such effects on the pest include necrosis, death, retarded growth, diminished mobility or lessened ability to remain on or in the host animal, reduced feeding and inhibition of reproduction. These effects on invertebrate parasite pests provide control (including prevention, reduction or elimination) of parasitic infestation or infection of the animal.

The compounds of the present disclosure may be present either in pure form or as mixtures of different possible isomeric forms such as stereoisomers or constitutional isomers. The various stereoisomers include enantiomers, diastereomers, chiral isomers, atropisomers, conformers, rotamers, tautomers, optical isomers, polymorphs, and geometric isomers. Any desired mixtures of these isomers fall within the scope of the claims of the present disclosure. One skilled in the art will appreciate that one stereoisomer may be more active and/or may exhibit beneficial effects when enriched relative to the other isomer(s) or when separated from the other isomer(s). Additionally, the person skilled in the art knows processes or methods or technology to separate, enrich, and/or to selectively prepare said isomers.

The meaning of various terms used in the description shall now be illustrated:

In the above description, the term “alkyl”, used either alone or in compound words such as “alkylthio” or “haloalkyl” or —N(alkyl) or alkylcarbonylalkyl or alkylsuphonylamino includes straight-chain or branched C₁ to C₂₄ alkyl, preferably C₁ to C₁₅ alkyl, more preferably C₁ to C₁₀ alkyl, most preferably C₁ to C₆ alkyl. Non limiting examples of alkyl include methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl or the different isomers. If the alkyl is at the end of a composite substituent, as, for example, in alkylcycloalkyl, the part of the composite substituent at the start, for example the cycloalkyl, may be mono- or polysubstituted identically or differently and independently by alkyl.

The same also applies to composite substituents in which other radicals, for example alkenyl, alkynyl, hydroxyl, halogen, carbonyl, carbonyloxy and the like, are at the end.

The term “alkenyl”, used either alone or in compound words includes straight-chain or branched C₂ to C₂₄ alkenes, preferably C₂ to C₁₅ alkenes, more preferably C₂ to C₁₀ alkenes, most preferably C₂ to C₆ alkenes. Non limiting examples of alkenes include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl and the different isomers. The term“Alkenyl” also includes polyenes such as 1,2-propadienyl and 2,4-hexadienyl. This definition also applies to alkenyl as a part of a composite substituent, for example haloalkenyl and the like, unless defined specifically elsewhere.

The term “alkynyl”, used either alone or in compound words includes branched or straight-chain C₂ to C₂₄ alkynes, preferably C₂ to C₁₅ alkynes, more preferably C₂ to C₁₀ alkynes, most preferably C₂ to C₆ alkynes. Non limiting examples of alkynes include ethynyl, 1-propynyl, 2-propynyl, 1-butynyl, 2-butyryl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl and the different isomers. This definition also applies to alkynyl as a part of a composite substituent, for example haloalkynyl etc., unless specifically defined elsewhere. The term“Alkynyl” can also include moieties comprised of multiple triple bonds such as 2,5-hexadiynyl.

The term “cyclic alkyl” or “cycloalkyl” means alkyl closed to form a ring. Non limiting examples include but are not limited to cyclopropyl, cyclobutyl, cyclopentyl and cyclohexyl. This definition also applies to cycloalkyl as a part of a composite substituent, for example cycloalkylalkyl etc., unless specifically defined elsewhere.

The term “cycloalkenyl” means alkenyl closed to form a ring including monocyclic, partially unsaturated hydrocarbyl groups. Non limiting examples include but are not limited to cyclopentenyl and cyclohexenyl. This definition also applies to cycloalkenyl as a part of a composite substituent, for example cycloalkenylalkyl etc., unless specifically defined elsewhere.

The term “cycloalkynyl” means alkynyl closed to form a ring including monocyclic, partially unsaturated groups. This definition also applies to cycloalkynyl as a part of a composite substituent, for example cycloalkynylalkyl etc., unless specifically defined elsewhere.

The term “cycloalkoxy”, “cycloalkenyloxy” and the like are defined analogously. Non limiting examples of cycloalkoxy include cyclopropyloxy, cyclopentyloxy and cyclohexyloxy. This definition also applies to cycloalkoxy as a part of a composite substituent, for example cycloalkoxy alkyl etc., unless specifically defined elsewhere.

The term “alkoxy” used either alone or in compound words included C₁ to C₂₄ alkoxy, preferably C₁ to C₁₅ alkoxy, more preferably C₁ to C₁₀ alkoxy, most preferably C₁ to C₆ alkoxy. Examples of alkoxy include methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy and the different isomers. This definition also applies to alkoxy as a part of a composite substituent, for example haloalkoxy, alkynylalkoxy, etc., unless specifically defined elsewhere.

The term “alkylthio” includes branched or straight-chain alkylthio moieties such as methylthio, ethylthio, prop ylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylprop ylthio, 1-ethylpropylthio, hexylthio, 1,1-dimethylpropylthio, 1,2-dimethylprop ylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio and the different isomers.

The term “hydroxy” means —OH, “amino” means —NRR, wherein R can be H or any possible substituent such as alkyl; “carbonyl” means —C(O)—, “carbonyloxy” means —OC(O)—, “sulfinyl” means SO, “sulfonyl” means S(O)₂.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different.

Non-limiting examples of “haloalkyl” include chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 1,1-dichloro-2,2,2-trifluoroethyl, and 1,1,1-trifluoroprop-2-yl. This definition also applies to haloalkyl as a part of a composite substituent, for example haloalkylaminoalkyl etc., unless specifically defined elsewhere.

The terms “haloalkenyl” and “haloalkynyl” are defined analogously except that, instead of alkyl groups, alkenyl and alkynyl groups are present as a part of the substituent.

The term “haloalkoxy” means straight-chain or branched alkoxy groups where some or all of the hydrogen atoms in these groups may be replaced by halogen atoms as specified above. Non-limiting examples of haloalkoxy include chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy. This definition also applies to haloalkoxy as a part of a composite substituent, for example haloalkoxyalkyl etc., unless specifically defined elsewhere.

The terms “haloalkylthio” or “haloalkylsulfanyl” means straight-chain or branched alkylthio groups where some or all of the hydrogen atoms in these groups may be replaced by halogen atoms as specified above. Non-limiting examples of haloalkylthio include chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio. This definition also applies to haloalkylthio as a part of a composite substituent, for example haloalkylthioalkyl etc., unless specifically defined elsewhere.

Non limiting examples of “haloalkylsulfinyl” include CF₃S(O), CCl₃S(O), CF₃CH₂S(O) and CF₃CF₂S(O). Non limiting examples of “haloalkylsulfonyl” include CF₃S(O)₂, CCl₃S(O)₂, CF₃CH₂S(O)₂ and CF₃CF₂S(O)₂.

The term “alkylthioalkyl” denotes alkylthio substitution on alkyl. Non limiting examples of “alkylthioalkyl” include —CH₂SCH₂, —CH₂SCH₂CH₂, CH₃CH₂SCH₂, CH₃CH₂CH₂CH₂SCH₂ and CH₃CH₂SCH₂CH₂. The term “Alkylthioalkoxy” denotes alkylthio substitution on alkoxy. The term “cycloalkylalkylamino” denotes cycloalkyl substitution on alkyl amino.

The terms alkoxyalkoxyalkyl, alkylaminoalkyl, dialkylaminoalkyl, cycloalkylaminoalkyl, cycloalkylaminocarbonyl and the like, are defined analogously to “alkylthioalkyl” or cycloalkylalkylamino.

The term “alkoxycarbonyl” is an alkoxy group bonded to a skeleton via a carbonyl group (—CO—). This definition also applies to alkoxycarbonyl as a part of a composite substituent, for example cycloalkylalkoxycarbonyl and the like, unless specifically defined elsewhere.

The term “alkoxycarbonylalkylamino” denotes alkoxy carbonyl substitution on alkyl amino.

The term “Alkylcarbonylalkylamino” denotes alkyl carbonyl substitution on alkyl amino.

The terms alkylthioalkoxycarbonyl, cycloalkylalkylaminoalkyl and the like are defined analogously.

Non limiting examples of “alkylsulfinyl” include but are not limited to methylsulphinyl, ethylsulphinyl, propylsulphinyl, 1-methylethylsulphinyl, butylsulphinyl, 1-methylpropylsulphinyl, 2-methylpropylsulphinyl, 1,1-dimethylethylsulphinyl, pentylsulphinyl, 1-methylbutylsulphinyl, 2-methylbutylsulphinyl, 3-methylbutylsulphinyl, 2,2-dimethylpropylsulphinyl, 1-ethylpropylsulphinyl, hexylsulphinyl, 1,1-dimethylpropylsulphinyl, 1,2-dimethylpropylsulphinyl, 1-methylpentylsulphinyl, 2-methylpentylsulphinyl, 3-methylpentylsulphinyl, 4-methylpentylsulphinyl, 1,1-dimethylbutylsulphinyl, 1,2-dimethylbutylsulphinyl, 1,3-dimethylbutylsulphinyl, 2,2-dimethylbutylsulphinyl, 2,3-dimethylbutylsulphinyl, 3,3-dimethylbutylsulphinyl, 1-ethylbutylsulphinyl, 2-ethylbutylsulphinyl, 1,1,2-trimethylpropylsulphinyl, 1,2,2-trimethylpropylsulphinyl, 1-ethyl-1-methylpropylsulphinyl and 1-ethyl-2-methylpropylsulphinyl and the different isomers. The term “arylsulfinyl” includes Ar—S(O), wherein Ar can be any carbocyle or heterocylcle. This definition also applies to alkylsulphinyl as a part of a composite substituent, for example haloalkylsulphinyl etc., unless specifically defined elsewhere.

Non limiting examples of “alkylsulfonyl” include but are not limited to methylsulphonyl, ethylsulphonyl, propylsulphonyl, 1-methylethylsulphonyl, butylsulphonyl, 1-methylpropylsulphonyl, 2-methylpropylsulphonyl, 1,1-dimethylethylsulphonyl, pentylsulphonyl, 1-methylbutylsulphonyl, 2-methylbutylsulphonyl, 3-methylbutylsulphonyl, 2,2-dimethylpropylsulphonyl, 1-ethylpropylsulphonyl, hexylsulphonyl, 1,1-dimethylpropylsulphonyl, 1,2-dimethylpropylsulphonyl, 1-methylpentylsulphonyl, 2-methylpentylsulphonyl, 3-methylpentylsulphonyl, 4-methylpentylsulphonyl, 1,1-dimethylbutylsulphonyl, 1,2-dimethylbutylsulphonyl, 1,3-dimethylbutylsulphonyl, 2,2-dimethylbutylsulphonyl, 2,3-dimethylbutylsulphonyl, 3,3-dimethylbutylsulphonyl, 1-ethylbutylsulphonyl, 2-ethylbutylsulphonyl, 1,1,2-trimethylpropylsulphonyl, 1,2,2-trimethylpropylsulphonyl, 1-ethyl-1-methylpropylsulphonyl and 1-ethyl-2-methylpropylsulphonyl and the different isomers. The term “arylsulfonyl” includes Ar—S(O)₂, wherein Ar can be any carbocyle or heterocylcle. This definition also applies to alkylsulphonyl as a part of a composite substituent, for example alkylsulphonylalkyl etc., unless defined elsewhere.

The term “Alkylamino”, “dialkylamino”, and the like, are defined analogously to the above examples.

The term “ring” or “ring system” or “Cy” as a component of formula I is carbocyclyl or heterocyclyl.

The term “ring system” denotes one or more rings.

The term “bicyclic ring or ring system” denotes a ring system consisting of two or more common atom.

The term “aromatic” indicates that the Hueckel rule is satisfied and the term “non-aromatic” indicates that the Hueckel rule is not satisfied.

The terms “carbocycle” or “carbocyclic” or “carbocyclyl” include “aromatic carbocyclic ring system” and “nonaromatic carbocylic ring system” or polycyclic or bicyclic (spiro, fused, bridged, nonfused) ring compounds in which the ring may be aromatic or non-aromatic (where aromatic indicates that the Huckel rule is satisfied and non-aromatic indicates that the Huckel rule is not satisfied).

Non limiting examples of non-aromatic carbocyclic ring system are cyclopropyl, cyclobutyl, cyclopentyl, norbornyl and the like.

Non limiting examples of aromatic carbocyclic ring system are phenyl, naphthyl and the like.

The term “aryl” as used herein is a group that contains any carbon-based aromatic group including, but not limited to phenyl, naphthalene, biphenyl, anthracene, and the like. The aryl group can be substituted or unsubstituted. In addition, the aryl group can be a single ring structure or comprise multiple ring structures that are either fused ring structures or attached via one or more bridging groups such as a carbon-carbon bond.

The term “aralkyl” refers to aryl hydrocarbon radicals including an alkyl portion as defined above. Examples include benzyl, phenylethyl, and 6-napthylhexyl. As used herein, the term “aralkenyl” refers to aryl hydrocarbon radicals including an alkenyl portion, as defined above, and an aryl portion, as defined above. Examples include styryl, 3-(benzyl) prop-2-enyl, and 6-napthylhex-2-enyl.

The term “hetero” in connection with rings refers to a ring in which at least one ring atom is not carbon and which can contain 1 to 4 heteroatoms independently selected from the group consisting of nitrogen, oxygen and sulfur, provided that each ring contains no more than 4 nitrogens, no more than 2 oxygens and no more than 2 sulfurs.

The terms “heterocycle” or “heterocyclic” includes “aromatic heterocycle” or “heteroaryl ring system” and “nonaromatic heterocycle ring system” or polycyclic or bicyclic (spiro, fused, bridged, non-fused) ring compounds in which ring may be aromatic or non-aromatic, wherein the heterocycle ring contains at least one heteroatom selected from N, O, S(O)₀₋₂, and or C ring member of the heterocycle may be replaced by C(═O), C(═S), C(═CR*R*) and C═NR*, * indicates integers.

The terms “non-aromatic heterocycle” or “non-aromatic heterocyclic” means three- to fifteen-membered, preferably three- to twelve-membered, saturated or partially unsaturated heterocycle containing one to four heteroatoms from the group of oxygen, nitrogen and sulphur: mono, bi- or tricyclic heterocycles which contain, in addition to carbon ring members, one to three nitrogen atoms and/or one oxygen or sulphur atom or one or two oxygen and/or sulphur atoms; if the ring contains more than one oxygen atom, they are not directly adjacent; for example (but not limited to) oxiranyl, aziridinyl, oxetanyl, azetidinyl, thietanyl, 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 1-pyrazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 1-imidazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-1-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, pyrrolinyl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, piperidinyl, 2-piperidinyl, 3-piperidinyl, 4-piperidinyl, pyrazynyl, morpholinyl, thiomorphlinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl, 1,2,4-hexahydrotriazin-3-yl, cycloserines, 2,3,4,5-tetrahydro[1H]azepin-1- or -2- or -3- or -4- or -5- or -6- or -7-yl, 3,4,5,6-tetra-hydro[2H]azepin-2- or -3- or -4- or -5- or -6- or -7-yl, 2,3,4,7-tetrahydro[1H]azepin-1- or -2- or -3- or -4- or -5- or -6- or -7-yl, 2,3,6,7-tetrahydro[1H]azepin-1- or -2- or -3- or -4- or -5- or -6- or -7-yl, hexahydroazepin-1- or -2- or -3- or -4-yl, tetra- and hexahydrooxepinyl such as 2,3,4,5-tetrahydro[1H]oxepin-2- or -3- or -4- or -5- or -6- or -7-yl, 2,3,4,7-tetrahydro[1H]oxepin-2- or -3- or -4- or -5- or -6- or -7-yl, 2,3,6,7-tetrahydro[1H]oxepin-2- or -3- or -4- or -5- or -6- or -7-yl, hexahydroazepin-1- or -2- or -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl. This definition also applies to heterocyclyl as a part of a composite substituent, for example heterocyclylalkyl etc., unless specifically defined elsewhere.

The term “heteroaryl” means 5 or 6-membered, fully unsaturated monocyclic ring system containing one to four heteroatoms from the group of oxygen, nitrogen and sulphur; if the ring contains more than one oxygen atom, they are not directly adjacent; 5-membered heteroaryl containing one to four nitrogen atoms or one to three nitrogen atoms and one sulphur or oxygen atom: 5-membered heteroaryl groups which, in addition to carbon atoms, may contain one to four nitrogen atoms or one to three nitrogen atoms and one sulphur or oxygen atom as ring members, for example (but not limited thereto) furyl, thienyl, pyrrolyl, isoxazolyl, isothiazolyl, pyrazolyl, oxazolyl, thiazolyl, imidazolyl, 1,2,4-oxadiazolyl, 1,2,4-thiadiazolyl, 1,2,4-triazolyl, 1,3,4-oxadiazolyl, 1,3,4-thiadiazolyl, 1,3,4-triazolyl, tetrazolyl; nitrogen-bonded 5-membered heteroaryl containing one to four nitrogen atoms, or benzofused nitrogen-bonded 5-membered heteroaryl containing one to three nitrogen atoms: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example (but not limited to) 1-pyrrolyl, 1-pyrazolyl, 1,2,4-triazolyl, 1-imidazolyl, 1,2,3-triazolyl and 1,3,4-triazolyl.

6-membered heteroaryl which contains one to four nitrogen atoms: 6-membered heteroaryl groups which, in addition to carbon atoms, may contain, respectively, one to three and one to four nitrogen atoms as ring members, for example (but not limited thereto) 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl and 1,2,4,5-tetrazin-3-yl; benzofused 5-membered heteroaryl containing one to three nitrogen atoms or one nitrogen atom and one oxygen or sulphur atom: for example (but not limited to) indol-1-yl, indol-2-yl, indol-3-yl, indol-4-yl, indol-5-yl, indol-6-yl, indol-7-yl, benzimidazol-1-yl, benzimidazol-2-yl, benzimidazol-4-yl, benzimidazol-5-yl, indazol-1-yl, indazol-3-yl, indazol-4-yl, indazol-5-yl, indazol-6-yl, indazol-7-yl, indazol-2-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,3-benzoxazol-2-yl, 1,3-benzoxazol-4-yl, 1,3-benzoxazol-5-yl, 1,3-benzoxazol-6-yl and 1,3-benzoxazol-7-yl; benzofused 6-membered heteroaryl which contains one to three nitrogen atoms: for example (but not limited to) quinolin-2-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl and isoquinolin-8-yl.

Non-limiting examples of fused 6-5-membered heteroaryl include Indolizinyl; pyrazolo[1,5-a]pyridinyl; imidazo[1,2-a]pyridinyl; pyrrolo[1,2-a]pyrimidinyl; pyrazolo[1,5-a]pyrimidinyl; imidazo[1,2-a]pyrimidinyl; pyrrolo[1,2-a]pyrazinyl; pyrazolo[1,5-a]pyrazinyl; imidazo[1,2-a]pyrazinyl and the like.

This definition also applies to heteroaryl as a part of a composite substituent, for example heteroarylalkyl etc., unless specifically defined elsewhere.

The term “Trialkylsilyl” includes 3 branched and/or straight-chain alkyl radicals attached to and linked through a silicon atom such as trimethylsilyl, triethylsilyl and t-butyl-dimethylsilyl. The term“Halotrialkylsilyl” denotes at least one of the three alkyl radicals is partially or fully substituted with halogen atoms which may be the same or different. The term“Alkoxytrialkylsilyl” denotes at least one of the three alkyl radicals is substituted with one or more alkoxy radicals which may be the same or different. The term “Trialkylsilyloxy” denotes a trialkylsilyl moiety attached through oxygen. Non limiting examples of “alkylcarbonyl” include C(O)CH₃, C(O)CH₂CH₂CH₃ and C(O)CH(CH₃)₂. Examples of “alkoxycarbonyl” include CH₃OC(═O), CH₃CH₂OC(═O), CH₃CH₂CH₂OC(═O), (CH₃)₂CHOC(═O) and the different butoxy or pentoxycarbonyl isomers. Examples of “alkylaminocarbonyl” include CH₃NHC(═O), CH₃CH₂NHC(═O), CH₃CH₂CH₂NHC(═O), (CH₃)₂CHNHC(═O) and the different butylamino- or pentylaminocarbonyl isomers. Examples of “dialkylaminocarbonyl” include (CH₃)₂NC(═O), (CH₃CH₂)₂NC(═O), CH₃CH₂(CH₃)NC(═O), CH₃CH₂CH₂(CH₃)NC(═O) and (CH₃)₂CHN(CH₃)C(═O). Examples of “alkoxyalkylcarbonyl” include CH₃OCH₂C(═O), CH₃OCH₂CH₂C(═O), CH₃CH₂OCH₂C(═O), CH₃CH₂CH₂CH₂OCH₂C(═O) and CH₃CH₂OCH₂CH₂C(═O). Examples of “alkylthioalkylcarbonyl” include CH₃SCH₂C(═O), CH₃SCH₂CH₂C(═O), CH₃CH₂SCH₂C(═O), CH₃CH₂CH₂CH₂SCH₂C(═O) and CH₃CH₂SCH₂CH₂C(═O). The term haloalkylsufonylaminocarbonyl, alkylsulfonylaminocarbonyl, alkylthioalkoxycarbonyl, alkoxycarbonylalkyl amino and the like are defined analogously.

Non limiting examples of “alkylaminoalkylcarbonyl” include CH₃NHCH₂C(═O), CH₃NHCH₂CH₂C(═O), CH₃CH₂NHCH₂C(═O), CH₃CH₂CH₂CH₂NHCH₂C(═O) and CH₃CH₂NHCH₂CH₂C(═O).

The term “amide” means A-R′C═ONR″—B, wherein R′ and R″ indicates substituents and A and B indicate any group.

The term “thioamide” means A-R′C═SNR″—B, wherein R′ and R″ indicates substituents and A and B indicate any group.

The total number of carbon atoms in a substituent group is indicated by the “Cᵢ-Cⱼ” prefix where i and j are numbers from 1 to 21. For example, C₁-C₃ alkoxy designates methoxy through propoxy. In the above recitations, when a compound of formula (I) is comprised of one or more heterocyclic rings, all substituents are attached to these rings through any available carbon or nitrogen by replacement of a hydrogen on said carbon or nitrogen.

When a compound is substituted with a substituent bearing a subscript that indicates the number of said substituents can exceed 1, said substituents (when they exceed 1) are independently selected from the group of defined substituents. Further, when the subscript indicates a range, e. g. (R)ᵢ₋ⱼ, then the number of substituents may be selected from the integers between i and j inclusive.

When a group contains a substituent which can be hydrogen, for example R¹ or R², then, when this substituent is taken as hydrogen, it is recognized that this is equivalent to said group being unsubstituted.

Accordingly, the present invention provides a compound of general formula (I);

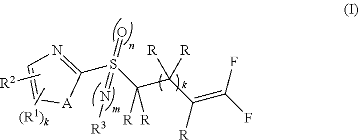

wherein;

A represent O, NR⁴ or S;

n, m and k represent integers, wherein n=0-2, m=0-1 and k=0-2; R is selected from the group consisting of hydrogen, halogen and C₁-C₃-alkyl;

R¹ is selected from the group consisting of R^{1a}, SCN, SF₅, NO₂, C₁-C₈-alkyl-S(O)₀₋₂R⁴, C₁-C₆-alkyl-OR⁴, C₁-C₈-alkyl-(C═O)—R⁴, C(R^{4a})═NR⁴, S(O)₀₋₂C₅-C₁₂-aryl, S(O)₀₋₂C₇-C₁₉-aralkyl, C₃-C₁₀-halocycloalkyl, C₄-C₁₀-cycloalkenyl, C₅-C₁₀-cycloalkynyl, C₁-C₈-alkyloxy-C₃-C₁₀-cycloalkyl, C₁-C₈-alkylthio-C₃-C₁₀-cycl ° alkyl, C₆-C₁₀-aryl, C₇-C₉-aralkyl, C₅-C₁₂-bicycloalkyl and C₇-C₁₂-bicycloalkenyl; wherein one or more carbon atoms of the cyclic ring system may be replaced by heteroatoms selected from the group consisting of N, O, S and optionally including 1 to 3 ring members selected from the group consisting of C(═O), C(═S), S(O)₀₋₂ and Si(R⁴)₂; R¹ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
wherein R^{1a} is selected from the group consisting of hydrogen, X, CN, (C═O)—R⁴, OR⁴, N(R⁴)₂, S(O)₀₋₂R⁴, Si(R⁴)₃, C₁-C₁₂-alkyl, C₂-C₁₂-alkenyl, C₂-C₁₂-alkynyl, C₁-C₁₂-haloalkyl, C₂-C₁₂-haloalkenyl, C₂-C₁₂-haloalkynyl, C₃-C₁₀-cycloalkyl; R^{1a} may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;

R² represent following fragment G

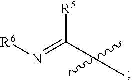

wherein R⁵ is selected from the group consisting of hydrogen, X, CN, C₁-C₁₂-alkyl, C₂-C₁₂-alkenyl, C₂-C₁₂-alkynyl, C₁-C₁₂-haloalkyl, C₂-C₁₂-haloalkenyl, C₂-C₁₂-haloalkynyl, C₃-C₁₀-cycloalkyl, C₃-C₁₀-halocycloalkyl, C₄-C₁₀-cycloalkenyl, C₅-C₁₀-cycloalkynyl, C₄-C₁₀-halocycloalkylalkyl, C₆-C₁₀-aryl and C₇-C₁₉-aralkyl; R⁵ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R⁶ is selected from the group consisting of hydrogen, OR⁴, N(R⁴)₂, Si(R⁴)₃, (C═O)—R⁴, S(O)₀₋₂R⁴, C₁-C₈-alkyl-S(O)₀₋₂R⁴, C₁-C₈-alkyl-(C═O)—R⁴, S(O)₀₋₂C₅-C₁₉-aryl, S(O)₀₋₂ C₇-C₁₉-aralkyl, C₁-C₁₂-alkyl, C₂-C₁₂-alkenyl, C₂-C₁₂-alkynyl, C₁-C₁₂-haloalkyl, C₂-C₁₂-haloalkenyl, C₂-C₁₂-haloalkynyl, C₃-C₁₀-cyclo alkyl, C₃-C₁₀-halocycloalkyl C₄-C₁₀-cycloalkenyl, C₅-C₁₀-cycloalkynyl, C₃-C₁₀-cycloalkyloxy, C₃-C₁₀-cycloalkylthio, C₆-C₁₀-aryl, C₇-C₁₉-aralkyl, C₅-C₁₂-bicycloalkyl, C₇-C₁₂-bicycloalkenyl and C₃-C₁₀-heterocyclyl; or
R⁵ and R⁶ together with the atom to which they are attached or together with further atoms selected from the group consisting of C, N, O, S and optionally including 1 to 3 ring members selected from the group consisting of C(═O), C(═S), S(O)₀₋₂ and Si(R⁴)₂, may form a four to seven membered ring, which for its part may be substituted by one or more R⁷;
R⁷ is selected from the group consisting of hydrogen, X, CN, SCN, SF₅, R⁴, OR⁴, NO₂, N(R⁴)₂, Si(R⁴)₃, (C═O)—R⁴, S(O)₀₋₂R⁴, C₁-C₈-alkyl-S(O)₀₋₂R⁴, C₁-C₈-alkyl-(C═O)—R⁴, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₃-C₁₀-cycloalkyl, C₃-C₁₀-halocycloalkyl, C₄-C₁₀-cycloalkenyl, C₅-C₁₀-cycloalkynyl, C₆-C₁₀-aryl, C₇-C₁₉-aralkyl and C₃-C₁₀-heterocyclyl; each of R⁶ and R⁷ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R³ is selected from the group consisting of hydrogen, CN, (C═O)—R⁴, OR⁴, N(R⁴)₂, S(O)₀₋₂R⁴, Si(R⁴)₃, C₁-C₁₂-alkyl, C₂-C₁₂-alkenyl, C₂-C₁₂-alkynyl, C₁-C₁₂-haloalkyl, C₂-C₁₂-haloalkenyl, C₂-C₁₂-haloalkynyl, C₃-C₁₀-cycloalkyl, C₄-C₁₀-cycloalkenyl and C₅-C₁₀-cycloalkynyl; R³ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R⁴ is selected from the group consisting of hydrogen, OR^{4a}, N(R^{4a})₂, C₁-C₆-alkyl, C₁-C₆-alkenyl, C₁-C₆-alkynyl, C₁-C₆-haloalkyl, C₂-C₆-haloalkenyl, C₂-C₆-haloalkynyl, C₃-C₁₂-cycloalkyl, C₃-C₁₀-halocycloalkyl, C₄-C₁₂-cycloalkenyl, C₅-C₁₂-cycloalkynyl, C₆-C₁₀-aryl, C₇-C₁₉-aralkyl and C₃-C₁₂-heterocyclyl; R⁴ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂,
R^{4a} is selected from the group consisting of hydrogen, C₁-C₆-alkyl and C₃-C₆-cycloalkyl;

wherein; when m=1 then R² may or may not be present;

X represents halogen;

and/or stereoisomer or agriculturally acceptable salts or tautomers or N-oxides thereof.

In a preferred embodiment, the compound of formula (I) is represented by formula Ia;

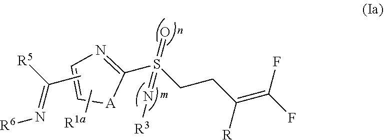

wherein;

A represent O or S;

n and m represent integers wherein n=0-2 and m=0-1;

R^{1a} is selected from the group consisting of hydrogen, X, CN, OR⁴, N(R⁴)₂, (C═O)—R⁴, S(O)₀₋₂R⁴, C(R^{4a})═NR⁴, C₁-C₁₂-alkyl, C₂-C₁₂-alkenyl, C₂-C₁₂-alkynyl, C₁-C₁₂-haloalkyl, C₂-C₁₂-haloalkenyl, C₂-C₁₂-haloalkynyl, C₃-C₁₀-cycloalkyl, C₃-C₁₀-halocycloalkyl, C₄-C₁₀-cycloalkenyl and C₇-C₁₉-aralkyl; wherein one or more carbon atoms of the cyclic ring system may be replaced by heteroatoms selected from the group consisting of N, O, S and optionally including 1 to 3 ring members selected from the group consisting of C(═O), C(═S), S(O)₀₋₂ and Si(R⁴)₂; R^{1a} may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;

R³ is selected from the group consisting of hydrogen, CN, (C═O)—R⁴, S(O)₀₋₂R⁴, Si(R⁴)₃, C₁-C₁₂-alkyl, C₂-C₁₂-alkenyl, C₁-C₁₂-haloalkyl, C₂-C₁₂-haloalkenyl, C₃-C₁₀-cycloalkyl and C₄-C₁₀-cycloalkylalkyl; R³ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;

R⁴ is selected from the group consisting of hydrogen, OR^{4a}, N(R^{4a})₂, C₁-C₆-alkyl, C₁-C₆-alkenyl, C₁-C₆-alkynyl, C₁-C₆-haloalkyl, C₂-C₆-haloalkenyl, C₃-C₁₂-cycloalkyl, C₃-C₁₀-halocycloalkyl, C₄-C₁₂-cycloalkenyl, C₆-C₁₀-aryl, C₇-C₁₉-aralkyl and C₃-C₁₂-heterocyclyl; R⁴ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R^{4a} is selected from the group consisting of hydrogen, C₁-C₆-alkyl and C₃-C₆-cycloalkyl;

R⁵ is selected from the group consisting of hydrogen, X, CN, C₁-C₁₂-alkyl, C₂-C₁₂-alkenyl, C₂-C₁₂-alkynyl, C₁-C₁₂-haloalkyl, C₂-C₁₂-haloalkenyl, C₃-C₁₀-cycloalkyl, C₃-C₁₀-halocycloalkyl, C₄-C₁₀-cycloalkylalkyl, C₆-C₁₀-aryl and C₇-C₁₉-aralkyl; R⁵ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;

R⁶ is selected from the group consisting of hydrogen, OR⁴, N(R⁴)₂, Si(R⁴)₃, (C═O)—R⁴, S(O)₀₋₂R⁴, C₁-C₈-alkyl-S(O)₀₋₂R⁴, C₁-C₈-alkyl-(C═O)—R⁴, S(O)₀₋₂C₅-C₁₉-aryl, S(O)₀₋₂ C₇-C₁₉-aralkyl, C₁-C₁₂-alkyl, C₂-C₁₂-alkenyl, C₂-C₁₂-alkynyl, C₁-C₁₂-haloalkyl, C₂-C₁₂-haloalkenyl, C₂-C₁₂-haloalkynyl, C₃-C₁₀-cycloalkyl, C₃-C₁₀-halocycloalkyl C₄-C₁₀-cycloalkenyl, C₆-C₁₀ aryl, C₇-C₁₉-aralkyl, C₅-C₁₂-bicycloalkyl, C₇-C₁₂-bicycloalkenyl and C₃-C₁₀-heterocyclyl; or

R⁵ and R⁶ together with the atom to which they are attached or together with further atoms selected from the group consisting of C, N, O, S and optionally including 1 to 3 ring members selected from the group consisting of C(═O), C(═S), S(O)₀₋₂ and Si(R⁴)₂, may form a four to seven membered ring, which for its part may be substituted by one or more R⁷;
R⁷ is selected from the group consisting of hydrogen, X, CN, SCN, SF₅, R⁴, OR⁴, NO₂, N(R⁴)₂, Si(R⁴)₃, (C═O)—R⁴, S(O)₀₋₂R⁴, C₁-C₅-alkyl-S(O)₀₋₂R⁴, C₁-C₅-alkyl-(C═O)—R⁴, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₃-C₁₀-cycloalkyl, C₃-C₁₀-halocycloalkyl, C₄-C₁₀-cycloalkenyl, C₅-C₁₀-cycloalkynyl, aryl, C₇-C₁₉-aralkyl and C₃-C₁₀-heterocyclyl; R⁶ and R⁷ are may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;

X represents halogen;

and/or stereoisomer or agriculturally acceptable salts or tautomers or N-oxides thereof.

In another preferred embodiment, the compound of formula (I) is represented by formula Ib;

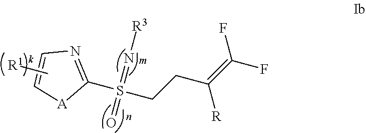

wherein

A represent O, NR⁴ or S;

n, m and k represent integers wherein n=0-1, m=1 and k=0-2;

R¹ is selected from the group consisting of R^{1a}, SCN, SF₅, NO₂, C₁-C₅-alkyl-S(O)₀₋₂R⁴, C₁-C₆-alkyl-OR⁴, C₁-C₈-alkyl-(C═O)—R⁴, C(R^{4a})═NR⁴, S(O)₀₋₂C₅-C₁₂-aryl, S(O)₀₋₂C₇-C₁₉-aralkyl, C₃-C₁₀-halocycloalkyl, C₄-C₁₀-cycloalkenyl, C₅-C₁₀-cycloalkynyl, C₁-C₈-alkyloxy-C₃-C₁₀-cycloalkyl, C₁-C₈-alkylthio-C₃-C₁₀-cycloalkyl, C₆-C₁₀-aryl, C₇-C₁₉-aralkyl, C₅-C₁₂-bicycloalkyl and C₇-C₁₂-bicycloalkenyl; wherein one or more carbon atoms of the cyclic ring system may be replaced by heteroatoms selected from the group consisting of N, O, S and optionally including 1 to 3 ring members selected from the group consisting of C(═O), C(═S), S(O)₀₋₂ and Si(R⁴)₂; R¹ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;

R^{1a} is selected from the group consisting of hydrogen, X, CN, (C═O)—R⁴, OR⁴, N(R⁴)₂, S(O)₀₋₂R⁴, Si(R⁴)₃, C₁-C₆-alkyl, C₂-C₆-alkenyl, C₂-C₆-alkynyl, C₁-C₆-haloalkyl, C₂-C₆-haloalkenyl, C₂-C₆-haloalkynyl and C₃-C₆-cycloalkyl; R^{1a} may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;

R³ is selected from the group consisting of hydrogen, CN, (C═O)—R⁴, OR⁴, N(R⁴)₂, S(O)₀₋₂R⁴, Si(R⁴)₃, C₁-C₁₂-alkyl, C₂-C₁₂-alkenyl, C₂-C₁₂-alkynyl, C₁-C₁₂-haloalkyl, C₂-C₁₂-haloalkenyl, C₂-C₁₂-haloalkynyl, C₃-C₁₀-cycloalkyl and C₄-C₁₀-cycloalkenyl; R³ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;

R⁴ is selected from the group consisting of hydrogen; OR^{4a}, N(R^{4a})₂, C₁-C₆-alkyl, C₁-C₆-alkenyl, C₁-C₆-alkynyl, C₁-C₆-haloalkyl, C₂-C₆-haloalkenyl, C₂-C₆-haloalkynyl, C₃-C₁₂-cycloalkyl, C₃-C₁₀-halocycloalkyl, C₄-C₆-cycloalkenyl, C₆-C₁₀-aryl, C₇-C₁₉-aralkyl and C₃-C₆-heterocyclyl; R⁴ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂,
R^{4a} is selected from the group consisting of hydrogen, C₁-C₆-alkyl and C₃-C₆-cycloalkyl;

X represents halogen;

and/or stereoisomer or agriculturally acceptable salts or tautomers or N-oxides thereof.

In yet another preferred embodiment, the compound of formula (I) is represented by formula Ic;

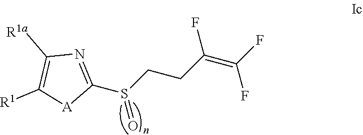

wherein;

A represent O, NR⁴ or S;

n represents integers of 0-2;

R¹ is selected from the group consisting of CN, C₂-C₁₂-alkynyl, C₁-C₆-haloalkyl, C₂-C₆-haloalkenyl, C₂-C₆-haloalkynyl, C₃-C₆-cycloalkyl, C₃-C₆-halocycloalkyl, C₄-C₆-cycloalkenyl, C₁-C₆-haloalkyloxy, C₁-C₆-haloalkylthio, C₁-C₆-haloalkylamino, C₁-C₆-dihaloalkylamino, C₁-C₆-haloalkyloxy-C₁-C₆-alkyl, C₁-C₆-haloalkylamino-C₁-C₆-alkyl, C₁-C₆-dihaloalkylamino-C₁-C₆-alkyl and C₁-C₆-haloalkylthio-C₁-C₆-alkyl; wherein one or more carbon atoms of the cyclic ring system may be replaced by heteroatoms selected from the group consisting of N, O, S and optionally including 1 to 3 ring members selected from the group consisting of C(═O), C(═S), S(O)₀₋₂ and Si(R⁴)₂; R¹ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;

R^{1a} is selected from the group consisting of hydrogen, X, CN, (C═O)—R⁴, OR⁴, N(R⁴)₂, S(O)₀₋₂R⁴, Si(R⁴)₃, C₁-C₆-alkyl, C₂-C₆-alkenyl, C₂-C₆-alkynyl, C₁-C₆-haloalkyl, C₂-C₆-haloalkenyl, C₂-C₆-haloalkynyl and C₃-C₆-cycloalkyl; R^{1a} may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;

R³ is selected from the group consisting of hydrogen, CN, (C═O)—R⁴, OR⁴, N(R⁴)₂, S(O)₀₋₂R⁴, Si(R⁴)₃, C₁-C₁₂-alkyl, C₂-C₁₂-alkenyl, C₂-C₁₂-alkynyl, C₁-C₁₂-haloalkyl, C₂-C₁₂-haloalkenyl, C₂-C₁₂-haloalkynyl, C₃-C₁₀-cycloalkyl and C₄-C₁₀-cycloalkenyl; R³ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;

R⁴ is selected from the group consisting of hydrogen, OR^{4a}, N(R^{4a})₂, C₁-C₆-alkyl, C₁-C₆-alkenyl, C₁-C₆-alkynyl, C₁-C₆-haloalkyl, C₂-C₆-haloalkenyl, C₂-C₆-haloalkynyl, C₃-C₁₂-cycloalkyl, C₃-C₁₀-halocycloalkyl, C₄-C₆-cycloalkenyl, C₆-C₁₀-aryl, C₇-C₁₉-aralkyl and C₃-C₆-heterocyclyl; R⁴ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂,
R^{4a} is selected from the group consisting of hydrogen, C₁-C₆-alkyl and C₃-C₆-cycloalkyl;

X represents halogen;

and/or stereoisomer or agriculturally acceptable salts or tautomers or N-oxides thereof.

In more preferred embodiment, the compound of formula (I) is selected from (E)-2-((3,4,4-trifluorobut-3-en-1-yl)thio)thiazole-5-carbaldehyde O-methyl oxime, (E)-2-((3,4,4-trifluorobut-3-en-1-yl)thio)thiazole-5-carbaldehyde O-methyl oxime, (Z)-2-((3,4,4-trifluorobut-3-en-1-yl)thio)oxazole-5-carbaldehyde O-methyl oxime, (E)-2-((3,4,4-trifluorobut-3-en-1-yl)sulfinyl)thiazole-5-carbaldehyde O-methyl oxime, (Z)-2-((3,4,4-trifluorobut-3-en-1-yl)sulfinyl)thiazole-5-carbaldehyde O-methyl oxime, (E)-2-((3,4,4-trifluorobut-3-en-1-yl)thio)oxazole-5-carbaldehyde O-methyl oxime, (E)-2-((3,4,4-trifluorobut-3-en-1-yl)sulfinyl)oxazole-5-carbaldehyde O-methyl oxime, (Z)-2-((3,4,4-trifluorobut-3-en-1-yl)sulfinyl)oxazole-5-carbaldehyde O-methyl oxime, (E)-2-((3,4,4-trifluorobut-3-en-1-yl)sulfonyl)oxazole-5-carbaldehyde O-methyl oxime, 2-((3,4,4-trifluorobut-3-en-1-yl)thio)thiazole-5-carbaldehyde O-ethyl oxime, (Z)-2-((3,4,4-trifluorobut-3-en-1-yl)sulfonyl)thiazole-5-carbaldehyde O-methyl oxime, (E)-2-((3,4,4-trifluorobut-3-en-1-yl)thio)thiazole-5-carbaldehyde O-ethyl oxime, (Z)-2-((3,4,4-trifluorobut-3-en-1-yl)thio)oxazole-5-carbaldehyde O-ethyl oxime, (E)-2-((((2-((3,4,4-trifluorobut-3-en-1-yl)thio)thiazol-5-yl)methylene)amino)oxy)acetonitrile, (E)-2-((3,4,4-trifluorobut-3-en-1-yl)thio)thiazole-5-carbaldehyde O-isopropyl oxime, (E)-2-((3,4,4-trifluorobut-3-en-1-yl)thio)thiazole-5-carbaldehyde O-cyclopropylmethyl oxime, (E)-2-((3,4,4-trifluorobut-3-en-1-yl)thio)oxazole-5-carbaldehyde O-ethyl oxime, (Z)-2-((3,4,4-trifluorobut-3-en-1-yl)sulfinyl)oxazole-5-carbaldehyde O-ethyl oxime, (Z)-4-phenyl-2-((3,4,4-trifluorobut-3-en-1-yl)thio)thiazole-5-carbaldehyde O-methyl oxime, (E)-4-phenyl-2-((3,4,4-trifluorobut-3-en-1-yl)thio)thiazole-5-carbaldehyde O-methyl oxime, (Z)-2-((3,4,4-trifluorobut-3-en-1-yl)thio)thiazole-5-carbaldehyde O-ethyl oxime, (E)-2-((3,4,4-trifluorobut-3-en-1-yl)sulfinyl)thiazole-5-carbaldehyde O-ethyl oxime, (E)-2-((3,4,4-trifluorobut-3-en-1-yl)sulfonyl)thiazole-5-carbaldehyde O-ethyl oxime, (Z)-4-phenyl-2-((3,4,4-trifluorobut-3-en-1-yl)thio)thiazole-5-carbaldehyde O-ethyl oxime, (E)-4-phenyl-2-((3,4,4-trifluorobut-3-en-1-yl)thio)thiazole-5-carbaldehyde O-ethyl oxime, (E)-4-methyl-2-((3,4,4-trifluorobut-3-en-1-yl)thio)thiazole-5-carbaldehyde O-methyl oxime, (Z)-4-methyl-2-((3,4,4-trifluorobut-3-en-1-yl)thio)thiazole-5-carbaldehyde O-methyl oxime, (Z)-4-phenyl-2-((3,4,4-trifluorobut-3-en-1-yl)sulfinyl)thiazole-5-carbaldehyde O-methyl oxime, (E)-4-phenyl-2-((3,4,4-trifluorobut-3-en-1-yl)sulfonyl)thiazole-5-carbaldehyde O-methyl oxime, (E)-4-phenyl-2-((3,4,4-trifluorobut-3-en-1-yl)sulfinyl)thiazole-5-carbaldehyde O-methyl oxime, (Z)-2-((3,4,4-trifluorobut-3-en-1-yl)sulfonyl)oxazole-5-carbaldehyde O-ethyl oxime, (E)-2-((3,4,4-trifluorobut-3-en-1-yl)sulfonyl)oxazole-5-carbaldehyde O-ethyl oxime, (E)-4-(tert-butyl)-2-((3,4,4-trifluorobut-3-en-1-yl)thio)thiazole-5-carbaldehyde O-methyl oxime, (E)-2-((3,4,4-trifluorobut-3-en-1-yl)thio)thiazole-5-carbaldehyde O-isopentyl oxime, (E)-2-((3,4,4-trifluorobut-3-en-1-yl)thio)thiazole-5-carbaldehyde O-cyclohexyl oxime, (E)-2-((3,4,4-trifluorobut-3-en-1-yl)sulfinyl)oxazole-5-carbaldehyde O-ethyl oxime, (E)-2-((3,4,4-trifluorobut-3-en-1-yl)sulfonyl)thiazole-5-carbaldehyde O-cyclohexyl oxime, (E)-1-(2-((3,4,4-trifluorobut-3-en-1-yl)thio)thiazol-5-yl)ethan-1-one O-methyl oxime, (E)-4-methyl-2-((3,4,4-trifluorobut-3-en-1-yl)sulfinyl)thiazole-5-carbaldehyde O-methyl oxime, (E)-4-methyl-2-((3,4,4-trifluorobut-3-en-1-yl)sulfonyl)thiazole-5-carbaldehyde O-methyl oxime, (E)-4-methyl-2-((3,4,4-trifluorobut-3-en-1-yl)thio)thiazole-5-carbaldehyde O-ethyl oxime, (Z)-4-methyl-2-((3,4,4-trifluorobut-3-en-1-yl)thio)thiazole-5-carbaldehyde O-ethyl oxime, (Z)-2-((3,4,4-trifluorobut-3-en-1-yl)sulfonyl)thiazole-5-carbaldehyde O-cyclobutylmethyl oxime, (Z)-2-((3,4,4-trifluorobut-3-en-1-yl)thio)thiazole-5-carbaldehyde O-cyclobutylmethyl oxime, (E)-2-((3,4,4-trifluorobut-3-en-1-yl)thio)thiazole-5-carbaldehyde O-cyclobutylmethyl oxime, (E)-2-((3,4,4-trifluorobut-3-en-1-yl)thio)oxazole-5-carbaldehyde O-isopentyl oxime, (Z)-2-((3,4,4-trifluorobut-3-en-1-yl)thio)oxazole-5-carbaldehyde O-isopentyl oxime, (Z)-2-((3,4,4-trifluorobut-3-en-1-yl)sulfinyl)oxazole-5-carbaldehyde O-isopentyl oxime, (Z)-2-((3,4,4-trifluorobut-3-en-1-yl)sulfonyl)oxazole-5-carbaldehyde O-isopentyl oxime, (E)-2-((3,4,4-trifluorobut-3-en-1-yl)sulfonyl)oxazole-5-carbaldehyde O-isopentyl oxime, (E)-2-((3,4,4-trifluorobut-3-en-1-yl)sulfinyl)oxazole-5-carbaldehyde O-isopentyl oxime, (E)-2-((3,4,4-trifluorobut-3-en-1-yl)thio)thiazole-4-carbaldehyde O-methyl oxime, (E)-1-(2-((3,4,4-trifluorobut-3-en-1-yl)sulfonyl)thiazol-5-yl)ethan-1-one O-methyl oxime, (E)-1-(2-((3,4,4-trifluorobut-3-en-1-yl)sulfinyl)thiazol-5-yl)ethan-1-one O-methyl oxime, (Z)-1-(2-((3,4,4-trifluorobut-3-en-1-yl)thio)thiazol-5-yl)ethan-1-one O-methyl oxime, (E)-1-(2-((3,4,4-trifluorobut-3-en-1-yl)thio)thiazol-5-yl)ethan-1-one O-ethyl oxime, (Z)-1-(2-((3,4,4-trifluorobut-3-en-1-yl)thio)thiazol-5-yl)ethan-1-one O-ethyl oxime, (E)-1-(2-((3,4,4-trifluorobut-3-en-1-yl)sulfinyl)thiazol-5-yl)ethan-1-one O-ethyl oxime, (E)-1-(2-((3,4,4-trifluorobut-3-en-1-yl)sulfonyl)thiazol-5-yl)ethan-1-one O-ethyl oxime, (Z)-1-(2-((3,4,4-trifluorobut-3-en-1-yl) sulfinyl)thiazol-5-yl)ethan-1-one O-ethyl oxime, (Z)-1-(2-((3,4,4-trifluorobut-3-en-1-yl)sulfonyl)thiazol-5-yl)ethan-1-one O-ethyl oxime, (Z)-2-((3,4,4-trifluorobut-3-en-1-yl)thio)thiazole-4-carbaldehyde O-methyl oxime, (Z)-2-((3,4,4-trifluorobut-3-en-1-yl)thio)thiazole-4-carbaldehyde O-ethyl oxime, (E)-2-((3,4,4-trifluorobut-3-en-1-yl)sulfinyl)thiazole-4-carbaldehyde O-methyl oxime, (E)-2-((3,4,4-trifluorobut-3-en-1-yl)sulfinyl)thiazole-4-carbaldehyde O-ethyl oxime, (E)-2-((3,4,4-trifluorobut-3-en-1-yl)sulfonyl)thiazole-4-carbaldehyde O-methyl oxime, (E)-2-((3,4,4-trifluorobut-3-en-1-yl)sulfonyl)thiazole-4-carb aldehyde O-ethyl oxime, (E)-5-chloro-2-((3,4,4-trifluorobut-3-en-1-yl)thio)thiazole-4-carbaldehyde O-methyl oxime, (E)-5-chloro-2-((3,4,4-trifluorobut-3-en-1-yl)sulfonyl)thiazole-4-carbaldehyde O-methyl oxime, (E)-2-((3,4,4-trifluorobut-3-en-1-yl)sulfonyl)thiazole-5-carbaldehyde O-methyl oxime, (Z)-2-((3,4,4-trifluorobut-3-en-1-yl)sulfonyl)oxazole-5-carbaldehyde O-methyl oxime, (E)-2-((3,4,4-trifluorobut-3-en-1-yl)thio)oxazole-5-carbaldehyde O-cyclopropylmethyl oxime, N-((5-((Z)-(methoxyimino)methyl)thiazol-2-yl) (3,4,4-trifluorobut-3-en-1-yl)-λ⁴-sulfaneylidene)cyanamide, (E)-4-methyl-2-((3,4,4-trifluorobut-3-en-1-yl)sulfonyl)thiazole-5-carbaldehyde O-ethyl oxime, (E)-4-methyl-2-((3,4,4-trifluorobut-3-en-1-yl)sulfinyl)thiazole-5-carbaldehyde O-ethyl oxime, (E)-2-((3,4,4-trifluorobut-3-en-1-yl)thio)oxazole-5-carbaldehyde O-isopropyl oxime, (E)-2-((3,4,4-trifluorobut-3-en-1-yl)sulfinyl)oxazole-5-carbaldehyde O-isopropyl oxime, (E)-2-((3,4,4-trifluorobut-3-en-1-yl)sulfonyl)oxazole-5-carbaldehyde O-isopropyl oxime, (E)-2-((3,4,4-trifluorobut-3-en-1-yl)sulfinyl)oxazole-5-carbaldehyde O-cyclopropylmethyl oxime, (E)-2-((3,4,4-trifluorobut-3-en-1-yl)sulfonyl)oxazole-5-carbaldehyde O-cyclopropylmethyl oxime, (Z)-2-((3,4,4-trifluorobut-3-en-1-yl)sulfonyl)thiazole-5-carbaldehyde O-ethyl oxime, (Z)-2-((3,4,4-trifluorobut-3-en-1-yl)thio)oxazole-5-carbaldehyde O-cyclopropylmethyl oxime, (Z)-2-((3,4,4-trifluorobut-3-en-1-yl)sulfonyl)oxazole-5-carbaldehyde O-cyclopropyl methyl oxime, (Z)-1-(2-((3,4,4-trifluorobut-3-en-1-yl)thio)thiazol-5-yl)ethan-1-one O-cyclopropylmethyl oxime, (Z)-1-(2-((3,4,4-trifluorobut-3-en-1-yl)sulfonyl)thiazol-5-yl)ethan-1-one O-cyclopropylmethyl oxime, (Z)-1-(2-((3,4,4-trifluorobut-3-en-1-yl)sulfinyl)thiazol-5-yl)ethan-1-one O-cyclopropylmethyl oxime, (Z)-2-((3,4,4-trifluorobut-3-en-1-yl)sulfinyl)thiazole-5-carbaldehyde O-ethyl oxime, (Z)-2-((3,4,4-trifluorobut-3-en-1-yl)thio)thiazole-5-carbaldehyde 0-isopropyl oxime, (Z)-2-((3,4,4-trifluorobut-3-en-1-yl)sulfonyl)thiazole-5-carbaldehyde O-isopropyl oxime, (E)-2-((3,4,4-trifluorobut-3-en-1-yl)sulfinyl)thiazole-5-carbaldehyde O-isopropyl oxime, N-((E)-(4-((E)-(methoxyimino)methyl)thiazol-2-yl)(3,4,4-trifluorobut-3-en-1-yl)-14-sulfaneylidene) cyanamide, (E)-1-(2-((3,4,4-trifluorobut-3-en-1-yl)thio)thiazol-5-yl)ethan-1-one O-cyclopropylmethyl oxime, (E)-phenyl(2-((3,4,4-trifluorobut-3-en-1-yl)thio)thiazol-5-yl)methanone 0-methyl oxime, (E)-phenyl(2-((3,4,4-trifluorobut-3-en-1-yl)sulfinyl)thiazol-5-yl)methanone O-methyl oxime, (E)-phenyl(2-((3,4,4-trifluorobut-3-en-1-yl)thio)thiazol-5-yl)methanone O-ethyl oxime, (E)-phenyl(2-((3,4,4-trifluorobut-3-en-1-yl)sulfonyl)thiazol-5-yl)methanone O-methyl oxime, (E)-phenyl(2-((3,4,4-trifluorobut-3-en-1-yl)thio)thiazol-5-yl)methanone oxime, (E)-phenyl(2-((3,4,4-trifluorobut-3-en-1-yl)sulfinyl)thiazol-5-yl)methanone O-ethyl oxime, (E)-phenyl(2-((3,4,4-trifluorobut-3-en-1-yl)sulfonyl)thiazol-5-yl)methanone O-ethyl oxime, (E)-phenyl(2-((3,4,4-trifluorobut-3-en-1-yl)thio)thiazol-5-yl)methanone O-cyclopropylmethyl oxime, (E)-2-((3,4,4-trifluorobut-3-en-1-yl)thio)oxazole-5-carbaldehyde O-cyclobutylmethyl oxime, (Z)-2-((3,4,4-trifluorobut-3-en-1-yl)thio)oxazole-5-carbaldehyde O-isopropyl oxime, (Z)-2-((3,4,4-trifluorobut-3-en-1-yl)sulfinyl)oxazole-5-carbaldehyde O-cyclopropylmethyl oxime, (E)-phenyl(2-((3,4,4-trifluorobut-3-en-1-yl)sulfinyl)thiazol-5-yl)methanone O-cyclopropylmethyl oxime, (E)-phenyl(2-((3,4,4-trifluorobut-3-en-1-yl)sulfinyl)thiazol-5-yl)methanone oxime, (E)-phenyl(2-((3,4,4-trifluorobut-3-en-1-yl)sulfonyl)thiazol-5-yl)methanone O-cyclopropylmethyl oxime, (E)-phenyl(2-((3,4,4-trifluorobut-3-en-1-yl)thio)thiazol-5-yl) methanone O-prop yl oxime, (E)-phenyl(2-((3,4,4-trifluorobut-3-en-1-yl)thio)thiazol-5-yl)methanone O-isopentyl oxime, (E)-phenyl(2-((3,4,4-trifluorobut-3-en-1-yl)sulfonyl)thiazol-5-yl)methanone oxime, (E)-phenyl(2-((3,4,4-trifluorobut-3-en-1-yl) sulfinyl)thiazol-5-yl)methanone O-propyl oxime, (E)-phenyl(2-((3,4,4-trifluorobut-3-en-1-yl)sulfonyl)thiazol-5-yl)methanone O-propyl oxime, (E)-phenyl(2-((3,4,4-trifluorobut-3-en-1-yl)sulfinyl)thiazol-5-yl)methanone O-isopentyl oxime, (E)-phenyl(2-((3,4,4-trifluorobut-3-en-1-yl)sulfonyl)thiazol-5-yl)methanone O-isopentyl oxime, (E)-1-(2-((3,4,4-trifluorobut-3-en-1-yl)sulfinyl)thiazol-5-yl)ethan-1-one O-cyclopropylmethyl oxime, (E)-1-(2-((3,4,4-trifluorobut-3-en-1-yl)sulfonyl)thiazol-5-yl)ethan-1-one O-cyclopropylmethyl oxime, (Z)-1-(2-((3,4,4-trifluorobut-3-en-1-yl)thio)thiazol-5-yl)ethan-1-one O-isopropyl oxime, (E)-1-(2-((3,4,4-trifluorobut-3-en-1-yl)thio)thiazol-5-yl)ethan-1-one O-isopropyl oxime, (Z)-1-(2-((3,4,4-trifluorobut-3-en-1-yl)sulfinyl)thiazol-5-yl)ethan-1-one O-isopropyl oxime, (Z)-1-(2-((3,4,4-trifluorobut-3-en-1-yl)sulfonyl)thiazol-5-yl)ethan-1-one O-isopropyl oxime, (E)-1-(2-((3,4,4-trifluorobut-3-en-1-yl)sulfinyl)thiazol-5-yl)ethan-1-one O-isopropyl oxime, (E)-1-(2-((3,4,4-trifluorobut-3-en-1-yl)sulfonyl)thiazol-5-yl)ethan-1-one O-isopropyl oxime, (Z)-1-(2-((3,4,4-trifluorobut-3-en-1-yl)sulfinyl)thiazol-5-yl)ethan-1-one O-methyl oxime, (Z)-1-(2-((3,4,4-trifluorobut-3-en-1-yl)sulfonyl)thiazol-5-yl)ethan-1-one O-methyl oxime, (Z)-2-((3,4,4-trifluorobut-3-en-1-yl)sulfinyl)oxazole-5-carbaldehyde O-isopropyl oxime, (E)-2-((3,4,4-trifluorobut-3-en-1-yl)sulfinyl)oxazole-5-carbaldehyde O-cyclobutylmethyl oxime, (E)-2-((3,4,4-trifluorobut-3-en-1-yl)sulfonyl)oxazole-5-carbaldehyde O-cyclobutylmethyl oxime, (Z)-1-(2-((3,4,4-trifluorobut-3-en-1-yl)thio)oxazol-5-yl)ethan-1-one O-methyl oxime, (E)-1-(2-((3,4,4-trifluorobut-3-en-1-yl)thio)oxazol-5-yl)ethan-1-one O-methyl oxime, (E)-1-(2-((3,4,4-trifluorobut-3-en-1-yl)sulfonyl)oxazol-5-yl)ethan-1-one O-methyl oxime, (E)-1-(2-((3,4,4-trifluorobut-3-en-1-yl)sulfinyl)oxazol-5-yl)ethan-1-one O-methyl oxime, (Z)-2-((3,4,4-trifluorobut-3-en-1-yl)sulfonyl)oxazole-5-carbaldehyde O-isopropyl oxime, (E)-1-(2-((3,4,4-trifluorobut-3-en-1-yl)thio)oxazol-5-yl)ethan-1-one O-ethyl oxime, (E)-1-(2-((3,4,4-trifluorobut-3-en-1-yl)sulfonyl)oxazol-5-yl)ethan-1-one O-ethyl oxime, (Z)-1-(2-((3,4,4-trifluorobut-3-en-1-yl)sulfonyl)oxazol-5-yl)ethan-1-one O-methyl oxime, (Z)-1-(2-((3,4,4-trifluorobut-3-en-1-yl)thio)oxazol-5-yl)ethan-1-one O-ethyl oxime, (E)-2-((3,4,4-trifluorobut-3-en-1-yl)sulfinyl)thiazole-5-carb aldehyde O-isopentyl oxime, (E)-2-((3,4,4-trifluorobut-3-en-1-yl)sulfonyl)thiazole-5-carbaldehyde O-isopentyl oxime, (E)-1-(2-((3,4,4-trifluorobut-3-en-1-yl)thio)thiazol-5-yl)ethan-1-one O-isopentyl oxime, (E)-1-(2-((3,4,4-trifluorobut-3-en-1-yl)sulfinyl)thiazol-5-yl)ethan-1-one O-isopentyl oxime, (E)-1-(2-((3,4,4-trifluorobut-3-en-1-yl)sulfonyl)thiazol-5-yl)ethan-1-one O-isopentyl oxime, (E)-1-(2-((3,4,4-trifluorobut-3-en-1-yl)thio)thiazol-4-yl)ethan-1-one O-ethyl oxime, (E)-1-(2-((3,4,4-trifluorobut-3-en-1-yl)sulfinyl)thiazol-4-yl)ethan-1-one O-ethyl oxime, (E)-1-(2-((3,4,4-trifluorobut-3-en-1-yl)sulfonyl)thiazol-4-yl)ethan-1-one O-ethyl oxime, (Z)-1-(2-((3,4,4-trifluorobut-3-en-1-yl)thio)thiazol-5-yl)ethan-1-one O-isopentyl oxime, (Z)-1-(2-((3,4,4-trifluorobut-3-en-1-yl)sulfinyl)thiazol-5-yl)ethan-1-one O-isopentyl oxime, (Z)-1-(2-((3,4,4-trifluorobut-3-en-1-yl)sulfonyl)thiazol-5-yl)ethan-1-one O-isopentyl oxime, (E)-4-methyl-2-((3,4,4-trifluorobut-3-en-1-yl)thio)thiazole-5-carbaldehyde O-isopropyl oxime, (E)-1-(2-((3,4,4-trifluorobut-3-en-1-yl)thio)thiazol-4-yl)ethan-1-one O-methyl oxime, (E)-1-(2-((3,4,4-trifluorobut-3-en-1-yl)sulfinyl)thiazol-4-yl)ethan-1-one O-methyl oxime, (E)-1-(2-((3,4,4-trifluorobut-3-en-1-yl)sulfonyl)thiazol-4-yl)ethan-1-one O-methyl oxime, (E)-4-methyl-2-((3,4,4-trifluorobut-3-en-1-yl)thio)thiazole-5-carbaldehyde O-cyclopropylmethyl oxime, (E)-2-((3,4,4-trifluorobut-3-en-1-yl)thio)-4-(trifluoromethyl)thiazole-5-carbaldehyde O-methyl oxime, (E)-2-((3,4,4-trifluorobut-3-en-1-yl)sulfinyl)-4-(trifluoromethyl)thiazole-5-carbaldehyde O-methyl oxime, (E)-2-((3,4,4-trifluorobut-3-en-1-yl)sulfonyl)-4-(trifluoromethyl)thiazole-5-carbaldehyde O-methyl oxime, (E)-2-((3,4,4-trifluorobut-3-en-1-yl)thio)-4-(trifluoromethyl)thiazole-5-carbaldehyde O-ethyl oxime, (E)-2-((3,4,4-trifluorobut-3-en-1-yl)sulfinyl)-4-(trifluoromethyl)thiazole-5-carbaldehyde O-ethyl oxime, (E)-2-((3,4,4-trifluorobut-3-en-1-yl)sulfonyl)-4-(trifluoromethyl)thiazole-5-carbaldehyde O-ethyl oxime, (E)-4-methyl-2-((3,4,4-trifluorobut-3-en-1-yl)sulfinyl)thiazole-5-carbaldehyde O-cyclopropylmethyl oxime, (E)-4-methyl-2-((3,4,4-trifluorobut-3-en-1-yl)sulfonyl)thiazole-5-carbaldehyde O-cyclopropylmethyl oxime, (E)-4-methyl-2-((3,4,4-trifluorobut-3-en-1-yl)sulfinyl)thiazole-5-carbaldehyde O-isopropyl oxime, N-((5-chlorothiazol-2-yl)(3,4,4-trifluorobut-3-en-1-yl)-λ⁴-sulfaneylidene)cyanamide, N-(thiazol-2-yl(3,4,4-trifluorobut-3-en-1-yl)-λ⁴-sulfaneylidene)cyanamide, N-((5-chlorothiazol-2-yl)(oxo)(3,4,4-trifluorobut-3-en-1-yl)-λ⁶-sulfaneylidene)cyanamide, N-(oxo(thiazol-2-yl)(3,4,4-trifluorobut-3-en-1-yl)-λ⁶-sulfaneylidene)cyanamide, N-((5-bromothiazol-2-yl)(oxo)(3,4,4-trifluorobut-3-en-1-yl)-λ⁶-sulfaneylidene)cyanamide, imino(thiazol-2-yl)(3,4,4-trifluorobut-3-en-1-yl)-λ⁶-sulfanone, (ethylimino)(thiazol-2-yl)(3,4,4-trifluorobut-3-en-1-yl)-λ⁶-sulfanone, 2,2,2-trifluoro-N-(oxo (thiazol-2-yl)(3,4,4-trifluorobut-3-en-1-yl)-sulfaneylidene) acetamide, (5-chlorothiazol-2-yl)(imino)(3,4,4-trifluorobut-3-en-1-yl)-λ⁶-sulfanone, (5-chlorothiazol-2-yl)(ethylimino)(3,4,4-trifluorobut-3-en-1-yl)-λ⁶-sulfanone, (methylimino) (thiazol-2-yl)(3,4,4-trifluorobut-3-en-1-yl)-λ⁶-sulfanone, (propylimino)(thiazol-2-yl)(3,4,4-trifluorobut-3-en-1-yl)-λ⁶-sulfanone, ((cyclopropylmethyl)imino)(thiazol-2-yl)(3,4,4-trifluorobut-3-en-1-yl)-λ⁶-sulfanone, N-((5-bromothiazol-2-yl)(3,4,4-trifluorobut-3-en-1-yl)-λ⁴-sulfaneylidene)cyanamide, (5-bromothiazol-2-yl)(imino)(3,4,4-trifluorobut-3-en-1-yl)-λ⁶-sulfanone, (5-bromothiazol-2-yl)(ethylimino)(3,4,4-trifluorobut-3-en-1-yl)-λ⁶-sulfanone, (5-(difluoromethyl)thiazol-2-yl)(imino)(3,4,4-trifluorobut-3-en-1-yl)-λ⁶-sulfanone, 2-(3,4,4-trifluorobut-3-en-1-ylsulfonimidoyl)thiazole-5-carbonitrile, (5-bromothiazol-2-yl)(methylimino)(3,4,4-trifluorobut-3-en-1-yl)-λ⁶-sulfanone, (5-chlorothiazol-2-yl)(methylimino)(3,4,4-trifluorobut-3-en-1-yl)-λ⁶-sulfanone, N-((5-(difluoromethyl)thiazol-2-yl)(3,4,4-trifluorobut-3-en-1-yl)-λ⁴-sulfaneylidene)cyanamide, N-((4-methylthiazol-2-yl)(3,4,4-trifluorobut-3-en-1-yl)-λ⁴-sulfaneylidene)cyanamide, N-((4-methylthiazol-2-yl)(oxo)(3,4,4-trifluorobut-3-en-1-yl)-λ⁶-sulfaneylidene)cyanamide, N-((5-chloro-4-methylthiazol-2-yl)(3,4,4-trifluorobut-3-en-1-yl)-λ⁴-sulfaneylidene)cyanamide, N-((5-chloro-4-methylthiazol-2-yl)(oxo)(3,4,4-trifluorobut-3-en-1-yl)-λ⁶-sulfaneylidene)cyanamide, (5-chloro-4-methylthiazol-2-yl)(imino)(3,4,4-trifluorobut-3-en-1-yl)-λ⁶-sulfanone, N-((4-(tert-butyl)thiazol-2-yl)(3,4,4-trifluorobut-3-en-1-yl)-λ⁴-sulfaneylidene)cyanamide, (4-(tert-butyl)thiazol-2-yl)(imino)(3,4,4-trifluorobut-3-en-1-yl)-λ⁶-sulfanone, (4-(tert-butyl)thiazol-2-yl)(methylimino)(3,4,4-trifluorobut-3-en-1-yl)-λ⁶-sulfanone, imino(4-phenylthiazol-2-yl)(3,4,4-trifluorobut-3-en-1-yl)-λ⁶-sulfanone, (5-bromo-4-phenylthiazol-2-yl)(imino)(3,4,4-trifluorobut-3-en-1-yl)-λ⁶-sulfanone, (5-chloro-4-phenylthiazol-2-yl)(imino)(3,4,4-trifluorobut-3-en-1-yl)-6-sulfanone, N-((4-(tert-butyl)-5-chlorothiazol-2-yl)(3,4,4-trifluorobut-3-en-1-yl)-λ⁴-sulfaneylidene)cyanamide, (4-(tert-butyl)-5-chlorothiazol-2-yl)(imino)(3,4,4-trifluorobut-3-en-1-yl)-λ⁶-sulfanone, (4-(tert-butyl)-5-chlorothiazol-2-yl)(methylimino)(3,4,4-trifluorobut-3-en-1-yl)-λ⁶-sulfanone, (4-(tert-butyl)-5-chlorothiazol-2-yl)(ethylimino)(3,4,4-trifluorobut-3-en-1-yl)-λ⁶-sulfanone, N-((5-bromo-4-(tert-butyl)thiazol-2-yl)(3,4,4-trifluorobut-3-en-1-yl)-λ⁴-sulfaneylidene)cyanamide, (5-bromo-4-(tert-butyl)thiazol-2-yl)(imino)(3,4,4-trifluorobut-3-en-1-yl)-λ⁶-sulfanone, (5-bromo-4-(tert-butyl)thiazol-2-yl)(methylimino)(3,4,4-trifluorobut-3-en-1-yl)-λ⁶-sulfanone, (5-bromo-4-(tert-butyl)thiazol-2-yl)(ethylimino)(3,4,4-trifluorobut-3-en-1-yl)-λ⁶-sulfanone, (4,5-dimethylthiazol-2-yl)(imino)(3,4,4-trifluorobut-3-en-1-yl)-λ⁶-sulfanone, imino(4-methylthiazol-2-yl)(3,4,4-trifluorobut-3-en-1-yl)-λ⁶-sulfanone, (methylimino)(4-methylthiazol-2-yl)(3,4,4-trifluorobut-3-en-1-yl)-λ⁶-sulfanone, (ethylimino)(4-methylthiazol-2-yl)(3,4,4-trifluorobut-3-en-1-yl)-λ⁶-sulfanone, (5-bromo-4-phenylthiazol-2-yl)(methylimino)(3,4,4-trifluorobut-3-en-1-yl)-λ⁶-sulfanone, N-((4,5-dimethylthiazol-2-yl)(3,4,4-trifluorobut-3-en-1-yl)-λ⁴-sulfaneylidene)cyanamide, 5-(2-chlorophenyl)-3-(2-((3,4,4-trifluorobut-3-en-1-yl)thio)thiazol-5-yl)-4,5-dihydroisoxazole, 5-phenyl-3-(2-((3,4,4-trifluorobut-3-en-1-yl)thio)thiazol-5-yl)-4,5-dihydroisoxazole, 5-(2-chlorophenyl)-3-(2-((3,4,4-trifluorobut-3-en-1-yl)sulfinyl)thiazol-5-yl)-4,5-dihydroisoxazole, 5-(2-chlorophenyl)-3-(2-((3,4,4-trifluorobut-3-en-1-yl)sulfonyl)thiazol-5-yl)-4,5-dihydroisoxazole, 3-(2-((3,4,4-trifluorobut-3-en-1-yl)thio)thiazol-5-yl)-1,2,4-oxadiazole, 5-phenyl-3-(2-((3,4,4-trifluorobut-3-en-1-yl)sulfonyl)thiazol-5-yl)-4,5-dihydroisoxazole, 3-(2-((3,4,4-trifluorobut-3-en-1-yl)sulfinyl)thiazol-5-yl)-1,2,4-oxadiazole, 5-(4-chlorophenyl)-3-(2-((3,4,4-trifluorobut-3-en-1-yl)thio)thiazol-5-yl)isoxazole, 5-methyl-3-(2-((3,4,4-trifluorobut-3-en-1-yl)thio)thiazol-5-yl)-1,2,4-oxadiazole, 5-methyl-3-(2-((3,4,4-trifluorobut-3-en-1-yl)sulfinyl)thiazol-5-yl)-1,2,4-oxadiazole, 5-(2H-tetrazol-5-yl)-2-((3,4,4-trifluorobut-3-en-1-yl)thio)thiazole, 5-(4-chlorophenyl)-3-(2-((3,4,4-trifluorobut-3-en-1-yl)sulfonyl)thiazol-5-yl)isoxazole, 5-methyl-3-(2-((3,4,4-trifluorobut-3-en-1-yl)sulfonyl)thiazol-5-yl)-1,2,4-oxadiazole, 5-(2H-tetrazol-5-yl)-2-((3,4,4-trifluorobut-3-en-1-yl)sulfonyl)thiazole, 3-(2-((3,4,4-trifluorobut-3-en-1-yl)thio)thiazol-5-yl)-1,2,4-oxadiazol-5(4H)-one, 3-(2-((3,4,4-trifluorobut-3-en-1-yl)sulfinyl)thiazol-5-yl)-1,2,4-oxadiazol-5(4H)-one, 5-(2H-tetrazol-5-yl)-2-((3,4,4-trifluorobut-3-en-1-yl)sulfinyl)thiazole, 3-(2-((3,4,4-trifluorobut-3-en-1-yl)sulfonyl)thiazol-5-yl)-1,2,4-oxadiazol-5 (4H)-one, 5-(4-chlorophenyl)-3-(2-((3,4,4-trifluorobut-3-en-1-yl)thio)thiazol-4-yl)isoxazole, 3-(2-((3,4,4-trifluorobut-3-en-1-yl)thio)thiazol-4-yl)-5-(trifluoromethyl)-1,2,4-oxadiazole, 3-(2-((3,4,4-trifluorobut-3-en-1-yl)sulfinyl)thiazol-4-yl)-5-(trifluoromethyl)-1,2,4-oxadiazole, 3-(2-((3,4,4-trifluorobut-3-en-1-yl)sulfonyl)thiazol-4-yl)-5-(trifluoromethyl)-1,2,4-oxadiazole, 3-(2-((3,4,4-trifluorobut-3-en-1-yl)thio)thiazol-5-yl)-5-(trifluoromethyl)-1,2,4-oxadiazole, 5-methyl-3-(2-((3,4,4-trifluorobut-3-en-1-yl)thio)thiazol-4-yl)-1,2,4-oxadiazole, N-((4-(5-methyl-1,2,4-oxadiazol-3-yl)thiazol-2-yl)(3,4,4-trifluorobut-3-en-1-yl)-λ⁴-sulfaneylidene)cyanamide, N-((3,4,4-trifluorobut-3-en-1-yl)(5-(5-(trifluoromethyl)-1,2,4-oxadiazol-3-yl)thiazol-2-yl)-λ⁴-sulfaneylidene)cyanamide, (Z)-2-((4,4-difluorobut-3-en-1-yl)sulfonyl)thiazole-5-carbaldehyde O-ethyl oxime, (E)-2-((4,4-difluorobut-3-en-1-yl)thio)thiazole-5-carbaldehyde O-methyl oxime, (Z)-2-((4,4-difluorobut-3-en-1-yl)thio)thiazole-5-carbaldehyde O-methyl oxime, (Z)-2-((4,4-difluorobut-3-en-1-yl)sulfon yl)thiazole-5-carbaldehyde O-methyl oxime, (E)-2-((4,4-difluorobut-3-en-1-yl)sulfonyl)thiazole-5-carbaldehyde O-ethyl oxime, (E)-4-methyl-2-((3,4,4-trifluorobut-3-en-1-yl)thio)oxazole-5-carb aldehyde O-methyl oxime, (E)-4-methyl-2-((3,4,4-trifluorobut-3-en-1-yl)thio)oxazole-5-carbaldehyde O-ethyl oxime, (E)-4-(tert-butyl)-2-((3,4,4-trifluorobut-3-en-1-yl)thio)oxazole-5-carbaldehyde O-methyl oxime, (E)-4-(tert-butyl)-2-((3,4,4-trifluorobut-3-en-1-yl)thio)oxazole-5-carbaldehyde O-ethyl oxime, (E)-5-chloro-2-((3,4,4-trifluorobut-3-en-1-yl)thio)thiazole-4-carbaldehyde O-ethyl oxime, (E)-5-chloro-2-((3,4,4-trifluorobut-3-en-1-yl)sulfinyl)thiazole-4-carbaldehyde O-ethyl oxime, (E)-5-chloro-2-((3,4,4-trifluorobut-3-en-1-yl)sulfinyl)thiazole-4-carbaldehyde O-methyl oxime, 4-methoxy-2-((3,4,4-trifluorobut-3-en-1-yl)sulfonyl)thiazole-5-carbaldehyde O-ethyl oxime, (E)-5-((2-phenylhydrazineylidene)methyl)-2-((3,4,4-trifluorobut-3-en-1-yl)thio)thiazole, (E)-5-((2-methylhydrazineylidene)methyl)-2-((3,4,4-trifluorobut-3-en-1-yl)thio)thiazole, (E)-2-((3,4,4-trifluorobut-3-en-1-yl)thio)thiazole-5-carbaldehyde O-benzyl oxime, (E)-5-((2-phenylhydrazineylidene)methyl)-2-((3,4,4-trifluorobut-3-en-1-yl)thio)oxazole, (E)-2-((3,4,4-trifluorobut-3-en-1-yl)sulfinyl)thiazole-5-carbaldehyde O-benzyl oxime, 4-methoxy-2-((3,4,4-trifluorobut-3-en-1-yl)thio)thiazole-5-carbaldehyde O-methyl oxime, 4-methoxy-2-((3,4,4-trifluorobut-3-en-1-yl)sulfonyl)thiazole-5-carbaldehyde O-methyl oxime, (E)-2-((3,4,4-trifluorobut-3-en-1-yl)sulfonyl)thiazole-5-carb aldehyde O-benzyl oxime, (E)-1-(4-methyl-2-((3,4,4-trifluorobut-3-en-1-yl)thio)oxazol-5-yl)ethan-1-one O-methyl oxime, (E)-1-(4-methyl-2-((3,4,4-trifluorobut-3-en-1-yl)thio)oxazol-5-yl)ethan-1-one O-ethyl oxime, (E)-1-(4-methyl-2-((3,4,4-trifluorobut-3-en-1-yl)thio)oxazol-5-yl)propan-1-one O-methyl oxime, (E)-1-(4-(tert-butyl)-2-((3,4,4-trifluorobut-3-en-1-yl)thio)oxazol-5-yl)ethan-1-one O-methyl oxime, (E)-1-(4-(tert-butyl)-2-((3,4,4-trifluorobut-3-en-1-yl)thio)oxazol-5-yl)propan-1-one O-methyl oxime, (E)-1-(4-(tert-butyl)-2-((3,4,4-trifluorobut-3-en-1-yl)thio)oxazol-5-yl)ethan-1-one O-ethyl oxime, (E)-4-methoxy-2-((3,4,4-trifluorobut-3-en-1-yl)sulfinyl)thiazole-5-carbaldehyde O-methyl oxime, (E)-4-methoxy-2-((3,4,4-trifluorobut-3-en-1-yl)sulfinyl)thiazole-5-carb aldehyde O-ethyl oxime, (E)-4-(tert-butyl)-2-((3,4,4-trifluorobut-3-en-1-yl)thio)oxazole-5-carbaldehyde O-cyclopropylmethyl oxime, (E)-4-methyl-2-((3,4,4-trifluorobut-3-en-1-yl)thio)oxazole-5-carbaldehyde O-cyclopropylmethyl oxime, 2-methyl-1-(4-methyl-2-((3,4,4-trifluorobut-3-en-1-yl)thio)oxazol-5-yl)propan-1-one O-methyl oxime, 3-methyl-1-(4-methyl-2-((3,4,4-trifluorobut-3-en-1-yl)thio)oxazol-5-yl)butan-1-one O-methyl oxime, (E)-4-methyl-2-((3,4,4-trifluorobut-3-en-1-yl)sulfinyl)oxazole-5-carbaldehyde O-cyclopropylmethyl oxime, (E)-4-methyl-2-((3,4,4-trifluorobut-3-en-1-yl)sulfonyl)oxazole-5-carbaldehyde O-cyclopropylmethyl oxime, 5-(thiophen-2-yl)-3-(2-((3,4,4-trifluorobut-3-en-1-yl)sulfinyl)thiazol-5-yl)-1,2,4-oxadiazole, 5-cyclobutyl-3-(2-((3,4,4-trifluorobut-3-en-1-yl)thio)thiazol-5-yl)-1,2,4-oxadiazole, 5-(thiophen-2-yl)-3-(2-((3,4,4-trifluorobut-3-en-1-yl)sulfonyl)thiazol-5-yl)-1,2,4-oxadiazole, 5-cyclopropyl-3-(2-((3,4,4-trifluorobut-3-en-1-yl)thio)thiazol-5-yl)-1,2,4-oxadiazole, (E)-4-methyl-2-((3,4,4-trifluorobut-3-en-1-yl)thio)oxazole-5-carbaldehyde O-isobutyl oxime, (E)-2-methyl-1-(4-methyl-2-((3,4,4-trifluorobut-3-en-1-yl)sulfinyl)oxazol-5-yl)propan-1-one O-methyl oxime, 3-methyl-1-(4-methyl-2-((3,4,4-trifluorobut-3-en-1-yl)sulfinyl)oxazol-5-yl)butan-1-one O-methyl oxime, (E)-2-((3,4,4-trifluorobut-3-en-1-yl)thio)thiazole-5-carbaldehyde O-cyclopropylmethyl oxime, (E)-2-((3,4,4-trifluorobut-3-en-1-yl)sulfinyl)thiazole-5-carbaldehyde O-cyclopropylmethyl oxime, 5-cyclopropyl-3-(2-((3,4,4-trifluorobut-3-en-1-yl)sulfonyl)thiazol-5-yl)-1,2,4-oxadiazole, 5-cyclobutyl-3-(2-((3,4,4-trifluorobut-3-en-1-yl)sulfonyl)thiazol-5-yl)-1,2,4-oxadiazole, 3-(2-((3,4,4-trifluorobut-3-en-1-yl)sulfinyl)thiazol-5-yl)-5-(trifluoromethyl)-1,2,4-oxadiazole, 3-(2-((3,4,4-trifluorobut-3-en-1-yl)sulfonyl)thiazol-5-yl)-5-(trifluoromethyl)-1,2,4-oxadiazole, 4-cyclopropyl-2-((3,4,4-trifluorobut-3-en-1-yl)thio)thiazole-5-carbaldehyde O-methyl oxime, 4-cyclopropyl-2-((3,4,4-trifluorobut-3-en-1-yl)thio)thiazole-5-carbaldehyde O-ethyl oxime, 4-cyclopropyl-2-((3,4,4-trifluorobut-3-en-1-yl)sulfinyl)thiazole-5-carb aldehyde O-methyl oxime, 4-cyclopropyl-2-((3,4,4-trifluorobut-3-en-1-yl)sulfonyl)thiazole-5-carbaldehyde O-methyl oxime, 5-cyclopropyl-3-(2-((3,4,4-trifluorobut-3-en-1-yl)sulfinyl)thiazol-5-yl)-1,2,4-oxadiazole, 2-((4,4-difluorobut-3-en-1-yl)thio)thiazole-5-carbaldehyde O-methyl oxime, 4-cyclopropyl-2-((3,4,4-trifluorobut-3-en-1-yl)sulfinyl)thiazole-5-carbaldehyde O-ethyl oxime, 4-cyclopropyl-2-((3,4,4-trifluorobut-3-en-1-yl)sulfonyl)thiazole-5-carbaldehyde O-ethyl oxime, (Z)-2-((4,4-difluorobut-3-en-1-yl)thio)oxazole-5-carbaldehyde O-methyl oxime, (E)-2-((4,4-difluorobut-3-en-1-yl)thio)oxazole-5-carbaldehyde O-methyl oxime, (Z)-2-((4,4-difluorobut-3-en-1-yl)thio)oxazole-5-carbaldehyde O-ethyl oxime, (E)-2-((4,4-difluorobut-3-en-1-yl)thio)oxazole-5-carbaldehyde O-ethyl oxime, (E)-4-methyl-2-((3,4,4-trifluorobut-3-en-1-yl)sulfinyl)oxazole-5-carbaldehyde O-methyl oxime, (E)-4-methyl-2-((3,4,4-trifluorobut-3-en-1-yl)sulfonyl)oxazole-5-carbaldehyde O-methyl oxime, (E)-2-((3,4,4-trifluorobut-3-en-1-yl)sulfonyl)thiazole-5-carbaldehyde O-cyclopropylmethyl oxime, (E)-2-((4,4-difluorobut-3-en-1-yl)thio)thiazole-5-carbaldehyde 0-isopropyl oxime, (E)-2-((4,4-difluorobut-3-en-1-yl)sulfinyl)thiazole-5-carbaldehyde O-isopropyl oxime, 5-cyclobutyl-3-(2-((3,4,4-trifluorobut-3-en-1-yl)sulfinyl)thiazol-5-yl)-1,2,4-oxadiazole, (E)-2-((4,4-difluorobut-3-en-1-yl)thio)thiazole-5-carbaldehyde O-cyclopropylmethyl oxime, 2-((4,4-difluorobut-3-en-1-yl)sulfonyl)thiazole-5-carbaldehyde O-ethyl oxime, (E)-2-((4,4-difluorobut-3-en-1-yl)sulfinyl)oxazole-5-carb aldehyde O-methyl oxime, (E)-2-((4,4-difluorobut-3-en-1-yl)thio)thiazole-5-carbaldehyde O-cyclobutylmethyl oxime, (Z)-2-((4,4-difluorobut-3-en-1-yl)thio)thiazole-5-carbaldehyde O-cyclopropylmethyl oxime, (E)-2-((4,4-difluorobut-3-en-1-yl)sulfinyl)thiazole-5-carb aldehyde O-cyclobutylmethyl oxime, (Z)-2-((4,4-difluorobut-3-en-1-yl)sulfinyl)thiazole-5-carb aldehyde O-cyclopropylmethyl oxime, (Z)-2-((4,4-difluorobut-3-en-1-yl)sulfonyl)thiazole-5-carbaldehyde O-cyclopropylmethyl oxime, (E)-2-((4,4-difluorobut-3-en-1-yl)sulfonyl)thiazole-5-carbaldehyde O-cyclopropylmethyl oxime, (E)-2-((4,4-difluorobut-3-en-1-yl)sulfonyl)thiazole-5-carbaldehyde O-cyclobutylmethyl oxime, 5-ethyl-3-(2-((3,4,4-trifluorobut-3-en-1-yl)thio)thiazol-5-yl)-1,2,4-oxadiazole, 5-ethyl-3-(2-((3,4,4-trifluorobut-3-en-1-yl)sulfinyl)thiazol-5-yl)-1,2,4-oxadiazole, 5-ethyl-3-(2-((3,4,4-trifluorobut-3-en-1-yl)sulfonyl)thiazol-5-yl)-1,2,4-oxadiazole, 5-(tert-butyl)-3-(2-((3,4,4-trifluorobut-3-en-1-yl)thio)thiazol-5-yl)-1,2,4-oxadiazole, 5-(3,4-difluorophenyl)-3-(2-((3,4,4-trifluorobut-3-en-1-yl)thio)thiazol-5-yl)-1,2,4-oxadiazole, (E)-4-methyl-2-((3,4,4-trifluorobut-3-en-1-yl)thio)oxazole-5-carbaldehyde 0-cyclobutylmethyl oxime, 5-(tert-butyl)-3-(2-((3,4,4-trifluorobut-3-en-1-yl)sulfinyl)thiazol-5-yl)-1,2,4-oxadiazole, 5-(tert-butyl)-3-(2-((3,4,4-trifluorobut-3-en-1-yl)sulfonyl)thiazol-5-yl)-1,2,4-oxadiazole, 5-(3,4-difluorophenyl)-3-(2-((3,4,4-trifluorobut-3-en-1-yl)sulfinyl)thiazol-5-yl)-1,2,4-oxadiazole, 5-(2-chloro-6-fluorophenyl)-3-(2-((3,4,4-trifluorobut-3-en-1-yl)thio)thiazol-5-yl)isoxazole, 4-chloro-2-((3,4,4-trifluorobut-3-en-1-yl)thio)thiazole-5-carbaldehyde O-methyl oxime, 5-(2-bromo-6-fluorophenyl)-3-(2-((3,4,4-trifluorobut-3-en-1-yl)thio)thiazol-5-yl)isoxazole, (E)-4-(methoxymethyl)-2-((3,4,4-trifluorobut-3-en-1-yl)thio)thiazole-5-carbaldehyde O-methyl oxime, (E)-4-(methoxymethyl)-2-((3,4,4-trifluorobut-3-en-1-yl)sulfinyl)thiazole-5-carbaldehyde O-methyl oxime, (E)-4-(methoxymethyl)-2-((3,4,4-trifluorobut-3-en-1-yl)sulfonyl)thiazole-5-carbaldehyde O-methyl oxime, 4-(methyl amino)-2-((3,4,4-trifluorobut-3-en-1-yl)thio)thiazole-5-carb aldehyde O-methyl oxime, 5-(difluoromethyl)-2-((3,4,4-trifluorobut-3-en-1-yl)thio)thiazole, 5-(difluoromethyl)-2-((3,4,4-trifluorobut-3-en-1-yl)sulfinyl)thiazole, 2-((3,4,4-trifluorobut-3-en-1-yl)thio)thiazole-5-carbonitrile, 2-((3,4,4-trifluorobut-3-en-1-yl)sulfinyl)thiazole-5-carbonitrile, 5-(difluoromethyl)-2-((3,4,4-trifluorobut-3-en-1-yl)sulfonyl)thiazole, 5-(difluoromethyl)-2-((3,4,4-trifluorobut-3-en-1-yl)thio)oxazole, 2-((3,4,4-trifluorobut-3-en-1-yl)thio)oxazole-5-carbonitrile, 2-((3,4,4-trifluorobut-3-en-1-yl)sulfonyl)thiazole-5-carbonitrile, 2-((3,4,4-trifluorobut-3-en-1-yl)sulfinyl)oxazole-5-carbonitrile, 5-(difluoromethyl)-2-((3,4,4-trifluorobut-3-en-1-yl)sulfinyl)oxazole, 5-(difluoromethyl)-4-methyl-2-((3,4,4-trifluorobut-3-en-1-yl)thio)thiazole, 4-methyl-2-((3,4,4-trifluorobut-3-en-1-yl)thio)thiazole-5-carbonitrile, 5-(difluoromethyl)-4-methyl-2-((3,4,4-trifluorobut-3-en-1-yl)sulfinyl)thiazole, 4-methyl-2-((3,4,4-trifluorobut-3-en-1-yl)sulfinyl)thiazole-5-carbonitrile, 4-methyl-2-((3,4,4-trifluorobut-3-en-1-yl)sulfonyl)thiazole-5-carbonitrile, 5-(difluoromethyl)-4-methyl-2-((3,4,4-trifluorobut-3-en-1-yl)sulfonyl)thiazole, 5-(difluoromethyl)-4-methyl-2-((3,4,4-trifluorobut-3-en-1-yl)thio)oxazole, 4-(tert-butyl)-5-(difluoromethyl)-2-((3,4,4-trifluorobut-3-en-1-yl)thio)oxazole, 4-(tert-butyl)-5-(difluoromethyl)-2-((3,4,4-trifluorobut-3-en-1-yl)sulfonyl)oxazole, 4-methyl-2-((3,4,4-trifluorobut-3-en-1-yl)thio)oxazole-5-carbonitrile, 4-(tert-butyl)-2-((3,4,4-trifluorobut-3-en-1-yl)thio)oxazole-5-carbonitrile, 4-methoxy-2-((3,4,4-trifluorobut-3-en-1-yl)thio)thiazole-5-carbonitrile, 4-methoxy-2-((3,4,4-trifluorobut-3-en-1-yl)sulfinyl)thiazole-5-carbonitrile, 4-methyl-2-((3,4,4-trifluorobut-3-en-1-yl)sulfinyl)oxazole-5-carbonitrile, 4-(tert-butyl)-2-((3,4,4-trifluorobut-3-en-1-yl)sulfonyl)oxazole-5-carbonitrile, 5-(difluoromethyl)-4-methyl-2-((3,4,4-trifluorobut-3-en-1-yl)sulfonyl)oxazole, 4-cyclopropyl-5-(difluoromethyl)-2-((3,4,4-trifluorobut-3-en-1-yl)thio)thiazole, 4-cyclopropyl-5-(difluoromethyl)-2-((3,4,4-trifluorobut-3-en-1-yl)sulfinyl)thiazole, 4-cyclopropyl-5-(difluoromethyl)-2-((3,4,4-trifluorobut-3-en-1-yl)sulfonyl)thiazole, 4-cyclopropyl-2-((3,4,4-trifluorobut-3-en-1-yl)thio)thiazole-5-carbonitrile, 4-cyclopropyl-2-((3,4,4-trifluorobut-3-en-1-yl)sulfinyl)thiazole-5-carbonitrile, 4-cyclopropyl-2-((3,4,4-trifluorobut-3-en-1-yl)sulfonyl)thiazole-5-carbonitrile, 4-chloro-2-((3,4,4-trifluorobut-3-en-1-yl)thio)thiazole-5-carbonitrile, 4-cyclopropyl-2-((3,4,4-trifluorobut-3-en-1-yl)thio)thiazole, 4-cyclopropyl-2-((3,4,4-trifluorobut-3-en-1-yl)sulfinyl)thiazole, 4-cyclopropyl-2-((3,4,4-trifluorobut-3-en-1-yl)sulfonyl)thiazole, 4-methyl-2-((3,4,4-trifluorobut-3-en-1-yl)sulfonyl)oxazole-5-carbonitrile, (E)-1-methyl-2-((3,4,4-trifluorobut-3-en-1-yl)thio)-1H-imidazole-5-carb aldehyde O-ethyl oxime, (Z)-1-methyl-2-((3,4,4-trifluorobut-3-en-1-yl)thio)-1H-imidazole-5-carbaldehyde O-ethyl oxime, (E)-1-methyl-2-((3,4,4-trifluorobut-3-en-1-yl)thio)-1H-imidazole-5-carbaldehyde O-methyl oxime, (Z)-1-methyl-2-((3,4,4-trifluorobut-3-en-1-yl)thio)-1H-imidazole-5-carbaldehyde O-methyl oxime, (Z)-1-methyl-2-((3,4,4-trifluorobut-3-en-1-yl)sulfinyl)-1H-imidazole-5-carbaldehyde O-methyl oxime, 4-chloro-2-((3,4,4-trifluorobut-3-en-1-yl)thio)thiazole-5-carbaldehyde and (4-chloro-2-((3,4,4-trifluorobut-3-en-1-yl)thio)thiazol-5-yl)methanol.

It can be advantageous to isolate or synthesize in each case the biologically more effective isomer, for example enantiomer or diastereomer, or isomeric mixture, for example enantiomeric mixture or diastereomeric mixture, if the individual components have a different biological activity.

The compounds of formula (I) and wherever appropriate, the tautomers thereof, in each case in free form or in salt form, can, if appropriate, also be obtained in the form of hydrates and/or include other solvents, for example those which may have been used for the crystallization of compounds which are present in solid form.

In one embodiment, the present invention provides use of compound of general formula (I), stereoisomers, agriculturally acceptable salts, tuatomers or N-oxides thereof or composition or combination thereof for controlling or preventing agricultural crops and/or horticultural crops against phytopathogenic fungi, bacteria, insects, nematodes or mites.

In preferred embodiment, the present invention provides use of compound of general formula (I), stereoisomers, agriculturally acceptable salts, tuatomers or N-oxides thereof or composition or combination thereof for controlling or preventing agricultural crops and/or horticultural crops against nematodes and phytopathogenic fungi.

The agricultural crops are selected from cereals, corn, rice, soybean and other leguminous plants, fruits and fruit trees, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers, other vegetables or ornamentals.

The compounds according to the invention can be used for controlling or destroying pests such as nematodes and/or fungi which occur in particular on plants, especially on useful plants and ornamentals in agriculture, in horticulture and in forests, or on organs, such as fruits, flowers, foliage, stalks, tubers, seeds or roots, of such plants, and in some cases even plant organs which are formed at a later point in time remain protected against these pests.

The compounds of formula (I) according to the invention are preventively and/or curatively valuable active ingredients in the field of pest control, even at low rates of application, which can be used against pesticide resistant pests such as insects and fungi, which compounds of formula (I) have a very favorable biocidal spectrum and are well tolerated by warm-blooded species, fish and plants. Accordingly, the present invention also makes available a pesticidal composition comprising compounds of the invention, such as formula (I). It has now been found that the compounds of formula (I) according to the invention have, for practical purposes, a very advantageous spectrum of activities for protecting animals and useful plants against attack and damage by nematodes and phytopathogenic microorganisms like fungi or bacteria. Accordingly, the present invention also makes available a nematicidal composition comprising compounds of the invention, such as formula (I). It has also now been found that the compounds of formula I according to the invention have, for practical purposes, a very advantageous spectrum of activities for protecting animals and useful plants against attack and damage by fungi. Accordingly, the present invention also makes available a fungicidal composition comprising compounds of the invention, such as formula (I).

The compounds of the formula (I) can possess potent microbicidal activity and can be used for the control of unwanted microorganisms, such as fungi, insects, mites, nematodes and bacteria, in agricultural or horticultural crop protection and in the protection of such materials.

The compounds of the formula (I) can possess very good fungicidal properties and can be used in crop protection, for example for control of Plasmodiophoromycetes, Oomycetes, Chytridiomycetes, Zygomycetes, Ascomycetes, Basidiomycetes and Deuteromycetes.

The compounds of the formula (I) can be used as nematicides in crop protection, for example, for control of Tylenchida, Rhabditida, Dorylaimida, and Tryplonchida.

The compounds of the formula (I) can be used as insecticides in crop protection, for example, for control of Lepidoptera, Coleoptera, Hemiptera, Homoptera, Thysanoptera, Diptera, Orthoptera & Isoptera.

The compounds of the formula (I) can be used as acaricides in crop protection, for example, for control of Eriophyoidea, Tetranychoidea, Eupodoidea and Tarsonemidae.

The compounds of the formula (I) can be used as bactericides in crop protection, for example, for control of Pseudomonadaceae, Rhizobiaceae, Enterobacteriaceae, Corynebacteriaceae and Streptomycetaceae.

The compounds of the formula (I) can be used for curative or protective control of phytopathogenic fungi. The invention therefore also relates to curative and protective methods for controlling phytopathogenic fungi by the use of the inventive active ingredients or compositions, which are applied to the seed, the plant or plant parts, the fruit or the soil in which the plants grow.

The compounds of the formula (I) can be used for controlling or preventing against phytopathogenic fungi, bacteria, insects, nematodes, mites of agricultural crops and or horticultural crops.

The compounds of the formula (I) can be used in crop protection, wherein the agricultural crops are cereals, corn, rice, soybean and other leguminous plants, fruits and fruit trees, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers and other vegetables, and ornamentals.

The compounds of formula (I) are especially useful for the control of nematodes. Thus, in a further aspect, the invention also relates to a method of controlling damage to plant and parts thereof by plant parasitic nematodes (Endoparasitic, Semiendoparasitic and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Eelonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

Particularly, the nematode species Meloidogyne spp., Heterodera spp., Rotylenchus spp., Pratylenchus spp. and Radopholus spp. can be controlled by compounds of the invention.

The compound of formula (I) and compositions thereof, respectively, are also suitable for controlling harmful fungi in the protection of stored products or harvest and in the protection of materials.

The compound of formula (I) and compositions thereof, respectively, are particularly suitable for controlling the following plant diseases: Albugo spp. (white rust) on ornamentals, vegetables (e.g. A candida) and sunflowers (e.g. A. tragopogonis); Alternaria spp. (Alternaria leaf spot) on vegetables, rape (A. brassicola or brassicae), sugar beets (A. tenuis), fruits, rice, soybeans, potatoes (e. g. A. solanior A. alternata), tomatoes (e.g. A. solanior A. alternata) and wheat; Aphanomyces spp. on sugar beets and vegetables; Ascochyta spp. on cereals and vegetables, e.g. A. tritici(anthracnose) on wheat and A. hordei on barley; Bipolaris and Drechslera spp. (teleomorph: Cochliobolus spp.), e.g. Southern leaf blight (D. maydis) or Northern leaf blight (B. zeicola) on corn, e. g. spot blotch (B. sorokiniana) on cereals and e. g. B. oryzae on rice and turfs; Blumeria (formerly Erysiphe) graminis (powdery mildew) on cereals (e.g. on wheat or barley); Botrytis cinerea (teleomorph: Botryotinia fuceliana: grey mold) on fruits and berries (e. g. strawberries), vegetables (eg. lettuce, carrots, celery and cabbages), rape, flowers, vines, forestry plants and wheat; Bremialactucae (downy mildew) on lettuce; Ceratocystis (syn. Ophiostoma) spp. (rot or wilt) on broad leaved trees and evergreens, e. g. C. ulmi (Dutch elm disease) on elms; Cercospora spp. (Cercospora leaf spots) on corn (e. g. Gray leaf spot: C. zeae-maydis), rice, sugar beets (e. g. C. beticola), sugar cane, vegetables, coffee, soybeans (e. g. C. sojina or C. kikuchil) and rice; Cladosporium spp. on tomatoes (e. g. C. fu/vum: leaf mold) and cereals, e.g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e.g. C. miyabeanus, anamorph: H. oryzae); Colletotrichum (teleomorph: Glomerella) spp. (anthracnose) on cotton (e. g. C. gossypil), corn (e. g. C. graminicola: Anthracnose stalk rot), soft fruits, potatoes (e. g. C. coccodes: black dot), beans (e. g. C. lindemuthianum) and soybeans (e.g. C. truncatum or C. gloeosporioides); Corticium spp., e.g. C. sasakii (sheath blight) on rice; Corynespora cassiicola (leaf spots) on soybeans and ornamentals; Cycloconium spp., e.g. C. oleaginum on olive trees; Cylindrocarpon spp. (e.g. fruit tree canker or young vine decline, teleomorph: Nectria or Neonectria spp.) on fruit trees, vines (e.g. C. liriodendri, teleomorph: Neonectria liriodendrf. Black Foot Disease) and ornamentals; Dematophora (teleomorph: Rosellinia) necatrix (root and stem rot) on soybeans; Diaporthe spp., e.g. D. phaseolorum (damping off) on soybeans; Drechslera (syn. Helminthosporium, teleomorph: Pyrenophora) spp. on corn, cereals, such as barley (e.g. D. teres, net blotch) and wheat (e.g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (E. pyn), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e.g. E. pis,), such as cucurbits (e.g. E. cichoracearum), cabbages, rape (e.g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani (f. sp. glycines now syn. F. virguliforme) and F. tucumani-ae and F. brasiliense each causing sudden death syndrome on soybeans, and F. verticillioides on corn; Gaeumannomyces graminis (take-all) on cereals (e.g. wheat or barley) and corn; Gibberella spp. on cereals (e.g. G. zeae) and rice (e. g. G. fujikurof. Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e.g. G. sabinae (rust) on pears; Helminthosporium spp. (syn. Drechslera, teleomorph: Cochliobolus) on corn, cereals and rice; Hemileia spp., e.g. H. vastatrix(coffee leaf rust) on coffee; lsariopsis clavispora (syn. Cladosporium vitis) on vines; Macrophominaphaseolina (syn. phaseo/l) (root and stem rot) on soybeans and cotton; Microdochium (syn. Fusarium) nivale (pink snow mold) on cereals (e.g. wheat or barley); Microsphaera diffusa (powdery mildew) on soybeans; Monilinia spp., e.g. M. laxa, M. fructicola and M. fructigena (bloom and twig blight, brown rot) on stone fruits and other rosaceous plants; Mycosphaerella spp. on cereals, bananas, soft fruits and ground nuts, such as e.g. M. graminicola (anamorph: Septoria tritici, Septoria blotch) on wheat or M. fijiensis (black Sigatoka disease) on bananas; Peronospora spp. (downy mildew) on cabbage (e. g. P. brassicae), rape (e.g. P. parasitica), onions (e.g. P. destructor), tobacco (P. tabacina) and soybeans (e.g. P. manshurica); Phakopsora pachyrhizi and P. meibomiae (soybean rust) on soybeans; Phialophora spp. e. g. on vines (e.g. P. tracheiphila and P. tetraspora) and soybeans (e.g. P. gregata stem rot); Phoma lingam (root and stem rot) on rape and cabbage and P. betae (root rot, leaf spot and damping-off) on sugar beets; Phomopsis spp. on sunflowers, vines (e.g. P. viticola: can and leaf spot) and soybeans (e.g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsicl), soybeans (e. g. P. megasperma, syn. P. sojae), potatoes and tomatoes (e. g. P. infestans: late blight) and broad-leaved trees (e. g. P. ramorum: sudden oak death); Plasmodiophora brassicae (club root) on cabbage, rape, radish and other plants; Plasmopara spp., e.g. P. viticola (grapevine downy mildew) on vines and P. halstedii on sunflowers; Podosphaera spp. (powdery mildew) on rosaceous plants, hop, pome and soft fruits, e.g. P. leucotricha on apples; Polymyxa spp., e.g. on cereals, such as barley and wheat (P. graminis) and sugar beets (P. betae) and thereby transmitted viral diseases; Pseudocercosporella herpotrichoides (eyespot, teleomorph: Tapesia ya/lundae) on cereals, e.g. wheat or barley; Pseudoperonospora (downy mildew) on various plants, e. g. P. cubensis on cucurbits or P. humili on hop; Pseudopezicula tracheiphila (red fire disease or, rotbrenner′, anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e.g. wheat, barley or rye, P. kuehnii(orange rust) on sugar cane and P. asparagion asparagus; Pyrenophora (anamorph: Drechslera) tritici-repentis (tan spot) on wheat or P. teres (net blotch) on barley; Pyricularia spp., e.g. P. oryzae (teleomorph: Magnaporthe grisea, rice blast) on rice and P. grisea on turf and cereals; Pythium spp. (damping-off) on turf, rice, corn, wheat, cotton, grape, sunflowers, soybeans, sugar beets, vegetables and various other plants (e. g. P. ultimum or P. aphanidermatum); Ramu/aria spp., e.g. R. collo-cygni(Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e. g. S. sclerotiorum) and soybeans (e.g. S. rolfsiior S. sclerotiorum); Septoria spp. on various plants, e.g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tucken) on vines; Setospaeria spp. (leaf blight) on corn (e.g. S. turcicum, syn. Helminthosporium turcicum) and turf; Sphacelotheca spp. (smut) on corn, (e. g. S. reiliana: head smut), sorghum and sugar cane; Sphaerotheca fuliginea (powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e.g. T. deformans (leaf curl disease) on peaches and T. pruni (plum pocket) on plums; Thielaviopsis spp. (black root rot) on tobacco, pome fruits, vegetables, soybeans and cotton, e.g. T. basicola (syn. Chalara elegans); Tilletia spp. (common bunt or stinking smut) on cereals, such as e. g. T. tritici (syn. T. caries, wheat bunt) and T. controversa (dwarf bunt) on wheat; Typhula incarnata (grey snow mold) on barley or wheat; Urocystis spp., e.g. U. occulta (stem smut) on rye; Uromyces spp. (rust) on vegetables, such as beans (e.g. U. appendiculatus, syn. U. phaseo/J) and sugar beets (e. g. U. betae); Ustilago spp. (loose smut) on cereals (e.g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. dahliae on strawberries, rape, potatoes and tomatoes.

In one embodiment, the present invention provides a composition for controlling or preventing phytopathogenic microorganisms comprising a compound of general formula (I), stereoisomer, agriculturally acceptable salts, tautomers or N-oxides thereof and one or more inert carriers.

In another embodiment, the composition may additionally comprises one or more active compatible compounds selected from fungicides, insecticides, nematicides, acaricides, biopesticides, herbicides, plant growth regulators, antibiotics, nutrients or fertilizers.

The concentration of the compound of general formula (I) ranges from 1 to 90% by weight with respect to the total weight of the composition, preferably from 5 to 50% by weight with respect to the total weight of the composition.

The present invention further relates to a composition for controlling unwanted microorganisms comprising at least one of the compounds of the formula (I) and one or more inert carrier. The inert carrier further comprises agriculturally suitable auxiliaries, solvents, diluents, surfactants and/or extenders and the like.

The present invention further relates to a composition for controlling unwanted microorganisms, comprising at least one of the compounds of the formula (I) and/or one or more active compatible compound selected from fungicides, bactericides, acaricides, insecticides, nematicides, herbicides, biopesticides, plant growth regulators, antibiotics, fertilizers and/or mixtures thereof.

Generally, a compound of the present invention is used in the form of a composition (e.g. formulation) containing a carrier. A compound of the invention and compositions thereof can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra-low volume (ulv) liquid, ultra-low volume (ulv) suspension, water dispersible granules or tablets, water dispersible powder for slurry treatment, water soluble granules or tablets, water soluble powder for seed treatment and wettable powder.

A formulation typically comprises a liquid or solid carrier and optionally one or more customary formulation auxiliaries, which may be solid or liquid auxiliaries, for example unepoxidized or epoxidized vegetable oils (for example epoxidized coconut oil, rapeseed oil or soya oil), antifoams, for example silicone oil, preservatives, clays, inorganic compounds, viscosity regulators, surfactant, binders and/or tackifiers. The composition may also further comprise a fertilizer, a micronutrient donor or other preparations which influence the growth of plants as well as comprising a combination containing the compound of the invention with one or more other biologically active agents, such as bactericides, fungicides, nematicides, plant activators, acaricides, and insecticides.

Accordingly, the present invention also makes available a composition comprising a compound of the invention and an agronomical carrier and optionally one or more customary formulation auxiliaries.

The compositions are prepared in a manner known per se, in the absence of auxiliaries for example by grinding, screening and/or compressing a solid compound of the present invention and in the presence of at least one auxiliary for example by intimately mixing and/or grinding the compound of the present invention with the auxiliary (auxiliaries). In the case of solid compounds of the invention, the grinding/milling of the compounds is to ensure specific particle size. These processes for the preparation of the compositions and the use of the compounds of the invention for the preparation of these compositions are also a subject of the invention.

Examples of compositions for use in agriculture are emulsifiable concentrates, suspension concentrates, microemulsions, oil dispersibles, directly sprayable or dilutable solutions, spreadable pastes, dilute emulsions, soluble powders, dispersible powders, wettable powders, dusts, granules or encapsulations in polymeric substances, which comprise—at least—a compound according to the invention and the type of composition is to be selected to suit the intended aims and the prevailing circumstances.

Examples of suitable liquid carriers are unhydrogenated or partially hydrogenated aromatic hydrocarbons, preferably the fractions C₈ to C₁₂ of alkylbenzenes, such as xylene mixtures, alkylated naphthalenes or tetrahydronaphthalene, aliphatic or cycloaliphatic hydrocarbons, such as paraffins or cyclohexane, alcohols such as ethanol, propanol or butanol, glycols and their ethers and esters such as propylene glycol, dipropylene glycol ether, ethylene glycol or ethylene glycol monomethyl ether or ethylene glycol monoethyl ether, ketones, such as cyclohexanone, isophorone or diacetone alcohol, strongly polar solvents, such as N-methylpyrrolid-2-one, dimethyl sulfoxide or N,N-dimethylformamide, water, unepoxidized or epoxidized vegetable oils, such as unexpodized or epoxidized rapeseed, castor, coconut or soya oil, and silicone oils. Examples of solid carriers which are used for example for dusts and dispersible powders are, as a rule, ground natural minerals such as calcite, talc, kaolin, montmorillonite or attapulgite. To improve the physical properties, it is also possible to add highly disperse silicas or highly disperse adsorptive polymers. Suitable particulate adsorptive carriers for granules are porous types, such as pumice, brick grit, sepiolite or bentonite, and suitable non-sorptive carrier materials are calcite or sand. In addition, a large number of granulated materials of inorganic or organic nature can be used, in particular dolomite or comminuted plant residues. Suitable surface-active compounds are, depending on the type of the active ingredient to be formulated, non-ionic, cationic and/or anionic surfactants or surfactant mixtures which have good emulsifying, dispersing and wetting properties. The surfactants mentioned below are only to be considered as examples; a large number of further surfactants which are conventionally used in the art of formulation and suitable according to the invention are described in the relevant literature. Suitable non-ionic surfactants are, especially, polyglycol ether derivatives of aliphatic or (cyclo)aliphatic alcohols, of saturated or unsaturated fatty acids or of alkyl phenols which may contain approximately 3 to approximately 30 glycol ether groups and approximately 8 to approximately 20 carbon atoms in the (cyclo)aliphatic hydrocarbon radical or approximately 6 to approximately 18 carbon atoms in the alkyl moiety of the alkyl phenols. Also suitable are water-soluble polyethylene oxide adducts with polypropylene glycol, ethylenediaminopolypropylene glycol or alkyl polypropylene glycol having 1 to approximately 10 carbon atoms in the alkyl chain and approximately 20 to approximately 250 ethylene glycol ether groups and approximately 10 to approximately 100 propylene glycol ether groups. Normally, the abovementioned compounds contain 1 to approximately 5 ethylene glycol units per propylene glycol unit. Examples which may be mentioned are nonylphenoxypolyethoxyethanol, castor oil polyglycol ether, polypropylene glycol/polyethylene oxide adducts, tributylphenoxypolyethoxyethanol, polyethylene glycol or octylphenoxypolyethoxyethanol. Also suitable are fatty acid esters of polyoxyethylene sorbitan, such as polyoxyethylene sorbitan trioleate. The cationic surfactants are, especially, quarternary ammonium salts which generally have at least one alkyl radical of approximately 8 to approximately 22 Carbon atoms as substituents and as further substituents (unhalogenated or halogenated) lower alkyl or hydroxyalkyl or benzyl radicals. The salts are preferably in the form of halides, methylsulfates or ethylsulfates. Examples are stearyltrimethylammonium chloride and benzylbis(2-chloroethyl)ethylammonium bromide.

Examples of suitable anionic surfactants are water-soluble soaps or water-soluble synthetic surface-active compounds. Examples of suitable soaps are the alkali, alkaline earth or (unsubstituted or substituted) ammonium salts of fatty acids having approximately 10 to approximately 22 Carbon atoms, such as the sodium or potassium salts of oleic or stearic acid, or of natural fatty acid mixtures which are obtainable for example from coconut or tall oil; mention must also be made of the fatty acid methyl taurates. However, synthetic surfactants are used more frequently, in particular fatty sulfonates, fatty sulfates, sulfonated benzimidazole derivatives or alkylaryl sulfonates. As a rule, the fatty sulfonates and fatty sulfates are present as alkali, alkaline earth or (substituted or unsubstituted) ammonium salts and they generally have an alkyl radical of approximately 8 to approximately 22 Carbon atoms, alkyl also to be understood as including the alkyl moiety of acyl radicals; examples which may be mentioned are the sodium or calcium salts of lignosulfonic acid, of the dodecylsulphuric ester or of a fatty alcohol sulfate mixture prepared from natural fatty acids. This group also includes the salts of the sulphuric esters and sulfonic acids of fatty alcohol/ethylene oxide adducts. The sulfonated benzimidazole derivatives preferably contain 2 sulphonyl groups and a fatty acid radical of approximately 8 to approximately 22 Carbon atoms. Examples of alkylarylsulfonates are the sodium, calcium or triethanolammonium salts of decylbenzenesulfonic acid, of dibutylnaphthalenesulfonic acid or of a naphthalenesulfonic acid/formaldehyde condensate. Also possible are, furthermore, suitable phosphates, such as salts of the phosphoric ester of a p-nonylphenol/(4-14)ethylene oxide adduct, or phospholipids.

As a rule, the compositions comprise 0.1 to 99%, especially 0.1 to 95%, of compound according to the present invention and 1 to 99.9%, especially 5 to 99.9%, of at least one solid or liquid carrier, it being possible as a rule for 0 to 25%, especially 0.1 to 20%, of the composition to be surfactants (% in each case meaning percent by weight). Whereas concentrated compositions tend to be preferred for commercial goods, the end consumer as a rule uses dilute compositions which have substantially lower concentrations of active ingredient.

Examples of foliar formulation types for pre-mix compositions are:

GR: granules EW: emulsions, oil in water

WP: wettable powders ME: micro-emulsion

WG: water dispersable granules (powders) SC: aqueous suspension concentrate

SG: water soluble granules CS: aqueous capsule suspension

SL: soluble concentrates OD: oil-based suspension concentrate, and

EC: emulsifiable concentrate SE: aqueous suspo-emulsion.

Whereas, examples of seed treatment formulation types for pre-mix compositions are:

WS: wettable powders for seed treatment slurry FS: suspension concentrates for seed treatment

LS: solution for seed treatment WG: water dispersible granules, and

ES: emulsions for seed treatment CS: aqueous capsule suspension.

Examples of formulation types suitable for tank-mix compositions are solutions, dilute emulsions, suspensions, or a mixture thereof, and dusts.

As with the nature of the formulations, the methods of application, such as foliar, drench, spraying, atomizing, dusting, scattering, coating or pouring, are chosen in accordance with the intended objectives and the prevailing circumstances.

The tank-mix compositions are generally prepared by diluting with a solvent (for example, water) the one or more pre-mix compositions containing different pesticides, and optionally further auxiliaries. Suitable carriers and adjuvants can be solid or liquid and are the substances ordinarily employed in formulation technology, e.g. natural or regenerated mineral substances, solvents, dispersants, wetting agents, tackifiers, thickeners, binders or fertilizers.

Generally, a tank-mix formulation for foliar or soil application comprises 0.1 to 20%, especially 0.1 to 15%, of the desired ingredients, and 99.9 to 80%, especially 99.9 to 85%, of a solid or liquid auxiliaries (including, for example, a solvent such as water), where the auxiliaries can be a surfactant in an amount of 0 to 20%, especially 0.1 to 15%, based on the tank-mix formulation. Typically, a pre-mix formulation for foliar application comprises 0.1 to 99.9%, especially 1 to 95%, of the desired ingredients, and 99.9 to 0.1%, especially 99 to 5%, of a solid or liquid adjuvant (including, for example, a solvent such as water), where the auxiliaries can be a surfactant in an amount of 0 to 50%, especially 0.5 to 40%, based on the pre-mix formulation.

Normally, a tank-mix formulation for seed treatment application comprises 0.25 to 80%, especially 1 to 75%, of the desired ingredients, and 99.75 to 20%, especially 99 to 25%, of a solid or liquid auxiliaries (including, for example, a solvent such as water), where the auxiliaries can be a surfactant in an amount of 0 to 40%, especially 0.5 to 30%, based on the tank-mix formulation.

Typically, a pre-mix formulation for seed treatment application comprises 0.5 to 99.9%, especially 1 to 95%, of the desired ingredients, and 99.5 to 0.1%, especially 99 to 5%, of a solid or liquid adjuvant (including, for example, a solvent such as water), where the auxiliaries can be a surfactant in an amount of 0 to 50%, especially 0.5 to 40%, based on the pre-mix formulation whereas commercial products will preferably be formulated as concentrates (e.g., pre-mix composition (formulation)), the end user will normally employ dilute formulations (e.g., tank mix composition).

Preferred seed treatment pre-mix formulations are aqueous suspension concentrates. The formulation can be applied to the seeds using conventional treating techniques and machines, such as fluidized bed techniques, the roller mill method, roto static seed treaters, and drum coaters. Other methods, such as spouted beds may also be useful. The seeds may be pre sized before coating. After coating, the seeds are typically dried and then transferred to a sizing machine for sizing. Such procedures are known in the art. The compounds of the present invention are particularly suited for use in soil and seed treatment applications.

In general, the pre-mix compositions of the invention contain 0.5 to 99.9% especially 1 to 95%, advantageously 1 to 50%, by mass of the desired ingredients, and 99.5 to 0.1%, especially 99 to 5%, by mass of a solid or liquid adjuvant (including, for example, a solvent such as water), where the auxiliaries (or adjuvant) can be a surfactant in an amount of 0 to 50%, especially 0.5 to 40%, by mass based on the mass of the pre-mix formulation.

A compound of the formula (I) in a preferred embodiment, independent of any other embodiments, is in the form of a plant propagation material treating (or protecting) composition, wherein said plant propagation material protecting composition may comprises additionally a coloring agent. The plant propagation material protecting composition or mixture may also comprise at least one polymer from water-soluble and water-dispersible film-forming polymers that improve the adherence of the active ingredients to the treated plant propagation material, which polymer generally has an average molecular weight of at least 10,000 to about 100,000.

In an embodiment, the present invention provides a method of controlling or preventing infestation of useful plants by phytopathogenic microorganisms in agricultural crops and/or horticultural crops, wherein the compound of general formula (I) and/or stereoisomers or agriculturally acceptable salts or tuatomers or N-oxides thereof or composition or combination thereof, is applied to the plants, to parts thereof or a locus thereof.

In another embodiment, the present invention provides a method of controlling or preventing infestation of useful plants by phytopathogenic microorganisms in agricultural crops and/or horticultural crops, wherein the compound of general formula (I) and/or stereoisomers or agriculturally acceptable salts or tuatomers or N-oxides thereof or composition or combination thereof is applied to a seeds of plants.

In yet another embodiment, the present invention provides a method of controlling or preventing phytopathogenic microorganisms in agricultural crops and/or horticultural crops using the compound of general formula (I) and/or stereoisomers or agriculturally acceptable salts or tuatomers or N-oxides thereof or composition or combination thereof comprises a step of applying an effective dosage of the compound or the composition or the combination, in amounts ranging from 1 g to 5 kg per hectare of agricultural and/or horticultural crops.

Examples of application methods for the compounds of the invention and compositions thereof, that is the methods of controlling pests in the agriculture, are spraying, atomizing, dusting, brushing on, dressing, scattering or pouring—which are to be selected to suit the intended aims of the prevailing circumstances.

One method of application in agriculture is application to the foliage of the plants (foliar application), it being possible to select frequency and rate of application to match the danger of infestation with the pest or fungi in question. Alternatively, the active ingredient can reach the plants via the root system (systemic action), by applying the compound to the locus of the plants, for example by application of a liquid composition of the compound into the soil (by drenching), or by applying a solid form of the compound in the form of granules to the soil (soil application). In the case of paddy rice plants, such granules can be metered into the flooded paddy-field. The application of the compounds of the present invention to the soil is a preferred application method.

Typical rates of application per hectare is generally 1 to 2000 g of active ingredient per hectare, in particular 10 to 1000 g/ha, preferably 10 to 600 g/ha, such as 50 to 300 g/ha.

In one embodiment, the present invention provides a seed comprising compound of formula (I) and/or stereoisomers, agriculturally acceptable salts, tuatomers, N-oxides thereof or composition or combination thereof, wherein the amount of the compound of the formula (I) or an N-oxide or an agriculturally acceptable salt thereof is ranging from 0.1 g to 10 kg per 100 kg of seed.

The compounds of the invention and compositions thereof are also suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds. The application of the compounds of the present invention to seeds is a preferred application method.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound formula I, which is a preferred application method, can be carried out by any known methods, such as spraying or by dusting the seeds before sowing or during the sowing/planting of the seeds.

Suitable target plants are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous plants, such as beans, lentils, peas or soya; oil plants, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family, latex plants and ornamentals (such as flowers, and lawn grass or turf). In an embodiment, the plant is selected from cereals, corn, soybean, rice, sugarcane, vegetables and oil plants.

The term “plant” is to be understood as including also plants which have been so transformed by the use of recombinant DNA techniques that they are capable of synthesizing one or more selectively acting toxins, such as are known, for example, from toxin-producing bacteria, especially those of the genus Bacillus and also plants which have been selected or hybridized to preserve and/or attain a desired trait, such as insect, fungi and/or nematode resistance. Toxins that can be expressed by such transgenic plants include, for example, insecticidal proteins from Bacillus cereus or Bacillus popilliae; or insecticidal proteins from Bacillus thuringiensis, such as 8-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases. In the context of the present invention there are to be understood by 8-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810). Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073. The processes for the preparation of such transgenic plants are generally known to the person skilled in the art and are described, for example, in the publications mentioned above. Cry1-type deoxyribonucleic acids and their preparation are known, for example, from WO 95/34656, EP-A-0 367474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera). Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1 Ab and a Cry3Bb1 toxin); Starlink® (maize variety that expresses a Cry9C toxin); HerculexI® (maize variety that expresses a Cry1Fa2 toxin and the enzyme phosphinothricine N-acetyltransferase(PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1 Ab toxin); Newleaf® (potato variety that expresses a Cry3A toxin); NatureGard®, 25Agrisure® GT Advantage (GA21 glyphosate-tolerant trait), Agrisure® CB Advantage (Bt11 corn borer (CB) trait) and Protecta®. Further examples of such transgenic plants are: i) Bt11 Maize from Syngenta Seeds SAS, Chemin de l'Hobit 27, F-31 790 St Sauveur, France, registration number C/FR/96/05/10. Genetically modified Zea mays which has been rendered resistant to attack by the European corn borer (Ostrinia nubi/alis and Sesamia nonagrioides) by transgenic expression of a truncated Cry1Ab toxin. Bt11 maize also transgenically expresses the enzyme PAT to achieve tolerance to the herbicide glufosinate ammonium; ii)Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubi/alis and Sesamia nonagrioides) by transgenic expression of a Cry1Ab toxin. Bt176 maize also transgenically expresses the enzyme PAT to achieve tolerance to the herbicide glufosinate ammonium; iii) MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810; iv) MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects; v) IPC 531 Cotton from Monsanto Europe S.A. 270-272 Avenue de Tervuren, B-1150 Brussels, Belgium, registration number C/ES/96/02; vi) 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium; vii) NK603 x MON 810 Maize from Monsanto Europe S.A. 270-272 Avenue de Tervuren, B-1150 Brussels, Belgium, registration number C/GB/02/M3/03. Consists of conventionally breed hybrid maize varieties by crossing the genetically modified varieties NK603 and MON 810. NK603 x MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

In one embodiment, the present invention provides a combination comprising the compound of general formula (I), stereoisomer, agriculturally acceptable salts, tautomers or N-oxides thereof and one or more active compatible compound selected from fungicides, insecticides, nematicides, acaricides, biopesticides, herbicides, plant growth regulators, antibiotics, nutrients or fertilizers.

Compounds of this invention are effective for controlling nematodes, acarine pests and/or fungal pathogens of agronomic plants, both growing and harvested, when employed alone, they may also be used in combination with other biological active agents used in agriculture, such as one or more nematicides, insecticides, acaricides, fungicides, bactericides, plant activator, molluscicide, and pheromones (whether chemical or biological). Mixing the compounds of the invention or the compositions thereof in the use form as pesticides with other pesticides frequently results in a broader pesticidal spectrum of action. For example, the formula (I) compounds of this invention may be used effectively in conjunction or combination with pyrethroids, neonicotinoids, macrolides, diamides, phosphates, carbamates, cyclodienes, formamidines, phenol tin compounds, chlorinated hydrocarbons, benzoylphenyl ureas, pyrroles and the like.

The activity of the compositions according to the invention can be broadened considerably, and adapted to prevailing circumstances, by adding, for example, one or more insecticidally, acaricidally, nematicidally and/or fungicidally active agents. The combinations compounds of formula (I) with other insecticidally, acaricidally, nematicidally and/or fungicidally active agents may also have further surprising advantages. For example, better tolerance by plants, reduced phytotoxicity, pests or fungi can be controlled in their different development stages or better behavior during their production, for example during grinding or mixing, during their storage or during their use.

The following list of pesticides together with which the compounds according to the invention can be used, is intended to illustrate the possible combinations by way of example.

The following combination of the compounds of formula (I) with other active compounds is an adjuvant selected from the group of substances consisting of all named mixing partners of the classes (A) to (O) as described below can, if their functional groups enable this, optionally form salts with suitable bases or acids appear as stereoisomers, even if not specifically mentioned in each case, or as polymorphs. They are also understood as being included herein. These examples are:

A) Inhibitors of the ergosterol biosynthesis, for example (A01) aldimorph, (A02) azaconazole, (A03) bitertanol, (A04) bromuconazole, (A05) cyproconazole, (A06) diclobutrazole, (A07) difenoconazole, (A08) diniconazole, (A09) diniconazole-M, (A10) dodemorph, (A11) dodemorph acetate, (A12) epoxiconazole, (A13) etaconazole, (A14) fenarimol, (A15) fenbuconazole, (A16) fenhexamid, (A17) fenpropidin, (A18) fenpropimorph, (A19) fluquinconazole, (A20) flurprimidol, (A21) flusilazole, (A22) flutriafol, (A23) furconazole, (A24) furconazole-cis, (A25) hexaconazole, (A26) imazalil, (A27) imazalil sulfate, (A28) imibenconazole, (A29) ipconazole, (A30) metconazole, (A31) myclobutanil, (A32) naftifine, (A33) nuarimol, (A34) oxpoconazole, (A35) paclobutrazol, (A36) pefiirazoate, (A37) penconazole, (A38) piperalin, (A39) prochloraz, (A40) propiconazole, (A41) prothioconazole, (A42) pyributicarb, (A43) pyrifenox, (A44) quinconazole, (A45) simeconazole, (A46) spiroxamine, (A47) tebuconazole, (A48) terbinafine, (A49) tetraconazole, (A50) triadimefon, (A51) triadimenol, (A52) tridemorph, (A53) triflumizole, (A54) triforine, (A55) triticonazole, (A56) uniconazole, (A57) uniconazole-p, (A58) viniconazole, (A59) voriconazole, (A60) 1-(4-chlorophenyl)-2-(1H-1,2,4-triazol-1-yl)cycloheptanol, (A61) methyl 1-(2,2-dimethyl-2,3-dihydro-1H-inden-1-yl)-1H-imidazole-5-carboxylate, (A62) N′-{5-(difluoromethyl)-2-methyl-4-[3-(trimethylsilyl) propoxy]phenyl}-N-ethyl-N-methylimidoformamide, (A63) N-ethyl-N-methyl-N′-{2-methyl-5-(trifluoromethyl)-4-[3-(trimethylsilyl)propoxy]phenyl} imidoformamide, (A64) 0-[1-(4-methoxyphenoxy)-3,3-dimethylbutan-2-yl]-1H-imidazole-1-carbothioate, (A65) Pyrisoxazole, (A66) 2-{[3-(2-chlorophenyl)-2-(2,4-difluorophenyl)oxiran-2-yl]methyl}-2,4-dihydro-3H-1,2,4-triazole-3-thione, (A67) 1-{[3-(2-chlorophenyl)-2-(2,4-difluorophenyl)oxiran-2-yl]methyl}-1H-1,2,4-triazol-5-yl thiocyanate, (A68) 5-(allylsulfanyl)-1-{[3-(2-chlorophenyl)-2-(2,4-difluorophenyl)oxiran-2-yl]methyl}-1H-1,2,4-triazole, (A69) 2-[1-(2,4-dichlorophenyl)-5-hydroxy-2,6,6-trimethylheptan-4-yl]-2,4-dihydro-3H-1,2,4-triazole-3-thione, (A70) 2-{[rel(2R,3S)-3-(2-chlorophenyl)-2-(2,4-difluorophenyl) oxiran-2-yl]methyl}-2,4-dihydro-3H-1,2,4-triazole-3-thione, (A71) 2-{[rel(2R,3R)-3-(2-chlorophenyl)-2-(2,4-difluorophenyl)oxiran-2-yl]methyl}-2,4-dihydro-3H-1,2,4-triazole-3-thione, (A72) 1-{[rel(2R,3S)-3-(2-chlorophenyl)-2-(2,4-difluorophenyl)oxiran-2-yl]methyl}-1H-1,2,4-triazol-5-yl thiocyanate, (A73) 1-{[rel(2R,3R)-3-(2-chlorophenyl)-2-(2,4-difluorophenyl)oxiran-2-yl]methyl}-1H-1,2,4-triazol-5-yl thiocyanate, (A74) 5-(allylsulfanyl)-1-{[rel(2R,3S)-3-(2-chlorophenyl)-2-(2,4-difluorophenyl)oxiran-2-yl]methyl}-1H-1,2,4-triazole, (A75) 5-(allylsulfanyl)-1-{[rel(2R,3R)-3-(2-chlorophenyl)-2-(2,4-difluorophenyl) oxiran-2-yl]methyl}-1H-1,2,4-triazole, (A76) 2-[(2S,4S,5S)-1-(2,4-dichlorophenyl)-5-hydroxy-2,6,6-trimethylheptan-4-yl]-2,4-dihydro-3H-1,2,4-triazole-3-thione, (A77) 2-[(2R,4S,5S)-1-(2,4-dichlorophenyl)-5-hydroxy-2,6,6-trimethylheptan-4-yl]-2,4-dihydro-3H-1,2,4-triazole-3-thione, (A78) 2-[(2R,4R,5R)-1-(2,4-dichlorophenyl)-5-hydroxy-2,6,6-trimethylheptan-4-yl]-2,4-dihydro-3H-1,2,4-triazole-3-thione, (A79) 2-[(2S,4R,5R)-1-(2,4-dichlorophenyl)-5-hydroxy-2,6,6-trimethylheptan-4-yl]-2,4-dihydro-3H-1,2,4-triazole-3-thione, (A80) 2-[(2S,4S,5R)-1-(2,4-dichlorophenyl)-5-hydroxy-2,6,6-trimethylheptan-4-yl]-2,4-dihydro-3H-1,2,4-triazole-3-thione, (A81) 2-[(2R,4S,5R)-1-(2,4-dichlorophenyl)-5-hydroxy-2,6,6-trimethylheptan-4-yl]-2,4-dihydro-3H-1,2,4-triazole-3-thione, (A82) 2-[(2R,4R,5S)-1-(2,4-dichlorophenyl)-5-hydroxy-2,6,6-trimethylheptan-4-yl]-2,4-dihydro-3H-1,2,4-triazole-3-thione, (A83) 2-[(2S,4R,5S)-1-(2,4-dichlorophenyl)-5-hydroxy-2,6,6-trimethylheptan-4-yl]-2,4-dihydro-3H-1,2,4-triazole-3-thione, (A84) 2-[4-(4-chlorophenoxy)-2-(trifluoromethyl)phenyl]-1-(1H-1,2,4-triazol-1-yl)propan-2-ol, (A85) 2-[4-(4-chlorophenoxy)-2-(trifluoromethyl)phenyl]-1-(1H-1,2,4-triazol-1-yl)butan-2-ol, (A86) 2-[4-(4-chlorophenoxy)-2-(trifluoromethyl)phenyl]-1-(1H-1,2,4-triazol-1-yl)pentan-2-ol, (A87) 2-[2-chloro-4-(4-chlorophenoxy)phenyl]-1-(1H-1,2,4-triazol-1-yl)butan-2-ol, (A88) 2-[2-chloro-4-(2,4-dichlorophenoxy)phenyl]-1-(1H-1,2,4-triazol-1-yl)propan-2-ol, (A89) (2R)-2-(1-chlorocyclopropyl)-4-[(1R)-2,2-dichlorocyclopropyl]-1-(1H-1,2,4-triazol-1-yl)butan-2-ol, (A90) (2R)-2-(1-chlorocyclopropyl)-4-[(1S)-2,2-dichlorocyclopropyl]-1-(1H-1,2,4-triazol-1-yl)butan-2-ol, (A91) (2S)-2-(1-chlorocyclopropyl)-4-[(1S)-2,2-dichlorocyclopropyl]-1-(1H-1,2,4-triazol-1-yl)butan-2-ol, (A92) (2S)-2-(1-chlorocyclopropyl)-4-[(1R)-2,2-dichlorocyclopropyl]-1-(1H-1,2,4-triazol-1-yl)butan-2-ol, (A93) (1S,2R,5R)-5-(4-chlorobenz yl)-2-(chloromethyl)-2-methyl-1-(1H-1,2,4-triazol-1-ylmethyl)cyclopentanol, (A94) (1R,2S,5S)-5-(4-chlorobenzyl)-2-(chloromethyl)-2-methyl-1-(1H-1,2,4-triazol-1-ylmethyl)cyclopentanol, (A95) 5-(4-chlorobenz yl)-2-(chloromethyl)-2-methyl-1-(1H-1,2,4-triazol-1-ylmethyl)cyclopentanol, Other Sterol biosynthesis inhibitors: (A96) chlorphenomizole

B) Inhibitors of the respiratory chain at complex I or II, for example (B01) bixafen, (B02) boscalid, (B03) carboxin, (B04) cypropamide, (B05) diflumetorim, (B06) fenfuram, (B07) fluopyram, (B08) flutolanil, (B09) fluxapyroxad, (B10) furametpyr, (B11) furmecyclox, (B12) isopyrazam (mixture of syn-epimeric racemate 1RS,4SR,9RS and anti-epimeric racemate 1RS,4SR,9SR), (B13) isopyrazam (anti-epimeric racemate 1RS,4SR,9SR), (B14) isopyrazam (anti-epimeric enantiomer 1R,4S,9S), (B15) isopyrazam (anti-epimeric enantiomer 1S,4R,9R), (B16) isopyrazam (syn-epimeric racemate 1RS,4SR,9RS), (B17) isopyrazam (syn-epimeric enantiomer 1R,4S,9R), (B18) isopyrazam (syn-epimeric enantiomer 1S,4R,9S), (B19) mepronil, (B20) oxycarboxin, (B21) penflufen, (B22) penthiopyrad, (B23) pydiflumetofen, (B24) sedaxane, (B25) thifluzamide, (B26) 1-methyl-N-[2-(1,1,2,2-tetrafluoroethoxy)phenyl]-3-(trifluoromethyl)-1H-pyrazole-4-carboxamide, (B27) 3-(difluoromethyl)-1-methyl-N-[2-(1,1,2,2-tetrafluoroethoxy)phenyl]-1H-pyrazole-4-carboxamide, (B28) 3-(difluoromethyl)-N-[4-fluoro-2-(1,1,2,3,3,3-hexafluoropropoxy)phenyl]-1-methyl-1H-pyrazole-4-carboxamide, (B29) N-[1-(2,4-dichlorophenyl)-1-methoxypropan-2-yl]-3-(difluoromethyl)-1-methyl-1H-pyrazole-4-carboxamide, (B30) 5,8-difluoro-N-[2-(2-fluoro-4-{[4-(trifluoromethyl)pyridin-2-yl]oxy}pyhenyl)ethyl]quinazolin-4-amine, (B31) benzovindiflupyr, (B32) N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, (B33) N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (B34) 3-(difluoromethyl)-1-methyl-N-(1,1,3-trimethyl-2,3-dihydro-1H-inden-4-yl)-1H-pyrazole-4-carboxamide, (B35) 1,3,5-trimethyl-N-(1,1,3-trimethyl-2,3-dihydro-1H-inden-4-yl)-1H-pyrazole-4-carb oxamide, (B36) 1-methyl-3-(trifluoromethyl)-N-(1,1,3-trimethyl-2,3-dihydro-1H-inden-4-yl)-1H-pyrazole-4-carboxamide, (B37) 1-methyl-3-(trifluoromethyl)-N-[(3R)-1,1,3-trimethyl-2,3-dihydro-1H-inden-4-yl]-1H-pyrazole-4-carboxamide, (B38) 1-methyl-3-(trifluoromethyl)-N-[(3S)-1,1,3-trimethyl-2,3-dihydro-1H-inden-4-yl]-1H-pyrazole-4-carboxamide, (B39) 3-(difluoromethyl)-1-methyl-N-[(3S)-1,1,3-trimethyl-2,3-dihydro-1H-inden-4-yl]-1H-pyrazole-4-carboxamide, (B40) 3-(difluoromethyl)-1-methyl-N-[(3R)-1,1,3-trimethyl-2,3-dihydro-1H-inden-4-yl]-1H-pyrazole-4-carboxamide, (B41) 1,3,5-trimethyl-N-[(3R)-1,1,3-trimethyl-2,3-dihydro-1H-inden-4-yl]-1H-pyrazole-4-carboxamide, (B42) 1,3,5-trimethyl-N-[(3S)-1,1,3-trimethyl-2,3-dihydro-1H-inden-4-yl]-1H-pyrazole-4-carboxamide, (B43) benodanil, (B44) 2-chloro-N-(1,1,3-trimethyl-2,3-dihydro-1H-inden-4-yl)pyridine-3-carboxamide, (B45) Isofetamid, (B46) 1-methyl-3-(trifluoromethyl)-N-[2′-(trifluoromethyl)biphenyl-2-yl]-1H-pyrazole-4-carboxamide, (B47) N-(4′-chlorobiphenyl-2-yl)-3-(difluoromethyl)-1-methyl-1H-pyrazole-4-carboxamide, (B48) N-(2′,4′-dichlorobiphenyl-2-yl)-3-(difluoromethyl)-1-methyl-1H-pyrazole-4-carboxamide, (B49) 3-(difluoromethyl)-1-methyl-N-[4′-(trifluoromethyl)biphenyl-2-yl]-1H-pyrazole-4-carboxamide, (B50) N-(2′,5′-difluorobiphenyl-2-yl)-1-methyl-3-(trifluoromethyl)-1H-pyrazole-4-carboxamide, (B51) 3-(difluoromethyl)-1-methyl-N-[4′-(prop-1-yn-1-yl)biphenyl-2-yl]-1H-pyrazole-4-carboxamide, (B52) 5-fluoro-1,3-dimethyl-N-[4′-(prop-1-yn-1-yl)biphenyl-2-yl]-1H-pyrazole-4-carboxamide, (B53) 2-chloro-N-[4′-(prop-1-yn-1-yl)biphenyl-2-yl]nicotinamide, (B54) 3-(difluoromethyl)-N-[4′-(3,3-dimethylbut-1-yn-1-yl)biphenyl-2-yl]-1-methyl-1H-pyrazole-4-carboxamide, (B55) N-[4′-(3,3-dimethylbut-1-yn-1-yl)biphenyl-2-yl]-5-fluoro-1,3-dimethyl-1H-pyrazole-4-carboxamide, (B56) 3-(difluoromethyl)-N-(4′-ethynylbiphenyl-2-yl)-1-methyl-1H-pyrazole-4-carboxamide, (B57) N-(4′-ethynylbiphenyl-2-yl)-5-fluoro-1,3-dimethyl-1H-pyrazole-4-carboxamide, (B58) 2-chloro-N-(4′-ethynylbiphenyl-2-yl)nicotinamide, (B59) 2-chloro-N-[4′-(3,3-dimethylbut-1-yn-1-yl)biphenyl-2-yl]nicotinamide, (B60) 4-(difluoromethyl)-2-methyl-N-[4′-(trifluoromethyl)biphenyl-2-yl]-1,3-thiazole-5-carboxamide, (B61) 5-fluoro-N-[4′-(3-hydroxy-3-methylbut-1-yn-1-yl)biphenyl-2-yl]-1,3-dimethyl-1H-pyrazole-4-carboxamide, (B62) 2-chloro-N-[4′-(3-hydroxy-3-methylbut-1-yn-1-yl)biphenyl-2-yl]nicotinamide, (B63) 3-(difluoromethyl)-N-[4′-(3-methoxy-3-methylbut-1-yn-1-yl)biphenyl-2-yl]-1-methyl-1H-pyrazole-4-carboxamide, (B64) 5-fluoro-N-[4′-(3-methoxy-3-methylbut-1-yn-1-yl)biphenyl-2-yl]-1,3-dimethyl-1H-pyrazole-4-carboxamide, (B65) 2-chloro-N-[4′-(3-methoxy-3-methylbut-1-yn-1-yl)biphenyl-2-yl]nicotinamide, (B66) 1,3-dimethyl-N-(1,1,3-trimethyl-2,3-dihydro-1H-inden-4-yl)-1H-pyrazole-4-carboxamide, (B67) 1,3-dimethyl-N-[(3R)-1,1,3-trimethyl-2,3-dihydro-1H-inden-4-yl]-1H-pyrazole-4-carboxamide, (B68) 1,3-di methyl-N-[(3S)-1,1,3-trimethyl-2,3-dihydro-1H-inden-4-yl]-1H-pyrazole-4-carboxamide, (B69) 3-(difluoromethyl)-N-methoxy-1-methyl-N-[1-(2,4,6-trichlorophenyepropan-2-yl]-1H-pyrazole-4-carboxamide, (B70) 3-(difluoromethyl)-N-(7-fluoro-1,1,3-trimethyl-2,3-dihydro-1H-inden-4-yl)-1-methyl-1H-pyrazole-4-carboxamide, (B71) 3-(difluoromethyl)-N-[(3,R)-7-fluoro-1,1,3-trimethyl-2,3-dihydro-1H-inden-4-yl]-1-methyl-1H-pyrazole-4-carboxamide, (B72) 3-(difluoromethyl)-N-[(3S)-7-fluoro-1,1,3-trimethyl-2,3-dihydro-1H-inden-4-yl]-1-methyl-1H-pyrazole-4-carboxamide.

C) Inhibitors of the respiratory chain at complex III, for example (C01) ametoctradin, (C02) amisulbrom, (C03) azoxystrobin, (C04) cyazofamid, (C05) coumethoxystrobin, (C06) coumoxystrobin, (C07) dimoxystrobin, (C08) enoxastrobin, (C09) famoxadone, (C10) fenamidone, (C11) fenaminstrobin, (C12) flufenoxystrobin, (C13) fluoxastrobin, (C14) kresoxim-methyl, (C15) metominostrobin, (C16) mandestrobin, (C17) orysastrobin, (C18) picoxystrobin, (C19) pyraclostrobin, (C20) pyrametostrobin, (C21) pyraoxystrobin, (C22) pyribencarb, (C23) triclopyricarb, (C24) trifloxystrobin, (C25) (2E)-2-(2-{[6-(3-chloro-2-methylphenoxy)-5-fluoropyrimidin-4-yl]oxy}phenyl)-2-(methoxyimino)-N-methylacetamide, (C26) (2E)-2-(methoxyimino)-N-methyl-2-(2-{[({(1E)-1-[3-(trifluoromethyl)phenyl]ethylidene} amino)oxy]methyl} phenyl) acetamide, (C27) (2E)-2-(methoxyimino)-N-methyl-2-{2-[(E)-({1-[3-(trifluoromethyl)phenyl]ethoxy}imino)methyl]phenyl}acetamide, (C28) (2E)-2-{2-[({[(1E)-1-(3-{[(E)-1-fluoro-2-phenylvinyl]oxy} phenyl)ethylidene] amino}oxy)methyl]phenyl}-2-(methoxyimino)-N-methylacetamide, (C29) Fenaminostrobin, (C30) 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, (C31) methyl (2E)-2-{2-[({cyclopropyl[(4-methoxyphenyl)imino]methyl}sulfanyl)methyl]phenyl}-3-methoxyacrylate, (C32) N-(3-ethyl-3,5,5-trimethylcyclohexyl)-3-formamido-2-hydroxybenzamide, (C33) 2-{2-[(2,5-dimethylphenoxy)methyl]phenyl}-2-methoxy-N-methylacetamide, (C34) 2-{2-[(2,5-dimethylphenoxy)methyl]phenyl}-2-methoxy-N-methylacetamide, (C35) (2E,3Z)-5-{[1-(4-chlorophenyl)-1H-pyrazol-3-yl]oxy}-2-(methoxyimino)-N,3-dimethylpent-3-enamide

D) Inhibitors of the mitosis and cell division, for example (D01) benomyl, (D02) carbendazim, (D03) chlorfenazole, (D04) diethofencarb, (D05) ethaboxam, (D06) fluopicolide, (D07) fiiberidazole, (D08) pencycuron, (D09) thiabendazole, (D10) thiophanate-methyl, (D11) thiophanate, (D12) zoxamide, (D13) 5-chloro-7-(4-methylpiperidin-1-yl)-6-(2,4,6-trifluorophenyl)[1,2,4]triazolo[1,5-a]pyrimidine, (D14) 3-chloro-5-(6-chloropyridin-3-yl)-6-methyl-4-(2,4,6-trifluorophenyl) pyridazine, (D15) 3-chloro-4-(2,6-difluorophenyl)-6-methyl-5-phenyl-pyridazine, (D16) 3-chloro-6-methyl-1-phenyl-4-(2,4,6-trifluorophenyl)pyridazine, (D17) N-ethyl-2-[(3-thynyl-8-methyl-6-quinolyl)oxy]butanamide, (D18) N-ethyl-2-[(3-ethynyl-8-methyl-6-quinolyl)oxy]-2-methylsulfanyl-acetamide, (D19) 2-[(3-ethynyl-8-methyl-6-quinolyl)oxy]-N-(2-fluoroethyl)butanamide, (D20) 2-[(3-ethynyl-8-methyl-6-quinolyl)oxy]-N-(2-fluoroethyl)-2-methoxy-acetamide, (D21) 2-[(3-ethynyl-8-methyl-6-quinolyl)oxy]-N-propyl-butanamide, (D22) 2-[(3-ethynyl-8-methyl-6-quinolyl)oxy]-2-methoxy-N-propyl-acetamide, (D23) 2-[(3-ethynyl-8-methyl-6-quinolyl)oxy]-2-methylsulfanyl-N-propyl-acetamide, (D24) 2-[(3-ethynyl-8-methyl-6-quinolyl)oxy]-N-(2-fluoroethyl)-2-methylsulfanyl-acetamide, (D25) 4-(2-bromo-4-fluoro-phenyl)-N-(2-chloro-6-fluoro-phenyl)-2,5-dimethyl-pyrazol-3-amine;

E) Compounds capable to have a multisite action, for example (E01) bordeaux mixture, (E02) captafol, (E03) captan, (E04) chlorothalonil, (E05) copper hydroxide, (E06) copper naphthenate, (E07) copper oxide, (E08) copper oxychloride, (E09) copper(2+) sulfate, (E10) dichlofluanid, (E11) dithianon, (E12) dodine, (E13) dodine free base, (E14) ferbam, (E15) fluorofolpet, (E16) folpet, (E17) guazatine, (E18) guazatine acetate, (E19) iminoctadine, (E20) iminoctadine albesilate, (E21) iminoctadine triacetate, (E22) mancopper, (E23) mancozeb, (E24) maneb, (E25) metiram, (E26) metiram zinc, (E27) oxine-copper, (E28) propamidine, (E29) propineb, (E30) sulfur and sulfur preparations including calcium polysulfide, (E31) thiram, (E32) tolylfluanid, (E33) zineb, (E34) ziram, (E35) anilazine.

F) Compounds capable to induce a host defence, for example (F01) acibenzolar-S-methyl, (F02) isotianil, (F03) probenazole, (F04) tiadinil, (F05) laminarin, (F06) 4-cyclopropyl-N-(2,4-dimethoxyphenyl)thiadiazole-5-carboxamide.

G) Inhibitors of the amino acid and/or protein biosynthesis, for example (G01) andoprim, (G02) blasticidin-S, (G03) cyprodinil, (G04) kasugamycin, (G05) kasugamycin hydrochloride hydrate, (G06) mepanipyrim, (G07) pyrimethanil, (G08) 3-(5-fluoro-3,3,4,4-tetramethyl-3,4-dihydroisoquinolin-1-yl)quinoline, (G09)oxytetracycline, (G10) streptomycin.

H) Inhibitors of the ATP production, for example (H01) fentin acetate, (H02) fentin chloride, (H03) fentinhydroxide, (H04) silthiofam.

I) Inhibitors of the cell wall synthesis, for example (I01) benthiavalicarb, (I02) dimethomorph, (I03) flumorph, (I04) iprovalicarb, (I05) mandipropamid, (I06) polyoxins, (I07) polyoxorim, (I08) validamycin A, (I09) valifenalate, (I10) polyoxin B, (I11) (2E)-3-(4-tert-butylphenyl)-3-(2-chloropyridin-4-yl)-1-(morpholin-4-yl)prop-2-en-1-one, (I12) (2Z)-3-(4-tert-butylphenyl)-3-(2-chloropyridin-4-yl)-1-(morpholin-4-yl)prop-2-en-1-one.

J) Inhibitors of the lipid and membrane synthesis, for example (J01) biphenyl, (J02) chloroneb, (J03) dicloran, (J04) edifenphos, (J05) etridiazole, (J06) iodocarb, (J07) iprobenfos, (J08) isoprothiolane, (J09) propamocarb, (J10) propamocarb hydrochloride, (J11) prothiocarb, (J12) pyrazophos, (J13) quintozene, (J14) tecnazene, (J15) toclofos-methyl.

K) Inhibitors of the melanin biosynthesis, for example (K01) carpropamid, (K02) diclocymet, (K03) fenoxanil, (K04) phthalide, (K05) pyroquilon, (K06) tolprocarb, (K07)tricyclazole.

L) Inhibitors of the nucleic acid synthesis, for example (L01) benalaxyl, (L02) benalaxyl-M (kiralaxyl), (L03) bupirimate, (L04) clozylacon, (L05) dimethirimol, (L06) ethirimol, (L07) furalaxyl, (L08) hymexazol, (L09) metalaxyl, (L10) metalaxyl-M (mefenoxam), (L11) ofurace, (L12) oxadixyl, (L13) oxolinic acid, (L14)octhilinone.

M) Inhibitors of the signal transduction, for example (M01) chlozolinate, (M02) fenpiclonil, (M03) fludioxonil, (M04) iprodione, (M05) procymidone, (M06) quinoxyfen, (M07) vinclozolin, (M08) proquinazid.

N) Compounds capable to act as an uncoupler, for example (N01) binapacryl, (N02) dinocap, (N03) ferimzone, (N04) fluazinam, (N05) meptyldinocap.

O) Further compounds, for example (O01) benthiazole, (O02) bethoxazin, (O03) capsimycin, (O04) carvone, (O05) chinomethionat, (O06) pyriofenone (chlazafenone), (O07) cufraneb, (O08) cyflufenamid, (O09) cymoxanil, (O10) cyprosulfamide, (O11) dazomet, (O12) debacarb, (O13) dichlorophen, (O14) dichlobentiazox, (O15) diclomezine, (O16) difenzoquat, (O17) difenzoquat metilsulfate, (O18) diphenylamine, (O19) ecomate, (O20) fenpyrazamine, (O21) fenhexamine, (O22) flumetover, (O23) fluoroimide, (O24) flusulfamide, (O25) flutianil, (O26) fosetyl-aluminium, (O27) fosetyl-calcium, (O28) fosetyl-sodium, (O29) hexachlorobenzene, (O30) irumamycin, (O31) isothianil, (O32) methasulfocarb, (O33) methyl isothiocyanate, (O34) metrafenone, (O35) mildiomycin, (O36) natamycin, (O37) nickel dimethyldithiocarbamate, (O38) nitrothal-isopropyl, (O39) oxamocarb, (O40) oxyfenthiin, (O41) pentachlorophenol and salts, (O42) phenothrin, (O43) picarbutrazox (O44) phosphorous acid and its salts, (O45) propamocarb-fosetylate, (O46) propanosine-sodium, (O47) pyrimorph, (O48) pyraziflumid (O49) pyrrolnitrine, (O50) tebufloquin, (O51) tecloftalam, (O52) tolnifanide, (O53) triazoxide, (O54) trichlamide, (O55) zarilamid, (O56) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (O57) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (O58) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (O59) dichlobentiazox, (O60) 1-(4-methoxyphenoxy)-3,3-dimethylbutan-2-yl-1H-imidazole-1-carboxylate, (O61) 2,3,5,6-tetrachloro-4-(methylsulfonyl)pyridine, (O62) 2,3-dibutyl-6-chlorothieno[2,3-d]pyrimidin-4(3H)-one, (O63) 2,6-dimethyl-1H,5H-[1,4]dithiino[2,3-c:5,6-c′]dipyrrole-1,3,5,7(2H,6H)-tetrone, (O64) 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone, (O65) 2-[5-methyl-3-(trifluoromethyl)-1H-pyrazol-1-yl]-1-(4-{4-[(5S)-5-phenyl-4,5-dihydro-1,2-oxazol-3-yl]-1,3-thiazol-2-yl}piperidin-1-yl)ethanone, (O66) 2-[5-methyl-3-(trifluoromethyl)-1H-pyrazol-]-yl]-1-{4-[4-(5-phenyl-4,5-dihydro-1,2-oxazol-3-yl)-1,3-thiazol-2-yl]piperidin-1-yl}ethanone, (O67) 2-butoxy-6-iodo-3-prop yl-4H-chromen-4-one, (O68) 2-chloro-5-[2-chloro-1-(2,6-difluoro-4-methoxyphenyl)-4-methyl-1H-imidazol-5-yl]pyridine, (O69) 2-phenylphenol and salts, (O70) 3-(4,4,5-trifluoro-3,3-dimethyl-3,4-dihydroisoquinolin-1-yl)quinoline, (O71) 3,4,5-trichloropyridine-2,6-dicarbonitrile, (O72) 3-chloro-5-(4-chlorophenyl)-4-(2,6-difluorophenyl)-6-methylpyridazine, (O73) 4-(4-chlorophenyl)-5-(2,6-difluorophenyl)-3,6-dimethylpyridazine, (O74) 3-chloro-4-(2,6-difluorophenyl)-6-methyl-5-phenylpyridazine, (O75) 5-amino-1,3,4-thiadiazole-2-thiol, (O76) 5-chloro-N′-phenyl-N′-(prop-2-yn-1-yl)thiophene-2-sulfonohydrazide, (O77) 5-fluoro-2-[(4-fluorobenzyl)oxy]pyrimidin-4-amine, (O78) 5-fluoro-2-[(4-methylbenzyl)oxy]pyrimidin-4-amine, (O79) 5-methyl-6-octyl[1,2,4]triazolo[1,5-a]pyrimidin-7-amine, (O80) ethyl (2Z)-3-amino-2-cyano-3-phenylacrylate, (O81) N′-(4-{[3-(4-chlorobenzyl)-1,2,4-thiadiazol-5-yl]oxy}-2,5-dimethylphenyl)-N-ethyl-N-methylimidoformamide, (O82) N-(4-chlorobenzyl)-3-[3-methoxy-4-(prop-2-yn-1-yloxy)phen yl]propanamide, (O83) N-[(4-chlorophen yl)(cyano)methyl]-3-[3-methoxy-4-(prop-2-yn-1-yloxy)phen yl]propanamide, (O84) N-[(5-bromo-3-chloropyridin-2-yl)methyl]-2,4-dichloronicotinamide, (O85) N-[1-(5-bromo-3-chloropyridin-2-yl)ethyl]-2,4-dichloronicotinamide, (O86) N-[1-(5-bromo-3-chloropyridin-2-yl)ethyl]-2-fluoro-4-iodonicotinamide, (O87) N-{(E)-[(cyclopropylmethoxy)imino][6-(difluoromethoxy)-2,3-difluorophenyl]methyl}-2-phenylacetamide, (O88) N—{(Z)-[(cyclopropylmethoxy)imino][6-(difluoromethoxy)-2,3-difluorophenyl] methyl}-2-phenylacetamide, (O89) N′-{4-[(3-tert-butyl-4-cyano-1,2-thiazol-5-yl)oxy]-2-chloro-5-methylphenyl}-N-ethyl-N-methylimidoformamide, (O90) N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide, (O91) N-methyl-2-(1-{[5-methyl-3-(trifluoromethyl)-1H-pyrazol-1-yl]acetyl}piperidin-4-yl)-N-[(1R)-1,2,3,4-tetrahydronaphthalen-1-yl]-1,3-thiazole-4-carboxamide, (O92) N-methyl-2-(1-{[5-methyl-3-(trifluoromethyl)-1H-pyrazol-1-yl]acetyl}piperidin-4-yl)-N-[(1S)-1,2,3,4-tetrahydronaphthalen-1-yl]-1,3-thiazole-4-carboxamide, (O93) pentyl {6-[({[(1-methyl-1H-tetrazol-5-yl)(phenyl)methylene]amino}oxy)methyl]pyridin-2-yl} carbamate, (O94) phenazine-1-carboxylic acid, (O95) quinolin-8-ol, (O96) quinolin-8-ol sulfate (2:1), (O97) tert-butyl {6-[({[(1-methyl-1H-tetrazol-5-yl)(phenyl) methylene]amino}oxy)methyl]pyridin-2-yl}carbamate, (O98) (5-bromo-2-methoxy-4-methylpyridin-3-yl)(2,3,4-trimethoxy-6-methylphenyl) methanone, (O99) N-[2-(4-{[3-(4-chlorophenyl)prop-2-yn-1-yl]oxy}-3-methoxyphenyl)thyl]-N2-(methylsulfonyl)valinamide, (O100) 4-oxo-4-[(2-phenylethyl)amino]butanoic acid, (O101) but-3-yn-1-yl {6-[(1 [(Z)-(1-methyl-1H-tetrazol-5-yl)(phenyl)methylene]aminol oxy)methyl]pyridin-2-yl} carbamate, (O102) 4-amino-5-fluoropyrimidin-2-ol (tautomeric form: 4-amino-5-fluoropyrimidin-2(1H)-one), (O103) prop yl 3,4,5-trihydroxybenzoate, (O104) [3-(4-chloro-2-fluorophenyl)-5-(2,4-difluorophenyl)-1,2-oxazol-4-yl](pyridin-3-yl)methanol, (O105) (S)-[3-(4-chloro-2-fluorophenyl)-5-(2,4-difluorophenyl)-1,2-oxazol-4-yl](pyridin-3-yl)methanol, (O106) (R)-[3-(4-chloro-2-fluorophenyl)-5-(2,4-difluorophenyl)-1,2-oxazol-4-yl](pyridin-3-yl)methanol, (O107) 2-fluoro-6-(trifluoromethyl)-N-(1,1,3-trimethyl-2,3-dihydro-1H-inden-4-yl)benzamide, (O108) 2-(6-benzylpyridin-2-yl)quinazoline, (O109) 2-[6-(3-fluoro-4-methoxyphenyl)-5-methylpyridin-2-yl]quinazoline, (O110) 3-(4,4-difluoro-3,3-dimethyl-3,4-dihydroisoquinolin-1-yl)quinoline, (O111) Abscisic acid, (O112) N′-[5-bromo-6-(2,3-dihydro-1H-inden-2-yloxy)-2-methylpyridin-3-yl]-N-ethyl-N-methylimidoformamide, (O113) N′-{5-bromo-6-[1-(3,5-difluorophenyl)ethoxy]-2-methylpyridin-3-yl}-N-ethyl-N-methylimidoformamide, (O114) N′-{5-bromo-6-[(1R)-1-(3,5-difluorophenyl)ethoxy]-2-methylpyridin-3-yl}-N-ethyl-N-methylimidoformamide, (O115) N′-{5-bromo-6-[(1S)-1-(3,5-difluorophenyl)ethoxy]-2-methylpyridin-3-yl}-N-ethyl-N-methylimidoformamide, (O116) N′-{5-bromo-6-[(cis-4-isopropylcyclohexyl)oxy]-2-methylpyridin-3-yl}-N-ethyl-N-methylimidoformamide, (O117) N′-{5-bromo-6-[(trans-4-isopropylcyclohexyl)oxy]-2-methylpyridin-3-yl}-N-ethyl-N-methylimidoformamide, (O118) N-cyclopropyl-3-(difluoromethyl)-5-fluoro-N-(2-isopropylbenzyl)-1-methyl-1H-pyrazole-4-carboxamide, (O119) N-cyclopropyl-N-(2-cyclopropylbenzyl)-3-(difluoromethyl)-5-fluoro-1-methyl-1H-pyrazole-4-carboxamide, (O120) N-(2-tert-butylbenzyl)-N-cyclopropyl-3-(difluoromethyl)-5-fluoro-1-methyl-1H-pyrazole-4-carboxamide, (O121) N-(5-chloro-2-ethylbenzyl)-N-cyclopropyl-3-(difluoromethyl)-5-fluoro-1-methyl-1H-pyrazole-4-carboxamide, (O122) N-(5-chloro-2-isopropylbenzyl)-N-cyclopropyl-3-(difluoromethyl)-5-fluoro-1-methyl-1H-pyrazole-4-carboxamide, (O123) N-cyclopropyl-3-(difluoromethyl)-N-(2-ethyl-5-fluorobenzyl)-5-fluoro-1-methyl-1H-pyrazole-4-carb oxamide, (O124) N-cyclopropyl-3-(difluoromethyl)-5-fluoro-N-(5-fluoro-2-isopropylbenzyl)-1-methyl-1H-pyrazole-4-carboxamide, (O125) N-cyclopropyl-N-(2-cyclopropyl-5-fluorobenz yl)-3-(difluoromethyl)-5-fluoro-1-methyl-1H-pyrazole-4-carboxamide, (O126) N-(2-cyclopentyl-5-fluorobenzyl)-N-cyclopropy-3-(difluoromethyl)-5-fluoro-1-methyl-1H-pyrazole-4-carboxamide, (O127) N-cyclopropyl-3-(difluoromethyl)-5-fluoro-N-(2-fluoro-6-isopropylbenzyl)-1-methyl-1H-pyrazole-4-carb oxamide, (O128) N-cyclopropyl-3-(difluoromethyl)-N-(2-ethyl-5-methylbenzyl)-5-fluoro-1-methyl-1H-pyrazole-4-carboxamide, (O129) N-cyclopropyl-3-(difluoromethyl)-5-fluoro-N-(2-isopropyl-5-methylbenzyl)-1-methyl-1H-pyrazole-4-carboxamide, (O130) N-cyclopropyl-N-(2-cyclopropyl-5-methylbenzyl)-3-(difluoromethyl)-5-fluoro-1-methyl-1H-pyrazole-4-carboxamide, (O131) N-(2-tert-butyl-5-methylbenzyl)-N-cyclopropyl-3-(difluoromethyl)-5-fluoro-1-methyl-1H-pyrazole-4-carboxamide, (O132) N-[5-chloro-2-(trifluoromethyl)benzyl]-N-cyclopropyl-3-(difluoromethyl)-5-fluoro-1-methyl-1H-pyrazole-4-carboxamide, (O133) N-cyclopropyl-3-(difluoromethyl)-5-fluoro-1-methyl-N-[5-methyl-2-(trifluoromethyl)benzyl]-1H-pyrazole-4-carboxamide, (O134) N-[2-chloro-6-(trifluoromethyl)benzyl]-N-cyclopropyl-3-(difluoromethyl)-5-fluoro-1-methyl-1H-pyrazole-4-carboxamide, (O135) N-[3-chloro-2-fluoro-6-(trifluoromethyl)benzyl]-N-cyclopropyl-3-(difluoromethyl)-5-fluoro-1-methyl-1H-pyrazole-4-carboxamide, (O136) N-cyclopropyl-3-(difluoromethyl)-N-(2-ethyl-4,5-dimethylbenzyl)-5-fluoro-1-methyl-1H-pyrazole-4-carboxamide, (O137) N-cyclopropyl-3-(difluoromethyl)-5-fluoro-N-(2-isopropylbenzyl)-1-methyl-1H-pyrazole-4-carbothioamide, (O138) N′-(2,5-dimethyl-4-phenoxyphenyl)-N-ethyl-N-methylimidoformamide, (O139) N′-{4-[(4,5-dichloro-1,3-thiazol-2-yl)oxy]-2,5-dimethylphenyl}-N-ethyl-N-methylimidoformamide, (O140) N-(4-chloro-2,6-difluorophenyl)-4-(2-chloro-4-fluorophenyl)-1,3-di methyl-1H-pyrazol-5-amine, (O141) 9-fluoro-2,2-dimethyl-5-(quinolin-3-yl)-2,3-dihydro-1,4-benzoxazepine, (O142) 2-{2-fluoro-6-[(8-fluoro-2-methylquinolin-3-yl)oxy]phenyl}propan-2-ol, (O143) 2-{2-[(7,8-difluoro-2-methylquinolin-3-yl)oxy]-6-fluorophenyl}propan-2-ol, (O144) 4-(2-chloro-4-fluorophenyl)-N-(2-fluorophenyl)-1,3-dimethyl-1H-pyrazol-5-amine, (O145) 4-(2-chloro-4-fluorophenyl)-N-(2,6-difluorophenyl)-1,3-dimethyl-1H-pyrazol-5-amine, (O146) 4-(2-chloro-4-fluorophenyl)-N-(2-chloro-6-fluorophenyl)-1,3-dimethyl-1H-pyrazol-5-amine, (O147) 4-(2-bromo-4-fluorophenyl)-N-(2-chloro-6-fluorophenyl)-1,3-dimethyl-1H-pyrazol-5-amine, (O148) N-(2-bromo-6-fluorophenyl)-4-(2-chloro-4-fluorophenyl)-1,3-dimethyl-1H-pyrazol-5-amine, (O149) 4-(2-bromo-4-fluorophenyl)-N-(2-bromophenyl)-1,3-dimethyl-1H-pyrazol-5-amine, (O150) 4-(2-bromo-4-fluorophenyl)-N-(2-bromo-6-fluorophenyl)-1,3-dimethyl-1H-pyrazol-5-amine, (O151) 4-(2-bromo-4-fluorophenyl)-N-(2-chlorophenyl)-1,3-dimethyl-1H-pyrazol-5-amine, (O152) N-(2-bromophenyl)-4-(2-chloro-4-fluorophenyl)-1, 3-dimethyl-1H-pyrazol-5-amine, (O153) 4-(2-chloro-4-fluorophenyl)-N-(2-chlorophenyl)-1,3-dimethyl-1H-pyrazol-5-amine, (O154) 4-(2-bromo-4-fluorophenyl)-N-(2,6-difluorophenyl)-1,3-dimethyl-1H-pyrazol-5-amine, (O155) 4-(2-bromo-4-fluorophenyl)-N-(2-fluorophenyl)-1,3-dimethyl-1H-pyrazol-5-amine, (O156) N′-(4-{3-[(difluoromethyl)sulfanyl]phenoxy}-2,5-dimethylphenyl)-N-ethyl-N-methylimidoformamide, (O157) N′-(2,5-dimethyl-4-{3-[(1,1,2,2-tetrafluoroethyl)sulfanyl]phenoxy}phenyl)-N-ethyl-N-methylimidoformamide, (O158) N′-(2,5-dimethyl-4-{3-[(2,2,2-trifluoroethyl)sulfanyl]phenoxy}phenyl)-N-ethyl-N-methylimidoformamide, (O159) N′-(2,5-dimethyl-4-{3-[(2,2,3,3-tetrafluoropropyl)sulfanyl]phenoxy}phenyl)-N-ethyl-N-methylimidoformamide, (O160) N′-(2,5-dimethyl-4-{3-[(pentafluoroethyl)sulfanyl]phenoxy}phenyl)-N-ethyl-N-methylimidoformamide, (O161) N′-(4-{[3-(difluoromethoxy)phenyl]sulfanyl}-2,5-dimethylphenyl)-N-ethyl-N-methylimidoformamide, (O162) N′-(2,5-dimethyl-4-{[3-(1,1,2,2-tetrafluoroethoxy)phenyl]sulfanyl}phenyl)-N-ethyl-N-methylimidoformamide, (O163) N′-(2,5-dimethyl-4-{[3-(2,2,2-trifluoroethoxy)phenyl]sulfanyl}phenyl)-N-ethyl-N-methylimidoformamide, (O164) N′-(2,5-dimethyl-4-{[3-(2,2,3,3-tetrafluoropropoxy)phenyl]sulfanyl}phenyl)-N-ethyl-N-methylimidoformamide, (O165) N′-(2,5-dimethyl-4-{[3-(pentafluoroethoxy)phenyl]sulfanyl}phenyl)-N-ethyl-N-methylimidoformamide, (O166) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (O167) 2-[3,5-bis(difluoromethyl)-1H-pyrazol-1-yl]-1-[4-(4-{5-[2-fluoro-6-(prop-2-yn-1-yloxy)phen yl]-4,5-dihydro-1,2-oxazol-3-yl}-1,3-thiazol-2-yl)piperidin-1-yl]ethanone, (O168) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (O169) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, (O170) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (O171) 2-[3,5-bis(difluoromethyl)-1H-pyrazol-1-yl]-1-[4-(4-{(5S)-5-[2-(prop-2-yn-1-yloxy)phenyl]-4,5-dihydro-1,2-oxazol-3-yl}-1,3-thiazol-2-yl)piperidin-1-yl]ethanone, (O172) 2-[3,5-bis(difluoromethyl)-1H-pyrazol-1-yl]-1-[4-(4-{(5R)-5-[2-(prop-2-yn-1-yloxy)phenyl]-4,5-dihydro-1,2-oxazol-3-yl}-1,3-thiazol-2-yl)piperidin-1-yl]ethanone, (O173) 2-[3,5-bis(difluoromethyl)-1H-pyrazol-1-yl]-1-[4-(4-{(5S)-5-[2-fluoro-6-(prop-2-yn-1-yloxy)phenyl]-4,5-dihydro-1,2-oxazol-3-yl}-1,3-thiazol-2-yl)piperidin-1-yl]ethanone, (O174) 2-[3,5-bis(difluoromethyl)-1H-pyrazol-1-yl]-1-[4-(4-{(5R)-5-[2-fluoro-6-(prop-2-yn-1-yloxy)phenyl]-4,5-dihydro-1,2-oxazol-3-yl}-1,3-thiazol-2-yl)piperidin-1-yl]ethanone, (O175) 2-[3,5-bis(difluoromethyl)-1H-pyrazol-1-yl]-1-[4-(4-{(5S)-5-[2-chloro-6-(prop-2-yn-1-yloxy)phenyl]-4,5-dihydro-1,2-oxazol-3-yl}-1,3-thiazol-2-yl)piperidin-1-yl]ethanone, (O176) 2-[3,5-bis(difluoromethyl)-1H-pyrazol-1-yl]-1-[4-(4-{(5R)-5-[2-chloro-6-(prop-2-yn-1-yloxy)phenyl]-4,5-dihydro-1,2-oxazol-3-yl}-1,3-thiazol-2-yl)piperidin-1-yl]ethanone, (O177) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, (O178) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, (O179) 2-{(5S)-3-[2-(1-[3,5-bis(difluoromethyl)-1H-pyrazol-1-yl]acetyl}piperidin-4-yl)-1,3-thiazol-4-yl]-4,5-dihydro-1,2-oxazol-5-yl-3-chlorophenylmethanesulfonate, (O180) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (O181) (3S,6S,7R,8R)-8-benzyl-3-{3-[(isobutyryloxy)methoxy]-4-methoxypicolinamido}-6-methyl-4,9-dioxo-1,5-dioxonan-7-yl isobutyrate, (O182) N′-(4-(4-chloro-3-trifluoromethyl-phenoxy)-2,5-dimethyl-phenyl)-N-ethyl-N-methyl formamidine, (O183) N′-(4-(4-fluoro-3-trifluoromethyl-phenoxy)-2,5-dimethyl-phenyl)-N-ethyl-N-methylformamidine, (O184) N′-[4-[[3-[(4-chlorophenyl)methyl]-1,2,4-thiadiazol-5-yl]oxy]-2,5-dimethyl-phenyl]-N-ethyl-N-methyl-formamidine, (O185) N′-(5-bromo-6-indan-2-yloxy-2-methyl-3-pyrid yl)-N-ethyl-N-methyl-formamidine, (O186) N′-[5-bromo-6-[1-(3,5-difluorophenyl)ethoxy]-2-methyl-3-pyridyl]-N-ethyl-N-methyl-formamidine, (O187) N′-[5-bromo-6-(4-isopropylcyclohexoxy)-2-methyl-3-pyridyl]-N-ethyl-N-methyl-formamidine, (O188) N′-[5-bromo-2-methyl-6-(1-phenylethoxy)-3-pyridyl]-N-ethyl-N-methylformamidine, (O189) N′-(2-methyl-5-trifluoromethyl-4-(3-trimethylsilanyl-propoxy)phenyl)-N-ethyl-N-methyl formamidine, (O190) N′-(5-difluoromethyl-2-methyl-4-(3-trimethylsilanyl-propoxy)-phenyl)-N-ethyl-N-methyl formamidine, (O191), (N-ethyl-N′-(4-(2-fluorophenoxy)-2,5-dimethylphenyl)-N-methylformimidamide, (O192) N′-(2-chloro-4-(2-fluorophenoxy)-5-methylphenyl)-N-ethyl-N-methylformimidamide, (O193) 2-[2-[(7,8-difluoro-2-methyl-3-quinolyl)oxy]-6-fluoro-phenyl]propan-2-01, (O194) 2-[2-fluoro-6-[(8-fluoro-2-methyl-3-quinolyl)oxy]-phen-yl]propan-2-ol, (O195) quinofumelin, (O196) 9-fluoro-2,2-dimethyl-5-(3-quinolyl)-3H-1,4-benzoxazepine, (O197) 2-(6-benzyl-2-pyridyl)quinazoline, (O198) 2-[6-(3-fluoro-4-methoxy-phenyl)-5-methyl-2-pyridyl]quinazoline, (O199) Fluopimomide, (O200) Florylpicoxamide.

P) Growth regulators, for example abscisic acid, amidochlor, ancymidol, 6-benzylaminopurine, brassinolide, butralin, chlormequat, chlormequat chloride, choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat, mepiquat chloride, naphthaleneacetic acid, N-6-benzyladenine, paclobutrazol, prohexadione, prohexadione-calcium, prohydrojasmon, thidiazuron, triapenthenol, tributyl phosphorotrithioate, 2,3,5-tri-iodobenzoic acid, trinexapac-ethyl and uniconazole;

As described above the compound of formula (I) can be mixed with one or more active compatible compound selected from insecticides/acaricides/nematicides class which are specified herein by their common names that are known and described, for example in The Pesticide Manual 17th Ed., or can be searched in the internee under www.alanwood.net/pesticides).

(1) Acetylcholinesterase (AChE) inhibitors such as carbamates, for example alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicatb, propoxur, thiodicarb, thiofanox, triazamate, trimethacarb, XMC and xylylcarb or organophosphates, such as acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfolon, EPN, ethion, ethoprophos, famphur, fenamiphos, fenitrothion, fenthion, fosthiazate, heptenophos, imicyafos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.

(2) GABA-gated chloride channel antagonists, such as cyclodiene organochlorines, for example chlordane and endosulfan or phenylpyrazoles (fiproles), for example ethiprole and fipronil.

(3) Sodium channel modulators/voltage-dependent sodium channel blockers, such as pyrethroids, for example acrinathrin, allethrin, d-cis-trans allethrin, d-irons allethrin, bifenthrin, bioallethrin, bioallethrin S-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans-isomers], deltamethrin, empenthrin [(EZ)-(1R)-isomers], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans-isomer), prallethrin, pyrethrins (pyrethrum), resmethrin, silafluofen, tefluthrin, tetramethrin, tetramethrin [(1R)-isomers)], tralomethrin and transfluthrin or DDT or methoxychlor.

(4) Nicotinic acetylcholine receptor (nAChR) competitive modulators, such as neonicotinoids, for example acetamiprid, clothianidin, dinotefuran, imidacloprid, nitenpyram, thiacloprid and thiamethoxam or nicotine or sulfoxaflor or flupyradifurone.

(5) Nicotinic acetylcholine receptor (nAChR) allosteric modulators, such as spinosyns, for example spinetoram and spinosad.

(6) Glutamate-gated chloride channel (GluCl) allosteric modulators, such as avermectins/milbemycins, for example abamectin, emamectin benzoate, lepimectin and milbemectin.

(7) Juvenile hormone mimics such as juvenile hormone analogues, for example hydroprene, kinoprene and methoprene or fenoxycarb or pyriproxyfen.

(8) Active compounds with unknown or non-specific mechanisms of action, such as alkyl halides for example as methyl bromide and other alkyl halides or chloropicrin or fluorides or borates or tartar emetic or methyl isocyanate generators.

(9) Chordotonal organ TRPV channel modulators such as pyridine azomethine derivatives, for example pymetrozine and pyrifluquinazon or flonicamid.

(10) Mite growth inhibitors, for example clofentezine, hexythiazox and diflovidazin or etoxazole.

(11) Microbial disruptors of insect gut midgut, for example Bacillus thuringiensis subspecies israelensis, Bacillus thuringiensis subspecies aizawai, Bacillus thuringiensis subspecies kurstaki, Bacillus thuringiensis subspecies tenebrionis and Bacillus sphaericus and BT crop proteins: CrylAb, Cry1Ac, Cry1Fa, Cry1A 105, Cry2Ab, Vip3a, mCry3A, Cry3Ab, Cry3Bb, Cry34Ab1/Cry35Ab1.

(12) Inhibitors of mitochondrial ATP synthase such as organotin miticides, for example azocyclotin, cyhexatin and fenbutatin oxide or diafenthiuron or propargite or tetradifon.

(13) Uncouplers of oxidative phosphorylation acting via disruption of the proton gradient, for example chlorfenapyr, DNOC and sulfluramid.

(14) Nicotinic acetylcholine receptor (nAChR) channel blockers, such as bensultap, cartap-hydrochloride, thiocyclam and thiosultap-sodium.

(15) Inhibitors of chitin biosynthesis, type 0, such as bistrifluoron, chlorfluazuron, diflubenzuron, flucycloxuron, flufenoxuron, hexaflumuron, lufenuron, novaluron, noviflumuron, teflubenzuron and triflumuron.

(16) Inhibitors of chitin biosynthesis, type 1, such as buprofezin.

(17) Molting disruptors (particularly in Dipteran), such as cyromazine.

(18) Ecdysone receptor agonists, such as chromafenozide, halofenozide, methoxyfenozide and tebufenozide.

(19) Octopamine receptor agonists, such as amitraz.

(20) Mitochondrial complex IB electron transport inhibitors such as hydramethylnon or acequinocyl or fluacrypyrim or bifenazate.

(21) Mitochondrial complex I electron transport inhibitors, for example, METI acaricides and insecticides, for example fenazaquin, fenpyroximate, pyrimidifen, pyridaben, tebufenpyrad and tolfenpyrad or rotenone (Derris).

(22) Voltage-dependent sodium channel blockers such as indoxacarb or metaflumizone.

(23) Inhibitors of acetyl CoA carboxylase, such as tetronic and tetramic acid derivatives, for example spirodiclofen, spiromesifen and spirotetramat.

(24) Mitochondrial complex IV electron transport inhibitors, such as phosphides, for example aluminum phosphide, calcium phosphide, zinc phosphide and phosphine or cyanides.

(25) Mitochondrial complex II electron transport inhibitors such as beta-ketonitrile derivatives, for example cyenopyrafen and cyflumetofen or carboxanilides.

(26) Ryanodine receptor modulators such as diamides, for example chlorantraniliprole, cyantraniliprole, flubendiamide, tetraniliprole, (R)-3-chloro-N-1-{2-methyl-4-[1,2,2,2-tetrafluoro-1-(trifluoromethyl)ethyl]phenyl}-N2-(1-methyl-2-methylsulfonylethyl)phthalamide, (S)-3-chloro-N1-{12-methyl-4-[1,2,2,2-tetrafluoro-1-(trifluoromethyl)ethyl]phenyl}-N2-(1-methyl-2-methylsulfonylethyl)phthalamide, methyl-2-[3,5-dibromo-2-({[3-bromo-1-(3-chloropyridin-2-yl)-1H-pyrazol-5-yl]carbonyl}amino)benzoyl]-1,2-dimethylhydrazinecarboxylate, N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide, N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide, N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide, N-[4,6-dichloro-2-[(di-2-propyl-lambda-4-sulfanylidene)carbamoyl]phenyl]-2-(3-chloro-2-pyridyl)-5-(trifluoromethyl)pyrazole-3-carboxamide, N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide, N-[2-(5-amino-1,3,4-thiadiazol-2-yl)-4-chloro-6-methylphenyl]-3-bromo-1-(3-chloro-2-pyridinyl)-1H-pyrazole-5-carboxamide; 3-chloro-1-(3-chloro-2-pyridinyl)-N-[2,4-dichloro-6-[[(1-cyano-1-methylethyl)amino]carbonyl]phenyl]-1H-pyrazole-5-carboxamide, 3-bromo-N-[2,4-dichloro-6-(methylcarbamoyl)phenyl]-1-(3,5-dichloro-2-pyridyl)-1H-pyrazole-5-carboxamide, N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide and cyhalodiamide.

(27) Chordotonal organ modulators on undefined target site such as flonicamid.

Further active ingredients with unknown or indeterminate mode of action, such as afidopyropen, afoxolaner, azadirachtin, benclothiaz, benzoximate, bifenazate, broflanilide, bromopropylate, chinomethionat, cryolite, cyclaniliprole, cycloxaprid, cyhalodiamide, dicloromezotiaz, dicofol, diflovidazin, flometoquin, fluazaindolizine, fluensulfone, flufenerim, flufenoxysirobin, flufiprole, fluhexafon, fluopyram, fluralaner, fluxameiamide, fufenozide, guadipyr, heptafluthrin, imidaclothiz, iprodione, lotilaner, meperfluthrin, paichongding, pyflubumide, pyridalyl, pyrifluquinazon, pyriminostrobin, sarolaner, tetramethylfluthrin, tetraniliprole, tetrachlorantraniliprole, tioxazafen, triflumezopyrim and iodomethane; furthermore, preparations based on Bacillus firmus (I-1582, BioNeem, Votivo), and the following known active compounds: 1-{2-fluoro-4-methyl-5-[(2,2,2-trifluoroethyl)sulfinyl]phenyl}-3-(trifluoromethyl)-1H-1,2,4-triazol-5-amine (known from WO2006043635), {1′-[(2E)-3-(4-chlorophenyl)prop-2-ene-1-yl]-5-fluorospiro[indole-3,4′-piperidine]-1(2H)-yl}(2-chloropyridin-4-yl)methanone (known from WO2003106457), 2-chloro-N-[2-{1-[(2E)-3-(4-chlorophenyl)prop-2-en-1-yl]piperidin-4-yl-}-4-(trifluoromethyl)phenyl]isonicotinamide (known from WO2006003494), 3-(2,5-dimethylphenyl)-4-hydroxy-8-methoxy-1,8-diazaspiro[4.5]dec-3-en-2-one (known from WO2009049851), 3-(2,5-dimethylphenyl)-8-methoxy-2-oxo-1,8-diazaspiro[4.5]dec-3-en-4-yl ethylcarbonate (known from WO2009049851), 4-(but-2-in-1-yloxy)-6-(3,5-dimethylpiperidin-1-yl)-5-fluoropyrimidine (known from WO2004099160), 4-(but-2-in-1-yloxy)-6-(3-chlorophenyl)pyrimidine (known from WO2003076415), PF1364 (CAS-Reg. No. 1204776-60-2), methyl-2-[2-({[3-bromo-1-(3-chloropyridin-2-yl)-1H-pyrazol-5-yl]carbonyl}amino)-5-chloro-3-methylbenzoyl]-2-methylhydrazincarboxylate (known from WO2005085216), methyl-2-[2-({[3-bromo-1-(3-chloropyridin-2-yl)-1H-pyrazol-5-yl]carbonyl}amino)-5-cyano-3-methylbenzoyl]-2-ethylhydrazincarboxylate (known from WO2005085216), methyl-2-[2-({[3-bromo-1-(3-chloro-pyridin-2-yl)-1H-pyrazol-5-yl]carbonyl}amino)-5-cyano-3-methylbenzoyl]-2-methylhydrazincarboxylate (known from WO2005085216), methyl-2-[3,5-dibromo-2-({[3-bromo-1-(3-chloro-pyridin-2-yl)-1H-pyrazol-5-yl]carbonyl}amino)benzoyl]-2-ethylhydrazincarboxylate (known from WO2005085216), N-[2-(5-amino-1,3,4-thiadiazol-2-yl)-4-chloro-6-methylphenyl]-3-bromo-1-(3-chloropyridin-2-yl)-1H-pyrazole-5-carboxamide (known from CN102057925), 4-[5-(3,5-dichlorophenyl)-5-(trifluoromethyl)-4,5-dihydro-1,2-oxazol-3-yl]-2-methyl-N-(1-oxidothietan-3-yl)benzamide (known from WO2009080250), N-[(2E)-1-[(6-chloropyridine-3-yl)methyl]pyridin-2(1H)-ylidene]-2,2,2-trifluoroacetamide (known from WO2012029672), 1-[(2-chloro-1,3-thiazol-5-yl)methyl]-4-oxo-3-phenyl-4H-pyrido[1,2-a]pyrimidin-1-ium-2-olate (known from WO2009099929), 1-[(6-chloropyridin-3-yl)methyl]-4-oxo-3-phenyl-4H-pyrido[1,2-a]pyrimidin-1-ium-2-olate (known from WO2009099929), 4-(3-{2,6-dichloro-4-[(3,3-dichloroprop-2-en-1-yl)oxy]phenoxy}propoxy)-2-methoxy-6-(trifluoromethyl)pyrimidine (known from CN101337940), N-[2-(tert-butylcarbamoyl)-4-chloro-6-methylphenyl]-1-(3-chloropyridin-2-yl)-3-(fluoromethoxy)-1H-pyrazole-5-carboxamide (known from WO2008134969), butyl-[2-(2,4-dichlorophenyl)-3-oxo-4-oxaspiro[4.5]dec-1-en-1-yl]carbonate (disclosed in CN102060818), 3(E)-3-[1-[(6-chloro-3-pyridyl)methyl]-2-pyridyliden]-1,1,1-trifluoropropan-2-one (known from WO2013144213), N-(methylsulfonyl)-6-[2-(pyridin-3-yl)-1,3-thiazol-5-yl]pyridine-2-carboxamide (known from WO20120008%), N-[3-(benzylcarbamoyl)-4-chlorophenyl]-1-methyl-3-(pentafluoroethyl)-4-(trifluoromethyl)-1H-pyrazole-5-carboxamide (known from WO2010051926).

Other insecticidal active compounds of unknown or uncertain mode of action: afidopyropen, afoxolaner, azadirachtin, amidoflumet, benzoximate, bifenazate, broflanilide, bromopropylate, chinomethionat, cryolite, dicloromezotiaz, dicofol, flufenerim, flometoquin, fluensulfone, fluhexafon, fluopyram, flupyradifurone, fluralaner, metoxadiazone, piperonyl butoxide, pyflubumide, pyridalyl, pyrifluquinazon, sulfoxaflor, tioxazafen, triflumezopyrim, 11-(4-chloro-2,6-dimethylphenyl)-12-hydroxy-1,4-dioxa-9-azadispiro[4.2.4.2]-tetradec-11-en-10-one, 3-(4′-fluoro-2,4-dimethylbiphenyl-3-yl)-4-hydroxy-8-oxa-1-azaspiro[4.5]dec-3-en-2-one, 1-[2-fluoro-4-methyl-5-[(2,2,2-trifluoroethyl)sulfinyl]phenyl]-3-(trifluoromethyl)-1H-1,2,4-triazole-5-amine, Bacillus firmus; (E/Z)—N-[1-[(6-chloro-3-pyridyl) methyl]-2-pyridylidene]-2,2,2-trifluoro-acetamide; (E/Z)—N-[1-[(6-chloro-5-fluoro-3-pyridyl)methyl]-2-pyridylidene]-2,2,2-trifluoro-acetamide; (E/Z)-2,2,2-trifluoro-N-[1-[(6-fluoro-3-pyridyl)methyl]-2-pyridylidene]acetamide; (E/Z)—N-[1-[(6-bromo-3-pyridyl)methyl]-2-pyridylidene]-2,2,2-trifluoroacetamide; (E/Z)—N-[1-[1-(6-chloro-3-pyridyl)ethyl]-2-pyridylidene]-2,2,2-trifluoroacetamide; (E/Z)—N-[1-[(6-chloro-3-pyridyl)methyl]-2-pyridylidene]-2,2-difluoro-acetamide; (E/Z)-2-chloro-N-[1-[(6-chloro-3-pyridyl) methyl]-2-pyridylidene]-2,2-difluoro-acetamide; (E/Z)—N-[1-[(2-chloropyrimidin-5-yl)methyl]-2-pyridylidene]-2,2,2-trifluoro-acetamide; (E/Z)—N-[1-[(6-chloro-3-pyridyl)methyl]-2-pyridylidene]-2,2,3,3,3-pentafluoro-propanamide); N-[1-[(6-chloro-3-pyridyl)methyl]-2-pyridylidene]-2,2,2-trifluoro-thioacetamide; N-[1-[(6-chloro-3-pyridyl)methyl]-2-pyridylidene]-2,2,2-trifluoro-N′-isopropyl-acetamidine; fluazaindolizine; 4-[5-(3,5-dichlorophenyl)-5-(trifluoromethyl)-4H-isoxazol-3-yl]-2-methyl-N-(1-oxothietan-3-yl)benzamide; fluxametamide; 5-[3-[2,6-dichloro-4-(3,3-dichloroallyloxy)phenoxy]propoxy]-1H-pyrazole; 3-(benzoylmethylamino)-N-[2-bromo-4-[1,2,2,3,3,3-hexafluoro-1-(trifluoromethyl)propyl]-6-(trifluoromethyl)phenyl]-2-fluoro-benzamide; 3-(benzoylmethylamino)-2-fluoro-N-[2-iodo-4-[1,2,2,2-tetrafluoro-1-(trifluoromethyl)ethyl]-6-(trifluoromethyl)phenyl]-benzamide; N-[3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide; N-[3-[[[2-bromo-4-[1,2,2,2-tetrafluoro-1-(trifluoromethyl)ethyl]-6-(trifluoromethyl)phenyl]amino]carbonyl]-2-fluorophenyl]-4-fluoro-N-methylbenzamide; 4-fluoro-N-[2-fluoro-3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide; 3-fluoroN-[2-fluoro-3-[[[2-iodo-4-[1,2,2,2-tetrafluoro-1-(trifluoromethyl)ethyl]-6(trifluoromethyl)phenyl]amino]carbonyl]phenyl]-N-methyl-benzamide; 2-chloro-N-[3-[[[2-iodo-4-[1,2,2,2-tetrafluoro-1-(trifluoromethyl)ethyl]-6-(trifluoromethyl)phenyl]amino]carbonyl]phenyl]-3-pyridinecarboxamide; 4-cyano-N-[2-cyano-5-[[2,6-dibromo-4-[1,2,2,3,3,3-hexafluoro-1-(trifluoro methyl) propyl]phenyl]carbamoyl]phenyl]-2-methyl-benzamide; 4-cyano-3-[(4-cyano-2-methyl-benzoyl)amino]-N-[2,6-dichloro-4-[1,2,2,3,3,3-hexafluoro-1-(trifluoromethyl) propyl]phenyl]-2-fluoro-benzamide; N-[5-[[2-chloro-6-cyano-4-[1,2,2,3,3,3-hexafluoro-1-(trifluoromethyl)propyl]phenyl]carbamoyl]-2-cyano-phenyl]-4-cyano-2-methyl-benzamide; N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide; N-[5-[[2-bromo-6-chloro-4-[1,2,2,3,3,3-hexafluoro-1-(trifluoromethyl) propyl]phenyl]carbamoyl]-2-cyano-phenyl]-4-cyano-2-methyl-benzamide; 4-cyano-N-[2-cyano-5-[[2,6-dichloro-4-[1,2,2,3,3,3-hexafluoro-1-(trifluoromethyl)propyl]phenyl]carbamoyl]phenyl]-2-methyl-benzamide; 4-cyano-N-[2-cyano-5-[[2,6-dichloro-4-[1,2,2,2-tetrafluoro-1-(trifluoromethyl)ethyl]phenyl]carbamoyl]phenyl]-2-methyl-benzamide; N-[5-[[2-bromo-6-chloro-4-[1,2,2,2-tetrafluoro-1-(trifluoromethyl)ethyl]phenyl]carbamoyl]-2-cyano-phenyl]-4-cyano-2-methyl-benzamide; 2-(1,3-dioxan-2-yl)-6-[2-(3-pyridinyl)-5-thiazolyl]-pyridine; 2-[6-[2-(5-fluoro-3-pyridinyl)-5-thiazolyl]-2-pyridinyl]-pyrimidine; 2-[6-[2-(3-pyridinyl)-5-thiazolyl]-2-pyridinyl]-pyrimidine; N-methylsulfonyl-6-[2-(3-pyridyl)thiazol-5-yl]pyridine-2-carboxamide; N-methylsLilfonyl-6-[2-(3-pyridyl)thiazol-5-yl]pyridine-2-carboxamide; N-ethyl-N-[4-methyl-2-(3-pyridyl)thiazol-5-yl]-3-methylthio-propan amide; N-methyl-N-[4-methyl-2-(3-pyridyl)thiazol-5-yl]-3-methylthio-propanamide; N,2-dimethyl-N-[4-methyl-2-(3-pyridyl)thiazol-5-yl]-3-methylthiopropanamide; N-ethyl-2-methyl-N-[4-methyl-2-(3-pyridyl)thiazol-5-yl]-3-methylthio-propanamide; N-[4-chloro-2-(3-pyridyl)thiazol-5-yl]-N-ethyl-2-methyl-3-m ethylthio-propanamide; N-[4-chloro-2-(3-pyridyl)thiazol-5-yl]-N,2-dimethyl-3-methylthio-propanamide; N-[4-chloro-2-(3-pyridyl)thiazol-5-yl]-N-methyl-3-methylthio-propanamide; N-[4-chloro-2-(3-pyridyl)thiazol-5-yl]-N-ethyl-3-methylthio-propanamide; 1-[(6-chloro-3-pyridinyl)methyl]-1,2,3,5,6,7-hexahydro-5-methoxy-7-methyl-8-nitro-imidazo[1,2-a]pyridine; 1-[(6-chloropyridin-3-yl)methyl]-7-methyl-8-nitro-1,2,3,5,6,7-hexahydroimidazo[1,2-a]pyridin-5-ol; 1-isopropyl-N,5-dimethyl-N-pyridazin-4-yl-pyrazole-4-carboxamide; 1-(1,2-di methyl propyl)-N-ethyl-5-methyl-N-pyridazin-4-yl-pyrazole-4-carboxamide; N,5-dimethyl-N-pyridazin-4-yl-1-(2,2,2-trifluoro-1-methyl-ethyl)pyrazole-4-carboxamide; 1-[1-(1-cyanocyclopropyl)ethyl]-N-ethyl-5-methyl-N-pyridazin-4-yl-pyrazole-4-carboxamide; N-ethyl-1-(2-fluoro-1-methyl-propyl)-5-methyl-N-pyridazin-4-yl-pyrazole-4-carboxamide; 1-(1,2-dimethylpropyl)-N,5-dimethyl-N-pyridazin-4-yl-pyrazole-4-carboxamide; 1-[1-(1-cyanocyclopropyl)ethyl]-N,5-dimethyl-N-pyridazin-4-yl-pyrazole-4-carboxamide; N-methyl-1-(2-fluoro-1-methyl-propyl]-5-methyl-N-pyridazin-4-yl-pyrazole-4-carboxamide; 1-(4,4-difluorocyclohexyl)-N-ethyl-5-methyl-N-pyridazin-4-yl-pyrazole-4-carboxamide; 1-(4,4-difluorocyclohexyl)-N,5-dimethyl-N-pyridazin-4-yl-pyrazole-4-carboxamide, N-(1-methylethyl)-2-(3-pyridinyl)-2H-indazole-4-carboxamide; N-cyclopropyl-2-(3-pyridinyl)-2H-indazole-4-carboxamide; N-cyclohexyl-2-(3-pyridinyl)-2H-indazole-4-carboxamide; 2-(3-pyridinyl)-N-(2,2,2-trifluoroethyl)-2H-indazole-4-carboxamide; 2-(3-pyridinyl)-N-[(tetrahydro-2-furanyl)methyl]-2H-indazole-5-carboxamide; methyl 2-[[2-(3-pyridinyl)-2H-indazol-5-yl]carbonyl]hydrazinecarboxylate; N-[(2,2-difluorocyclopropyl)methyl]-2-(3-pyridinyl)-2H-indazole-5-carboxamide; N-(2,2-difluoropropyl)-2-(3-pyridinyl)-2H-indazole-5-carboxamide; 2-(3-pyridinyl)N-(2-pyrimidinylmethyl)-2H-indazole-5-carboxamide; N-[(5-methyl-2-pyrazinyl)methyl]-2-(3-pyridinyl)-2H-indazole-5-carboxamide, N-[3-chloro-1-(3-pyridyl) pyrazol-4-yl]N-ethyl-3-(3,3,3-trifluoropropylsulfanyl) propanamide; N-[3-chloro-1-(3-pyridyl) pyrazol-4-yl]-N-ethyl-3-(3,3,3-trifluoropropylsulfinyl)propanamide; N-[3-chloro-1-(3-pyridyl)pyrazol-4-yl]-3-[(2,2-difluorocyclopropyl)methylsulfanyl]-N-ethyl-propanamide; N-[3-chloro-1-(3-pyridyl)pyrazol-4-yl]-3-[(2,2-difluorocyclopropyl)methylsulfinyl]-N-ethyl-propanamide; sarolaner, lotilaner. The active substances referred above, their preparation and their activity e.g. against harmful fungi is known (cf.: http://www.alanwood.net/pesticides/); these substances are commercially available. The compounds described by IUPAC nomenclature, their preparation and their pesticidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A1 035 122; EP-A 1 201 648; EP-A 1 122 244, JP 2002316902; DE 19650197; DE 10021412; DE 102005009458; U.S. Pat. Nos. 3,296,272; 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 10/139271, WO 11/028657, WO 12/168188, WO 07/006670, WO 11/77514; WO 13/047749, WO 10/069882, WO 13/047441, WO 03/16303, WO 09/90181, WO 13/007767, WO 13/010862, WO 13/127704, WO 13/024009, WO 13/24010, WO 13/047441, WO 13/162072, WO 13/092224, WO 11/135833, CN 1907024, CN 1456054, CN 103387541, CN 1309897, WO 12/84812, CN 1907024, WO 09094442, WO 14/60177, WO 13/116251, WO 08/013622, WO 15/65922, WO 94/01546, EP 2865265, WO 07/129454, WO 12/165511, WO 11/081174, WO 13/47441).

The mass ratio of any two ingredients in each combination is selected as to give the desired effect, for example, enhanced activity. In general, the mass ratio would vary depending on the specific ingredient and how many ingredients are present in the combination. Generally, the mass ratio between any two ingredients in any combination of the present invention, independently of one another, is from 100:1 to 1:100, including from 99:1, 98:2, 97:3, 96:4, 95:5, 94:6, 93:7, 92:8, 91:9, 90:10, 89:11, 88:12, 87:13, 86:14, 85:15, 84:16, 83:17, 82:18, 81:19, 80:20, 79:21, 78:22, 77:23, 76:24, 75:25, 74:26, 73:27, 72:28, 71:29, 70:30, 69:31, 68:32, 67:33, 66:34, 65:45, 64:46, 63:47, 62:48, 61:49, 60:40, 59:41, 58:42, 57:43, 56:44, 55:45, 54:46, 53:47, 52:48, 51:49, 50:50, 49:51, 48:52, 47:53, 46:54, 45:55, 44:56, 43:57, 42:58, 41:59, 40:60, 39:61, 38:62, 37:63, 36:64, 35:65, 34:66, 33:67, 32:68, 31:69, 30:70, 29:71, 28:72, 27:73, 26:74, 25:75, 24:76, 23:77, 22:78, 21:79, 20:80, 19:81, 18:82, 17:83, 16:84, 15:85, 14:86, 13:87, 12:88, 11:89, 10:90, 9:91, 8:92, 7:93, 6:94, 5:95, 4:96, 3:97, 2:98, to 1:99. Preferred mass ratios between any two components of present invention are from 75:1 to 1:75, more preferably, 50:1 to 1.50, especially 25:1 to 1:25, advantageously 10:1 to 1:10, such as 5:1 to 1:5, for example 1:3 to 3:1. The mixing ratios are understood to include, on the one hand, ratios by mass and also, on other hand, molar ratios.

The combinations of the present invention (i.e. those comprising a compound of the present invention and one or more other biological active agents) may be applied simultaneously or sequentially.

In the event, the ingredients of a combination are applied sequentially (i.e., one after the other), the ingredients are applied sequentially within a reasonable period of each other to attain the biological performance, such as within a few hours or days. The order of applying the ingredients in the combination, i.e., whether the compounds of formula (I) should be applied first or not is not essential for working the present invention.

In the event ingredients of the combinations are applied simultaneously in the present invention, they may be applied as a composition containing the combination, in which case (A) the compound of formula (I) and the one or more other ingredients in the combinations can be obtained from separate formulation sources and mixed together (known as a tank-mix, ready-to-apply, spray broth, or slurry), or (B) the compound of formula (I) and the one or more other ingredients can be obtained as single formulation mixture source (known as a pre-mix, ready-mix, concentrate, or formulated product). In an embodiment, independent of other embodiments, a compound according to the present invention is applied as a combination. Accordingly, the present invention also provides a composition comprising a compound according to the invention as herein described and one or more other biological active agents, and optionally one or more customary formulation auxiliaries; which may be in the form of a tank-mix or pre-mix composition.

The compounds of formula (I) are particularly useful for controlling and preventing helminth and nematode endo and ecto-parasitic infestations and infections in warm-blooded animals such as cattle, sheep, swine, camels, deer, horses, poultry, fish, rabbits, goats, mink, fox, chinchillas, dogs and cats as well as humans. In the context of control and prevention of infestation and infections in warm-blooded animals, compounds of invention are especially useful for the control of helminths and nematodes. Examples for helminths are members of the class Trematoda, commonly known as flukes or flatworms, especially members of the genera Fasciola, Fascioloides, Paramphistomu, Dicrocoelium, Eurytrema, Ophisthorchis, Fasciolopsis, Echinostoma and Paragonimus. Nematodes which can be controlled by the formula (I) compounds include the genera Haemonchus, Ostertagia, Cooperia, Oesphagastomu, Nematodirus, Dictyocaulus, Trichuris, Dirofilaria, Ancyclostoma, Ascaria and the like. For oral administration to warm-blooded animals, the compounds of the invention may be formulated as animal feeds, animal feed premixes, animal feed concentrates, pills, solutions, pastes, suspensions, drenches, gels, tablets, boluses and capsules. In addition, the compounds of the invention may be administered to the animals in their drinking water. For oral administration, the dosage form chosen should provide the animal with about 0.01 mg/kg to 100 g/kg of animal body weight per day of the compound of the invention. Alternatively, the compounds of the invention may be administered to animals parenterally, for example, by intraruminal, intramuscular, intravenous or subcutaneous injection. The compounds of the invention may be dispersed or dissolved in a physiologically acceptable carrier for subcutaneous injection. Alternatively, the compounds of the invention may be formulated into an implant for subcutaneous administration. In addition the compounds of the invention may be transdermally administered to animals. For parenteral administration, the dosage form chosen should provide the animal with about 0.01 mg/kg to 100 mg/kg of animal body weight per day of the compound of the invention.

The compounds of the invention may also be applied topically to the animals in the form of dips, dusts, powders, collars, medallions, sprays and pour-on formulations. For topical application, dips and sprays usually contain about 0.5 ppm to 5,000 ppm and preferably about 1 ppm to 3,000 ppm of the compound of the invention. In addition, the compounds of the invention may be formulated as ear tags for animals, particularly quadrupeds such as cattle and sheep. In an embodiment, independent of any other embodiments, a compound of formula (I) is an anti-helminth compound. In an embodiment, independent of any other embodiments, a compound of formula (I) is a pesticidal compound, preferably a nematicidal compound. Temperatures are given in degrees Celsius.

The compounds of the present invention not only control insect pests effectively but also show positive crop response such as plant growth enhancement effects like enhanced crop vigor, enhanced root growth, enhanced tolerant to drought, high salt, high temperature, chill, frost or light radiation, improved flowering, efficient water & nutrient utilization (such as improved nitrogen assimilation), enhanced quality plant product, more number of productive tillers, enhanced resistance to fungi, insects, pests and the like, which results in higher yields.

Any of the compounds according to the invention can exist in one or more optical, geometric or chiral isomer forms depending on the number of asymmetric centres in the compound. The invention thus relates equally to all the optical isomers and to their racemic or scalemic mixtures (the term “scalemic” denotes a mixture of enantiomers in different proportions), and to the mixtures of all the possible stereoisomers, in all proportions. The diastereoisomers and/or the optical isomers can be separated according to the methods which are known per se by a person ordinary skilled in the art.

Any of the compounds according to the invention can also exist in one or more geometric isomer forms depending on the number of double bonds in the compound. The invention thus relates equally to all geometric isomers and to all possible mixtures, in all proportions. The geometric isomers can be separated according to general methods, which are known per se by a person ordinary skilled in the art.

Any of the compounds according to the invention, can also exist in one or more amorphic or isomorphic or polymorphic forms, depending on their preparation, purification storage and various other influencing factors. The invention thus relates all the possible amorphic, isomorphic and polymorphic forms, in all proportions. The amorphic, isomorphic and polymorphic forms can be prepared and/or separated and/or purified according to general methods, which are known per se by a person ordinary skilled in the art. In one embodiment, the present invention provides a process for preparing a compound of formula (I) and/or a salt thereof which comprises at least one of the following steps (a) to (p):
a) converting substituted thioether compound of formula (4) to afford compound of formula (2) followed by alkylating or acylating the compound of formula (2) to afford compound of formula (1) according to reaction scheme as depicted below:

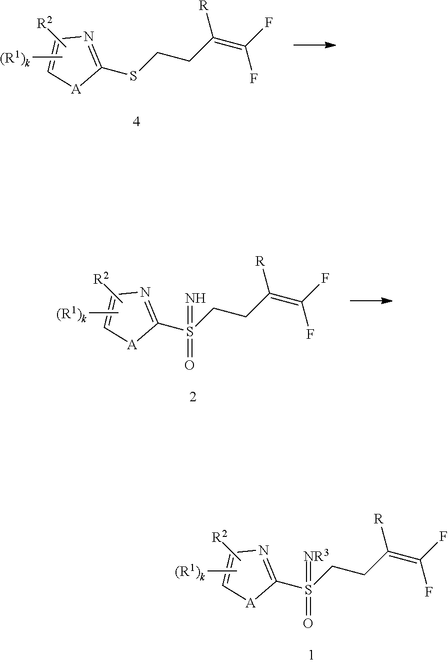

b) converting sulfoxide compound of formula (5) to sulfoximine compound of formula (2) followed by alkylating or acylating the compound of formula (2) to afford compound of formula (1) according to reaction scheme as depicted below:

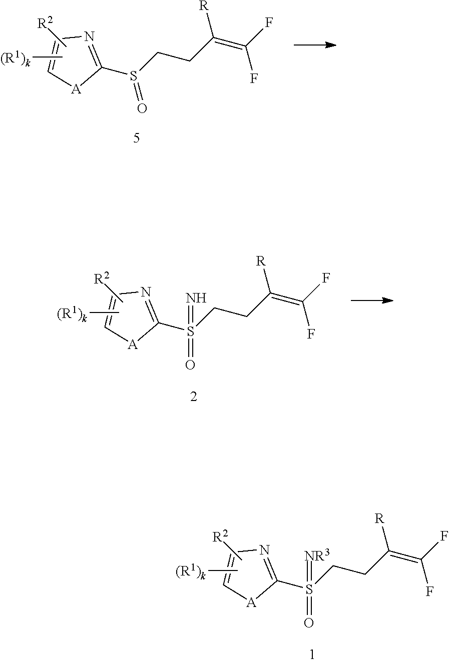

c) reacting substituted thioether compound of formula (4) with cyanamide compound to afford compound of formula (3) followed by oxidizing the compound of formula (3) to afford compound of formula (1) according to reaction scheme as depicted below:

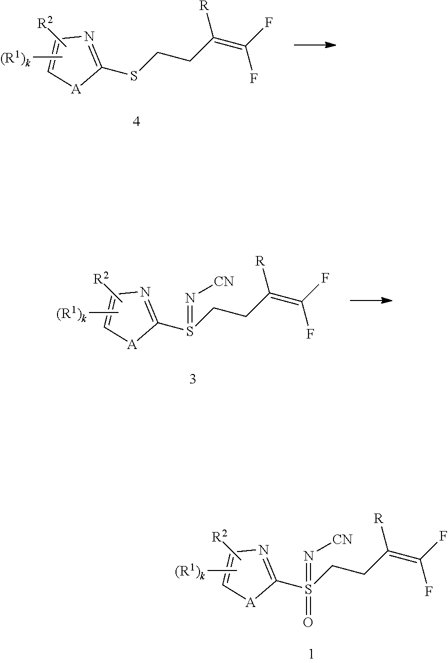

d) oxidizing substituted thioether of formula (4) to afford compound of formula (5) or (6) according to reaction scheme as depicted below:

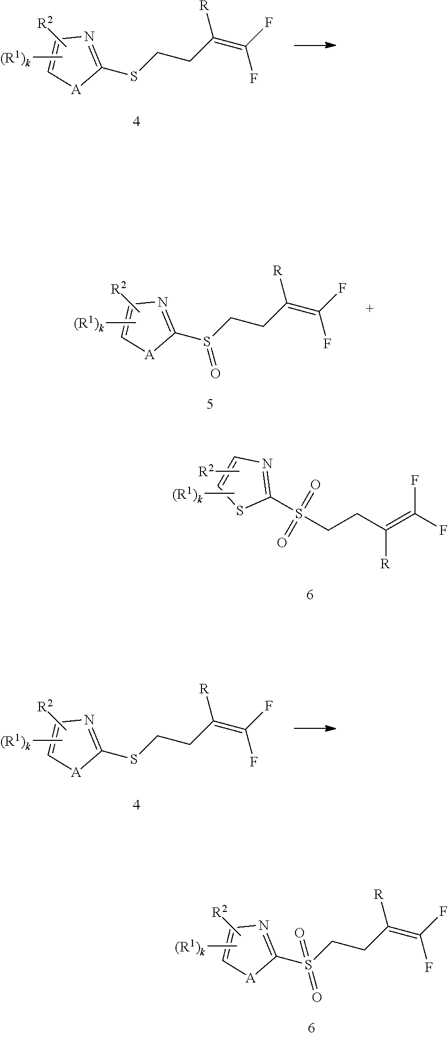

e) reducing ester compound of formula (15) to alcohol compound of formula (16) followed by oxidizing the compound of formula (16) to afford corresponding aldehyde compound of formula (4a or 4b) according to reaction scheme as depicted below:

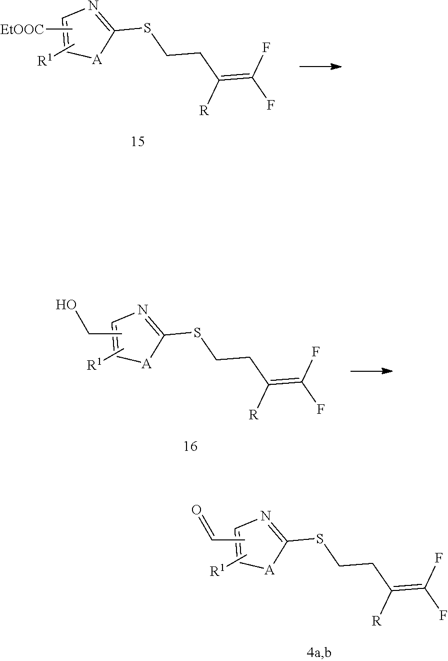

f) formylating thio compound of formula (18) to afford aldehyde compound of formula (4b) according to reaction scheme as depicted below:

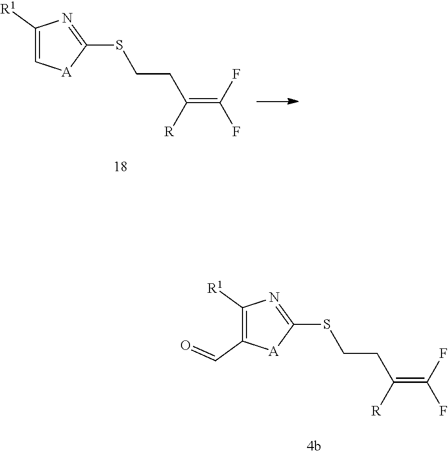

g) converting halide compound of formula (21) to thio compound of formula (22) followed by alkylation with compound of formula (14) to afford compound of formula (4a or 4b) according to reaction scheme as depicted below:

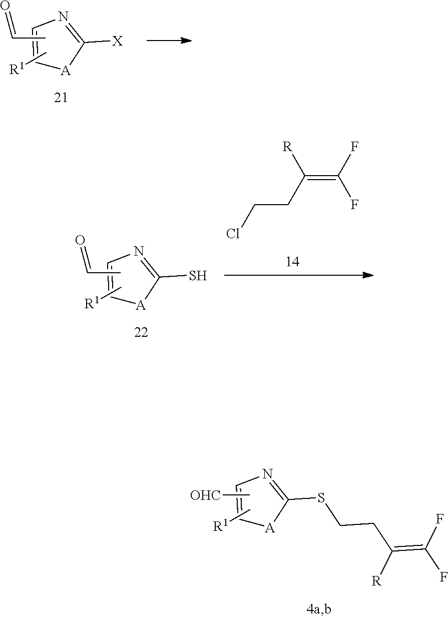

h) reacting aldehyde compound of formula (4a or 4b) with Grignard reagent to afford compound of formula (9) followed by oxidizing the compound of formula (9) to afford corresponding ketone compound of formula (10) according to reaction scheme as depicted below:

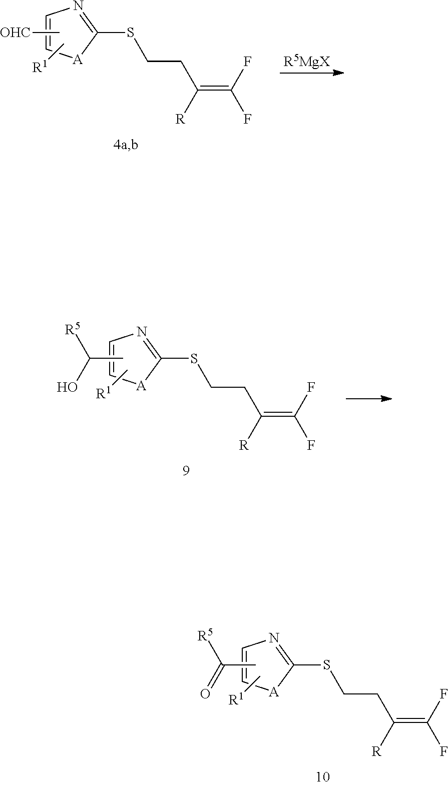

i) reacting aldehyde compound of formula (4a or 4b) with (un)substituted hydroxyl amine optionally followed by reaction with alkyl halide to afford compound of formula (8) which is then converted to compound of formula 1a following step (a) according to reaction scheme as depicted below:
j) reacting ketone compound of formula (10) with substituted hydroxyl amine to afford compound of formula (11) followed by reaction with alkyl halide to afford compound of formula (12) which is then converted to compound of formula 1a following step (a) according to reaction scheme as depicted below:

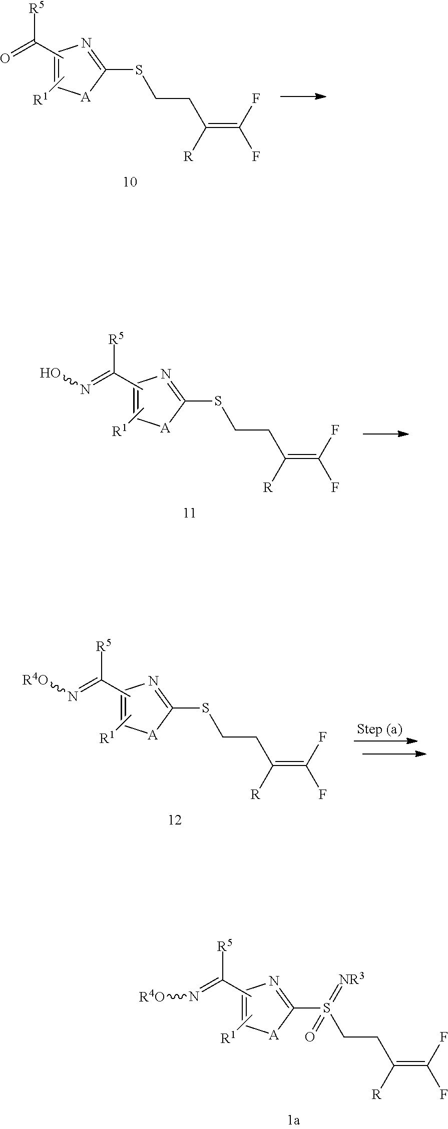

k) reacting substituted thioether compound of formula (18) with cyanamide compound to afford compound of formula (21) followed by oxidizing the compound of formula (21) to afford compound of formula (1a) according to reaction scheme as depicted below:

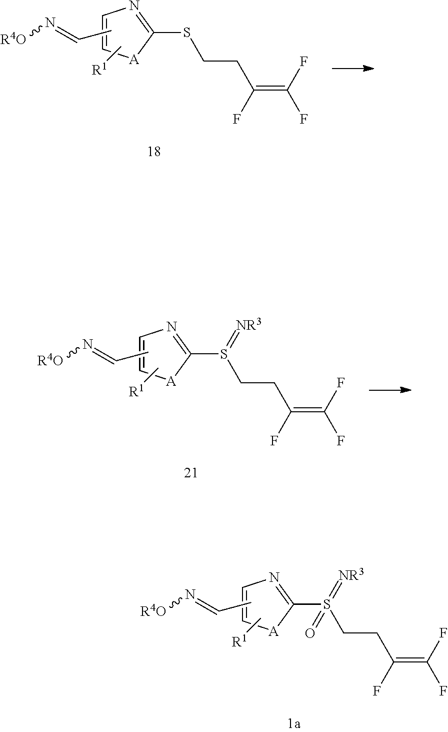

l) reacting compound of formula (4a or 4b) with suitable fluorinating agent to afford compound of formula (32) which is then converted to compound of formula (1b) following step (a) according to reaction scheme as depicted below:

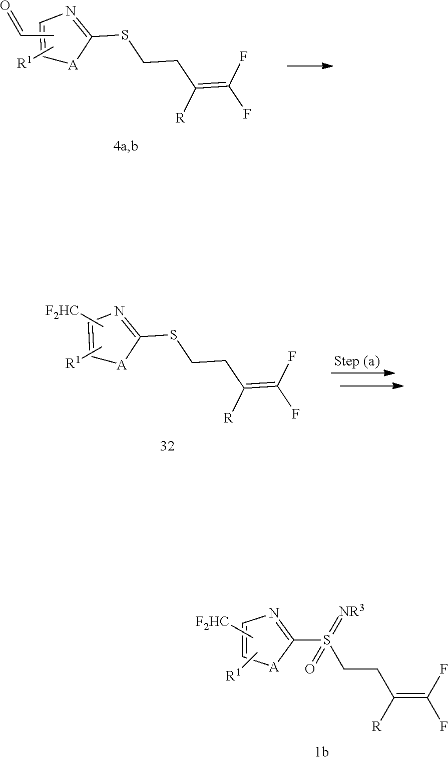

m) reacting compound of formula (7) with substituted styrene of formula (23) to afford compound of formula (24) which is then converted to compound of formula (1a) following step (a) respectively according to reaction schemes as depicted below:

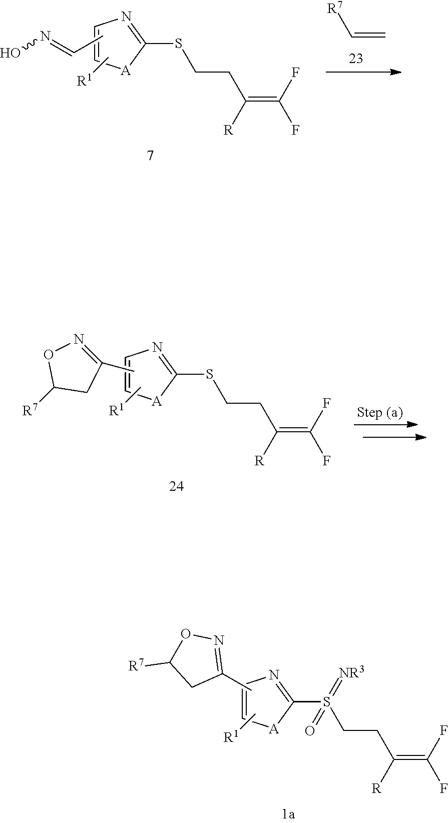

n) reacting compound of formula (7) with substituted alkyne of formula (25) to afford compound of formula (26) which is then converted to compound of formula (1a) following step (a) according to reaction scheme as depicted below:

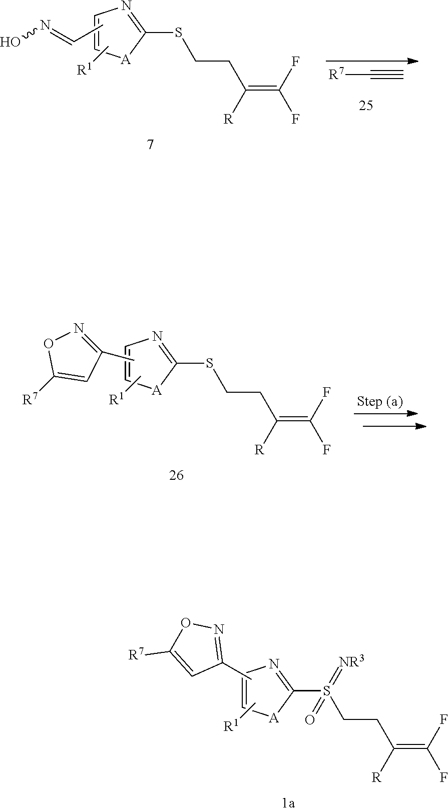

o) reacting oxime compound of formula (7) with phosphonic anhydride compound and further reacting with hydroxyl amine to afford compound of formula (28) followed by reaction with acid chloride/acid anhydride (29 or 29a) to afford compound of formula (30) which is then converted to compound of formula (1a) following step (a) according to reaction schemes as depicted below:

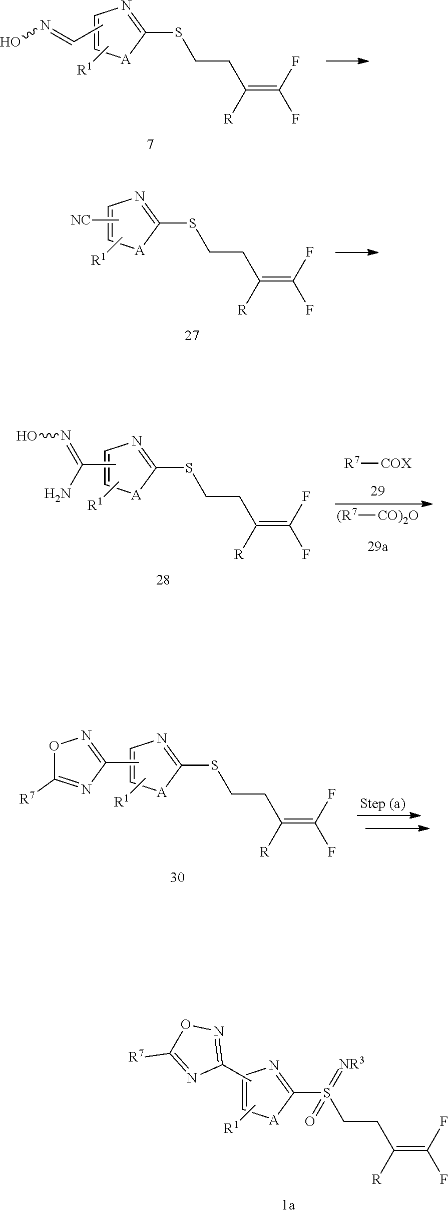

p) reacting compound of formula (28) with carbonylating agent to afford compound of formula (31) followed by converting the compound of formula (31) to compound of formula (1a) following step (a) according to reaction schemes as depicted below:

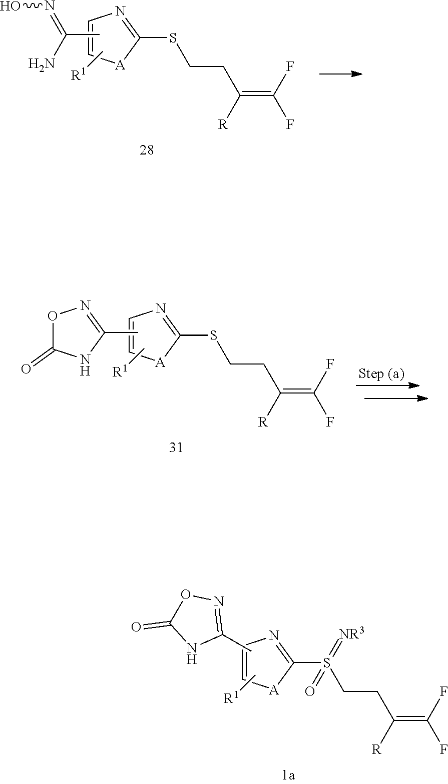

There are large number of suitable known standard methods, such as alkylation, halogenation, acylation, amidation, oximation, oxidation and reduction. The choice of the preparation methods which are suitable are depending on the properties (reactivity) of the substituents in the intermediates. These reactions can be conveniently performed in a solvent. These reactions can be conveniently performed at various temperatures. These reactions can be conveniently performed in an inert atmosphere. The reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropyl amide, potassium bis(trimethylsilyl)amide, calcium hydride, triethyl amine, diisopropylethylamine, triethylenediamine, cyclohex ylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reaction according to Scheme-1 to 13 is preferably carried out in a solvent selected from standard solvents which are inert under the prevailing reaction conditions. Preference is given to aliphatic, alicyclic or aromatic hydrocarbons, such as, petroleum ether, hexane, toluene; halogenated hydrocarbons, such as, chlorobenzene, dichloromethane, chloroform, carbon tetrachloride or dichloroethane; ethers, such as, diethyl ether, diisopropyl ether, methyl t-butyl ether (MTBE), dioxane, tetrahydrofuran or 1,2-dimethoxyethane; nitriles, such as, acetonitrile or propionitrile, or, amides, such as, N,N-dimethyl formamide (DMF), N,N-dimethylacetamide, N-methylformanilide, N-methylpyrrolidone (NMP) or hexamethylenephosphoric triamide; esters, such as, for example, methyl acetate or ethyl acetate; sulfoxides, such as, dimethyl sulfoxide (DMSO); sulfones, such as, sulfolane; alcohols, such as, methanol, ethanol, nor isopropanol, 1,1-, iso-, sec- or tert-butanol, ethanediol, propane-1,2-diol, ethoxyethanol, methoxyethanol, diethylene glycol monomethyl ether, diethylene glycol monoethyl ether or mixtures of these.

The reactants can be reacted with each other as such, i.e. without adding a solvent or diluent. The reaction is advantageously carried out in a temperature range from approximately −80° C. to approximately +140° C., preferably from approximately −30° C. to approximately +100° C., in many cases in the range between ambient temperature and approximately +80° C.

A compound of formula (I) can be converted in a manner known per se into another compound of formula (I) by replacing one or more substituents of the starting compound of formula (I) in the customary manner by (an)other substituent(s) according to the invention. Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step. Salts of compounds of formula (I) can be prepared in a manner known per se. Thus, for example, acid addition salts of compounds of formula (I) are obtained by treatment with a suitable acid or a suitable ion exchanger reagent and salts with bases are obtained by treatment with a suitable base or with a suitable ion exchanger reagent A salt is chosen depending on its tolerances for compound's use, such as agricultural or physiological tolerance. Salts of compounds of formula (I) can be converted in the customary manner into the free compounds I, add addition salts, for example, by treatment with a suitable basic compound or with a suitable ion exchanger reagent and salts with bases, for example, by treatment with a suitable acid or with a suitable ion exchanger reagent. Salts of compounds of formula (I) can be converted in a manner known per se into other salts of compounds of formula (I), add addition salts, for example, into other add addition salts, for example by treatment of a salt of inorganic acid such as hydrochloride with a suitable metal salt such as a sodium, barium or silver salt, of an add, for example with silver acetate, in a suitable solvent in which an inorganic salt which forms, for example silver chloride, is insoluble and thus precipitates from the reaction mixture.

In one embodiment, the present invention provides a compound of general formula (II),

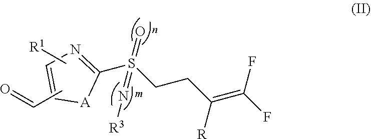

Wherein;

A represent O, NR⁴ or S;

n, m, and k represents integers wherein n=0-2, m=0-1 and k=0-2;

R is selected from the group consisting of hydrogen, halogen and C₁-C₃-alkyl;

R¹ is selected from the group consisting of hydrogen, X, CN, SCN, SF₅, OR⁴, NO₂, N(R⁴)₂, Si(R⁴)₃, (C═O)—R⁴, S(O)₀₋₂R⁴, C₁-C₈-alkyl-S(O)₀₋₂R⁴, C₁-C₆-alkyl-OR⁴, C₁-C₈-alkyl-(C═O)—R⁴, C(R^{4a})═NR⁴, S(O)₀₋₂ C₅-C₁₂-aryl, S(O)₀₋₂C₇-C₁₉-aralkyl, C₁-C₁₂-alkyl, C₂-C₁₂-alkenyl, C₂-C₁₂-alkynyl, C₁-C₁₂-haloalkyl, C₂-C₁₂-haloalkenyl, C₂-C₁₂-haloalkynyl, C₃-C₁₀-cycloalkyl, C₃-C₁₀-halocycloalkyl, C₄-C₁₀-cycloalkenyl, C₅-C₁₀-cycloalkynyl, C₁-C₈-alkyloxy-C₃-C₁₀-cycloalkyl, C₁-C₈-alkylthio-C₃-C₁₀-cycloalkyl, C₇-C₁₉-aralkyl, C₅-C₁₂-bicycloalkyl and C₇-C₁₂-bicycloalkenyl; wherein the cyclic ring system one or more carbon atoms may be replaced by heteroatoms selected from the group consisting of N, O, S and optionally including 1 to 3 ring members selected from the group consisting of C(═O), C(═S), S(O)₀₋₂ and Si(R⁴)₂; R¹ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;

R⁴ is selected from the group consisting of hydrogen, OR^{4a}, N(R^{4a})₂, C₁-C₆-alkyl, C₁-C₆-alkenyl, C₁-C₆-alkynyl, C₁-C₆-haloalkyl, C₂-C₆-haloalkenyl, C₂-C₆-haloalkynyl, C₃-C₁₂-cycloalkyl, C₃-C₁₀-halocycloalkyl, C₄-C₁₂-cycloalkenyl, C₅-C₁₂-cycloalkynyl and C₃-C₁₂-heterocyclyl; R⁴ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂,
R^{4a} is selected from the group consisting of hydrogen, C₁-C₆-alkyl and C₃-C₆-cycloalkyl;
X represents halogen.

The compounds of formula I as disclosed in the present invention can be synthesized by using following synthetic schemes:

Preparation of Sulfoximine Derivatives of Formula 1:

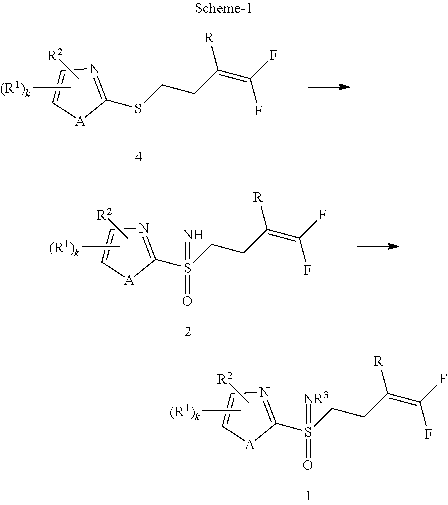

As shown in scheme 1, compounds of formula 1 can be prepared by a reaction of thioether of formula 4 in the presence of imidating agent (e g ammonium carbamate and iodobenzene diacetate) in suitable solvents (e.g. dichloromethane or methanol) at 0-50° C. to get the intermediate 2. Further, corresponding R³ group can be introduced in compound of formula 2 by alkylation or acylation using suitable reagents.

Alternative Method for Preparation Sulfoximine Derivatives of Formula 1:

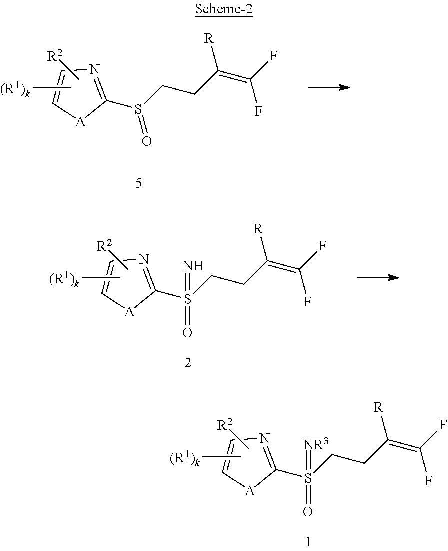

As shown in scheme 2, compounds of formula 1 can also be prepared by a reaction of sulfoxide of formula 5 in the presence of imidating agent (e.g. sodium azide and sulfuric acid) to get the compound of formula 2. Further, R³ group can be introduced by alkylation or acylation using suitable reagent.

Preparation of Sulfilimine/Sulfoximine Derivatives of Formula 1, Wherein R³═CN)

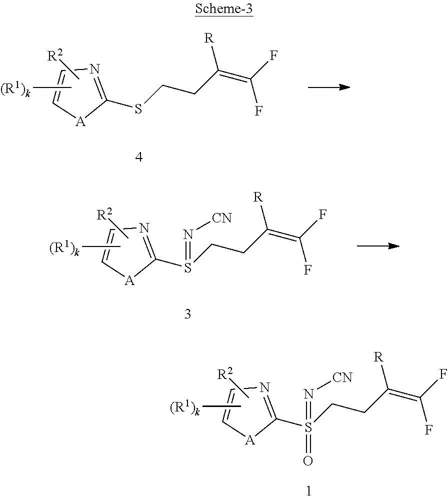

Compound of formula 3 and 1 can be prepared by following scheme 3. Intermediate of formula 4 can be converted to 3 by using imidating agent (e.g. cyanamide) with oxidizing reagent (e.g. iodobenzene diacetate) in suitable solvents (e.g. acetonitrile, tetrahydrofuran) at temperature between 0° C. to 25° C. Further, compound of formula 3 can be oxidized to compound of formula 1 in the presence of oxidizing reagents (e.g. m-chloroperbenzoic acid, oxone) in suitable solvents (e.g. dichloromethane, chloroform or methanol) at 25° C.

Preparation of Sulfoxide/Sulfone Derivatives of Formula 5 & 6:

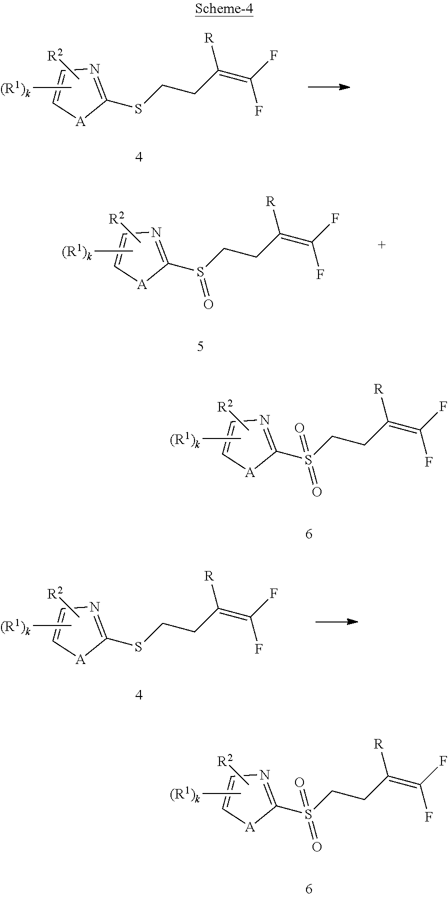

As shown in scheme 4, compounds of formula 5 and 6 can be prepared by the oxidation of substituted thioether compounds of formula 4 with appropriate equivalents of suitable oxidizing reagent (e.g. oxone or m-chloroperbenzoic acid) in solvents (e.g. dichloromethane or methanol).

Preparation of Aldoxime Derivatives of Formula 1a:

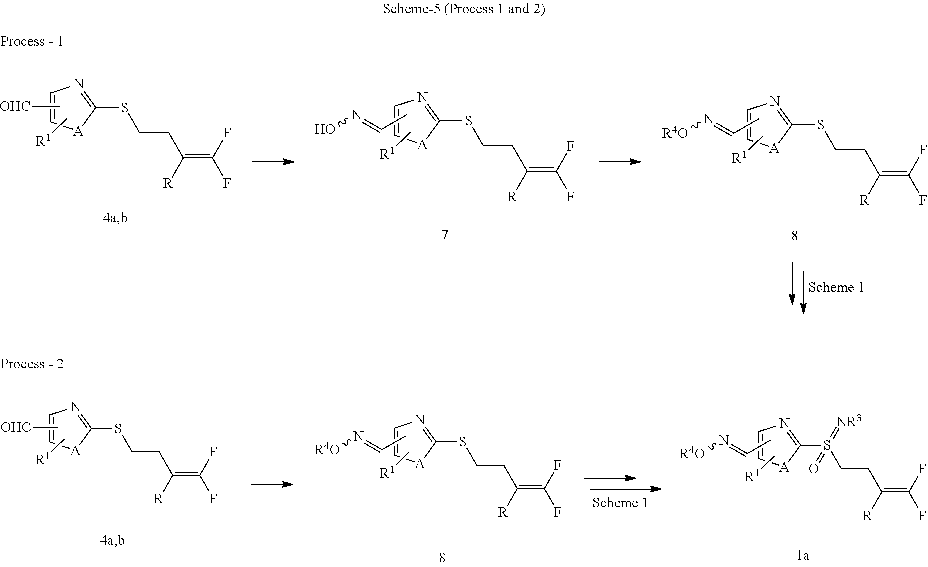

As shown in scheme-5 compounds of formula 8 can be prepared by following process 1 and 2. The condensation of hydroxyl amine hydrochloride with aldehydes of formula 4a,b in suitable solvents (e.g. methanol, ethanol or pyridine) at 10-80° C. to get compound of formula 7, followed by alkylation using corresponding alkylating agents resulted in to compound of formula 8 as per the process 1. The compound of formula 8 can also be synthesized from compound of formula 4a,b following process 2 using suitably substituted hydroxyl amine hydrochloride salt. Further, compound of formula 8 can be converted to compound of formula 1a following synthetic protocol given in schemes 1 and 3.

Preparation of Ketoxime Derivatives of Formula 1a:

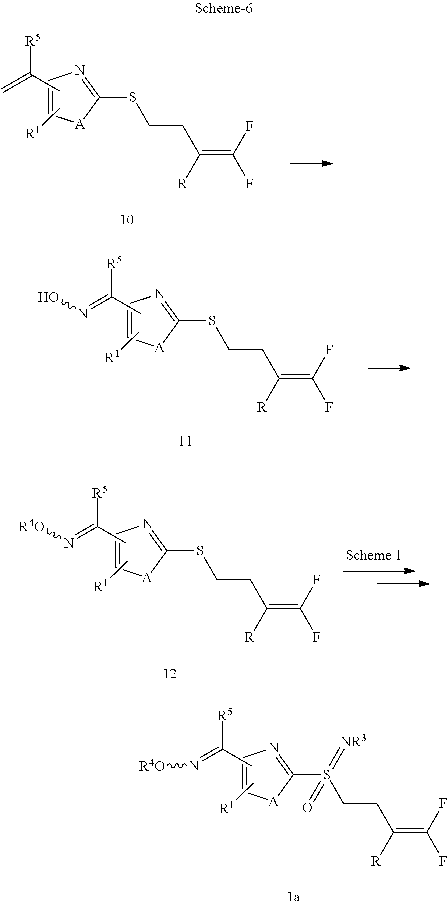

As shown in scheme 6, compounds of formula 11 can be prepared by the condensation of hydroxyl amine hydrochloride salt with ketone of formula 10 in suitable solvents (e.g. methanol, ethanol or pyridine) at a temperature between 25° C. to 100° C. The resultant oxime derivatives of formula 11 can be converted into a compound of formula 12 using corresponding alkyl halide optionally in the presence of organic or inorganic bases (e.g. potassium carbonate, silver carbonate, N,N-diisopropylethylamine or sodium hydroxide etc.) in suitable solvents (e.g. toluene, acetonitrile or N,N-dimethylformamide etc.). Further, compound of formula 12 can be converted to compound of formula 1a following synthetic protocol given in schemes 1 and 3.

Preparation of Ketone Intermediates of Formula 10:

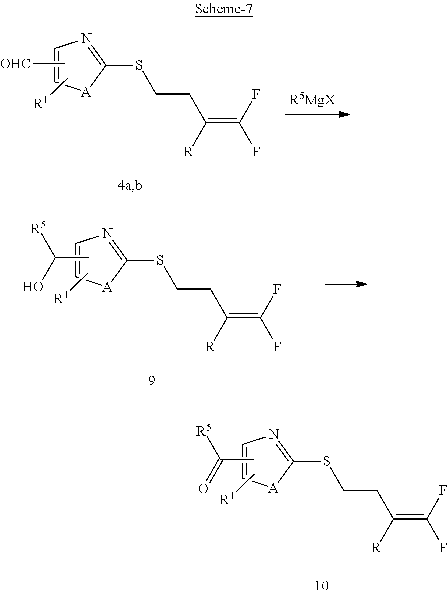

As shown in scheme 7, compounds of formula 4a,b can be converted to compound of formula 9 by addition of Grignard reagent R⁵MgX in a suitable solvents (e.g. tetrahydrofuran, diethyl ether, methyl tertiary butylether etc). The compound of formula 9 can be oxidized to compound of formula 10 using oxidizing agents (e.g. Dess-martin periodinane or manganese dioxide) in suitable solvents (e.g. dichloromethane, acetonitrile chloroform etc.) at a temperature between 0° C. to 25° C.

Preparation of Aldehyde Intermediates of Formula 4a,b:

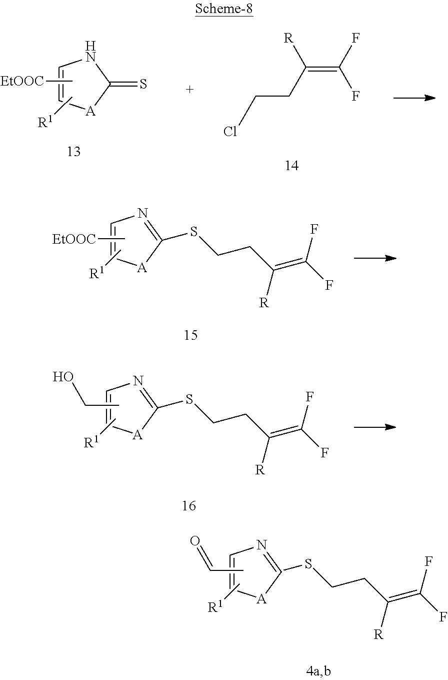

As shown in scheme 8, compounds of formula 4a,b can be prepared by a reaction sequence starting with alkylation of thiol of formula 13 with alkenyl halide 14 in the presence of organic or inorganic bases (e.g. N,N-diisopropyl ethylamine, triethylamine, potassium carbonate, silver carbonate, sodium hydroxide, & sodium hydride etc.) in suitable solvents (acetonitrile, acetone, toluene and N,N-dimethylformamide etc.) under heating condition. The resulting compound of formula 15 can be reduced to alcohol of formula 16 using reducing reagents (e.g sodium borohydride, diisobutylaluminium hydride etc) in suitable solvents (e.g. methanol, tetrahydrofuran, diethyl ether etc). The compound of formula 16 can be oxidized to the corresponding aldehyde derivatives of formula 4a,b by using oxidizing reagent (e.g. Dess Martin periodinane or manganese dioxide) in suitable solvents (e.g. dichloromethane, acetonitrile chloroform etc.) at temperature between 0° C. to 35° C.

Preparation of Aldehyde Intermediates of Formula 4b:

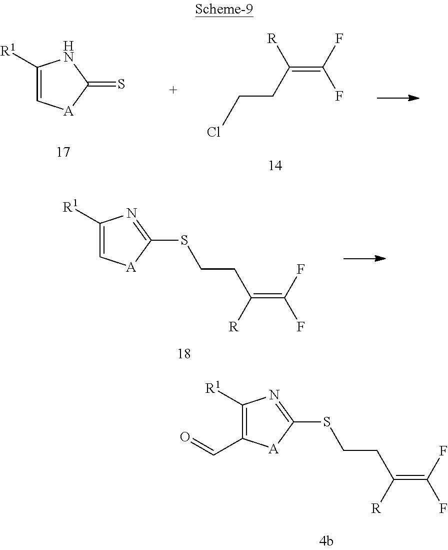

As shown in scheme 9, compounds of formula 4b can be prepared by a reaction sequence starting with alkylation of thiol of formula 17 with alkenyl halide 14 in the presence of organic or inorganic base (e.g. N,N-diisopropyl ethylamine, triethylamine, potassium carbonate, silver carbonate, sodium hydroxide, & sodium hydride etc.) in suitable solvents (acetonitrile, acetone, toluene and N,N-dimethylformamide etc.) under heating condition. The compound of formula 18 can be converted in to compound of formula 4b through Vilsmeier-Haack formylation using phosphorus oxychloride and N,N-dimethylformamide.

Alternative Method for Preparation of Aldehyde Intermediate of Formula 4a,b:

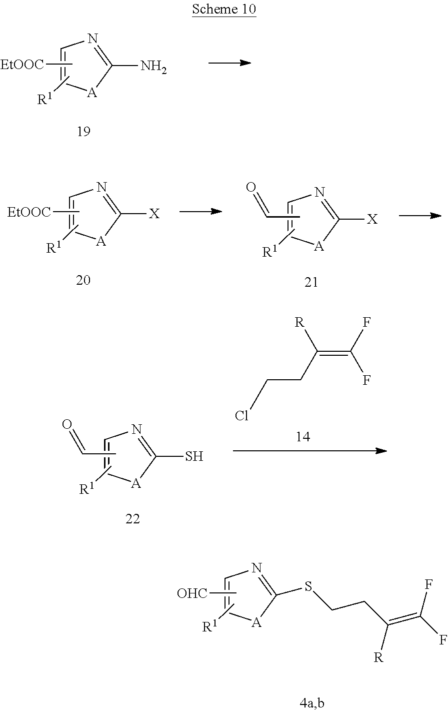

Compounds of formula 4a,b can be prepared by following synthetic scheme 10. The compound of formula 19 can be converted in to formula of 20 through sandmayer reaction using diazotizing agents (e.g. tertiary butylnitrite, sodium nitrite etc) and copper halide in suitable solvents (e.g. acetonitrile acetone etc.). Next compound of formula 20 can be converted to compound of formula 21 by reduction followed by oxidation using methods known to a person skilled in the art. Further, compound of formula 21 can be converted to compound of formula 22 by reacting with suitable thiolating reagent (e.g. thiourea, sodium sulphide, or potassium thioacetate) in suitable solvents (e.g methanol, ethanol and isopropanol etc.) at a temperature between 25° C. to 100° C. The resulting compound of formula 22 can be alkylated with compound of formula 14 in the presence of organic or inorganic base (e.g. N,N-diisopropyl ethylamine, triethylamine, potassium carbonate, silver carbonate, sodium hydroxy, & sodium hydride etc.) in suitable solvents (e.g. acetonitrile or N, N-dimethylformamide etc.) under heating conditions to afford compound of formula 4a,b.

Preparation of Isoxazoline Derivatives of Formula 1a:

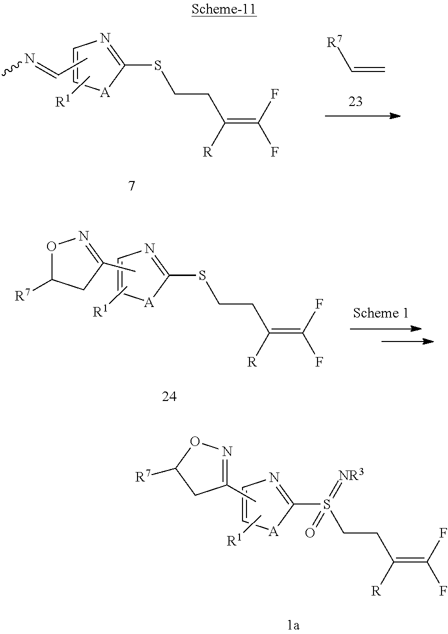

As shown in scheme 11, compound of formula 24 can be prepared by reacting compound of formula 7 with substituted styrene of formula 23 in suitable solvents (e.g. NA-dimethylformamide, dichloromethane or ethyl acetate) using suitable chlorinating agents (e.g. N-chlorosuccinimide or sodium hypochlorite etc.) at suitable temperature. Further, compound of formula 24 can be converted to compound of formula 1a following synthetic protocol given in schemes 1 and 3.

Preparation of Isoxazole Derivatives of Formula 1a:

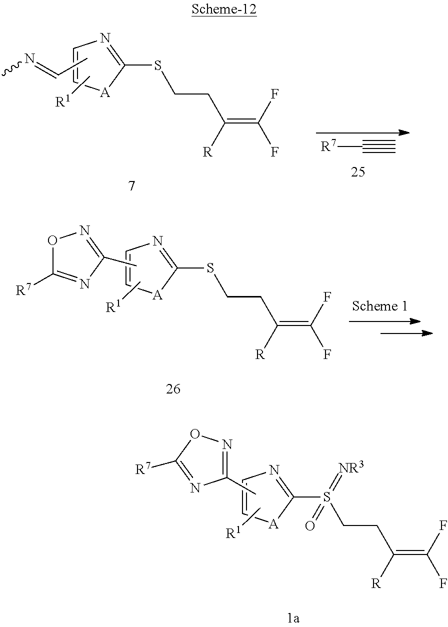

As shown in scheme 12, compounds of formula 26 can be prepared by reacting compound of formula 7 with substituted alkyne of formula 25 in suitable solvents (e.g. N,N-dimethylformamide, dichloromethane or ethyl acetate) using suitable chlorinating agents (e.g. N-chlorosuccinimide or sodium hypochlorite etc.) at 10-35° C. Further, compound of formula 26 can be converted to compound of formula 1a following synthetic protocol given in schemes 1 and 3.

Preparation of Oxadiazole Derivatives of Formula 1a:

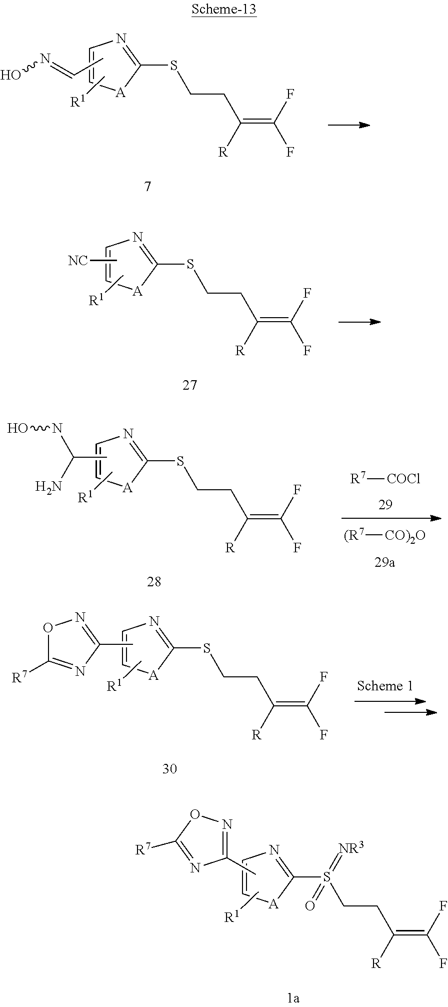

As shown in scheme 13, compounds of formula 30 can be prepared by a reaction sequence starting with dehydration of oxime 7 with dehydrating agent (e.g propylphosphonic anhydride (T₃P) solution in N, N-dimethylformamide) under heating condition. The resulting compound of formula 27 treated with hydroxyl amine hydrochloride salt in suitable solvents (e.g methanol, ethanol or pyridine) at a temperature between 25° C. to 100° C. afford compound of formula 28. The compound of formula 28 was treated with suitable acid chloride or acid anhydride of formula 29 & 29a in suitable solvents (e.g. toluene, dimethyl sulfoxide or acetic acid) at 25° C. to 100° C. furnished compound of the formula 30. Further, compound of formula 30 can be converted to compound of formula 1a following synthetic protocol given in schemes 1 and 3.

Preparation of Oxadiazolone Derivatives of Formula 1a:

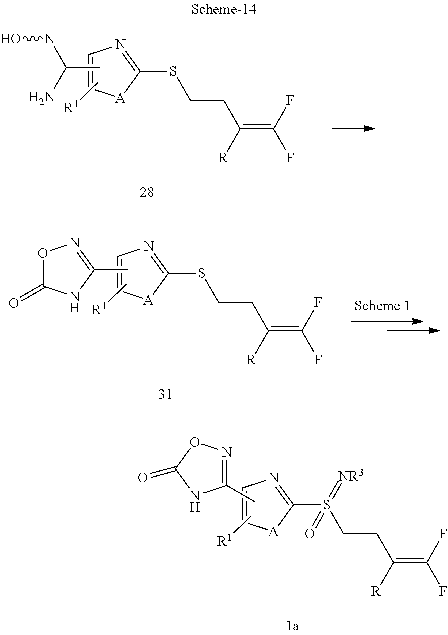

As shown in Scheme 14, compounds of formula 31 can be prepared by reaction of compounds of formula 28 with suitable reagent (eg. triphosgene or carbonyldiimidazole) in suitable solvents (e.g. dichloromethane or tetrahydrofuran) at 10-35° C. Further, compound of formula 31 can be converted to compound of formula 1a following synthetic protocol given in schemes 1 and 3.

Preparation of Difluoromethyl Derivatives of Formula 1b:

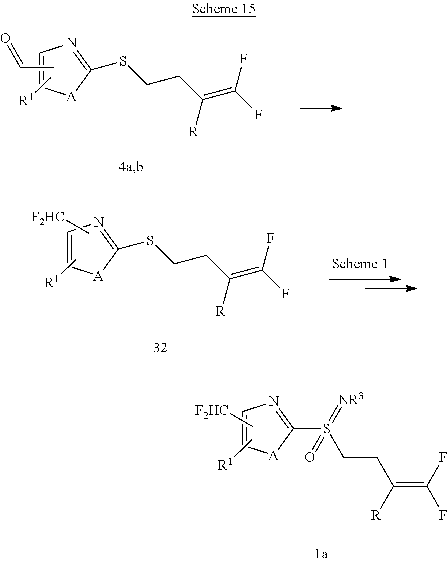

As shown in scheme 15, compounds of formula 32 can be prepared by reaction of compound of formula 4a,b with fluorinating reagent (e.g. diethylaminosulfur trifluoride) in a suitable solvent. Further, compound of formula 32 can be converted to compound of formula 1b following synthetic protocol given in schemes 1 and 3.

The invention is now illustrated in further details by the following examples, without imposing any limitation thereto.

### CHEMISTRY EXAMPLES

Synthesis of Intermediates

### a) Preparation of 2-bromo-4-methoxythiazole-5-carbonitrile

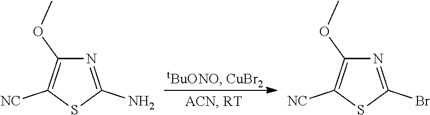

To a stirred solution of tert-butyl nitrite (0.99 g, 9.67 mmol) in acetonitrile (30 mL), copper (II) bromide (1.72 g, 1.2 mmol) was added at 0° C. The reaction mixture was stirred for 15 min. 2-Amino-4-methoxythiazole-5-carbonitrile (1.00 g, 6.44 mmol) was added to the reaction mixture, which was stirred for another 4 h. After completion of the reaction, the reaction mixture was quenched by addition of saturated aq. ammonium chloride solution (50 mL) and further extracted with ethyl acetate (50 mL×2). The combined ethyl acetate layers were washed with water and brine (each 50 mL), dried over anhydrous sodium sulphate, filtered and concentrated under reduced pressure to obtain a crude product. The crude product was purified by column chromatography using 5% ethyl acetate in hexane mixture to obtain 2-bromo-4-methoxythiazole-5-carbonitrile (0.9 g, 63.8% yield).

### b) Preparation of 2-mercapto-4-methoxythiazole-5-carbonitrile

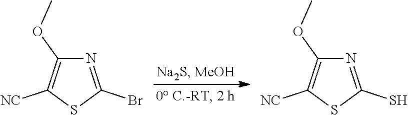

To a stirred solution of 2-bromo-4-methoxythiazole-5-carbonitrile (3.8 g, 17.4 mmol) in methanol (40 mL), sodium sulfide nonahydrate (3.3 g, 13.9 mmol) was added portion-wise at 0° C. After completion of the reaction, the solvent was evaporated, and dilute hydrochloric acid (50 mL) was added to the remaining residue at 0° C. The precipitated solid was filtered off to obtain 2-mercapto-4-methoxythiazole-5-carbonitrile (2.45 g, 82% yield) which was directly used for the next step without further purification.

### c) Preparation of Ethyl 2-bromo-4-cyclopropylthiazole-5-carboxylate

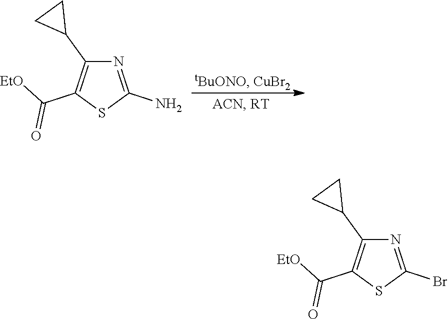 [SMILES: CCOC(=O)c1sc(N)nc1C1CC1 ; CCOC(=O)c1sc(Br)nc1C1CC1]

To a solution of tert-butyl nitrite (7.3 g, 70.7 mmol) in acetonitrile (100 mL), copper (II) bromide (8.42 g, 37.7 mmol) was added at 0° C. and the reaction mixture was stirred for 15 min. Ethyl 2-amino-4-cyclopropylthiazole-5-carboxylate (10 g, 47.1 mmol) was added dropwise to the reaction mixture, which was stirred further for 4 h. After completion of the reaction, the reaction was quenched by addition of saturated aq. ammonium chloride solution (50 mL) and the resulting mixture was extracted with ethyl acetate (50 mL×2). The combined ethyl acetate layers were washed with water and brine (each 50 mL), dried over anhydrous sodium sulphate, and after filtration the organic phase was concentrated under reduced pressure to obtain a crude product. The crude product was purified by column chromatography using 5% ethyl acetate in hexane mixture to obtain ethyl 2-bromo-4-cyclopropylthiazole-5-carboxylate (10 g, 77% yield).

### d) Preparation of (4-cyclopropyl-2-((3,4,4-trifluorobut-3-en-1-yl)thio)thiazol-5-yl)methanol

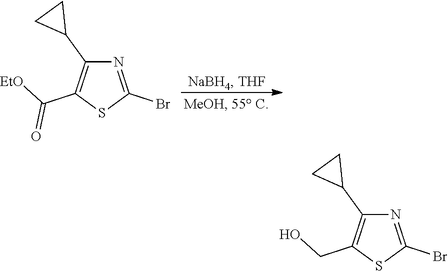 [SMILES: CCOC(=O)c1sc(Br)nc1C1CC1 ; OCc1sc(Br)nc1C1CC1]

To a solution of ethyl 2-bromo-4-cyclopropylthiazole-5-carboxylate (5 g, 18.2 mmol) in tetrahydrofuran (50 mL), sodium borohydride (3.4 g, 90.8 mmol) was added portion-wise at 0° C. The reaction mixture was allowed to reach 55° C. Methanol (20 mL) was added drop wise to the reaction mixture, and stirring was continued further for 15 min at 55° C. After completion of the reaction, the reaction mixture was cooled to 25° C., acidified using dilute hydrochloric acid (50 mL) and extracted with ethyl acetate (50 mL×2). The combined ethyl acetate layers were washed with water and brine (50 mL each), dried over anhydrous sodium sulphate, and after filtration the organic phase was concentrated under reduced pressure to obtain a crude product. The crude product was purified by column chromatography using 40% ethyl acetate in hexane mixture to yield (4-cyclopropyl-2-((3,4,4-trifluorobut-3-en-1-yl)thio)thiazol-5-yl)methanol (2.74 g, 65% yield).

### e) Preparation of 2-bromo-4-cyclopropylthiazole-5-carbaldehyde

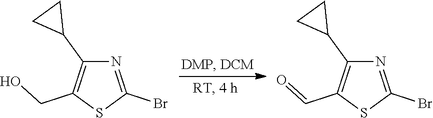 [SMILES: OCc1sc(Br)nc1C1CC1 ; O=Cc1sc(Br)nc1C1CC1]

To a cooled solution of (2-bromo-4-cyclopropylthiazol-5-yl) methanol (3.0 g, 12.81 mmol) in dichloromethane (30 mL), Dess-Martin periodinone (16.3 g, 38.4 mmol) was added portion-wise. The reaction mixture was stirred at 25° C. for 12 h. After completion of the reaction, the reaction mixture was filtered through celite. The dichloromethane layer was washed with saturated sodium bicarbonate solution (50 mL), dried over anhydrous sodium sulphate and concentrated under reduced pressure to get a crude product. The crude product was purified by column chromatography using a 10% ethyl acetate in hexane mixture to obtain 2-bromo-4-cyclopropylthiazole-5-carbaldehyde (2.3 g, 77% yield).

### f) Preparation of 4-cyclopropyl-2-mercaptothiazole-5-carbaldehyde

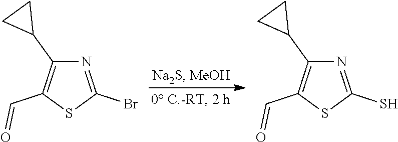 [SMILES: O=Cc1sc(Br)nc1C1CC1 ; O=Cc1sc(S)nc1C1CC1]

To a solution of 2-bromo-4-cyclopropylthiazole-5-carbaldehyde (3.0 g, 12.9 mmol) in methanol (100 mL), sodium sulfide nonahydrate (2.5 g, 10.3 mmol) was added slowly at 0° C., and the reaction mixture was stirred for 1.5 h. After completion of the reaction, methanol was removed by evaporation. 2N-hydrochloric acid was added to the residue at 0° C. Solid was filtered to obtain 4-cyclopropyl-2-mercaptothiazole-5-carbaldehyde (2.0 g, 84% yield).

### Example 1: Preparation of 2-((3,4,4-trifluorobut-3-en-1-yl)thio)thiazole-5-carbaldehyde O-ethyl Oxime

### Step A: Preparation of 2-((3,4,4-trifluorobut-3-en-1-yl)thio)thiazole

To a solution of 2((3,4,4-trifluorobut-3-en-1-yl)thio)thiazole (1 g, 8.5 mmol) in acetonitrile (10 mL) was added potassium carbonate (1.76 g, 12.8 mmol) at 25° C. After stirring for 30 min., 4-chloro-1,1,2-trifluorobut-1-ene (1.34 g, 9.35 mmol) was added to the reaction mixture and then stirred at 60° C. for 4 h. The reaction mixture was cooled to 25° C. and diluted with ethyl acetate (30 mL) and water (30 mL) and then acidified to pH 4 using 1N hydrochloric acid. The organic layer was separated and the aqueous layer was again extracted with ethyl acetate (3×30 mL). The combined organic layers were dried over anhydrous sodium sulphate, filtered and the filtrate was concentrated under reduced pressure to get the crude product. The crude product was purified by column chromatography using 10% ethyl acetate in hexane to get the title compound (1.63 g, 85% yield) as pale yellow liquid.

### Step B: Preparation of 2-((3,4,4-trifluorobut-3-en-1-yl)thio)thiazole-5-carbaldehyde

Phosphorus oxychloride (5.1 g, 33.3 mmol) was added dropwise to ice cooled dry N,N-dimethylformamide (2.4 g, mmol) and stirred for 30 min. The solution of 2-((3,4,4-trifluorobut-3-en-1-yl)thio)thiazole (1.5 g, 6.66 mmol) in 10 mL of N,N-dimethylformamide was added dropwise to the reaction mixture and stirred for 20 min. The reaction mixture was heated to 80° C. for 12 h. After completion of reaction, phosphorus oxychloride was evaporated and reaction mixture cooled to 25° C. and then neutralized using 1N sodium hydroxide solution. The reaction mixture was diluted with water (30 mL) and extracted with ethyl acetate (2×30 mL). The combined organic layers were dried over anhydrous sodium sulphate, filtered and concentrated under reduced pressure to get the crude product. The crude product was purified by column chromatography using 10% ethyl acetate in hexane to get the title compound (1.01 g, 60% yield) as yellow liquid. ¹H-NMR (400 MHz, DMSO-d₆) δ 9.92 (s, 1H), 8.57 (s, 1H), 2.54 (t, J=7 Hz, 2H), 2.47-2.90 (m, 2H)

### Step B (Alternative Method): Preparation of 2-((3,4,4-Trifluorobut-3-en-1-yl)thio)thiazole-5-carbaldehyde

To a solution of 2-mercaptothiazole-5-carbaldehyde (1 g, 6.94 mmol) in acetonitrile (10 mL) at 25° C., potassium carbonate (1.76 g, 12.8 mmol) was added and stirred for 30 min. 4-Chloro-1,1,2-trifluorobut-1-ene (1.34 g, 9.3 mmol) was added to the reaction mixture then stirred at 60° C. for 12 h. After completion of the reaction, the reaction mixture was diluted with ethyl acetate (30 mL) and water (30 mL) and acidified to pH 4 using 1N hydrochloric acid. The ethyl acetate layer was separated and the aqueous layer was extracted again with ethyl acetate (30 mL×3). The combined ethyl acetate layers were dried over anhydrous sodium sulphate, filtered and the filtrate was concentrated under reduced pressure to get the crude product. The crude product was purified by column chromatography using a 10% ethyl acetate in hexane to obtain 2-((3,4,4-trifluorobut-3-en-1-yl)thio)thiazole-5-carbaldehyde (1.23 g, 72% yield).

### Step C: Preparation of (Z)-2-((3,4,4-trifluorobut-3-en-1-yl)thio)thiazole-5-carbaldehyde O-ethyl Oxime

To a solution of 2-((3,4,4-trifluorobut-3-en-1-yl)thio)thiazole-5-carbaldehyde (1 g, 3.95 mmol) in methanol (10 mL) at 0° C. was added sodium acetate (0.32 g, 5.9 mmol) and stirred for 15 min. O-ethylhydroxylamine hydrochloride (0.4 g, 4.34 mmol) was added to the reaction mixture and stirred at 0° C., allowed to reach 25° C. and stirred for 2 h. The solvent was concentrated then diluted with ethyl acetate and water. Organic layer was separated and the aqueous layer was further extracted with ethyl acetate (2×30 mL). The combined organic layers were dried over anhydrous sodium sulphate filtered and concentrated under reduced pressure to get the crude product. The crude product was purified by column chromatography using 10% ethyl acetate in hexane to get the title compound (0.46 g, 40% yield). ¹H-NMR (400 MHz, CDCl₃): δ 8.55 (s, 1H), 7.82 (s, 1H), 4.20-4.28 (m, 2H), 3.41-3.44 (m, 2H), 2.81-2.90 (m, 2H), 1.32-1.37 (m, 3H).

### Example 2: Preparation of (Z)-2-((3,4,4-trifluorobut-3-en-1-yl)sulfinyl)thiazole-5-carbaldehyde O-ethyl Oxime

To a solution of (Z)-2-((3,4,4-trifluorobut-3-en-1-yl)thio)thiazole-5-carbaldehyde O-ethyl oxime (0.2 g, 0.67 mmol) in methanol (20 mL) at 0° C. was added oxone (0.4 g, 0.67 mmol), the reaction mixture was slowly allowed to reach 25° C. and stirred for 2 h. After completion of reaction, solvent was evaporated, reaction mixture was neutralized using 10% sodium bicarbonate solution and extracted with ethyl acetate (2×30 mL). The combined organic layers were dried over anhydrous sodium sulphate, filtered and concentrated under reduced pressure to get the crude product. The crude product was purified by column chromatography using 30% ethyl hexane in hexane to get the title compound. (0.13 g, 62% yield). ¹H-NMR (400 MHz, CDCl₃): δ 8.21 (s, 1H), 7.26 (s, 1H), 4.23 (q, J=7.1 Hz, 2H), 3.38-3.45 (m, 1H), 3.26-3.33 (m, 1H), 2.89-2.97 (m, 1H), 2.57-2.65 (m, 1H), 1.27-1.33 (m, 3H).

### Example 3: Preparation of (Z)-2-((3,4,4-trifluorobut-3-en-1-yl)sulfonyl)thiazole-5-carbaldehyde O-ethyl Oxime

To a solution of (Z)-2-((3,4,4-trifluorobut-3-en-1-yl)sulfinyl)thiazole-5-carbaldehyde O-ethyl oxime (0.2 g, 0.607 mmol) in methanol (20 mL) at 0° C. was added oxone (0.8 g, 1.3 mmol), the reaction mixture was slowly allowed to reach 25° C. and stirred for 2 h. After completion of reaction, solvent was evaporated, reaction mixture was neutralized using 10% sodium bicarbonate solution and extracted with ethyl acetate (2×30 mL). The combined organic layers were dried over anhydrous sodium sulphate, filtered and concentrated under reduced pressure to get the crude product. The crude product was purified by column chromatography using 30% ethyl acetate/hexane to get the title compound (0.19 g, 85% yield). ¹H-NMR (400 MHz, CDCl₃): δ 8.37-8.36 (s, 1H), 7.98 (s, 1H), 4.66 (q, J=7.1 Hz, 2H), 3.83-3.70 (m, 2H), 3.15-2.98 (m, 2H), 1.61-1.54 (t, J=7.1 Hz, 3H).

### Example 4: Preparation of (Z)-2-((3,4,4-trifluorobut-3-en-1-yl)sulfinyl)thiazole-5-carbaldehyde O-methyl Oxime

(Z)-2-((3,4,4-trifluorobut-3-en-1-yl)sulfinyl)thiazole-5-carbaldehyde O-methyl oxime was synthesized by following the same procedure as described in example-2, using (Z)-2-((3,4,4-trifluorobut-3-en-1-yl)thio)thiazole-5-carbaldehyde O-methyl oxime and oxone. ¹H-NMR (400 MHz, DMSO-d₆) δ 8.56 (s, 1H), 8.27 (s, 1H), 4.07 (s, 3H), 3.87 (t, J=7.1 Hz, 1H), 2.77-2.84 (m, 2H).

### Example 5: Preparation of N-((E)-(5-((Z)-(methoxyimino)methyl)thiazol-2-yl)(3,4,4-trifluorobut-3-en-1-yl)-λ⁴-sulfaneylidene)cyanamide

To a solution of (Z)-2-((3,4,4-trifluorobut-3-en-1-yl)thio)thiazole-5-carbaldehyde O-methyl oxime (0.5 g, 1.771 mmol) in acetonitrile (45 mL), cyanamide (0.223 g, 5.31 mmol) and iodobenzene diacetate (2.56 g, 7.98 mmol) were added and stirred at 25° C. for 16 h. After completion of reaction, reaction mixture was filtered through celite, washed with ethyl acetate and concentrated. The crude product was purified by column chromatography to get N-((E)-(5-((Z)-(methoxyimino)methyl)thiazol-2-yl) (3,4,4-trifluorobut-3-en-1-yl)-λ⁴-sulfanylidene)cyanamide (0.040 g, 0.124 mmol, 12%).

### Example 6: Preparation of ((Z)-2-((3,4,4-trifluorobut-3-en-1-yl)thio)thiazole-4-carbaldehyde O-ethyl Oxime

(Z)-2-((3,4,4-trifluorobut-3-en-1-yl)thio)thiazole-4-carbaldehyde O-methyl oxime was synthesized by following the same procedure as described in example-1 (Step-C), using 2-((3,4,4-trifluorobut-3-en-1-yl)thio)thiazole-4-carbaldehyde and O-ethylhydroxylamine hydrochloride. ¹H-NMR (400 MHz, DMSO-d₆): δ 8.41 (s, 1H), 7.65 (s, 1H), 4.25 (q, J=7.1 Hz, 2H), 3.46 (t, J=6.7 Hz, 2H), 2.78-2.89 (m, 2H), 1.30 (t, J=7.1 Hz, 3H).

### Example 7: Preparation of (E)-2-((3,4,4-trifluorobut-3-en-1-yl)sulfinyl)thiazole-4-carbaldehyde O-ethyl Oxime

(E)-2-((3,4,4-trifluorobut-3-en-1-yl)sulfinyl)thiazole-5-carbaldehyde O-ethyl oxime was synthesized by following the same procedure as described in example-2, using (E)-2-((3,4,4-trifluorobut-3-en-1-yl)thio)thiazole-4-carbaldehyde O-ethyl oxime and meta-chloroperbenzoic acid. ¹H-NMR (400 MHz, DMSO-d₆): δ 8.32 (s, 1H), 8.31 (s, 1H), 4.16 (q, J=7.1 Hz, 2H), 3.54-3.61 (m, 1H), 3.28-3.36 (m, 1H), 2.77-2.89 (m, 1H), 2.59-2.71 (m, 1H), 1.23 (t, J=7.1 Hz, 3H).

### Example 8: Preparation of (E)-2-((3,4,4-trifluorobut-3-en-1-yl)sulfonyl)thiazole-4-carbaldehyde O-ethyl Oxime

(E)-2-((3,4,4-trifluorobut-3-en-1-yl)sulfonyl)thiazole-4-carbaldehyde O-ethyl oxime was synthesized by following the same procedure as described in example-3, using (E)-2-((3,4,4-trifluorobut-3-en-1-yl)thio)thiazole-4-carbaldehyde O-ethyl oxime and oxone. ¹H-NMR (400 MHz, DMSO-d₆): δ 8.47 (s, 1H), 8.37 (s, 1H), 4.19 (q, J=7.1 Hz, 2H), 3.90 (t, J=7.0 Hz, 2H), 2.80-2.90 (m, 2H), 1.25 (t, J=7.1 Hz, 3H).

### Example 9: Preparation of 5-(difluoromethyl)-2-((3,4,4-trifluorobut-3-en-1-yl)sulfonyl)thiazole

### Step A: 5-(difluoromethyl)-2-((3,4,4-trifluorobut-3-en-1-yl)thio)thiazole (Compd No 1)

To a cooled solution of 2-((3,4,4-trifluorobut-3-en-1-yl)thio)thiazole-5-carbaldehyde (0.66 g, 2.3 mmol) in dichloromethane (10 mL), diethylaminosulfur trifluoride (0.76 g, 4.6 mmol) was added and stirred at 25° C. for 6 h. After completion of the reaction, the reaction mixture was diluted with dichloromethane (30 mL), washed with saturated sodium bicarbonate solution (30 mL) and water (30 mL). The dichloromethane layer was dried over anhydrous sodium sulphate and concentrated under reduced pressure to get the crude product. The crude product was purified by column chromatography using a 20% ethyl acetate in hexane mixture to obtain 5-(difluoromethyl)-2-((3,4,4-trifluorobut-3-en-1-yl)thio)thiazole (0.43 g, 60% yield).

### Step B: 5-(Difluoromethyl)-2-((3,4,4-trifluorobut-3-en-1-yl)sulfinyl)thiazole

To a solution of 5-(difluoromethyl)-2-((3,4,4-trifluorobut-3-en-1-yl)thio)thiazole (0.2 g, 0.07 mmol) in dichloromethane (20 mL) at 0° C., meta-chloroperbenzoic acid (0.16 g, 0.07 mmol) was added portion-wise. The reaction mixture was slowly allowed to reach 25° C. and stirred for 4 h. After completion of the reaction, the solvent was evaporated, the reaction mixture was neutralized using 10% sodium bicarbonate solution and extracted with ethyl acetate (30 mL×2). The combined organic layers were dried over anhydrous sodium sulphate, filtered and the filtrate was concentrated under reduced pressure to get the crude product. The crude product was purified by column chromatography using 30% ethyl hexane in hexane to obtain 5-(difluoromethyl)-2-((3,4,4-trifluorobut-3-en-1-yl)sulfinyl)thiazole (0.15 g, 73% yield).

### Step C: 5-(Difluoromethyl)-2-((3,4,4-trifluorobut-3-en-1-yl)sulfonyl)thiazole

To a solution of 5-(difluoromethyl)-2-((3,4,4-trifluorobut-3-en-1-yl)thio)thiazole (0.2 g, 0.07 mmol) in dichloromethane (20 mL) at 0° C., meta-chloroperbenzoic acid (0.35 g, 0.14 mmol) was added portion-wise, the reaction mixture was slowly allowed to reach 25° C. and stirred for 4 h. After completion of the reaction, the solvent was evaporated, the reaction mixture was neutralized using 10% sodium bicarbonate solution and extracted with ethyl acetate (30 mL×2). The combined organic layers were dried over anhydrous sodium sulphate, filtered and the filtrate was concentrated under reduced pressure to give the crude product. The crude product was purified by column chromatography using 30% ethyl hexane in hexane to obtain 5-(difluoromethyl)-2-((3,4,4-trifluorobut-3-en-1-yl)sulfinyl)thiazole (0.19 g, 85% yield).

### Example 10: Preparation of N-45-(difluoromethyl)thiazol-2-yl)(3,4,4-trifluorobut-3-en-1-yl)-λ⁴-sulfaneylidene)cyanamide

To a solution of 5-(difluoromethyl)-2-((3,4,4-trifluorobut-3-en-1-yl)thio)thiazole (0.50 g, 1.8 mmol) in acetonitrile (10 mL) at 0° C., iodobenzene diacetate (0.88 g, 2.8 mmol) and cyanamide (0.16 g, 3.6 mmol) were added. The reaction mixture was slowly allowed to reach 25° C. and stirred for 4 h. After completion of the reaction, the reaction mixture was evaporated, diluted with water (30 mL) and extracted with ethyl acetate (30 mL×2). The combined organic layers were dried over anhydrous sodium sulphate and concentrated under reduced pressure to give a crude product. The crude product was purified by column chromatography using 80% ethyl acetate in hexane to obtain N-((5-(difluoromethyl)thiazol-2-yl)(3,4,4-trifluorobut-3-en-1-yl)-λ⁴-sulfaneylidene)cyanamide (0.12 g, 21% yield).

### Example 11: Preparation of (5-(difluoromethyl)thiazol-2-yl)(imino)(3,4,4-trifluorobut-3-en-1-yl)-λ⁶-sulfanone

To a solution of 5-(difluoromethyl)-2-((3,4,4-trifluorobut-3-en-1-yl)thio)thiazole (0.50 g, 1.8 mmol) in methanol (10 mL) at 0° C., iodobenzene diacetate (0.88 g, 3.6 mmol) was added, followed by ammonium carbamate (0.3 g, 3.6 mmol). The reaction mixture was allowed to reach 25° C. and stirred for 4 h. After completion of the reaction, the reaction mixture was evaporated, diluted with water (30 mL) and extracted with ethyl acetate (30 mL×2). The combined organic layers were dried over anhydrous sodium sulphate and concentrated under reduced pressure to give a crude product. The crude product was purified by column chromatography using 80% ethyl acetate in hexane to obtain (5-(difluoromethyl)thiazol-2-yl)(imino)(3,4,4-trifluorobut-3-en-1-yl)-λ⁶-sulfanone (0.46 g, 83% yield).

### Example 12: Preparation of N-((5-chlorothiazol-2-yl)(3,4,4-trifluorobut-3-en-1-yl)-λ⁴-sulfaneylidene)cyanamide

To a solution of 5-chloro-2-((3,4,4-trifluorobut-3-en-1-yl)thio)thiazole (0.50 g, 1.92 mmol) in acetonitrile (10 mL) at 0° C. was added iodobenzene diacetate (0.92 g, 2.88 mmol) followed by cyanamide (0.24 g, 5.77 mmol), the reaction mixture was slowly allowed to reach 25° C. and stirred for 4 h. After completion of reaction, reaction mixture was evaporated, diluted with water (30 mL) and extracted with ethyl acetate (30 mL×2). The combined organic layer was dried over anhydrous sodium sulfate and concentrated under reduced pressure to get the crude product. The crude product was purified by column chromatography using 80% ethyl acetate in hexane to get the title compound (0.30 g, 53% yield). ¹H-NMR (400 MHz, CDCl₃): δ 7.83 (s, 1H), 3.73-3.67 (m, 2H), 3.02-2.96 (m, 1H), 2.97-2.84 (m, 1H).

### Example 13: Preparation of N-((5-chlorothiazol-2-yl)(oxo)(3,4,4-trifluorobut-3-en-1-yl)-λ⁶-sulfaneylidene)cyanamide

To a solution of N-((5-chlorothiazol-2-yl)(oxo)(3,4,4-trifluorobut-3-en-1-yl)-λ⁶-sulfaneylidene)cyanamide (0.2 g, 0.66 mmol) in methanol (20 mL) at 0° C. was added oxone (0.61 g, 1.5 mmol), the reaction mixture was slowly allowed to reach 25° C. and stirred for 4 h. After completion of reaction, reaction mixture was evaporated and diluted with water (30 mL) and extracted with ethyl acetate (30 mL×2). The combined organic layers were dried over anhydrous sodium sulphate and concentrated under reduced pressure to get the crude product. The crude product was purified by column chromatography using 50% ethyl acetate in hexane to get the title compound (0.13 g, 65% yield). ¹H-NMR (400 MHz, CDCl₃): δ 8.01 (s, 1H), 3.94-3.84 (m, 2H), 3.05-2.95 (m, 2H).

### Example 14: Preparation of 5-phenyl-3-(2-((3,4,4-trifluorobut-3-en-1-yl)thio)thiazol-5-yl)-4,5-dihydroisoxazole

To the stirred solution of 2-((3,4,4-trifluorobut-3-en-1-yl)thio)thiazole-5-carbaldehyde oxime (0.50 g, 1.86 mmol) in N,N-dimethylformamide (5 mL) was added N-chlorosuccinimide (0.30 g, 2.2 mmol) at 25° C. and reaction was stirred for 1 h. 1,8-Diazabicyclo(5.4.0)undec-7-ene (0.28 g, 1.86 mmol) and styrene (0.23 g, 2.2 mmol) were added and reaction mixture was heated to 60° C. for 8 h. After completion of the reaction, water (20 mL) was added and extracted with ethyl acetate (3×20 mL). The combined organic layer was dried on anhydrous sodium sulfate and concentrated under reduced pressure to get crude product. The crude was purified by column chromatography using 20% ethyl acetate in hexane to get the title compound (0.49 g, 71% yield). ¹H-NMR (400 MHz, CDCl₃): δ 7.62-7.66 (m, 1H), 7.31-7.41 (m, 5H), 5.76 (dd, J=11.0, 8.4 Hz, 1H), 3.75 (dd, J=16.4, 10.9 Hz, 1H), 3.45 (t, J=7.0 Hz, 2H), 3.32 (q, J=8.3 Hz, 1H), 2.76-2.87 (m, 2H).

### Example 15: Preparation of 5-phenyl-3-(2-((3,4,4-trifluorobut-3-en-1-yl)sulfonyl)thiazol-5-yl)-4,5-dihydroisoxazole

5-phenyl-3-(2-((3,4,4-trifluorobut-3-en-1-yl)sulfonyl)thiazol-5-yl)-4,5-dihydroisoxazole was synthesized by following the same procedure as described in example-3, using 5-phenyl-3-(2-((3,4,4-trifluorobut-3-en-1-yl)thio)thiazol-5-yl)-4,5-dihydroisoxazole. ¹H-NMR (400 MHz, CDCl₃) δ: 8.00-8.01 (m, 1H), 7.34-7.43 (m, 5H), 5.87 (dd, J=11.1, 8.5 Hz, 1H), 3.78-3.86 (m, 1H), 3.61-3.66 (m, 2H), 3.33-3.41 (m, 1H), 2.86-2.97 (m, 2H).

### Example 16: Preparation of 5-(4-chlorophenyl)-3-(2-((3,4,4-trifluorobut-3-en-1-yl)thio)thiazol-4-yl)isoxazole

To the stirred solution of 2-((3,4,4-trifluorobut-3-en-1-yl)thio)thiazole-4-carbaldehyde oxime (0.50 g, 1.86 mmol) in N,N-dimethylformamide (5 ml) was added N-chlorosuccinimide (0.30 g, 2.2 mmol) at 25° C. and reaction was stirred for 0.5-1 h. 1,8-Diazabicyclo[5.4.0]undec-7-ene (0.28 g, 1.86 mmol) and 1-chloro-4-ethynylbenzene (0.30 g, 2.2 mmol) were added and reaction mixture was heated at 60° C. for 8 h. After completion of the reaction, water (20 ml) was added and extracted with ethyl acetate (3×20 ml). The combined organic layer were dried over anhydrous sodium sulphate and concentrated under reduced pressure to get the crude product. The crude product was purified by column chromatography using 20% ethyl acetate/hexane to get the title compound (0.47 g, 63% yield). ¹H-NMR (400 MHz, DMSO-d₆) δ: 8.25 (s, 1H), 7.94-7.97 (m, 2H), 7.64 (dt, J=9.0, 2.2 Hz, 2H), 7.50 (s, 1H), 3.52 (t, J=6.8 Hz, 2H), 2.83-2.94 (m, 2H).

### Example 17: Preparation of 2-((3,4,4-trifluorobut-3-en-1-yl)thio)thiazole-5-carbonitrile

To a solution of 2((3,4,4-trifluorobut-3-en-1-yl)thio)oxazole-5-carbaldehyde (200 mg, 0.84 mmol) in N,N-dimethylformamide (3 mL), hydroxylamine hydrochloride (64.5 mg, 0.92 mmol) was added, followed by addition of 1-propanephosphonic acid cyclic anhydride (295 mg, 0.93 mmol) and triethylamine (0.13 mL, 0.92 mmol). The reaction mixture was stirred at 100° C. for 3 h. After completion of the reaction, the reaction mixture was diluted with ethyl acetate (30 mL) and washed with water (30 mL×2). The combined ethyl acetate layers were washed with brine solution (30 mL) and dried over anhydrous sodium sulphate, filtered and concentrated under reduced pressure to give a crude product. The crude product was purified by column chromatography using 20% ethyl acetate in hexane mixture to obtain 5-(difluoromethyl)-2-((3,4,4-trifluorobut-3-en-1-yl)thio)thiazole (155 mg, 78% yield).

### Example 18: Preparation of 5-cyclopropyl-3-(2-((3,4,4-trifluorobut-3-en-1-yl)thio)thiazol-5-yl)-1,2,4-oxadiazole

### Step A: N-hydroxy-2-((3,4,4-trifluorobut-3-en-1-yl)thio)thiazole-5-carboximidamide

Hydroxylamine hydrochloride (0.67 g, 9.59 mmol) and sodium acetate (1.311 g, 15.98 mmol) were added to the solution of 2-((3,4,4-trifluorobut-3-en-1-yl)thio)thiazole-5-carbonitrile (2.0 g, 7.99 mmol) in methanol (15 mL) at 25° C. and stirred for 2 h. The reaction mixture was evaporated under reduced pressure, water was added and extracted using ethyl acetate and the organic solvent was evaporated to get N-hydroxy-2-((3,4,4-trifluorobut-3-en-1-yl)thio)thiazole-5-carboximidamide as a crude product.

### Step B: N′-((cyclopropanecarbonyl)oxy)-2-((3,4,4-trifluorobut-3-en-1-yl)thio)thiazole-5-carboximidamide

N-hydroxy-2-((3,4,4-trifluorobut-3-en-1-yl)thio)thiazole-5-carboximidamide (1.2 g, 4.24 mmol) was dissolved in N,N-dimethylformamide (8 mL). Triethyl amine (0.590 ml, 4.24 mmol) and cyclopropanecarboxylic acid chloride (0.443 ml, 4.66 mmol) were added at 0° C. and stirred for 2 h. The reaction mixture was poured into ice-water, the solid obtained was filtered to get N′-((cyclopropanecarbonyl)oxy)-2-((3,4,4-trifluorobut-3-en-1-yl)thio)thiazole-5-carboximidamide (1.0 g, 2.85 mmol, 67.2% yield) as desired compound.

### Step C: 5-cyclopropyl-3-(2-((3,4,4-trifluorobut-3-en-1-yl)thio)thiazol-5-yl)-1,2,4-oxadiazole

N′-((cyclopropanecarbonyl)oxy)-2-((3,4,4-trifluorobut-3-en-1-yl)thio)thiazole-5-carboximidamide (0.8 g, 2.28 mmol) was dissolved in dimethyl sulfoxide (5 ml) and potassium hydroxide (0.13 g, 2.28 mmol) was added. The reaction mixture was stirred at 25° C. for 1 h. The reaction mixture was poured into ice water and extracted with ethyl acetate. The solvent was concentrated and purification on column chromatography to get 5-cyclopropyl-3-(2-((3,4,4-trifluorobut-3-en-1-yl)thio)thiazol-5-yl)-1,2,4-oxadiazole as oily compound.

### Example 19: Preparation of (Z)-2-((3,4,4-trifluorobut-3-en-1-yl)thio)oxazole-5-carbaldehyde O-methyl Oxime

### Step A: 2-((3,4,4-trifluorobut-3-en-1-yl)thio)oxazole-5-carbaldehyde

Phosphorous oxychloride (5.1 g, 33.3 mmol) was added drop-wise to ice cooled dry N,N-dimethylformamide (2.4 g, mmol) and stirred for 30 min. A solution of 2-((3,4,4-trifluorobut-3-en-1-yl)thio)thiazole (1.5 g, 6.6 mmol) in dry N,N-dimethylformamide (10 mL) was added drop-wise to the reaction mixture and stirring was continued for another 20 min. The reaction mixture was allowed to reach 25° C. and heated to 80° C. under stirring for 12 h. After completion of the reaction, phosphorous oxychloride was evaporated the reaction mass was cooled to 25° C. and neutralized using 1N sodium hydroxide solution, diluted with water (30 mL) and extracted with ethyl acetate (30 mL×2). The combined organic layers were dried over anhydrous sodium sulphate, filtered and concentrated under reduced pressure to give a crude product. The crude product was purified by column chromatography using 10% ethyl acetate in hexane to obtain 2-((3,4,4-Trifluorobut-3-en-1-yl)thio)oxazole-5-carbaldehyde (1.01 g, 60% yield). ¹H-NMR (400 MHz, CDCl₃) δ 9.66 (s, 1H), 7.80 (s, 1H), 3.41-3.49 (m, 2H), 2.86-2.91 (m, 2H).

### Step B: (Z)-2-((3,4,4-trifluorobut-3-en-1-yl)thio)oxazole-5-carbaldehyde O-methyl Oxime

To a solution of 2-((3,4,4-trifluorobut-3-en-1-yl)thio)oxazole-5-carbaldehyde (200 mg, 0.843 mmol) in methanol (10 mL) were added sodium acetate (138 mg, 1.686 mmol), methoxylamine hydrochloride (85 mg, 1.0 mmol) and then stirred for 2 h. The reaction mixture was quenched with ice-water and extracted with ethyl acetate, organic layer was washed with water, brine, dried over anhydrous sodium sulphate and the solvent was concentrated to afford the crude product. The crude product was purified by combiflash column to get the desired product (90 mg, 40% yield).

The following table illustrates in a non-limiting manner examples of compounds according to the invention.

In the following examples, ¹H-NMR data of selected examples are written in form of ¹H-NMR-peak lists. To each signal peak are listed the 6-value in ppm and the no of proton in round brackets.

For calibrating chemical shift for ¹H spectra, we use tetramethylsilane and/or the chemical shift of the solvent used, especially in the case of spectra measured in DMSO. Therefore in NMR peak lists, tetramethylsilane peak can occur but not necessarily.

TABLE 1
NMR data of synthesized compounds of formula (I)

| Sr. | | |
|---|---|---|
| No | Compound Name | NMR DATA |
| 1 | (E)-2-((3,4,4-trifluorobut-3-en-1- | ¹H-NMR (400 MHz, CDCl₃) δ 8.14 (s, 1H), 7.65 (s, 1H), |
| | yl)thio)thiazole-5-carbaldehyde O- | 3.93 (s, 3H), 3.42 (t, J = 7.1 Hz, 2H), 2.75-2.86 (m, 2H) |
| | methyl oxime | |
| 2 | (E)-2-((3,4,4-trifluorobut-3-en-1- | ¹H-NMR (400 MHz, CDCl₃) δ 7.86 (s, 1H), 7.60 (s, 1H), |
| | yl)thio)thiazole-5-carbaldehyde O- | 4.05 (s, 3H), 3.43 (t, J = 7.0 Hz, 2H), 2.75-2.85 (m, 2H); |
| | methyl oxime | |
| 3 | (Z)-2-((3,4,4-trifluorobut-3-en-1- | ¹H-NMR (400 MHz, CDCl₃) δ 7.71 (s, 1H), 7.40 (s, 1H), |
| | yl)thio)oxazole-5-carbaldehyde O- | 4.08 (s, 3H), 3.37 (t, J = 7.0 Hz, 2H), 2.77-2.88 (m, 2H); |
| | methyl oxime | |
| 4 | (E)-2-((3,4,4-trifluorobut-3-en-1- | ¹H-NMR (400 MHz, CDCl₃) δ 7.71 (s, 1H), 7.40 (s, 1H), |
| | yl)sulfinyl)thiazole-5-carbaldehyde O- | 4.08 (s, 3H), 3.37 (t, J = 7.0 Hz, 2H), 2.77-2.88 (m, 2H); |
| | methyl oxime | |
| 5 | (Z)-2-((3,4,4-trifluorobut-3-en-1- | ¹H-NMR (400 MHz, DMSO-d₆) δ 8.56 (s, 1H), 8.27 (s, |
| | yl)sulfinyl)thiazole-5-carbaldehyde O- | 1H), 4.07 (s, 3H), 3.87 (t, J = 7.1 Hz, 2H), 2.77-2.84 (m, |
| | methyl oxime | 2H); |
| 6 | (E)-2-((3,4,4-trifluorobut-3-en-1- | ¹H-NMR (400 MHz, CDCl₃) δ 7.71 (s, 1H), 7.40 (s, 1H), |
| | yl)thio)oxazole-5-carbaldehyde O- | 4.08 (s, 3H), 3.37 (t, J = 7.0 Hz, 2H), 2.77-2.88 (m, 2H); |
| | methyl oxime | |
| 7 | (E)-2-((3,4,4-trifluorobut-3-en-1- | ¹H-NMR (400 MHz, DMSO-d₆) δ 8.35 (s, 1H), 7.76 (s, |
| | yl)sulfinyl)oxazole-5-carbaldehyde O- | 1H), 3.93 (s, 3H), 3.62-3.69 (m, 1H), 3.49-3.56 (m, 1H), |
| | methyl oxime | 2.83-2.88 (m, 2H); |
| 8 | (Z)-2-((3,4,4-trifluorobut-3-en-1- | ¹H-NMR (400 MHz, DMSO-d₆) δ 8.03 (s, 1H), 7.94 (s, |
| | yl)sulfinyl)oxazole-5-carbaldehyde O- | 1H), 4.04 (s, 3H), 3.63-3.70 (m, 2H), 3.48-3.55 (m, 2H); |
| | methyl oxime | |
| 9 | (E)-2-((3,4,4-trifluorobut-3-en-1- | ¹H-NMR (400 MHz, DMSO-d₆) δ 8.03 (s, 1H), 7.94 (s, |
| | yl)sulfonyl)oxazole-5-carbaldehyde O- | 1H), 4.04 (s, 3H), 3.71-3.61 (m, 2H), 3.57-3.46 (m, 2H); |
| | methyl oxime | |
| 10 | 2-((3,4,4-trifluorobut-3-en-1- | ¹H-NMR (400 MHz, CDCl₃) δ 8.55 (s, 1H), 7.82 (s, 1H), |
| | yl)thio)thiazole-5-carbaldehyde O- | 4.20-4.28 (m, 2H), 3.41-3.44 (m, 2H), 2.81-2.90 (m, 2H), |
| | ethyl oxime | 1.32-1.37 (m, 3H); |
| 11 | (Z)-2-((3,4,4-trifluorobut-3-en-1- | ¹H-NMR (400 MHz, CDCl₃) δ 7.96 (s, 1H), 7.31 (s, 1H), |
| | yl)sulfonyl)thiazole-5-carbaldehyde O- | 4.23-4.31 (m, 2H), 4.07 (s, 3H), 3.40-3.45 (m, 2H); |
| | methyl oxime | |
| 12 | (E)-2-((3,4,4-trifluorobut-3-en-1- | ¹H-NMR (400 MHz, CDCl₃) δ 8.02 (s, 1H), 7.85 (s, 1H), |
| | yl)thio)thiazole-5-carbaldehyde O- | 4.01 (q, J = 7.3 Hz, 2H), 3.42 (t, J = 7.0 Hz, 2H), 2.74-2.85 |
| | ethyl oxime | (m, 2H), 1.54 (t, J = 7.0 Hz, 3H); |
| 13 | (Z)-2-((3,4,4-trifluorobut-3-en-1- | ¹H-NMR (400 MHz, CDCl₃) δ 8.29 (s, 1H), 7.57 (s, 1H), |
| | yl)thio)oxazole-5-carbaldehyde O- | 3.98 (q, J = 7.2 Hz, 2H), 3.40-3.36 (m, 2H), 2.88-2.77 (m, |
| | ethyl oxime | 2H), 1.34 (t, J = 7.2 Hz, 3H); |
| 14 | (E)-2-((((2-((3,4,4-trifluorobut-3-en-1- | ¹H-NMR (400 MHz, DMSO-d₆) δ 8.51 (s, 1H), 8.35 (s, |
| | yl)thio)thiazol-5- | 1H), 5.41 (s, 2H), 3.48 (t, J = 6.8 Hz, 2H), 2.78-2.87 (m, |
| | yl)methylene)amino)oxy)acetonitrile | 2H); |
| 15 | (E)-2-((3,4,4-trifluorobut-3-en-1- | ¹H-NMR (400 MHz, DMSO-d₆) δ 8.49 (s, 1H), 8.20 (s, |
| | yl)thio)thiazole-5-carbaldehyde O- | 1H), 4.32-4.39 (m, 1H), 3.44 (t, J = 6.8 Hz, 2H), 2.76-2.87 |
| | isopropyl oxime | (m, 2H), 1.31 (d, J = 6.8 Hz, 6H); |
| 16 | (E)-2-((3,4,4-trifluorobut-3-en-1- | ¹H-NMR (400 MHz, DMSO-d₆) δ 8.46 (s, 1H), 8.22 (s, |
| | yl)thio)thiazole-5-carbaldehyde O- | 1H), 3.78 (d, J = 7.3 Hz, 2H), 3.45 (t, J = 6.8 Hz, 2H), |
| | cyclopropylmethyl oxime | 2.76-2.87 (m, 2H), 1.30-1.37 (m, 1H), 0.55-0.60 (m, 2H), |
| | | 0.35-0.39 (m, 2H); |
| 17 | (E)-2-((3,4,4-trifluorobut-3-en-1- | ¹H-NMR (400 MHz, CDCl₃) δ 7.82 (s, 1H), 7.32 (s, 1H), |
| | yl)thio)oxazole-5-carbaldehyde O- | 4.22 (q, J = 7.2 Hz, 2H), 3.41-3.44 (m, 2H), 2.81-2.90 (m, |
| | ethyl oxime | 2H), 1.35 (t, J = 7.2 Hz, 3H); |
| 18 | (Z)-2-((3,4,4-trifluorobut-3-en-1- | ¹H-NMR (400 MHz, CDCl₃) δ 7.96 (s, 1H), 7.31 (s, 1H), |
| | yl)sulfinyl)oxazole-5-carbaldehyde O- | 4.23-4.31 (m, 2H), 3.40-3.45 (m, 2H), 2.83-2.94 (m, 2H), |
| | ethyl oxime | 1.33-1.38 (m, 3H); |
| 19 | (Z)-4-phenyl-2((3,4,4-trifluorobut-3- | ¹H-NMR (400 MHz, DMSO-d₆) δ 8.27 (s, 1H), 7.62 (dd, |
| | en-1-yl)thio)thiazole-5-carbaldehyde | J = 7.9, 1.5 Hz, 2H), 7.46-7.52 (m, 3H), 3.86 (s, 3H), 3.52 (t, |
| | O-methyl oxime | J = 6.9 Hz, 2H), 2.85-2.95 (m, 2H). |
| 20 | (E)-4-phenyl-2((3,4,4-trifluorobut-3- | ¹H-NMR (400 MHz, DMSO-d₆) δ 7.72 (s, 1H), 7.63 (dd, |
| | en-1-yl)thio)thiazole-5-carbaldehyde | J = 7.7, 1.6 Hz, 2H), 7.47-7.55 (m, 3H), 3.99 (s, 3H), 3.52 (t, |
| | O-methyl oxime | J = 6.9 Hz, 2H), 2.83-2.94 (m, 2H); |
| 21 | (Z)-2-((3,4,4-trifluorobut-3-en-1- | ¹H-NMR (400 MHz, DMSO-d₆) δ 8.42 (s, 1H), 7.92 (s, |
| | yl)thio)thiazole-5-carbaldehyde O- | 1H), 4.09 (q, J = 7.0 Hz, 2H), 3.47 (t, J = 6.8 Hz, 2H), |
| | ethyl oxime | 2.77-2.88 (m, 2H), 1.20 (t, J = 7.0 Hz, 3H); |
| 22 | (E)-2-((3,4,4-trifluorobut-3-en-1- | ¹H-NMR (400 MHz, DMSO-d₆) δ 8.47 (s, 1H), 8.16 (s, |
| | yl)sulfinyl)thiazole-5-carbaldehyde O- | 1H), 4.31 (q, J = 7.0 Hz, 2H), 3.57-3.64 (m, 1H), 3.35 (q, |
| | ethyl oxime | J = 7.0 Hz, 1H), 2.76-2.89 (m, 1H), 2.55-2.69 (m, 1H), 1.30 |
| | | (t, J = 7.0 Hz, 3H); |
| 23 | (E)-2-((3,4,4-trifluorobut-3-en-1- | ¹H-NMR (400 MHz, DMSO-d₆) δ 8.58 (s, 1H), 8.30 (s, |
| | yl)sulfonyl)thiazole-5-carbaldehyde O- | 1H), 4.36 (q, J = 7.0 Hz, 2H), 3.90 (t, J = 7.0 Hz, 2H), |
| | ethyl oxime | 2.78-2.89 (m, 2H), 1.32 (t, J = 7.0 Hz, 3H); |
| 24 | (Z)-4-phenyl-24(3,4,4-trifluorobut-3- | ¹H-NMR (400 MHz, DMSO-d₆) δ 8.13 (s, 1H), 7.96 (s, |
| | en-1-yl)thio)thiazole-5-carbaldehyde | 1H), 4.21 (t, J = 6.6 Hz, 2H), 3.47 (t, J = 6.8 Hz, 2H), 2.91- |
| | O-ethyl oxime | 2.74 (2H), 1.78-1.62 (1H), 1.56 (d, J = 6.7 Hz, 2H), 0.90 |
| | | (d, J = 6.6 Hz, 6H); |
| 25 | (E)-4-phenyl-2((3,4,4-trifluorobut-3- | ¹H-NMR (400 MHz, DMSO-d₆) δ 8.13 (s, 1H), 7.97 (s, |
| | en-1-yl)thio)thiazole-5-carbaldehyde | 1H), 4.23 (s, 1H), 3.47 (t, J = 6.8 Hz, 2H), 2.82 (ddd, J = |
| | O-ethyl oxime | 23.1, 4.0, 2.8 Hz, 2H), 1.86 (dd, J = 8.3, 3.8 Hz, 2H), 1.66 |
| | | (dd, J = 6.0, 2.4 Hz, 2H), 1.48-1.53 (m, 3H), 1.32-1.37 (m, |
| | | 3H); |
| 26 | (E)-4-methyl-2-((3,4,4-trifluorobut-3- | ¹H-NMR (400 MHz, CDCl₃) δ 7.58 (s, 1H), 4.03 (s, 3H), |
| | en-1-yl)thio)thiazole-5-carbaldehyde | 3.43 (t, J = 7.2 Hz, 2H), 2.84-2.75 (m, 2H), 2.53 (s, 3H); |
| | O-methyl oxime | |
| 27 | (Z)-4-methyl-2-((3,4,4-trifluorobut-3- | ¹H-NMR (400 MHz, CDCl₃) δ 8.14 (s, 1H), 4.08 (s, 3H), |
| | en-1-yl)thio)thiazole-5-carbaldehyde | 3.42 (t, J = 7.2 Hz, 2H), 2.85-2.75 (m, 2H), 2.53 (s, 3H); |
| | O-methyl oxime | |
| 28 | (Z)-4-phenyl-2((3,4,4-trifluorobut-3- | ¹H-NMR (400 MHz, DMSO-d₆) δ 8.37 (s, 1H), 7.62-7.65 |
| | en-1-yl)sulfinyl)thiazole-5- | (m, 2H), 7.48-7.55 (m, 3H), 3.94 (d, J = 10.4 Hz, 3H), |
| | carbaldehyde O-methyl oxime | 3.62-3.69 (m, 1H), 3.39-3.46 (m, 1H), 2.72-2.90 (m, 2H); |
| 29 | (E)-4-phenyl-2((3,4,4-trifluorobut-3- | ¹H-NMR (400 MHz, DMSO-d₆) δ 8.00 (s, 1H), 7.66-7.70 |
| | en-1-yl)sulfonyl)thiazole-5- | (m, 2H), 7.53-7.60 (m, 3H), 4.14 (s, 3H), 3.96 (t, J = 7.0 |
| | carbaldehyde O-methyl oxime | Hz, 2H), 2.84-2.95 (m, 2H); |
| 30 | (E)-4-phenyl-24(3,4,4-trifluorobut-3- | ¹H-NMR (400 MHz, DMSO-d₆) δ 7.88 (d, J = 5.3 Hz, 1H), |
| | en-1-yl)sulfinyl)thiazole-5-carbaldehyde | 7.62-7.67 (m, 2H), 7.50-7.57 (m, 3H), 4.08 (s, 3H), 3.62- |
| | O-methyl oxime | 3.69 (m, 1H), 3.38-3.45 (m, 1H), 2.81-2.92 (m, 1H), 2.64- |
| | | 2.77 (m, 1H); |
| 31 | (Z)-2-((3,4,4-trifluorobut-3-en-1- | ¹H-NMR (400 MHz, CDCl₃) δ 7.90-7.97 (m, 1H), 7.49- |
| | yl)sulfonyl)oxazole-5-carbaldehyde O- | 7.59 (m, 1H), 4.35-4.46 (m, 3H), 3.62-3.68 (m, 2H), 2.88- |
| | ethyl oxime | 2.99 (m, 2H), 1.36-1.41 (m, 3H); |
| 32 | (E)-2-((3,4,4-trifluorobut-3-en-1- | ¹H-NMR (400 MHz, CDCl₃) δ 7.89-8.04 (m, 1H), 7.46- |
| | yl)sulfonyl)oxazole-5-carbaldehyde O- | 7.57 (m, 1H), 4.37-4.43 (m, 1H), 4.26-4.32 (m, 1H), 3.59- |
| | ethyl oxime | 3.70 (m, 2H), 2.87-2.99 (m, 2H), 1.30-1.41 (m, 3H); |
| 33 | (E)-4-(tert-butyl)-2((3,4,4-trifluorobut- | ¹H-NMR (400 MHz, DMSO-d₆) δ 8.16 (s, 1H), 3.94 (s, |
| | 3-en-l-yl)thio)thiazole-5-carbaldehyde | 3H), 3.43 (t, J = 7.0 Hz, 2H), 2.81-2.88 (m, 2H), 1.40 (s, |
| | O-methyl oxime | 9H); |
| 34 | (E)-2-((3,4,4-trifluorobut-3-en-1- | ¹H-NMR (400 MHz, DMSO-d₆) δ 8.13 (s, 1H), 7.96 (s, |
| | yl)thio)thiazole-5-carbaldehyde O- | 1H), 4.21 (t, J = 6.6 Hz, 2H), 3.47 (t, J = 6.8 Hz, 2H), 2.91- |
| | isopentyl oxime | 2.74 (m,2H), 1.78-1.62 (m, 1H), 1.56 (d, J = 6.7 Hz, 2H), |
| | | 0.90 (d, J = 6.6 Hz, 6H); |
| 35 | (E)-2-((3,4,4-trifluorobut-3-en-1- | ¹H-NMR (400 MHz, DMSO-d₆) δ 8.13 (s, 1H), 7.97 (s, |
| | yl)thio)thiazole-5-carbaldehyde O- | 1H), 4.23 (s, 1H), 3.47 (t, J = 6.8 Hz, 2H), 2.82 (ddd, J = |
| | cyclohexyl oxime | 23.1, 4.0, 2.8 Hz, 2H), 1.86 (dd, J = 8.3, 3.8 Hz, 2H), 1.66 |
| | | (dd, J = 6.0, 2.4 Hz, 2H), 1.48-1.53 (m, 3H), 1.32-1.37 (m, |
| | | 3H); |
| 36 | (E)-2-((3,4,4-trifluorobut-3-en-1- | ¹H-NMR (400 MHz, DMSO-d₆) δ 8.31 (s, 1H), 7.72 (s, |
| | yl)sulfinyl)oxazole-5-carbaldehyde O- | 1H), 4.16 (q, J = 7.1 Hz, 2H), 3.46-3.66 (m, 2H), 2.77-2.87 |
| | ethyl oxime | (m, 2H), 1.19-1.27 (m, 3H); |
| 37 | (E)-2-((3,4,4-trifluorobut-3-en-1- | ¹H-NMR (400 MHz, DMSO-d₆) δ 8.58 (s, 1H), 8.30 (s, |
| | yl)sulfonyl)thiazole-5-carbaldehyde O- | 1H), 4.36-4.42 (m, 1H), 3.90 (t, J = 7.0 Hz, 2H), 2.79-2.90 |
| | cyclohexyl oxime | (m, 2H), 1.91-1.96 (m, 2H), 1.67-1.72 (m, 2H), 1.54-1.62 |
| | | (m, 2H), 1.46-1.52 (m, 1H), 1.30-1.44 (m, 3H); |
| 38 | (E)-1-(2-((3,4,4-trifluorobut-3-en-1- | ¹H-NMR (400 MHz, DMSO-d₆) δ 8.21 (s, 1H), 3.91 (s, |
| | yl)thio)thiazol-5-yl)ethan-1-one O- | 3H), 3.45-3.49 (m, 2H), 2.77-2.88 (m, 2H), 2.26 (s, 3H); |
| | methyl oxime | |
| 39 | (E)-4-methyl-2-((3,4,4-trifluorobut-3- | ¹H-NMR (400 MHz, CDCl₃) δ 8.12 (s, 1H), 3.94 (s, 3H), |
| | en-1-yl)sulfinyl)thiazole-5- | 3.58-3.53 (m, 1H), 3.34-3.29 (m, 1H), 2.87- 2.77 (m, 1H) |
| | carbaldehyde O-methyl oxime | 2.67-2.54 (m, 1H), 2.56 (s, 3H); |
| 40 | (E)-4-methyl-2-((3,4,4-trifluorobut-3- | ¹H-NMR (400 MHz, CDCl₃) δ 7.76 (s, 1H), 4.15 (s, 3H), |
| | en-1-yl)sulfonyl)thiazole-5- | 3.62-3.58 (m, 2H), 2.94-2.84 (m, 2H), 2.65 (s, 3H); |
| | carbaldehyde O-methyl oxime | |
| 41 | (E)-4-methyl-2-((3,4,4-trifluorobut-3- | ¹H-NMR (400 MHz, CDCl₃) δ 7.60 (s, 1H), 4.35 (q, J = |
| | en-1-yl)thio)thiazole-5-carbaldehyde | 7.2 Hz, 2H), 3.51-3.47 (m, 2H), 2.84-2.75 (m, 2H), 2.57 (s, |
| | O-ethyl oxime | 3H), 1.35 (t, J = 7.2 Hz, 3H); |
| 42 | (Z)-4-methyl-2-((3,4,4-trifluorobut-3- | ¹H-NMR (400 MHz, CDCl₃) δ 8.15 (s, 1H), 4.17 (q, J = |
| | en-1-yl)thio)thiazole-5-carbaldehyde | 7.2 Hz, 2H), 3.41 (t, J = 7.2 Hz, 2H), 2.85-2.74 (m, 2H), |
| | O-ethyl oxime | 2.49 (s, 3H), 1.28 (t, J = 7.2 Hz, 3H); |
| 43 | (Z)-2-((3,4,4-trifluorobut-3-en-1- | ¹H-NMR (400 MHz, DMSO-d₆) δ 8.61 (s, 1H), 8.35 (s, |
| | yl)sulfonyl)thiazole-5-carbaldehyde O- | 1H), 4.13 (d, J = 6.9 Hz, 2H), 3.91 (t, J = 7.0 Hz, 2H), |
| | cyclobutylmethyl oxime | 2.79-2.89 (m, 2H), 2.60-2.67 (m, 1H), 1.97-2.05 (m, 2H), |
| | | 1.74-1.91 (m, 4H); |
| 44 | (Z)-2-((3,4,4-trifluorobut-3-en-1- | ¹H-NMR (400 MHz, DMSO-d₆) δ 8.43 (s, 1H), 7.90 (s, |
| | yl)thio)thiazole-5-carbaldehyde O- | 1H), 4.03 (d, J = 6.9 Hz, 2H), 3.47 (t, J = 6.7 Hz, 2H), |
| | cyclobutylmethyl oxime | 2.78-2.88 (m, 2H), 2.56-2.64 (m, 1H), 1.95-2.03 (m, 2H), |
| | | 1.81-1.89 (m, 2H), 1.71-1.79 (m, 2H); |
| 45 | (E)-2-((3,4,4-trifluorobut-3-en-1- | ¹H-NMR (400 MHz, DMSO-d₆) δ 8.13 (s, 1H), 7.96 (s, |
| | yl)thio)thiazole-5-carbaldehyde O- | 1H), 4.16 (d, J = 6.6 Hz, 2H), 3.47 (t, J = 6.8 Hz, 2H), |
| | cyclobutylmethyl oxime | 2.77-2.88 (m, 2H), 2.62-2.70 (m, 1H), 1.97-2.05 (m, 2H), |
| | | 1.75-1.94 (m, 4H); |
| 46 | (E)-2-((3,4,4-trifluorobut-3-en-1- | ¹H-NMR (400 MHz, DMSO-d₆) δ 7.72-7.70 (s, 1H), 7.70- |
| | yl)thio)oxazole-5-carbaldehyde O- | 7.67 (s, 1H), 4.28-4.13 (m, 2H), 3.44-3.35 (m, 2H), 2.95- |
| | isopentyl oxime | 2.71 (m, 2H), 1.69-1.43 (m, 3H), 0.91-0.83 (m, 6H); |
| 47 | (Z)-2-((3,4,4-trifluorobut-3-en-1- | ¹H-NMR (400 MHz, DMSO-d₆) δ 8.13 (s, 1H), 7.45 (s, |
| | yl)thio)oxazole-5-carbaldehyde O- | 1H), 4.08 (t, J = 6.8 Hz, 2H), 3.39 (t, J = 6.9 Hz, 2H), 2.77- |
| | isopentyl oxime | 2.88 (m, 2H), 1.60-1.67 (m, 1H), 1.48 (q, J = 6.8 Hz, 2H), |
| | | 0.83-0.87 (m, 6H); |
| 48 | (Z)-2-((3,4,4-trifluorobut-3-en-1- | ¹H-NMR (400 MHz, DMSO-d₆) δ 8.31 (s, 1H), 7.71 (s, |
| | yl)sulfinyl)oxazole-5-carbaldehyde O- | 1H), 4.15 (t, J = 6.8 Hz, 2H), 3.46-3.66 (m, 2H), 2.77-2.87 |
| | isopentyl oxime | (m, 2H), 1.48-1.68 (m, 3H), 0.87 (d, J = 6.6 Hz, 6H); |
| 49 | (Z)-2-((3,4,4-trifluorobut-3-en-1- | ¹H-NMR (400 MHz, DMSO-d₆) δ 8.34 (s, 1H), 7.77 (s, |
| | yl)sulfonyl)oxazole-5-carbaldehyde O- | 1H), 4.15-4.20 (m, 2H), 3.92-3.96 (m, 2H), 2.81-2.88 (m, |
| | isopentyl oxime | 2H), 1.50-1.67 (m, 3H), 0.79-0.88 (m, 6H); |
| 50 | (E)-2-((3,4,4-trifluorobut-3-en-1- | ¹H-NMR (400 MHz, DMSO-d₆) δ 8.07 (s, 1H), 8.00 (s, |
| | yl)sulfonyl)oxazole-5-carbaldehyde O- | 1H), 4.33 (t, J = 6.6 Hz, 2H), 3.99 (t, J = 7.1 Hz, 2H), 2.86- |
| | isopentyl oxime | 2.95 (m, 2H), 1.60-1.72 (m, 3H), 0.92 (d, J = 6.6 Hz, 6H); |
| 51 | (E)-2-((3,4,4-trifluorobut-3-en-1- | ¹H-NMR (400 MHz, DMSO-d₆) δ 8.16 (s, 1H), 7.43 (s, |
| | yl)sulfinyl)oxazole-5-carbaldehyde O- | 1H), 4.23 (t, J = 6.6 Hz, 2H), 3.43 (t, J = 6.8 Hz, 2H), 2.80- |
| | isopentyl oxime | 2.91 (m, 2H), 1.57 (q, J = 6.7 Hz, 2H), 0.82-0.94 (m, 7H); |
| 52 | (E)-2-((3,4,4-trifluorobut-3-en-1- | ¹H-NMR (400 MHz, DMSO-d₆) δ 8.18 (s, 1H), 7.90 (d, J = |
| | yl)thio)thiazole-4-carbaldehyde O- | 0.5 Hz, 1H), 3.87 (s, 3H), 3.44 (t, J = 6.8 Hz, 2H), 2.77- |
| | methyl oxime | 2.88 (m, 2H); |
| 53 | (E)-1-(2-((3,4,4-trifluorobut-3-en-1- | ¹H-NMR (400 MHz, DMSO-d₆) δ 8.67 (s, 1H), 4.05 (s, |
| | yl)sulfonyl)thiazol-5-yl)ethan-1-one O- | 3H), 3.90 (t, J = 7.0 Hz, 2H), 2.78-2.88 (m, 2H), 2.39 (s, |
| | methyl oxime | 3H); |
| 54 | (E)-1-(2-((3,4,4-trifluorobut-3-en-1- | ¹H-NMR (400 MHz, DMSO-d₆) δ 8.55 (s, 1H), 4.00 (s, |
| | yl)sulfinyl)thiazol-5-yl)ethan-1-one O- | 3H), 3.56-3.63 (m, 1H), 3.36 (dd, J = 7.6, 6.6 Hz, 1H), |
| | methyl oxime | 2.82-2.84 (m, 1H), 2.58-2.64 (m, 1H), 2.34 (s, 3H); |
| 55 | (Z)-1-(2-((3,4,4-trifluorobut-3-en-1- | ¹H-NMR (400 MHz, DMSO-d₆) δ 8.04 (s, 1H), 3.85 (s, |
| | yl)thio)thiazol-5-yl)ethan-1-one O- | 3H), 3.45 (t, J = 6.8 Hz, 2H), 2.77-2.87 (m, 2H), 2.17 (s, |
| | methyl oxime | 3H); |
| 56 | (E)-1-(2-((3,4,4-trifluorobut-3-en-1- | ¹H-NMR (400 MHz, DMSO-d₆) δ 8.68 (d, J = 0.7 Hz, 1H), |
| | yl)thio)thiazol-5-yl)ethan-1-one O- | 4.13 (d, J = 7.1 Hz, 2H), 3.97-3.99 (m, 2H), 2.80-2.90 (m, |
| | ethyl oxime | 2H), 2.28 (s, 3H), 0.52-0.57 (m, 3H); |
| 57 | (Z)-1-(2-((3,4,4-trifluorobut-3-en-1- | ¹H-NMR (400 MHz, DMSO-d₆) δ 8.18-8.20 (m, 1H), 4.15- |
| | yl)thio)thiazol-5-yl)ethan-1-one O- | 4.20 (m, 2H), 3.47 (t, J = 6.8 Hz, 2H), 2.77-2.88 (m, 2H), |
| | ethyl oxime | 2.26 (s, 3H), 1.22-1.28 (m, 3H); |
| 58 | (E)-1-(2-((3,4,4-trifluorobut-3-en-1- | ¹H-NMR (400 MHz, DMSO-d₆) δ 8.37 (s, 1H), 4.16 (q, J = |
| | yl)sulfinyl)thiazol-5-yl)ethan-1-one O- | 7.1 Hz, 2H), 3.54-3.61 (m, 1H), 3.33-3.37 (m, 1H), 2.76- |
| | ethyl oxime | 2.89 (m, 1H), 2.57-2.70 (m, 1H), 2.22 (d, J = 14.4 Hz, 3H), |
| | | 1.24 (t, J = 7.0 Hz, 3H); |
| 59 | (E)-1-(2-((3,4,4-trifluorobut-3-en-1- | ¹H-NMR (400 MHz, DMSO-d₆) δ 8.49 (s, 1H), 4.19 (q, J = |
| | yl)sulfonyl)thiazol-5-yl)ethan-1-one O- | 7.1 Hz, 2H), 3.90 (t, J = 7.1 Hz, 2H), 2.78-2.89 (m, 2H), |
| | ethyl oxime | 2.25 (d, J = 15.9 Hz, 3H), 1.25 (t, J = 7.1 Hz, 3H); |
| 60 | (Z)-1-(2-((3,4,4-trifluorobut-3-en-1- | ¹H-NMR (400 MHz, DMSO-d₆) δ 8.56 (s, 1H), 4.26-4.33 |
| | yl)sulfinyl)thiazol-5-yl)ethan-1-one O- | (m, 2H), 3.58-3.65 (m, 1H), 3.38 (m, 3H), 2.78-2.90 (m, |
| | ethyl oxime | 1H), 2.57-2.70 (m, 1H), 2.35 (d, J = 10.3 Hz, 3H), 1.32 (q, |
| | | J = 7.1 Hz, 3H); |
| 61 | (Z)-1-(2-((3,4,4-trifluorobut-3-en-1- | ¹H-NMR (400 MHz, CDCl₃) δ 8.28 (s, 1H), 4.41 (q, J = |
| | yl)sulfonyl)thiazol-5-yl)ethan-1-one O- | 7.1 Hz, 2H), 3.64-3.68 (m, 2H), 2.88-2.97 (m, 2H), 2.45 (s, |
| | ethyl oxime | 3H), 1.43 (t, J = 7.1 Hz, 3H); |
| 62 | (Z)-2-((3,4,4-trifluorobut-3-en-1- | ¹H-NMR (400 MHz, DMSO-d₆) δ 8.38 (s, 1H), 7.63 (s, |
| | yl)thio)thiazole-4-carbaldehyde O- | 1H), 3.98 (s, 3H), 3.44 (t, J = 6.7 Hz, 2H), 2.77-2.87 (m, |
| | methyl oxime | 2H); |
| 63 | (Z)-2-((3,4,4-trifluorobut-3-en-1- | ¹H-NMR (400 MHz, DMSO-d₆) δ 8.41 (s, 1H), 7.65 (s, |
| | yl)thio)thiazole-4-carbaldehyde O- | 1H), 4.25 (q, J = 7.1 Hz, 2H), 3.46 (t, J = 6.7 Hz, 2H), |
| | ethyl oxime | 2.78-2.89 (m, 2H), 1.30 (t, J = 7.1 Hz, 3H); |
| 64 | (E)-2-((3,4,4-trifluorobut-3-en-1- | ¹H-NMR (400 MHz, DMSO-d₆) δ 8.35 (s, 1H), 8.33 (s, |
| | yl)sulfinyl)thiazole-4-carbaldehyde O- | 1H), 3.91 (s, 3H), 3.56-3.63 (m, 1H), 3.29-3.38 (m, 1H), |
| | methyl oxime | 2.78-2.92 (m, 1H), 2.60-2.73 (m, 1H); |
| 65 | (E)-2-((3,4,4-trifluorobut-3-en-1- | ¹H-NMR (400 MHz, DMSO-d₆) δ 8.32 (s, 1H), 8.31 (s, |
| | yl)sulfinyl)thiazole-4-carbaldehyde O- | 1H), 4.16 (q, J = 7.1 Hz, 2H), 3.54-3.61 (m, 1H), 3.28-3.36 |
| | ethyl oxime | (m, 1H), 2.77-2.89 (m, 1H), 2.59-2.71 (m, 1H), 1.23 (t, J = |
| | | 7.1 Hz, 3H); |
| 66 | (E)-2-((3,4,4-trifluorobut-3-en-1- | ¹H-NMR (400 MHz, DMSO-d₆) δ 8.48 (s, 1H), 8.38 (s, |
| | yl)sulfonyl)thiazole-4-carbaldehyde O- | 1H), 3.90 (m, , 5H) (Singlet merging with triplet), 2.79- |
| | methyl oxime | 2.90 (m, 2H); |
| 67 | (E)-2-((3,4,4-trifluorobut-3-en-1- | ¹H-NMR (400 MHz, DMSO-d₆) δ 8.47 (s, 1H), 8.37 (s, |
| | yl)sulfonyl)thiazole-4-carbaldehyde O- | 1H), 4.19 (q, J = 7.1 Hz, 2H), 3.90 (t, J = 7.0 Hz, 2H), |
| | ethyl oxime | 2.80-2.90 (m, 2H), 1.25 (t, J = 7.1 Hz, 3H) |
| 68 | (E)-5-chloro-2-((3,4,4-trifluorobut-3- | ¹H-NMR (400 MHz, DMSO-d₆) δ 8.13 (s, 1H), 3.89-3.93 |
| | en-1-yl)thio)thiazole-4-carbaldehyde | (m, 3H), 3.45 (t, J = 6.7 Hz, 2H), 2.78-2.88 (m, 2H); |
| | O-methyl oxime | |
| 69 | (E)-5-chloro-2-((3,4,4-trifluorobut-3- | ¹H-NMR (400 MHz, DMSO-d₆) δ 8.33 (s, 1H), 3.94-3.98 |
| | en-1-yl)sulfonyl)thiazole-4- | (m, 5H) (singlet merging with triplet), 2.85-2.95 (m, 2H); |
| | carbaldehyde O-methyl oxime | |
| 70 | (E)-2-((3,4,4-trifluorobut-3-en-1- | ¹H-NMR (400 MHz, CDCl₃) δ 8.21 (s, 1H), 7.82 (s, 1H), |
| | yl)sulfonyl)thiazole-5-carbaldehyde O- | 4.18 (d, J = 1.2 Hz, 3H), 3.62-3.66 (m, 2H), 2.86-2.95 (m, |
| | methyl oxime | 2H); |
| 71 | (Z)-2-((3,4,4-trifluorobut-3-en-1- | ¹H-NMR (400 MHz, DMSO-d₆) δ 8.11 (s, 1H), 7.98 (s, |
| | yl)sulfonyl)oxazole-5-carbaldehyde O- | 1H), 4.06 (s, 3H), 3.97 (t, J = 7.0 Hz, 2H), 2.83-2.93 (m, |
| | methyl oxime | 2H); |
| 72 | (E)-2-((3,4,4-trifluorobut-3-en-1- | ¹H-NMR (400 MHz, DMSO-d₆) δ 8.19 (s, 1H), 7.49 (s, |
| | yl)thio)oxazole-5-carbaldehyde O- | 1H), 3.91 (d, J = 7.1 Hz, 2H), 3.41 (t, J = 6.8 Hz, 2H), |
| | cyclopropylmethyl oxime | 2.81-2.90 (m, 2H), 1.11 (t, J = 7.9 Hz, 1H), 0.48-0.53 (m, |
| | | 2H), 0.24-0.28 (m, 2H); |
| 73 | N-((5-((Z)- | ¹H-NMR (400 MHz, DMSO-d₆) δ 8.62 (s, 1H), 8.32 (s, |
| | (methoxyimino)methyl)thiazol-2- | 1H), 3.94-4.01 (m, 4H) (singlet merging with multiplet), |
| | yl)(3,4,4-trifluorobut-3-en-1-yl)-λ⁴- | 3.82-3.88 (m, 1H), 2.92-3.03 (m, 2H); |
| | sulfaneylidene)cyanamide | |
| 74 | (E)-4-methyl-2-((3,4,4-trifluorobut-3- | ¹H-NMR (400 MHz, CDCl₃) δ 8.25 (s, 1H), 4.35 (q, J = |
| | en-1-yl)sulfonyl)thiazole-5- | 7.2 Hz, 2H), 3.86 (t, J = 6.8 Hz, 2H), 2.87-2.78 (m, 2H), |
| | carbaldehyde O-ethyl oxime | 2.63 (s, 3H), 1.31 (t, J = 7.2 Hz, 3H); |
| 75 | (E)-4-methyl-2-((3,4,4-trifluorobut-3- | ¹H-NMR (400 MHz, CDCl₃) δ 8.12 (s, 1H), 4.30 (q, J = |
| | en-1-yl)sulfinyl)thiazole-5- | 7.2 Hz, 2H), 3.59-3.54 (m, 1H), 3.33-3.28 (m, 1H), 2.88- |
| | carbaldehyde O-ethyl oxime | 2.77 (m, 1H), 2.69-2.60 (m, 1H), 2.58 (s, 3H), 1.29 (t, J = |
| | | 7.2 Hz, 3H); |
| 76 | (E)-2-((3,4,4-trifluorobut-3-en-1- | ¹H-NMR (400 MHz, DMSO-d₆) δ 8.12 (s, 1H), 7.47 (d, J = |
| | yl)thio)oxazole-5-carbaldehyde O- | 1.8 Hz, 1H), 4.31-4.38 (m, 1H), 3.41 (t, J = 6.8 Hz, 2H), |
| | isopropyl oxime | 2.81-2.90 (m, 2H), 1.21 (d, J = 6.1 Hz, 6H); |
| 77 | (E)-2-((3,4,4-trifluorobut-3-en-1- | ¹H-NMR (400 MHz, DMSO-d₆) δ 8.30 (t, J = 3.7 Hz, 1H), |
| | yl)sulfinyl)oxazole-5-carbaldehyde O- | 7.73 (d, J = 4.2 Hz, 1H), 4.37-4.45 (m, 1H), 3.48-3.69 (m, |
| | isopropyl oxime | 2H), 2.74-2.92 (m, 2H), 1.20-1.24 (m, 6H); |
| 78 | (E)-2-((3,4,4-trifluorobut-3-en-1- | ¹H-NMR (400 MHz, DMSO-d₆) δ 8.30 (s, 1H), 7.73 (s, |
| | yl)sulfonyl)oxazole-5-carbaldehyde O- | 1H), 4.37-4.45 (m, 1H), 3.48-3.69 (m, 2H), 2.74-2.92 (m, |
| | isopropyl oxime | 2H), 1.20-1.24 (m, 6H); |
| 79 | (E)-2-((3,4,4-trifluorobut-3- en-1- | ¹H-NMR (400 MHz, DMSO-d₆) δ 8.36 (d, J = 2.8 Hz, 1H), |
| | yl)sulfinyl)oxazole-5-carbaldehyde O- | 7.74 (s, 1H), 3.98 (d, J = 7.3 Hz, 2H), 3.62-3.69 (m, 1H), |
| | cyclopropylmethyl oxime | 3.49-3.56 (m, 1H), 2.80-2.90 (m, 2H), 1.11-1.17 (m, 1H), |
| | | 0.50-0.54 (m, 2H), 0.27-0.30 (m, 2H); |
| 80 | (E)-2-((3,4,4-trifluorobut-3-en-1- | ¹H-NMR (400 MHz, DMSO-d₆) δ 8.40 (d, J = 2.4 Hz, 1H), |
| | yl)sulfonyl)oxazole-5-carbaldehyde O- | 7.82 (s, 1H), 3.94-4.01 (m, 4H), 2.83-2.93 (m, 2H), 2.34- |
| | cyclopropylmethyl oxime | 2.21 (0H), 1.29-1.21 (0H), 1.11-1.18 (m, 1H), 0.50-0.55 |
| | | (m, 2H), 0.27-0.31 (m, 2H); |
| 81 | (Z)-2-((3,4,4-trifluorobut-3-en-1- | ¹H-NMR (400 MHz, CDCl₃) δ 8.37-8.36 (1H), 7.99-7.97 |
| | yl)sulfonyl)thiazole-5-carbaldehyde O- | (1H), 4.66-4.55 (2H), 3.83-3.70 (2H), 3.15-2.98 (2H), |
| | ethyl oxime | 1.61-1.54 (3H); |
| 82 | (Z)-2-((3,4,4-trifluorobut-3-en-1- | ¹H-NMR (400 MHz, DMSO-d₆) δ 7.79 (s, 1H), 7.72 (s, |
| | yl)thio)oxazole-5-carbaldehyde O- | 1H), 4.03 (d, J = 7.2 Hz, 3H), 3.37-3.46 (m, 3H), 2.80-2.91 |
| | cyclopropylmethyl oxime | (m, 3H), 1.13-1.23 (m, 1H), 0.46-0.55 (m, 3H), 0.28-0.33 |
| | | (m, 3H); |
| 83 | (Z)-2-((3,4,4-trifluorobut-3-en-1- | ¹H-NMR (400 MHz, DMSO-d₆) δ 8.09 (s, 1H), 7.98 (s, |
| | yl)sulfonyl)oxazole-5-carbaldehyde O- | 1H), 4.11 (d, J = 7.3 Hz, 2H), 3.97 (t, J = 7.1 Hz, 2H), |
| | cyclopropylmethyl oxime | 2.83-2.94 (m, 2H), 1.17-1.25 (m, 2H), 0.52-0.56 (m, 2H), |
| | | 0.31-0.35 (m, 2H); |
| 84 | (Z)-1-(2-((3,4,4-trifluorobut-3-en-1- | ¹H-NMR (400 MHz, DMSO-d₆) δ 8.68 (d, J = 0.7 Hz, 1H), |
| | yl)thio)thiazol-5-yl)ethan-1-one O- | 4.13 (d, J = 7.1 Hz, 2H), 3.97-3.99 (m, 2H), 2.80-2.90 (m, |
| | cyclopropylmethyl oxime | 2H), 2.28 (s, 2H), 1.14-1.20 (m, 1H), 0.52-0.57 (m, 2H), |
| | | 0.29-0.32 (m, 2H); |
| 85 | (Z)-1-(2-((3,4,4-trifluorobut-3-en-1- | ¹H-NMR (400 MHz, DMSO-d₆) δ 8.68 (d, J = 0.7 Hz, 1H), |
| | yl)sulfonyl)thiazol-5-yl)ethan-1-one O- | 4.13 (d, J = 7.1 Hz, 2H), 3.92 (t, J = 7.0 Hz, 2H), 2.82-2.89 |
| | cyclopropylmethyl oxime | (m, 2H), 2.41 (s, 3H), 1.24 (s, 1H), 0.53-0.58 (m, 2H), |
| | | 0.33-0.36 (m, 2H); |
| 86 | (Z)-1-(2-((3,4,4-trifluorobut-3-en-1- | ¹H-NMR (400 MHz, DMSO-d₆) δ 8.56 (s, 1H), 4.08 (d, J = |
| | yl)sulfinyl)thiazol-5-yl)ethan-1-one O- | 7.3 Hz, 2H), 3.58-3.65 (m, 1H), 3.37 (q, J = 7.1 Hz, −10H), |
| | cyclopropylmethyl oxime | 2.79-2.90 (m, 1H), 2.58-2.70 (m, 1H), 1.17-1.24 (m, 1H), |
| | | 0.53-0.58 (m, 2H), 0.31-0.35 (m, 2H); |
| 87 | (Z)-2-((3,4,4-trifluorobut-3-en-1- | ¹H-NMR (400 MHz, CDCl₃) δ 8.21 (s, 1H), 7.26 (s, 1H), |
| | yl)sulfinyl)thiazole-5-carbaldehyde O- | 4.23 (q, J = 7.1 Hz, 2H), 3.38-3.45 (m, 1H), 3.26-3.33 (m, |
| | ethyl oxime | 1H), 2.89-2.97 (m, 1H), 2.57-2.65 (m, 1H), 1.27-1.33 (m, |
| | | 3H); |
| 88 | (Z)-2-((3,4,4-trifluorobut-3-en-1- | ¹H-NMR (400 MHz, CDCl₃) δ 8.12 (s, 1H), 7.63 (s, 1H), |
| | yl)thio)thiazole-5-carbaldehyde O- | 3.43-3.47 (m, 2H), 2.76-2.85 (m, 2H), 1.26-1.36 (m, 6H); |
| | isopropyl oxime | |
| 89 | (Z)-2-((3,4,4-trifluorobut-3-en-1- | ¹H-NMR (400 MHz, CDCl₃) δ 8.20 (s, 1H), 7.82 (s, 1H), |
| | yl)sulfonyl)thiazole-5-carbaldehyde O- | 4.63-4.69 (m, 1H), 3.62-3.66 (m, 2H), 2.86-2.96 (m, 2H), |
| | isopropyl oxime | 1.40 (d, J = 6.4 Hz, 6H); |
| 90 | (E)-2-((3,4,4-trifluorobut-3-en-1- | ¹H-NMR (400 MHz, CDCl₃) δ 8.13 (s, 1H), 7.74 (s, 1H), |
| | yl)sulfinyl)thiazole-5-carbaldehyde O- | 4.57-4.64 (m, 1H), 3.29-3.47 (m, 2H), 2.89-2.99 (m, 1H), |
| | isopropyl oxime | 2.56-2.66 (m, 1H), 1.60 (s, 2H), 1.37-1.41 (m, 6H); |
| 91 | N-((E)-(4-((E)- | ¹H-NMR (400 MHz, DMSO-d₆) δ 8.45 (S, 1H), 8.35 (S, |
| | (methoxyimino)methyl)thiazol-2- | 1H), 4.05-3.85 (m, 1H), 3.84 (S, 3H), 3.84-3.73 (1H), |
| | yl)(3,4,4-trifluorobut-3-en-1-yl)-14- | 3.04-2.79 (2H); |
| | sulfaneylidene)cyanamide | |
| 92 | (E)-1-(2-((3,4,4-trifluorobut-3-en-1- | ¹H-NMR (400 MHz, DMSO-d₆) δ 8.04 (d, J = 2.4 Hz, 1H), |
| | yl)thio)thiazol-5-yl)ethan-1-one O- | 3.91 (d, J = 7.1 Hz, 2H), 3.47 (t, J = 6.8 Hz, 2H), 2.84 |
| | cyclopropylmethyl oxime | (ddd, J = 23.0, 4.1, 2.8 Hz, 2H), 2.20 (d, J = 7.8 Hz, 3H), |
| | | 1.11 (s, 1H), 0.49-0.53 (m, 2H), 0.27-0.30 (m, 2H); |
| 93 | (E)-phenyl(2-((3,4,4-trifluorobut-3-en- | ¹H-NMR (400 MHz, CDCl₃) δ.78-7.65 (s, 1H), 7.59-7.38 |
| | 1-yl)thio)thiazol-5-yl)methanone O- | (m, 5H), 4.26-4.08 (s, 3H), 3.59-3.43 (m, 2H), 2.92-2.72 |
| | methyl oxime | (m, 2H); |
| 94 | (E)-phenyl(2-((3,4,4-trifluorobut-3-en- | ¹H-NMR (400 MHz, DMSO-d₆) δ 8.01 (s, 1H), 7.51-7.65 |
| | 1-yl)sulfinyl)thiazol-5-yl)methanone O- | (m, 5H), 4.12 (s, 3H), 3.60 (m, 1H), 3.35 (m, 1H), 2.62 (m, |
| | methyl oxime | 1H), 2.48 (m,1H); |
| 95 | (E)-phenyl(2-((3,4,4-trifluorobut-3-en- | ¹H-NMR (400 MHz, DMSO-d₆) δ 7.68 (s, 1H), 7.46-7.52 |
| | 1-yl)thio)thiazol-5-yl)methanone O- | (m, 5H), 4.30 (q, J = 7.1 Hz, 2H), 3.46 (m, 2H), 2.48-2.84 |
| | ethyl oxime | (m, 4H), 1.32 (t, J = 7.1 Hz, 3H); |
| 96 | (E)-phenyl(2-((3,4,4-trifluorobut-3-en- | ¹H-NMR (400 MHz, DMSO-d₆) δ 8.2 (1H), 7.46-7.58 (m, |
| | 1-yl)sulfonyl)thiazol-5-yl)methanone | 5H), 4.28 (s,3H), 3.9 (m, 2H), 2.80-2.91 (m, 2H); |
| | O-methyl oxime | |
| 97 | (E)-phenyl(2-((3,4,4-trifluorobut-3-en- | ¹H-NMR (400 MHz, DMSO-d₆) δ12.56 (s, 1H) 8.2 (s, 1H), |
| | 1-yl)thio)thiazol-5-yl)methanone oxime | 7.46-7.58 (m, 5H), 3.9 (m, 2H), 2.80-2.91 (m, 2H); |
| 98 | (E)-phenyl(2-((3,4,4-trifluorobut-3-en- | ¹H-NMR (400 MHz, DMSO-d₆) δ 8.01 (s, 1H), 7.51-7.65 |
| | 1-yl)sulfinyl)thiazol-5-yl)methanone O- | (m, 5H), 4.39 (q, J = 7.1 Hz, 2H), 3.60 (m, 1H), 3.37 (m, |
| | ethyl oxime | 1H),, 2.65 (m, 1H), 2.49 (m, 1H), 1.34 (t, J = 7.1 Hz, 3H); |
| 99 | (E)-phenyl(2-((3,4,4-trifluorobut-3-en- | ¹H-NMR (400 MHz, DMSO-d₆) δ 8.2 (s, 1H),, 7.54-7.60 |
| | 1-yl)sulfonyl)thiazol-5-yl)methanone | (m, 5H), 4.47 (q, J = 7.1 Hz, 2H), 3.93 (t, J = 7.1 Hz, 2H), |
| | O-ethyl oxime | 2.84 (s, 2H), 1.39 (t, J = 7.1 Hz, 3H); |
| 100 | (E)-phenyl(2-((3,4,4-trifluorobut-3-en- | ¹H-NMR (400 MHz, DMSO-d₆) δ 7.70 (d, J = 5.6 Hz, 1H), |
| | 1-yl)thio)thiazol-5-yl)methanone O- | 7.50-7.55 (m, 5H), 4.12 (d, J = 7.1 Hz, 2H), 3.50 (t, J = 6.8 |
| | cyclopropylmethyl oxime | Hz, 2H), 2.86 (ddd, J = 23.0, 4.2, 2.7 Hz, 2H), 2.50-2.52 |
| | | (m, 1H), 0.56-0.61 (m, 2H), 0.34-0.38 (m, 2H); |
| 101 | (E)-2-((3,4,4-trifluorobut-3-en-1- | ¹H-NMR (400 MHz, DMSO-d₆) δ 8.17 (s, 1H), 7.48 (s, |
| | yl)thio)oxazole-5-carbaldehyde O- | 1H), 4.07 (d, J = 7.1 Hz, 2H), 3.41 (t, J = 6.8 Hz, 2H), |
| | cyclobutylmethyl oxime | 2.80-2.90 (m, 2H), 2.57-2.66 (m, 2H), 1.96-2.03 (m, 2H), |
| | | 1.71-1.88 (m, 3H); |
| 102 | (Z)-2-((3,4,4-trifluorobut-3-en-1- | ¹H-NMR (400 MHz, DMSO-d₆) δ 7.75 (s, 1H), 7.71 (s, |
| | yl)thio)oxazole-5-carbaldehyde O- | 1H), 4.40-4.46 (m, 1H), 3.43 (t, J = 6.7 Hz, 2H), 2.80-2.91 |
| | isopropyl oxime | (m, 1H), 1.21-1.32 (m, 6H); |
| 103 | (Z)-2-((3,4,4-trifluorobut-3-en-1- | ¹H-NMR (400 MHz, DMSO-d₆) δ 8.02 (m, 1H), 7.94-7.95 |
| | yl)sulfinyl)oxazole-5-carbaldehyde O- | (m, 1H), 4.09 (d, J = 7.1 Hz, 2H), 3.49-3.70 (m, 2H), 2.79- |
| | cyclopropylmethyl oxime | 2.94 (m, 2H), 1.16-1.25 (m, 2H), 0.51-0.56 (m, 2H), 0.30- |
| | | 0.34 (m, 2H) |
| 104 | (E)-phenyl(2-((3,4,4-trifluorobut-3-en- | ¹H-NMR (400 MHz, DMSO-d₆) δ 8.04 (s, 1H), 7.52-7.58 |
| | 1-yl)sulfinyl)thiazol-5-yl)methanone O- | (m, 5H), 4.21 (d, J = 7.3 Hz, 2H), 3.63 (dd, J = 13.9, 6.4 |
| | cyclopropylmethyl oxime | Hz, 2H), 2.96-2.59 (2H), 1.24-1.29 (m, 1H), 0.57-0.62 (m, |
| | | 2H), 0.36-0.40 (m, 2H); |
| 105 | (E)-phenyl(2-((3,4,4-trifluorobut-3-en- | ¹H-NMR (400 MHz, DMSO-d₆) δ 8.04 (s, 1H), 7.52-7.58 |
| | 1-yl)sulfinyl)thiazol-5-yl)methanone | (m, 5H), 4.21 (d, J = 7.3 Hz, 2H), 3.63 (dd, J = 13.9, 6.4 |
| | oxime | Hz, 2H), 2.96-2.59 (m, 2H), 1.24-1.29 (m, 1H), 0.57-0.62 |
| | | (m, 2H), 0.36-0.40 (m, 2H); |
| 106 | (E)-phenyl(2-((3,4,4-trifluorobut-3-en- | ¹H-NMR (400 MHz, DMSO-d₆) δ 8.09-8.17 (m, 1H), 7.52- |
| | 1-yl)sulfonyl)thiazol-5-yl)methanone | 7.61 (m, 5H), 4.24 (m, 2H), 3.90-4.01 (m, 2H), 2.83-2.93 |
| | O-cyclopropylmethyl oxime | (m, 2H), 1.24-1.40 (m, 1H), 0.29-0.63 (m, 4H); |
| 107 | (E)-phenyl(2-((3,4,4-trifluorobut-3-en- | ¹H-NMR (400 MHz, DMSO-d₆) δ 8.09-8.17 (m, 1H), 7.52- |
| | 1-yl)thio)thiazol-5-yl)methanone O- | 7.61 (m, 5H), 4.24 (m, 2H), 3.90-4.01 (m, 2H), 2.83-2.93 |
| | propyl oxime | (m, 2H), 1.24-1.40 (m, 1H), 0.29-0.63 (m, 4H); |
| 108 | (E)-phenyl(2-((3,4,4-trifluorobut-3-en- | ¹H-NMR (400 MHz, DMSO-d₆) δ 7.97 (1H), 7.46-7.58 (m, |
| | 1-yl)thio)thiazol-5-yl)methanone O- | 5H), 4.24 (m, 2H), 3.91 (m, 2H), 2.80-2.91 (m, 2H), 1.60- |
| | isopentyl oxime | 1.83 (m, 2H), 0.92 (m, 3H); |
| 109 | (E)-phenyl(2-((3,4,4-trifluorobut-3-en- | ¹H-NMR (400 MHz, DMSO-d₆) δ 13.51-13.49 (s, 1H), |
| | 1-yl)sulfonyl)thiazol-5-yl)methanone | 8.11 (s, 1H), 7.51-7.60 (m, 5H), 3.91 (t, J = 7.1 Hz, 2H), |
| | oxime | 2.81-2.92 (m, 2H); |
| 110 | (E)-phenyl(2-((3,4,4-trifluorobut-3-en- | ¹H-NMR (400 MHz, DMSO-d₆) δ 7.83 (1H), 7.42-7.57 |
| | 1-yl)sulfinyl)thiazol-5-yl)methanone O- | (m, 5H), 4.20 (m, 2H), 3.58-3.66 (m, 1H), 3.34-3.40 (m, |
| | propyl oxime | 1H), 2.79-2.89 (m, 1H), 2.63-2.73 (m, 1H), 1.59-1.81 (m, |
| | | 2H), 0.83-1.03 (m, 3H); |
| 111 | (E)-phenyl(2-((3,4,4-trifluorobut-3-en- | ¹H-NMR (400 MHz, DMSO-d₆) δ 7.97 (s,1H), 7.46-7.58 |
| | 1-yl)sulfonyl)thiazol-5-yl)methanone | (m, 5H), 4.24 (m, 2H), 3.91 (m, 2H), 2.80-2.91 (m, 2H), |
| | O-propyl oxime | 1.60-1.83 (m, 2H), 0.92 (m, 3H); |
| 112 | (E)-phenyl(2-((3,4,4-trifluorobut-3-en- | ¹H-NMR (400 MHz, CDCl₃) δ 7.72 (s, 1H), 7.38-7.52 (m, |
| | 1-yl)sulfinyl)thiazol-5-yl)methanone O- | 5H), 4.14 (m, 3H), 3.48-3.53 (m, 3H), 2.78-2.87 (m, 2H), |
| | isopentyl oxime | 1.72 (m, 3H) 0.92 (d, J = 6.4 Hz, 6H); |
| 113 | (E)-phenyl(2-((3,4,4-trifluorobut-3-en- | ¹H-NMR (400 MHz, DMSO-d₆) δ 7.97 (d, 1H), 7.44-7.59 |
| | 1-yl)sulfonyl)thiazol-5-yl)methanone | (m, 5H), 4.31 (m, 2H), 3.88-3.93 (m, 2H), 2.80-2.91 (m, |
| | O-isopentyl oxime | 2H), 1.48-1.79 (m, 3H), 0.79-0.94 (m, 6H); |
| 114 | (E)-1-(2-((3,4,4-trifluorobut-3-en-1- | ¹H-NMR (400 MHz, DMSO-d₆) δ 8.40-8.30 (1H), 4.01- |
| | yl)sulfinyl)thiazol-5-yl)ethan-1-one O- | 3.89 (2H), 3.63-3.50 (1H), 2.93-2.76 (1H), 2.73-2.56 (1H), |
| | cyclopropylmethyl oxime | 2.32-2.22 (3H), 1.20-1.07 (1H), 0.61-0.48 (2H), 0.38-0.26 |
| | | (2H); |
| 115 | (E)-1-(2-((3,4,4-trifluorobut-3-en-1- | ¹H-NMR (400 MHz, DMSO-d₆) δ 8.52-8.50 (1H), 4.03- |
| | yl)sulfonyl)thiazol-5-yl)ethan-1-one O- | 3.97 (2H), 3.94-3.88 (2H), 2.92-2.77 (2H), 2.32-2.29 (3H), |
| | cyclopropylmethyl oxime | 1.24-1.09 (1H), 0.57-0.49 (2H), 0.35-0.27 (2H); |
| 116 | (Z)-1-(2-((3,4,4-trifluorobut-3-en-1- | ¹H-NMR (400 MHz, DMSO-d₆) δ 8.19 (s, 1H), 4.36-4.42 |
| | yl)thio)thiazol-5-yl)ethan-1-one O- | (m, 1H), 3.46 (t, J = 6.9 Hz, 2H), 2.77-2.88 (m, 2H), 2.27 |
| | isopropyl oxime | (s, 3H), 1.26 (d, J = 6.3 Hz, 6H); |
| 117 | (E)-1-(2-((3,4,4-trifluorobut-3-en-1- | ¹H-NMR (400 MHz, DMSO-d₆) δ 8.00 (s, 1H), 4.27-4.33 |
| | yl)thio)thiazol-5-yl)ethan-1-one O- | (m, 1H), 3.45 (t, J = 6.8 Hz, 2H), 2.78-2.86 (m, 2H), 2.16 |
| | isopropyl oxime | (s, 3H), 1.20 (d, J = 6.3 Hz, 6H); |
| 118 | (Z)-1-(2-((3,4,4-trifluorobut-3-en-1- | ¹H-NMR (400 MHz, DMSO-d₆) δ 8.56-8.54 (1H), 4.54- |
| | yl)sulfinyl)thiazol-5-yl)ethan-1-one O- | 4.45 (1H), 3.66-3.56 (1H), 3.41-3.32 (1H), 2.91-2.77 (1H), |
| | isopropyl oxime | 2.73-2.57 (1H), 2.39-2.33 (3H), 1.36-1.28 (6H); |
| 119 | (Z)-1-(2-((3,4,4-trifluorobut-3-en-1- | ¹H-NMR (400 MHz, DMSO-d₆) δ 8.69-8.65 (1H), 4.63- |
| | yl)sulfonyl)thiazol-5-yl)ethan-1-one O- | 4.50 (1H), 3.97-3.85 (2H), 2.94-2.77 (2H), 2.45-2.36 (3H), |
| | isopropyl oxime | 1.39-1.29 (6H); |
| 120 | (E)-1-(2-((3,4,4-trifluorobut-3-en-1- | ¹H-NMR (400 MHz, DMSO-d₆) δ 8.38-8.35 (1H), 4.44- |
| | yl)sulfinyl)thiazol-5-yl)ethan-1-one O- | 4.33 (1H), 3.63-3.51 (1H), 3.41-3.32 (1H), 2.93-2.75 (1H), |
| | isopropyl oxime | 2.73-2.56 (1H), 2.27-2.20 (3H), 1.29-1.21 (6H); |
| 121 | (E)-1-(2-((3,4,4-trifluorobut-3-en-1- | ¹H-NMR (400 MHz, DMSO-d₆) δ 8.51-8.48 (1H), 4.47- |
| | yl)sulfonyl)thiazol-5-yl)ethan-1-one O- | 4.34 (1H), 3.95-3.85 (2H), 2.92-2.76 (2H), 2.31-2.21 (3H), |
| | isopropyl oxime | 1.31-1.21 (6H); |
| 122 | (Z)-1-(2-((3,4,4-trifluorobut-3-en-1- | ¹H-NMR (400 MHz, DMSO-d₆) δ 8.55 (s, 1H), 4.00 (s, |
| | yl)sulfinyl)thiazol-5-yl)ethan-1-one O- | 3H), 3.56-3.63 (m, 1H), 3.32-3.37 (m, 1H), 2.77-2.88 (m, |
| | methyl oxime | 1H), 2.55-2.66 (m, 1H), 2.32-2.38 (m, 3H); |
| 123 | (Z)-1-(2-((3,4,4-trifluorobut-3-en-1- | ¹H-NMR (400 MHz, DMSO-d₆) δ 8.67 (s, 1H), 4.05 (s, |
| | yl)sulfonyl)thiazol-5-yl)ethan-1-one O- | 3H), 3.90 (t, J = 7.1 Hz, 2H), 2.78-2.89 (m, 2H), 2.39 (s, |
| | methyl oxime | 3H); |
| 124 | (Z)-2-((3,4,4-trifluorobut-3-en-1- | ¹H-NMR (400 MHz, DMSO-d₆) δ 8.01 (s, 1H), 4.09 (t, J = |
| | yl)sulfinyl)oxazole-5-carbaldehyde O- | 6.7 Hz, 2H), 3.44 (t, J = 6.8 Hz, 2H), 2.77-2.87 (m, 2H), |
| | isopropyl oxime | 2.16 (s, 3H), 1.63-1.70 (m, 1H), 1.50 (q, J = 6.8 Hz, 2H), |
| | | 0.86-0.89 (m, 6H); |
| 125 | (E)-2-((3,4,4-trifluorobut-3-en-1- | ¹H-NMR (400 MHz, DMSO-d₆) δ 8.34 (t, J = 6.0 Hz, 1H), |
| | yl)sulfinyl)oxazole-5-carbaldehyde O- | 7.72-7.74 (m, 1H), 4.13-4.17 (m, 2H), 3.49-3.69 (m, 2H), |
| | cyclobutylmethyl oxime | 2.74-2.96 (m, 2H), 2.61-2.70 (m, 1H), 1.97-2.06 (m, 2H), |
| | | 1.71-1.89 (m, 4H), 1.22 (d, J = 4.6 Hz, 1H); |
| 126 | (E)-2-((3,4,4-trifluorobut-3-en-1- | ¹H-NMR (400 MHz, DMSO-d₆) δ 8.40 (s, 1H), 7.83 (s, |
| | yl)sulfonyl)oxazole-5-carbaldehyde O- | 1H), 4.18 (d, J = 7.1 Hz, 2H), 3.99 (t, J = 7.1 Hz, 2H), |
| | cyclobutylmethyl oxime | 2.84-2.95 (m, 2H), 2.63-2.71 (m, 1H), 1.99-2.07 (m, 2H), |
| | | 1.73-1.91 (m, 4H), 1.24 (s, 1H); |
| 127 | (Z)-1-(2-((3,4,4-trifluorobut-3-en-1- | ¹H-NMR (400 MHz, DMSO-d₆) δ 7.85(s, 1H), 3.93-3.90 |
| | yl)thio)oxazol-5-yl)ethan-1-one O- | (m, 3H), 3.48-3.40 (m, 2H), 2.93-2.77 (m, 2H), 2.16 (s, |
| | methyl oxime | 3H); |
| 128 | (E)-1-(2-((3,4,4-trifluorobut-3-en-1- | ¹H-NMR (400 MHz, DMSO-d₆) δ 7.63-7.66 (m, 1H), 3.89 |
| | yl)thio)oxazol-5-yl)ethan-1-one O- | (s, 3H), 3.40 (t, J = 6.8 Hz, 2H), 2.79-2.90 (m, 2H), 2.05 (s, |
| | methyl oxime | 3H); |
| 129 | (E)-1-(2-((3,4,4-trifluorobut-3-en-1- | ¹H-NMR (400 MHz, DMSO-d₆) δ 8.00 (s, 1H), 3.96-3.98 |
| | yl)sulfonyl)oxazol-5-yl)ethan-1-one O- | (m, 5H), 2.82-2.93 (m, 2H), 2.15 (t, J = 4.5 Hz, 3H); |
| | methyl oxime | |
| 130 | (E)-1-(2-((3,4,4-trifluorobut-3-en-1- | ¹H-NMR (400 MHz, DMSO-d₆) δ 7.91 (s, 1H), 3.95 (s, |
| | yl)sulfinyl)oxazol-5-yl)ethan-1-one O- | 3H), 3.48-3.69 (m, 2H), 2.72 (m, 2H), 2.13 (s, 3H); |
| | methyl oxime | |
| 131 | (Z)-2-((3,4,4-trifluorobut-3-en-1- | ¹H-NMR (400 MHz, DMSO-d₆) δ 8.03-8.06 (m, 1H), 7.96 |
| | yl)sulfonyl)oxazole-5-carbaldehyde O- | (d, J = 4.9 Hz, 1H), 4.46-4.56 (m, 1H), 3.96 (q, J = 7.3 Hz, |
| | isopropyl oxime | 3H), 2.81-2.93 (m, 3H), 1.31 (d, J = 6.4 Hz, 6H); |
| 132 | (E)-1-(2-((3,4,4-trifluorobut-3-en-1- | ¹H-NMR (400 MHz, DMSO-d₆) δ 7.63 (d, J = 0.7 Hz, 1H), |
| | yl)thio)oxazol-5-yl)ethan-1-one O-ethyl | 4.12-4.17 (m, 2H), 3.38-3.43 (m, 2H), 2.79-2.90 (m, 2H), |
| | oxime | 2.05 (s, 3H), 1.21-1.24 (m, 3H); |
| 133 | (E)-1-(2-((3,4,4-trifluorobut-3-en-1- | ¹H-NMR (400 MHz, DMSO-d₆) δ 7.97 (s, 1H), 4.22 (t, J = |
| | yl)sulfonyl)oxazol-5-yl)ethan-1-one O- | 7.0 Hz, 2H), 3.95 (t, J = 7.0 Hz, 2H), 2.82-2.90 (m, 2H), |
| | ethyl oxime | 2.14 (s, 3H), 1.21-1.26 (m, 3H); |
| 134 | (Z)-1-(2-((3,4,4-trifluorobut-3-en-1- | ¹H-NMR (400 MHz, DMSO-d₆) δ 8.08 (s, 1H), 3.96 (s, |
| | yl)sulfonyl)oxazol-5-yl)ethan-1-one O- | 3H), 3.50-3.71 (m, 2H), 2.79-2.90 (m, 2H), 2.21 (s, 3H); |
| | methyl oxime | |
| 135 | (Z)-1-(2-((3,4,4-trifluorobut-3-en-1- | ¹H-NMR (400 MHz, DMSO-d₆) δ 7.83 (s, 1H), 4.13-4.19 |
| | yl)thio)oxazol-5-yl)ethan-1-one O-ethyl | (m, 2H), 3.43 (t, J = 6.8 Hz, 2H), 2.80-2.91 (m, 2H), 2.13 |
| | oxime | (s, 3H), 1.25 (t, J = 7.1 Hz, 3H); |
| 136 | (E)-2-((3,4,4-trifluorobut-3-en-1- | ¹H-NMR (400 MHz, DMSO-d₆) δ 8.49-8.43 (1H), 8.18- |
| | yl)sulfinyl)thiazole-5-carbaldehyde O- | 8.12 (1H), 4.36-4.26 (2H), 3.69-3.57 (1H), 3.40-3.33 (1H), |
| | isopentyl oxime | 2.90-2.74 (1H), 2.71-2.55 (1H), 1.79-1.64 (1H), 1.64-1.53 |
| | | (2H), 0.97-0.82 (6H); |
| 137 | (E)-2-((3,4,4-trifluorobut-3-en-1- | ¹H-NMR (400 MHz, CDCl₃) δ 8.20 (s, 1H), 7.81 (s, 1H), |
| | yl)sulfonyl)thiazole-5-carbaldehyde O- | 4.37-4.43 (m, 2H), 3.62-3.66 (m, 2H), 2.86-2.96 (m, 2H), |
| | isopentyl oxime | 1.65-1.79 (m, 2H), 1.26 (t, J = 7.1 Hz, 1H), 0.95 (dd, J = |
| | | 10.5, 6.6 Hz, 6H); |
| 138 | (E)-1-(2-((3,4,4-trifluorobut-3-en-1- | ¹H-NMR (400 MHz, DMSO-d₆) δ 8.01 (s, 1H), 4.09 (t, J = |
| | yl)thio)thiazol-5-yl)ethan-1-one O- | 6.7 Hz, 2H), 3.44 (t, J = 6.8 Hz, 2H), 2.77-2.87 (m, 2H), |
| | isopentyl oxime | 2.16 (s, 3H), 1.63-1.70 (m, 1H), 1.50 (q, J = 6.8 Hz, 2H), |
| | | 0.86-0.89 (m, 6H); |
| 139 | (E)-1-(2-((3,4,4-trifluorobut-3-en-1- | ¹H-NMR (400 MHz, DMSO-d₆) δ 8.40-8.35 (1H), 4.22- |
| | yl)sulfinyl)thiazol-5-yl)ethan-l-one O- | 4.13 (2H), 3.64-3.54 (1H), 3.39-3.30 (1H), 2.91-2.76 (1H), |
| | isopentyl oxime | 2.72-2.56 (1H), 2.28-2.20 (3H), 1.76-1.63 (1H), 1.60-1.49 |
| | | (2H), 0.96-0.88 (6H); |
| 140 | (E)-1-(2-((3,4,4-trifluorobut-3-en-1- | ¹H-NMR (400 MHz, DMSO-d₆) δ 8.51-8.49 (1H), 4.24- |
| | yl)sulfonyl)thiazol-5-yl)ethan-1-one O- | 4.16 (2H), 3.94-3.86 (2H), 2.91-2.78 (2H), 2.30-2.24 (3H), |
| | isopentyl oxime | 1.76-1.64 (1H), 1.60-1.52 (2H), 0.97-0.87 (6H); |
| 141 | (E)-1-(2-((3,4,4-trifluorobut-3-en-1- | ¹H-NMR (400 MHz, DMSO-d₆) δ 7.87 (S, 1H), 4.19-4.08 |
| | yl)thio)thiazol-4-yl)ethan-1-one O- | (q, J = 6.7 Hz, 2H), 3.33 (t, J = 6.0 Hz, 2H), 2.93-2.74 (m, |
| | ethyl oxime | 2H), 2.19 (S, 3H), 1.27-1.15 (t, J = 6.7 Hz, 3H); |
| 142 | (E)-1-(2-((3,4,4-trifluorobut-3-en-1- | ¹H-NMR (400 MHz, DMSO-d₆) δ 8.40 (s, 1H), 4.17-4.22 |
| | yl)sulfinyl)thiazol-4-yl)ethan-1-one O- | (m, 2H), 3.55-3.62 (m, 1H), 3.32-3.39 (m, 1H), 3.38 (s, |
| | ethyl oxime | 3H), 2.61-2.93 (m, 1H), 2.17-2.26 (m, 1H), 1.01-1.36 (t, |
| | | J = 6.7 Hz, 3H); |
| 143 | (E)-1-(2-((3,4,4-trifluorobut-3-en-1- | ¹H-NMR (400 MHz, DMSO-d₆) δ 7.83 (s, 1H), 3.89 (s, |
| | yl)sulfonyl)thiazol-4-yl)ethan-1-one O- | 3H), 3.43 (t, J = 6.7 Hz, 2H), 2.78-2.89 (m, 2H), 2.17 (s, |
| | ethyl oxime | 3H); |
| 144 | (Z)-1-(2-((3,4,4-trifluorobut-3-en-1- | ¹H-NMR (400 MHz, DMSO-d₆) δ 8.24-8.20 (1H), 4.24- |
| | yl)thio)thiazol-5-yl)ethan-1-one O- | 4.13 (2H), 3.53-3.42 (2H), 2.91-2.76 (2H), 2.34-2.22 (3H), |
| | isopentyl oxime | 1.77-1.64 (1H), 1.61-1.52 (2H), 0.99-0.82 (6H); |
| 145 | (Z)-1-(2-((3,4,4-trifluorobut-3-en-1- | ¹H-NMR (400 MHz, DMSO-d₆) δ 8.55 (s, 1H), 4.27 (t, J = |
| | yl)sulfinyl)thiazol-5-yl)ethan-1-one O- | 6.7 Hz, 2H), 3.58-3.65 (m, 1H), 3.37 (t, J = 7.1 Hz, 1H), |
| | isopentyl oxime | 2.84 (d, J = 3.4 Hz, 1H), 2.62-2.68 (m, 1H), 2.35 (s, 3H), |
| | | 1.69-1.75 (m, 1H), 1.60 (q, J = 6.8 Hz, 2H), 0.92 (d, J = 6.6 |
| | | Hz, 6H); |
| 146 | (Z)-1-(2-((3,4,4-trifluorobut-3-en-1- | ¹H-NMR (400 MHz, DMSO-d₆) δ 8.68 (s, 1H), 4.33 (t, J = |
| | yl)sulfonyl)thiazol-5-yl)ethan-1-one O- | 6.7 Hz, 2H), 3.91 (t, J = 7.0 Hz, 2H), 2.80-2.90 (m, 2H), |
| | isopentyl oxime | 2.35 (s, 3H), 1.72 (td, J = 13.4, 6.5 Hz, 1H), 1.62 (q, J = |
| | | 6.8 Hz, 2H), 0.91-0.94 (m, 6H); |
| 147 | (E)-4-methyl-2-((3,4,4-trifluorobut-3- | ¹H-NMR (400 MHz, DMSO-d₆) δ 7.95 (s, 1H), 4.46 (s, |
| | en-1-yl)thio)thiazole-5-carbaldehyde | 1H), 3.43 (t, J = 12.7 Hz, 2H), 2.91-2.77 (2H), 2.49 (s, |
| | O-isopropyl oxime | 3H), 1.27-1.29 (d, J = 1.0 Hz, 6H); |
| 148 | (E)-1-(2-((3,4,4-trifluorobut-3-en-1- | ¹H-NMR (400 MHz, DMSO-d₆) δ 8.30 (s, 1H), 3.94 (s, |
| | yl)thio)thiazol-4-yl)ethan-1-one O- | 3H), 3.53-3.60 (m, 1H), 3.33-3.39 (m, 1H), 2.78-2.89 (m, |
| | methyl oxime | 1H), 2.61-2.72 (m, 1H), 2.18 (s, 3H); |
| 149 | (E)-1-(2-((3,4,4-trifluorobut-3-en-1- | ¹H-NMR (400 MHz, DMSO-d₆) δ 8.44 (s, 1H), 3.94 (s, |
| | yl)sulfinyl)thiazol-4-yl)ethan-1-one O- | 3H), 3.89-3.90 (m, 2H), 2.83-2.90 (m, 2H), 2.23 (s, 3H); |
| | methyl oxime | |
| 150 | (E)-1-(2-((3,4,4-trifluorobut-3-en-1- | ¹H-NMR (400 MHz, DMSO-d₆) δ 8.44 (s, 1H), 3.94 (s, |
| | yl)sulfonyl)thiazol-4-yl)ethan-1-one O- | 3H), 3.89-3.92 (t, J = 6.7 Hz, 2H), 2.83-2.91 (m, 2H), 2.23 |
| | methyl oxime | (s, 3H); |
| 151 | (E)-4-methyl-2-((3,4,4-trifluorobut-3- | ¹H-NMR (400 MHz, DMSO-d₆) δ 7.95 (s, 1H), 4.00 (d, J = |
| | en-1-yl)thio)thiazole-5-carbaldehyde | 6.7 Hz, 2H), 3.44 (t, J = 7.0 Hz, 2H), 2.77-2.88 (m, 2H), |
| | O-cyclopropylmethyl oxime | 2.48 (s, 3H), 1.11-1.17 (m, 1H), 0.50-0.55 (m, 2H), 0.29 |
| | | (td, J = 5.2, 3.9 Hz, 2H); |
| 152 | (E)-2-((3,4,4-trifluorobut-3-en-1- | ¹H-NMR (400 MHz, DMSO-d₆) δ 8.05 (s, 1H), 4.05 (s, |
| | yl)thio)-4-(trifluoromethyl)thiazole-5- | 3H), 3.48-3.53 (m, 2H), 2.79-2.90 (m, 2H); |
| | carbaldehyde O-methyl oxime | |
| 153 | (E)-2-((3,4,4-trifluorobut-3-en-1- | ¹H-NMR (400 MHz, DMSO-d₆) δ 8.22 (s, 1H), 4.15 (s, |
| | yl)sulfinyl)-4-(trifluoromethyl)thiazole- | 3H), 3.62-3.69 (m, 1H), 3.37-3.44 (m, 1H), 2.80-2.92 (m, |
| | 5-carbaldehyde O-methyl oxime | 1H), 2.64-2.75 (m, 1H); |
| 154 | (E)-2-((3,4,4 -trifluorobut-3-en-1- | ¹H-NMR (400 MHz, DMSO-d₆) δ 8.37 (s, 1H), 4.22 (s, |
| | yl)sulfonyl)-4- | 3H), 4.00 (t, J = 7.0 Hz, 2H), 2.86-2.96 (m, 2H); |
| | (trifluoromethyl)thiazole-5- | |
| | carbaldehyde O-methyl oxime | |
| 155 | (E)-2-((3,4,4 -trifluorobut-3-en-1- | ¹H-NMR (400 MHz, DMSO-d₆) δ 8.05 (s, 1H), 4.33 (q, J = |
| | yl)thio)-4-(trifluoromethyl)thiazole-5- | 7.1 Hz, 2H), 3.51 (t, J = 7.0 Hz, 2H), 2.85 (ddt, J = 23.1, |
| | carbaldehyde O-ethyl oxime | 7.0, 3.0 Hz, 2H), 1.30 (t, J = 7.0 Hz, 3H); |
| 156 | (E)-2-((3,4,4 -trifluorobut-3-en-1- | ¹H-NMR (400 MHz, DMSO-d₆) δ 8.20 (s, 1H), 4.40 (q, J = |
| | yl)sulfinyl)-4-(trifluoromethyl)thiazole- | 7.1 Hz, 2H), 3.60-3.66 (m, 1H), 3.35-3.42 (m, 1H), 2.63- |
| | 5-carbaldehyde O-ethyl oxime | 2.88 (m, 2H), 1.31 (t, J = 7.0 Hz, 3H); |
| 157 | (E)-2-((3,4,4-trifluorobut-3-en-1- | ¹H-NMR (400 MHz, DMSO-d₆) δ 8.36 (s, 1H), 4.48 (q, J = |
| | yl)sulfonyl)-4- | 7.1 Hz, 2H), 3.98 (t, J = 7.0 Hz, 2H), 2.86-2.93 (m, 2H), |
| | (trifluoromethyl)thiazole-5- | 1.35 (t, J = 7.0 Hz, 3H); |
| | carbaldehyde O-ethyl oxime | |
| 158 | (E)-4-methyl-2-((3,4,4-trifluorobut-3- | ¹H-NMR (400 MHz, DMSO-d₆) δ 8.14 (s, 1H), 4.11 (d, J = |
| | en-1-yl)sulfinyl)thiazole-5- | 7.1 Hz, 2H), 3.55-3.62 (m, 1H), 2.79-2.89 (m, 1H), 2.64- |
| | carbaldehyde O-cyclopropylmethyl | 2.72 (m, 1H), 2.59 (d, J = 12.7 Hz, 3H), 1.16-1.24 (m, 1H), |
| | oxime | 0.53-0.58 (m, 2H), 0.32-0.35 (m, 2H); |
| 159 | (E)-4-methyl-2-((3,4,4-trifluorobut-3- | ¹H-NMR (400 MHz, DMSO-d₆) δ 8.26 (s, 1H), 4.15 (d, J = |
| | en-1-yl)sulfonyl)thiazole-5- | 7.3 Hz, 2H), 3.87 (t, J = 7.0 Hz, 2H), 2.78-2.89 (m, 2H), |
| | carbaldehyde O-cyclopropylmethyl | 2.65 (d, J = 7.3 Hz, 3H), 1.15-1.24 (m, 1H), 0.53-0.58 (m, |
| | oxime | 2H), 0.33 (td, J = 5.3, 3.7 Hz, 2H); |
| 160 | (E)-4-methyl-2-((3,4,4-trifluorobut-3- | ¹H-NMR (400 MHz, DMSO-d₆) δ 8.11 (s, 1H), 4.42-4.59 |
| | en-1-yl)sulfinyl)thiazole-5- | (m, 1H), 3.84-3.92 (m, 1H), 3.55 (td, J = 14.1, 7.3 Hz, 1H), |
| | carbaldehyde O-isopropyl oxime | 2.76-2.88 (m, 1H), 2.55-2.74 (m, 1H), 2.74 (s, 3H), 1.29- |
| | | 1.33 (m, 6H); |
| 161 | N-((5-chlorothiazol-2-yl)(3,4,4- | ¹H-NMR (400 MHz, CDCl₃) δ 7.83 (s, 1H), 3.73-3.67 (m, |
| | trifluorobut-3-en-l-yl)-λ⁴- | 2H), 3.02-2.96 (m, 1H), 2.97-2.84 (m, 1H) |
| | sulfaneylidene)cyanamide | |
| 162 | N-(thiazol-2-yl(3,4,4-trifluorobut-3-en- | ¹H-NMR (400 MHz, DMSO-d₆) δ 8.30 (d, J = 3.1 Hz, 1H), |
| | 1-yl)-λ⁴-sulfaneylidene)cyanamide | 8.19 (d, J = 3.1 Hz, 1H), 3.89-3.98 (m, 1H), 3.77-3.83 (m, |
| | | 1H), 2.76-2.99 (m, 2H); |
| 163 | N-((5-chlorothiazol-2-yl)(oxo)(3,4,4- | ¹H-NMR (400 MHz, CDCl₃) δ 8.01 (s, 1H), 3.94-3.84 (m, |
| | trifluorobut-3-en-1-yl)-λ⁶- | 2H), 3.05-2.95 (m, 2H); |
| | sulfaneylidene)cyanamide | |
| 164 | N-(oxo(thiazol-2-yl)(3,4,4-trifluorobut- | ¹H-NMR (400 MHz, DMSO-d₆) δ 8.56 (d, J = 2.9 Hz, 1H), |
| | 3-en-1-yl)-λ⁶-sulfaneylidene)cyanamide | 8.39 (d, J = 2.9 Hz, 1H), 4.39 (t, J = 6.9 Hz, 2H), 2.95 |
| | | (ddd, J = 21.4, 3.9, 2.9 Hz, 2H); |
| 165 | N-((5-bromothiazol-2-yl)(oxo)(3,4,4- | ¹H-NMR (400 MHz, DMSO-d₆) δ 8.56 (s, 1H), 4.38-4.42 |
| | trifluorobut-3-en-1-yl)-λ⁶- | (m, 2H), 2.93-3.00 (m, 2H); |
| | sulfaneylidene)cyanamide | |
| 166 | imino(thiazol-2-yl)(3,4,4-trifluorobut- | ¹H-NMR (400 MHz, CDCl₃) δ 8.02 (d, J = 2.8 Hz, 1H), |
| | 3-en-1-yl)-λ⁶-sulfanone | 7.74 (d, J = 2.8 Hz, 1H), 3.64-3.60 (m, 2H), 2.97-2.86 (m, |
| | | 2H); |
| 167 | (ethylimino)(thiazol-2-yl)(3,4,4- | ¹H-NMR (400 MHz, CDCl₃) δ 8.05 (d, J = 2.8 Hz, 1H), |
| | trifluorobut-3-en-1-yl)-λ⁶-sulfanone | 7.75 (d, J = 2.8 Hz, 1H), 3.63-3.59 (m, 2H), 3.22-3.16 (m, |
| | | 1H), 3.07-2.85 (m, 3H), 1.17 (t, J = 7.2 Hz, 3H); |
| 168 | 2,2,2-trifluoro-N-(oxo(thiazol-2- | ¹H-NMR (400 MHz, CDCl₃) δ 8.12 (d, J = 2.8 Hz, 1H), |
| | yl)(3,4,4-trifluorobut-3-en-1-yl)-λ⁶- | 7.92 (d, J = 2.8 Hz, 1H), 4.14-4.01 (m, 2H), 3.04-2.92 (m, |
| | sulfaneylidene)acetamide | 2H); |
| 169 | (5-chlorothiazol-2-yl)(imino)(3,4,4- | ¹H-NMR (400 MHz, CDCl₃) δ 7.82 (s, 1H), 3.61-3.53 (m, |
| | trifluorobut-3-en-1-yl)-λ⁶-sulfanone | 2H), 2.98-2.86 (m, 2H); |
| 170 | (5-chlorothiazol-2- | ¹H-NMR (400 MHz, CDCl₃) δ 7.83 (s, 1H), 7.75 (d, J = |
| | yl)(ethylimino)(3,4,4-trifluorobut-3-en- | 2.8 Hz, 1H), 3.57-3.53 (m, 1H), 3.22-3.16 (m, 1H), 3.21- |
| | 1-yl)-λ⁶-sulfanone | 2.84 (m, 3H), 1.17 (t, J = 7.2 Hz, 3H); |
| 171 | (methylimino)(thiazol-2-yl)(3,4,4- | ¹H-NMR (400 MHz, DMSO-d₆) δ 8.26 (d, J = 3.1 Hz, 1H), |
| | trifluorobut-3-en-1-yl)-λ⁶-sulfanone | 8.17 (d, J = 3.1 Hz, 1H), 3.72 (t, J = 7.2 Hz, 2H), 2.78-2.85 |
| | | (m, 2H), 2.59 (s, 3H); |
| 172 | (propylimino)(thiazol-2-yl)(3,4,4- | ¹H-NMR (400 MHz, DMSO-d₆) δ 8.23 (d, J = 3.1 Hz, 1H), |
| | trifluorobut-3-en-1-yl)-λ⁶-sulfanone | 8.15 (d, J = 3.1 Hz, 1H), 3.70 (t, J = 7.0 Hz, 2H), 2.75-2.91 |
| | | (m, 4H), 1.40 (q, J = 7.1 Hz, 2H), 0.81 (t, J = 7.3 Hz, 3H); |
| 173 | ((cyclopropylmethyl)imino)(thiazol-2- | ¹H-NMR (400 MHz, DMSO-d₆) δ 8.19 (dd, J = 31.9, 3.1 |
| | yl)(3,4,4-trifluorobut-3-en-1-yl)-λ⁶- | Hz, 2H), 3.70 (t, J = 7.1 Hz, 2H), 2.74-2.87 (m, 4H), 0.82- |
| | sulfanone | 0.87 (m, 1H), 0.28-0.33 (m, 2H), 0.02-0.09 (m, 2H); |
| 174 | N-((5-bromothiazol-2-yl)(3,4,4- | ¹H-NMR (400 MHz, DMSO-d₆) δ 8.33 (d, J = 4.6 Hz, 1H), |
| | trifluorobut-3-en-1-yl)-λ⁴- | 3.89-3.94 (m, 1H), 3.76-3.82 (m, 1H), 2.86-2.93 (m, 2H); |
| | sulfaneylidene)cyanamide | |
| 175 | (5-bromothiazol-2-yl)(imino)(3,4,4- | ¹H-NMR (400 MHz, DMSO-d₆) δ 8.38 (s, 1H), 3.90 (t, J = |
| | trifluorobut-3-en-1-yl)-λ⁶-sulfanone | 7.0 Hz, 2H), 2.79-2.90 (m, 2H); |
| 176 | (5-bromothiazol-2- | ¹H-NMR (400 MHz, DMSO-d₆) δ 8.29 (s, 1H), 3.70 (t, J = |
| | yl)(ethylimino)(3,4,4-trifluorobut-3-en- | 7.2 Hz, 2H), 2.97-3.05 (m, 1H), 2.87-2.94 (m, 1H), 2.77- |
| | 1-yl)-λ⁶-sulfanone | 2.87 (m, 2H), 1.03 (t, J = 7.2 Hz, 3H); |
| 177 | (5-(difluoromethyl)thiazol-2- | ¹H-NMR (400 MHz, DMSO-d₆) δ 8.43 (s, 1H), 7.49 (t, J = |
| | yl)(imino)(3,4,4-trifluorobut-3-en-1- | 54.3 Hz, 1H), 5.60 (s, 1H), 3.68-3.71 (m, 2H), 2.78-2.88 |
| | yl)-λ⁶-sulfanone | (m, 2H) |
| 178 | 2-(3,4,4-trifluorobut-3-en-1- | ¹H-NMR (400 MHz, DMSO-d₆) δ 8.90 (s, 1H), 5.77 (s, |
| | ylsulfonimidoyl)thiazole-5-carbonitrile | 1H), 3.71 (td, J = 7.2, 2.9 Hz, 2H), 2.79-2.88 (m, 2H) |
| 179 | (5-bromothiazol-2- | ¹H-NMR (400 MHz, CDCl₃) δ 7.94 (s, 1H), 3.57-3.53 (m, |
| | yl)(methylimino)(3,4,4-trifluorobut-3- | 2H), 2.98-2.81 (m, 2H), 2.78 (s, 3H); |
| | en-1-yl)-λ⁶-sulfanone | |
| 180 | (5-chlorothiazol-2- | ¹H-NMR (400 MHz, CDCl₃) δ 7.85 (s, 1H), 3.55-3.51 (m, |
| | yl)(methylimino)(3,4,4-trifluorobut-3- | 2H), 2.98-2.84 (m, 2H), 2.78 (d, J = 1.6 Hz, 3H); |
| | en-1-yl)-λ⁶-sulfanone | |
| 181 | N-((5-(difluoromethyl)thiazol-2- | ¹H-NMR (400 MHz, DMSO-d₆) δ 8.50 (t, J = 2.0 Hz, 1H), |
| | yl)(3,4,4-trifluorobut-3-en-1-yl)-λ⁴- | 7.49 (t, J = 54.2 Hz, 1H), 3.91-3.98 (m, 1H), 3.77-3.84 (m, |
| | sulfaneylidene)cyanamide | 1H), 2.83-2.95 (m, 2H) |
| 182 | N-((4-methylthiazol-2-yl)(3,4,4- | ¹H-NMR (400 MHz, CDCl₃) δ 7.36 (d, J = 0.8 Hz, 1H), |
| | trifluorobut-3-en-1-yl)-λ⁴- | 3.95-3.86 (m, 2H), 3.05-2.94 (m, 2H), 2.60 (d, J = 1 Hz, |
| | sulfaneylidene)cyanamide | 3H); |
| 183 | N-((4-methylthiazol-2-yl)(oxo)(3,4,4- | ¹H-NMR (400 MHz, CDCl₃) δ 7.51 (d, J = 1.2 Hz, 1H), |
| | trifluorobut-3-en-1-yl)-λ⁶- | 3.72-3.69 (m, 2H), 3.01-2.83 (m, 1H), 2.53 (s, 3H); |
| | sulfaneylidene)cyanamide | |
| 184 | N-((5-chloro-4-methylthiazol-2- | ¹H-NMR (400 MHz, CDCl₃) δ 3.71-3.66 (m, 2H), 3.05- |
| | yl)(3,4,4-trifluorobut-3-en-1-yl)-λ⁴- | 2.81 (m, 2H), 2.44 (s, 3H); |
| | sulfaneylidene)cyanamide | |
| 185 | N-((5-chloro-4-methylthiazol-2- | ¹H-NMR (400 MHz, CDCl₃) δ 3.85-3.83 (m, 2H), 3.03- |
| | yl)(oxo)(3,4,4-trifluorobut-3-en-1-yl)- | 2.96 (m, 2H), 2.52 (s, 3H); |
| | λ⁶-sulfaneylidene)cyanamide | |
| 186 | (5-chloro-4-methylthiazol-2- | ¹H-NMR (400 MHz, CDCl₃) δ 3.59-3.57 (m, 2H), 3.28 (s, |
| | yl)(imino)(3,4,4-trifluorobut-3-en-1- | 1H), 2.98-2.85 (m, 2H), 2.47 (s, 3H); |
| | yl)-λ⁶-sulfanone | |
| 187 | N-((4-(tert-butyl)thiazol-2-yl)(3,4,4- | ¹H-NMR (400 MHz, DMSO-d₆) δ 7.89 (s, 1H), 3.90-3.97 |
| | trifluorobut-3-en-1-yl)-λ⁴- | (m, 1H), 3.76-3.82 (m, 1H), 2.83-2.96 (m, 2H), 1.29 (s, |
| | sulfaneylidene)cyanamide | 9H); |
| 188 | (4-(tert-butyl)thiazol-2- | ¹H-NMR (400 MHz, DMSO-d₆) δ 7.74 (s, 1H), 3.57- |
| | yl(imino)(3,4,4-trifluorobut-3-en-1- | 3.60(m, 2H), 2.74-2.82 (m, 2H), 1.25 (s, 9H); |
| | yl)-λ⁶-sulfanone | |
| 189 | (4-(tert-butyl)thiazol-2- | ¹H-NMR (400 MHz, DMSO-d₆) δ 7.82 (s, 1H), 3.68-3.72 |
| | yl)(methylimino)(3,4,4-trifluorobut-3- | (m, 2H), 2.79-2.88 (m, 2H), 2.59 (s, 3H), 1.31 (s, 9H); |
| | en-1-yl)-λ⁶-sulfanone | |
| 190 | imino(4-phenylthiazol-2-yl)(3,4,4- | ¹H-NMR (400 MHz, DMSO-d₆) δ 3.89-3.95 (m, 1H), 3.73- |
| | trifluorobut-3-en-1-yl)-λ⁶-sulfanone | 3.80 (m, 1H), 2.86-2.95 (m, 2H), 1.36 (s, 9H); |
| 191 | (5-bromo-4-phenylthiazol-2- | ¹H-NMR (400 MHz, DMSO-d₆) δ 5.41 (s, 1H), 3.58-3.66 |
| | yl)(imino)(3,4,4-trifluorobut-3-en-1- | (m, 2H), 2.78-2.88 (m, 2H), 1.41 (s, 9H); |
| | yl)-λ⁶-sulfanone | |
| 192 | (5-chloro-4-phenylthiazol-2- | ¹H-NMR (400 MHz, DMSO-d₆) δ 3.73 (t, J = 7.0 Hz, 2H), |
| | yl)(imino)(3,4,4-trifluorobut-3-en-1- | 2.80-2.89 (m, 2H), 2.63 (s, 3H), 1.40 (s, 9H); |
| | yl)-λ⁶-sulfanone | |
| 193 | N-((4-(tert-butyl)-5-chlorothiazol-2- | ¹H-NMR (400 MHz, DMSO-d₆) δ 3.89-3.95 (m, 1H), 3.73- |
| | yl)(3,4,4-trifluorobut-3-en-1-yl)4,4- | 3.80 (m, 1H), 2.86-2.95 (m, 2H), 1.36 (s, 9H); |
| | sulfaneylidene)cyanamide | |
| 194 | (4-(tert-butyl)-5-chlorothiazol-2- | ¹H-NMR (400 MHz, DMSO-d₆) δ 5.41 (s, 1H), 3.58-3.66 |
| | yl)(imino)(3,4,4-trifluorobut-3-en-1- | (m, 2H), 2.78-2.88 (m, 2H), 1.41 (s, 9H); |
| | yl)-λ⁶-sulfanone | |
| 195 | (4-(tert-butyl)-5-chlorothiazol-2- | ¹H-NMR (400 MHz, DMSO-d₆) δ 3.73 (t, J = 7.0 Hz, 2H), |
| | yl)(methylimino)(3,4,4-trifluorobut-3- | 2.80-2.89 (m, 2H), 2.63 (s, 3H), 1.40 (s, 9H); |
| | en-1-yl)-λ⁶-sulfanone | |
| 196 | (4-(tert-butyl)-5-chlorothiazol-2- | ¹H-NMR (400 MHz, DMSO-d₆) δ 3.74-3.63 (m, 2H), 3.09- |
| | yl)(ethylimino)(3,4,4-trifluorobut-3-en- | 2.90 (m, 2H), 2.90-2.74 (m, 2H), 1.43-1.36 (s, 9H), 1.06- |
| | 1-yl)-λ⁶-sulfanone | 0.99 (t, J = 7.1 Hz, 3H); |
| 197 | N-((5-bromo-4-(tert-butyl)thiazo1-2- | ¹H-NMR (400 MHz, CDCl₃) δ 3.68 (t, J = 7.2 Hz, 2H), |
| | yl)(3,4,4-trifluorobut-3-en-1-yl)-λ⁴- | 2.86-3.00 (m, 2H), 1.45 (s, 9H); |
| | sulfaneylidene)cyanamide | |
| 198 | (5-bromo-4-(tert-butyl)thiazol-2- | ¹H-NMR (400 MHz, CDCl₃) δ 3.84 (t, J = 3.7 Hz, 1H), |
| | yl)(imino)(3,4,4-trifluorobut-3-en-1- | 3.60 (t, J = 7.7 Hz, 2H), 2.88-3.02 (m, 2H), 1.48 (s, 9H); |
| | yl)-λ⁶-sulfanone | |
| 199 | (5-bromo-4-(tert-butyl)thiazol-2- | ¹H-NMR (400 MHz, DMSO-d₆) δ 3.72 (t, J = 7.0 Hz, 2H), |
| | yl)(methylimino)(3,4,4-trifluorobut-3- | 2.80-2.88 (m, 2H), 2.62 (s, 3H), 1.44 (s, 9H); |
| | en-1-yl)-λ⁶-sulfanone | |
| 200 | (5-bromo-4-(tert-butyl)thiazol-2- | ¹H-NMR (400 MHz, DMSO-d₆) δ 3.69 (t, J = 7.0 Hz, 2H), |
| | yl)(ethylimino)(3,4,4-trifluorobut-3-en- | 2.90-3.06 (m, 2H), 2.79-2.89 (m, 2H), 1.40 (s, 9H), 1.03 (t, |
| | 1-yl)-λ⁶-sulfanone | J = 7.2 Hz, 3H); |
| 201 | (4,5-dimethylthiazol-2- | ¹H-NMR (400 MHz, DMSO-d₆) δ 5.15 (s, 1H), 3.49-3.57 |
| | yl)(imino)(3,4,4-trifluorobut-3-en-1- | (m, 2H), 2.71-2.82 (m, 2H), 2.42 (s, 3H), 2.32-2.35 (m, |
| | yl)-λ⁶-sulfanone | 3H); |
| 202 | imino(4-methylthiazol-2-yl)(3,4,4- | ¹H-NMR (400 MHz, CDCl₃) δ 7.29 (d, J = 0.8 Hz, 1H), |
| | trifluorobut-3-en-l-yl)-λ⁶-sulfanone | 3.62-3.60 (m, 2H), 2.98-2.85 (m, 2H), 2.51 (s, 3H), 2.45 (s, |
| | | 1H); |
| 203 | (methylimino)(4-methylthiazol-2- | ¹H-NMR (400 MHz, CDCl₃) δ 7.30 (d, J = 0.8 Hz, 1H), |
| | yl)(3,4,4-trifluorobut-3-en-1-yl)-λ⁶- | 3.75-3.60 (m, 2H), 3.02-2.84 (m, 2H), 2.82 (m, 3H); |
| | sulfanone | |
| 204 | (ethylimino)(4-methylthiazol-2- | ¹H-NMR (400 MHz, CDCl₃) δ 7.30 (d, J = 0.8 Hz, 1H), |
| | yl)(3,4,4-trifluorobut-3-en-1-yl)-λ⁶- | 3.73-3.59 (m, 1H), 3.22-3.14 (m, 1H), 3.09-2.86 (m, 3H), |
| | sulfanone | 1.19 (t, J = 7.2 Hz, 3H); |
| 205 | (5-bromo-4-phenylthiazol-2- | ¹H-NMR (400 MHz, DMSO-d₆) δ 7.84 (d, J = 7.8 Hz, 2H), |
| | yl)(methylimino)(3,4,4-trifluorobut-3- | 7.45-7.53 (m, 3H), 3.73-3.81 (m, 2H), 2.83-2.90 (m, 2H), |
| | en-1-yl)-λ⁶-sulfanone | 2.60-2.68 (m, 3H); |
| 206 | N-((4,5-dimethylthiazol-2-yl)(3,4,4- | ¹H-NMR (400 MHz, DMSO-d₆) δ 3.74-3.92 (m, 2H), 2.79- |
| | trifluorobut-3-en-1-yl)-λ⁴- | 2.93 (m, 2H), 2.47 (s, 3H), 2.31-2.39 (m, 3H); |
| | sulfaneylidene)cyanamide | |
| 207 | 5-(2-chlorophenyl)-3-(2-((3,4,4- | ¹H-NMR (400 MHz, CDCl₃) δ 7.60 (s, 1H), 7.48-7.51 (m, |
| | trifluorobut-3-en-1-yl)thio)thiazol-5- | 1H), 7.36-7.38 (m, 1H), 7.22-7.32 (m, 2H), 6.02 (dd, J = |
| | yl)-4,5-dihydroisoxazole | 11.1, 7.1 Hz, 1H), 3.89 (dd, J = 16.6, 11.1 Hz, 1H), 3.41 (t, |
| | | J = 7.0 Hz, 2H), 3.16 (dd, J = 16.7, 7.2 Hz, 1H), 2.72-2.83 |
| | | (m, 2H); |
| 208 | 5-phenyl-3-(2-((3,4,4-trifluorobut-3-en- | ¹H-NMR (400 MHz, CDCl₃) δ 7.62-7.66 (m, 1H), 7.31- |
| | 1-yl)thio)thiazol-5-yl)-4,5- | 7.41 (m, 5H), 5.76 (dd, J = 11.0, 8.4 Hz, 1H), 3.75 (dd, J = |
| | dihydroisoxazole | 16.4, 10.9 Hz, 1H), 3.45 (t, J = 7.0 Hz, 2H), 3.32 (q, J = 8.3 |
| | | Hz, 1H), 2.76-2.87 (m, 2H); |
| 209 | 5-(2-chlorophenyl)-3-(2-((3,4,4- | ¹H-NMR (400 MHz, CDCl₃) δ 7.94 (s, 1H), 7.53 (d, J = |
| | trifluorobut-3-en-1-yl)sulfinyl)thiazol- | 7.2 Hz, 1H), 7.40-7.43 (m, 1H), 7.28-7.33 (m, 2H), 6.12 |
| | 5-yl)-4,5-dihydroisoxazole | (dd, J = 11.1, 7.3 Hz, 1H), 3.96 (ddd, J = 16.7, 11.2, 2.9 |
| | | Hz, 1H), 3.20-3.47 (m, 3H), 2.88-3.00 (m, 1H), 2.54-2.68 |
| | | (m, 1H), 1.21-1.27 (m, 1H); |
| 210 | 5-(2-chlorophenyl)-3-(2-((3,4,4- | ¹H-NMR (400 MHz, CDCl₃) δ 7.94 (s, 1H), 7.53 (d, J = |
| | trifluorobut-3-en-1-yl)sulfonyl)thiazol- | 7.2 Hz, 1H), 7.40-7.43 (m, 1H), 7.28-7.33 (m, 2H), 6.12 |
| | 5-yl)-4,5-dihydroisoxazole | (dd, J = 11.1, 7.3 Hz, 1H), 3.96 (ddd, J = 16.7, 11.2, 2.9 |
| | | Hz, 1H), 3.20-3.47 (m, 3H), 2.88-3.00 (m, 1H), 2.54-2.68 |
| | | (m, 1H), 1.21-1.27 (m, 1H); |
| 211 | 3-(2-((3,4,4-trifluorobut-3-en-1- | ¹H-NMR (400 MHz, CDCl₃) δ 8.74-8.82 (m, 1H), 8.32 (d, |
| | yl)ethio)thiazol-5-yl)-1,2,4-oxadiazole | J = 14.2 Hz, 1H), 3.47-3.58 (m, 2H), 2.79-2.89 (m, 2H); |
| 212 | 5-phenyl-3-(2-((3,4,4-trifluorobut-3-en- | ¹H-NMR (400 MHz, CDCl₃) δ 8.00-8.01 (m, 1H), 7.34- |
| | 1-yl)sulfonyl)thiazol-5-yl)-4,5- | 7.43 (m, 5H), 5.87 (dd, J = 11.1, 8.5 Hz, 1H), 3.78-3.86 |
| | dihydroisoxazole | (m, 1H), 3.61-3.66 (m, 2H), 3.33-3.41 (m, 1H), 2.86-2.97 |
| | | (m, 2H); |
| 213 | 3-(2-((3,4,4-trifluorobut-3-en-1- | ¹H-NMR (400 MHz, CDCl₃) δ 8.82 (s, 1H), 8.59 (s, 1H), |
| | yl)sulfinyl)thiazol-5-yl)-1,2,4- | 3.45-3.52 (m, 1H), 3.33-3.40 (m, 1H), 2.90-3.04 (m, 1H), |
| | oxadiazole | 2.58-2.71 (m, 1H); |
| 214 | 5-(4-chlorophenyl)-3-(2-((3,4,4- | ¹H-NMR (400 MHz, CDCl₃) δ 8.01 (s, 1H), 7.74-7.78 (m, |
| | trifluorobut-3-en-1-yl)thio)thiazol-5- | 2H), 7.46-7.51 (m, 2H), 6.74 (s, 1H), 3.49 (t, J = 7.0 Hz, |
| | yl)isoxazole | 2H), 2.79-2.90 (m, 2H); |
| 215 | 5-methyl-3-(2-((3,4,4-trifluorobut-3-en- | ¹H-NMR (400 MHz, CDCl₃) δ 8.24 (s, 1H), 3.48 (t, J = 7.0 |
| | 1-yl)thio)thiazol-5-yl)-1,2,4-oxadiazole | Hz, 2H), 2.78-2.89 (m, 2H), 2.64 (s, 3H); |
| 216 | 5-methyl-3-(2-((3,4,4-trifluorobut-3-en- | ¹H-NMR (400 MHz, CDCl₃) δ 8.52 (s, 1H), 3.44-3.51 (m, |
| | 1-yl)sulfinyl)thiazol-5-yl)-1,2,4- | 1H), 3.31-3.38 (m, 1H), 2.89-3.02 (m, 1H), 2.57-2.71 (m, |
| | oxadiazole | 4H); |
| 217 | 5-(2H-tetrazol-5-yl)-2-((3,4,4- | ¹H-NMR (400 MHz, CDCl₃) δ 8.56 (s, 1H), 6.62-5.35 |
| | trifluorobut-3-en-l-yl)thio)thiazole | (1H), 3.54-3.62 (m, 2H), 2.86-2.93 (m, 2H); |
| 218 | 5-(4-chlorophenyl)-3-(2-((3,4,4- | ¹H-NMR (400 MHz, CDCl₃) δ 8.37-8.40 (m, 1H), 7.80 (dt, |
| | trifluorobut-3-en-1-yl)sulfonyl)thiazol- | J = 9.0, 2.2 Hz, 2H), 7.53 (dt, J = 9.0, 2.2 Hz, 2H),6.86 (d, |
| | 5-yl)isoxazole | J = 2.9 Hz, 1H), 3.69-3.75 (m, 2H), 2.92-3.03 (m, 2H); |
| 219 | 5-methyl-3-(2-((3,4,4-trifluorobut-3-en- | ¹H-NMR (400 MHz, CDCl₃) δ 8.58 (d, J = 7.3 Hz, 1H), |
| | 1-yl)sulfonyl)thiazol-5-yl)-1,2,4- | 3.67-3.70 (m, 2H), 2.86-2.99 (m, 2H), 2.70 (s, 3H); |
| | oxadiazole | |
| 220 | 5-(2H-tetrazol-5-yl)-2-((3,4,4- | ¹H-NMR (400 MHz, METHANOL-D4) δ 8.68 (d, J = 5.0 |
| | trifluorobut-3-en-1-yl)sulfonyl)thiazole | Hz, 1H), 3.84 (t, J = 7.2 Hz, 2H), 2.92-2.98 (m, 2H); |
| 221 | 3-(2-((3,4,4-trifluorobut-3-en-1- | ¹H-NMR (400 MHz, DMSO-d₆) δ 8.17 (s, 1H), 3.53 (t, J = |
| | yl)thio)thiazol-5-yl)-1,2,4-oxadiazol- | 6.8 Hz, 2H), 2.81-2.92 (m, 2H); |
| | 5(4H)-one | |
| 222 | 3-(2-((3,4,4-trifluorobut-3-en-1- | ¹H-NMR (400 MHz, DMSO-d₆) δ 14.51-12.90 (1H), 8.54 |
| | yl)sulfinyl)thiazol-5-yl)-1,2,4- | (d, J = 7.3 Hz, 1H), 3.65-3.71 (m, 1H), 3.38-3.45 (m, 1H), |
| | oxadiazol-5(4H)-one | 2.81-2.94 (m, 1H), 2.60-2.73 (m, 1H), 1.25 (d, J = 9.3 Hz, |
| | | 1H); |
| 223 | 5-(2H-tetrazol-5-yl)-2-((3,4,4- | ¹H-NMR (400 MHz, DMSO-d₆) δ 8.66 (t, J = 7.1 Hz, 1H), |
| | trifluorobut-3-en-1-yl)sulfinyl)thiazole | 3.63-3.70 (m, 1H), 3.37-3.44 (m, 1H), 2.79-2.94 (m, 1H), |
| | | 2.60-2.75 (m, 1H); |
| 224 | 3-(2-((3,4,4-trifluorobut-3-en-1- | ¹H-NMR (400 MHz, DMSO-d₆) δ 8.60 (s, 1H), 3.97 (t, J = |
| | yl)sulfonyl)thiazol-5-yl)-1,2,4- | 7.0 Hz, 2H), 2.80-2.91 (m, 2H); |
| | oxadiazol-5(4H)-one | |
| 225 | 5-(4-chlorophenyl)-3-(2-((3,4,4- | ¹H-NMR (400 MHz, DMSO-d₆) δ 8.25 (s, 1H), 7.94-7.97 |
| | trifluorobut-3-en-1-yl)thio)thiazol-4- | (m, 2H), 7.64 (dt, J = 9.0, 2.2 Hz, 2H), 7.50 (s, 1H), 3.52 |
| | yl)isoxazole | (t, J = 6.8 Hz, 2H), 2.83-2.94 (m, 2H); |
| 226 | 3-(2-((3,4,4-trifluorobut-3-en-1- | ¹H-NMR (400 MHz, DMSO-d₆) δ 8.61 (t, J = 4.2 Hz, 1H), |
| | yl)thio)thiazol-4-yl)-5- | 3.54 (t, J = 6.8 Hz, 2H), 2.85-2.95 (m, 2H); |
| | (trifluoromethyl)-1,2,4-oxadiazole | |
| 227 | 3-(2-((3,4,4-trifluorobut-3-en-1- | ¹H-NMR (400 MHz, DMSO-d₆) δ 9.05 (s, 1H), 3.66-3.71 |
| | yl)sulfinyl)thiazol-4-yl)-5- | (m, 1H), 3.40-3.47 (m, 1H), 2.82-2.94 (m, 1H), 2.65-2.76 |
| | (trifluoromethyl)-1,2,4-oxadiazole | (m, 1H); |
| 228 | 3-(2-((3,4,4-trifluorobut-3-en-1- | ¹H-NMR (400 MHz, DMSO-d₆) δ 9.16 (s, 1H), 4.02 (t, J = |
| | yl)sulfonyl)thiazol-4-yl)-5- | 7.0 Hz, 2H), 2.87-2.97 (m, 2H); |
| | (trifluoromethyl)-1,2,4-oxadiazole | |
| 229 | 3-(2-((3,4,4-trifluorobut-3-en-1- | ¹H-NMR (400 MHz, DMSO-d₆) δ 8.54 (s, 1H), 3.58 (t, J = |
| | yl)thio)thiazol-5-yl)-5- | 6.8 Hz, 2H), 2.84-2.95 (m, 2H); |
| | (trifluoromethyl)-1,2,4-oxadiazole | |
| 230 | 5-methyl-3-(2-((3,4,4-trifluorobut-3-en- | ¹H-NMR (400 MHz, DMSO-d₆) δ 8.36 (s, 1H), 3.52 (t, J = |
| | 1-yl)thio)thiazol-4-yl)-1,2,4-oxadiazole | 6.8 Hz, 2H), 2.83-2.94 (m, 2H), 2.66 (s, 3H); |
| 231 | N-((4-(5-methyl-1,2,4-oxadiazol-3- | ¹H-NMR (400 MHz, DMSO-d₆) δ 8.88 (s, 1H), 3.98-4.05 |
| | yl)thiazol-2-yl)(3,4,4-trifluorobut-3-en- | (m, 1H), 3.83-3.89 (m, 1H), 2.91-3.02 (m, 2H), 2.70 (s, |
| | 1-yl)-λ⁴-sulfaneylidene)cyanamide | 3H); |
| 232 | N-((3,4,4-trifluorobut-3-en-1-yl)(5-(5- | ¹H-NMR (400 MHz, DMSO-d₆) δ 8.97 (s, 1H), 4.01-4.08 |
| | (trifluoromethyl)-1,2,4-oxadiazol-3- | (m, 1H), 3.87-3.93 (m, 1H), 2.85-3.06 (m, 2H); |
| | yl)thiazo1-2-yl)4,4- | |
| | sulfaneylidene)cyanamide | |
| 233 | (Z)-2-((4,4-difluorobut-3-en-1- | ¹H-NMR (400 MHz, DMSO-d₆) δ 8.62 (s, 1H), 8.37 (d, J = |
| | yl)sulfonyl)thiazole-5-carbaldehyde O- | 0.5 Hz, 1H), 4.52 (dtd, J = 26.1, 7.8, 2.3 Hz, 1H), 4.20 (q, |
| | ethyl oxime | J = 7.1 Hz, 2H), 3.74 (t, J = 7.3 Hz, 2H), 2.39-2.45 (m, 2H), |
| | | 1.26 (t, J = 7.1 Hz, 3H); LCMS (M + 1): 311.15; |
| 234 | (E)-2-((4,4-difluorobut-3-en-1- | ¹H-NMR (400 MHz, DMSO-d₆) δ 8.15 (d, J = 6.6 Hz, 1H), |
| | yl)thio)thiazole-5-carbaldehyde O- | 7.98 (s, 1H), 4.60 (dtd, J = 26.7, 7.9, 2.6 Hz, 1H), 3.97 (s, |
| | methyl oxime | 3H), 3.36 (t, J = 7.2 Hz, −2H), 2.43 (ddt, J = 14.8, 6.9, 1.7 |
| | | Hz, 2H); LCMS (M + 1): 264.85; |
| 235 | (Z)-2-((4,4-difluorobut-3-en-1- | ¹H-NMR (400 MHz, DMSO-d₆) δ 8.45 (d, J = 5.6 Hz, 1H), |
| | yl)thio)thiazole-5-carbaldehyde O- | 7.93 (s, 1H), 4.60 (dtd, J = 26.7, 7.9, 2.6 Hz, 1H), 3.85 (s, |
| | methyl oxime | 3H), 3.32-3.35 (m, 2H), 2.43 (ddt, J = 14.8, 7.0, 1.7 Hz, |
| | | 2H); LCMS (M + 1): 264.90; |
| 236 | (Z)-2-((4,4-difluorobut-3-en-1- | ¹H-NMR (400 MHz, DMSO-d₆) δ 8.63 (q, J = 6.1 Hz, 1H), |
| | yl)sulfonyl)thiazole-5-carbaldehyde O- | 8.37-8.41 (m, 1H), 4.52 (dtd, J = 26.2, 7.8, 2.4 Hz, 1H), |
| | methyl oxime | 3.94 (s, 3H), 3.73-3.77 (m, 2H), 2.39-2.45 (m, 2H), |
| | | LCMS (M + 1): 296.90; |
| 237 | (E)-2-((4,4-difluorobut-3-en-1- | ¹H-NMR (400 MHz, DMSO-d₆) δ 8.47 (s, 1H), 8.17 (s, |
| | yl)sulfonyl)thiazole-5-carbaldehyde O- | 1H), 4.51-4.63 (m, 1H), 4.33 (q, J = 7.1 Hz, 2H), 3.37-3.49 |
| | ethyl oxime | (m, 1H), 3.18-3.27 (m, 1H), 2.41-2.49 (m, 1H), 2.18-2.28 |
| | | (m, 1H), 1.32 (t, J = 7.0 Hz, 3H); |
| 238 | (E)-4-methyl-2-((3,4,4-trifluorobut-3- | ¹H-NMR (400 MHz, CDCl₃) δ 7.95 (s, 1H), 3.99 (s, 3H), |
| | en-1-yl)thio)oxazole-5-carbaldehyde O- | 3.37 (t, J = 7.1 Hz, 2H), 2.79-2.89 (m, 2H), 2.50 (s, 3H); |
| | methyl oxime | |
| 239 | (E)-4-methyl-2-((3,4,4-trifluorobut-3- | ¹H-NMR (400 MHz, CDCl₃) δ 7.96 (s, 1H), 4.24 (q, J = |
| | en-l-yl)thio)oxazole-5-carbaldehyde O- | 7.1 Hz, 2H), 3.37 (t, J = 7.1 Hz, 2H), 2.79-2.89 (m, 2H), |
| | ethyl oxime | 2.41 (s, 3H), 1.17-1.60 (m, 3H); |
| 240 | (E)-4-(tert-butyl)-2-((3,4,4-trifluorobut- | ¹H-NMR (400 MHz, CDCl₃) δ 8.21 (s, 1H), 4.00 (s, 3H), |
| | 3-en-1-yl)thio)oxazole-5-carbaldehyde | 3.35 (m, 2H), 2.81-2.92 (m, 2H), 1.18 (s, 9H); |
| | O-methyl oxime | |
| 241 | (E)-4-(tert-butyl)-2-((3,4,4-trifluorobut- | ¹H-NMR (400 MHz, CDCl₃) δ 8.20 (s, 1H), 4.25 (q, J = |
| | 3-en-1-yl)thio)oxazole-5-carbaldehyde | 7.1 Hz, 2H), 3.34 (m, 2H), 2.81-2.92 (m, 2H), 1.29 (s, 9H), |
| | O-ethyl oxime | 1.10 (m,3H); |
| 242 | (E)-5-chloro-2-((3,4,4-trifluorobut-3- | ¹H-NMR (400 MHz, DMSO-d₆) δ 8.15 (s, 1H), 4.19 (q, J = |
| | en-1-yl)thio)thiazole-4-carbaldehyde | 7.1 Hz, 2H), 3.46 (t, J = 6.8 Hz, 2H), 2.81-2.90 (m, 2H), |
| | O-ethyl oxime | 1.25 (t, J = 7.1 Hz, 3H); LCMS (M + 1) = 330.85; |
| 243 | (E)-5-chloro-2-((3,4,4-trifluorobut-3- | ¹H-NMR (400 MHz, DMSO-d₆) δ 8.26 (s, 1H), 4.20 (q, J = |
| | en-1-yl)sulfinyl)thiazole-4- | 7.1 Hz, 2H), 3.58-3.65 (m, 1H), 3.34-3.41 (m, 1H), 2.80- |
| | carbaldehyde O-ethyl oxime | 2.93 (m, 1H), 2.66-2.78 (m, 1H), 1.27 (t, J = 7.1 Hz, 3H); |
| | | LCMS (M + 1) = 346.90; |
| 244 | (E)-5-chloro-2-((3,4,4-trifluorobut-3- | ¹H-NMR (400 MHz, DMSO-d₆) δ 8.27 (s, 1H), 3.95 (s, |
| | en-1-yl)sulfinyl)thiazole-4- | 3H), 3.59-3.65 (m, 1H), 3.34-3.41 (m, 1H), 2.97-2.80 (m, |
| | carbaldehyde O-methyl oxime | 1H), 2.79-2.61 (m, 1H); LCMS (M + 1) = 332.85; |
| 245 | 4-methoxy-2-((3,4,4-trifluorobut-3-en- | ¹H-NMR (400 MHz, DMSO-d₆) δ 7.94 (s, 1H), 4.34 (q, J = |
| | 1-yl)sulfonyl)thiazole-5-carbaldehyde | 7.0 Hz, 2H), 4.11 (s, 3H), 3.89 (t, J = 7.0 Hz, 2H), 2.83- |
| | O-ethyl oxime | 2.94 (m, 2H), 1.32 (t, J = 7.1 Hz, 3H); LCMS (M+) = |
| | | 358.55; |
| 246 | (E)-5-((2- | ¹H-NMR (400 MHz, DMSO-d₆) δ 10.48 (s, 1H), 7.97 (s, |
| | phenylhydrazineylidene)methyl)-2- | 1H), 7.79 (s, 1H), 7.18 (t, J = 7.9 Hz, 2H), 6.93 (d, J = 7.3 |
| | ((3,4,4-trifluorobut-3-en-1- | Hz, 2H), 6.71 (d, J = 7.3 Hz, 1H), 3.42 (t, J = 6.7 Hz, 2H), |
| | yl)thio)thiazole | 2.75-2.86 (m, 2H); LCMS (M + 1): 343.60; |
| 247 | (E)-5-((2- | ¹H-NMR (400 MHz, DMSO-d₆) δ 7.63 (d, J = 5.9 Hz, 2H), |
| | methylhydrazineylidene)methyl)-2- | 7.55 (s, 1H), 3.41 (t, J = 6.7 Hz, 2H), 2.80-2.87 (m, 2H), |
| | ((3,4,4-trifluorobut-3-en-1- | 2.77 (d, J = 4.2 Hz, 3H); LCMS (M + 1) = 282; |
| | yl)thio)thiazole | |
| 248 | (E)-2-((3,4,4-trifluorobut-3-en-1- | ¹H-NMR (400 MHz, DMSO-d₆) δ 8.16 (d, J = 1.2 Hz, 1H), |
| | yl)thio)thiazole-5-carbaldehyde O- | 8.03 (d, J = 1.2 Hz, 1H), 7.31-7.39 (m, 5H), 5.20 (d, J = |
| | benzyl oxime | 54.5 Hz, 2H), 3.48 (t, J = 6.8 Hz, 2H), 2.78-2.88 (m, 2H); |
| | | LCMS (M) = 358.6; |
| 249 | (E)-5-((2- | ¹H-NMR (400 MHz, DMSO-d₆) δ 10.53 (d, J = 4.9 Hz, |
| | phenylhydrazineylidene)methyl)-2- | 1H), 7.75 (s, 1H), 7.39 (s, 1H), 7.20-7.26 (m, 2H), 7.01 |
| | ((3,4,4-trifluorobut-3-en-1- | (dd, J = 8.7, 1.1 Hz, 2H), 6.76-6.80 (m, 1H), 3.43 (t, J = |
| | yl)thio)oxazole | 6.8 Hz, 2H), 2.83-2.94 (m, 2H); LCMS (M + 1): 328.10 |
| 250 | (E)-2-((3,4,4-trifluorobut-3-en-1- | ¹H-NMR (400 MHz, DMSO-d₆) δ 8.49 (s, 1H), 8.21 (s, |
| | yl)sulfinyl)thiazole-5-carbaldehyde O- | 1H), 7.32-7.43 (m, 5H), 5.36 (s, 2H), 3.58-3.63 (m, 1H), |
| | benzyl oxime | 3.37 (q, J = 7.0 Hz, 1H), 2.76-2.87 (m, 1H), 2.58-2.71 (m, |
| | | 1H); LCMS (M + 1) = 375; |
| 251 | 4-methoxy-2-((3,4,4-trifluorobut-3-en- | ¹H-NMR (400 MHz, DMSO-d₆) δ 8.08 (s, 1H), 7.60 (s, |
| | 1-yl)thio)thiazole-5-carbaldehyde O- | 1H), 4.00 (s, 3H), 3.96 (s, 3H), 3.89 (s, 3H), 3.78 (s, 3H), |
| | methyl oxime | 3.45 (t, J = 7.0 Hz, 4H), 2.85 (dt, J = 23.2, 3.1 Hz, 4H); |
| | | LCMS (M+) = 312.50; |
| 252 | 4-methoxy-2-((3,4,4-trifluorobut-3-en- | ¹H-NMR (400 MHz, DMSO-d₆) δ 7.93 (s, 1H), 4.10 (s, |
| | 1-yl)sulfonyl)thiazole-5-carbaldehyde | 3H), 4.07 (s, 3H), 3.87-3.91 (m, 2H), 2.83-2.93 (m, 2H) ; |
| | O-methyl oxime | LCMS (M + 1) = 345.00 |
| 253 | (E)-2-((3,4,4-trifluorobut-3-en-1- | ¹H-NMR (400 MHz, DMSO-d₆) δ 8.61 (s, 1H), 8.35 (t, J = |
| | yl)sulfonyl)thiazole-5-carbaldehyde O- | 5.6 Hz, 1H), 7.34-7.44 (m, 5H), 5.41 (s, 2H), 3.91 (t, J = |
| | benzyl oxime | 7.0 Hz, 2H), 2.80-2.90 (m, 2H); LCMS (M + 1) = 391; |
| 254 | (E)-1-(4-methyl-2-((3,4,4-trifluorobut- | 1 H-NMR (400 MHz, DMSO-d₆) δ 4.08 (s, 3H), 3.32-3.41 |
| | 3-en-1-yl)thio)oxazol-5-yl)ethan-1-one | (m, 2H), 2.67-2.91 (m, 2H), 2.27 (s, 3H), 2.11 (s, 3H); |
| | O-methyl oxime | |
| 255 | (E)-1-(4-methyl-2-((3,4,4-trifluorobut- | ¹H-NMR (400 MHz, DMSO-d₆) δ 4.18 (q, J = 7.1 Hz, 2H), |
| | 3-en-1-yl)thio)oxazol-5-yl)ethan-1-one | 3.39 (m, 2H), 2.68-2.91 (m, 2H), 2.20 (s, 3H), 1.91 (s, 3H), |
| | O-ethyl oxime | 1.12 (t, J = 7.1 Hz, 3H); |
| 256 | (E)-1-(4-methyl-2-((3,4,4-trifluorobut- | ¹H-NMR (400 MHz, DMSO-d₆) δ 3.84 (s, 3H), 3.40 (t, J = |
| | 3-en-1-yl)thio)oxazol-5-yl)propan-1- | 6.8 Hz, 2H), 2.86-2.90 (m, 1H), 2.80-2.85 (m, 1H), 2.58 |
| | one O-methyl oxime | (q, J = 7.6 Hz, 2H), 2.27 (s, 3H), 1.04 (t, , J = 7.6 Hz, 3H); |
| 257 | (E)-1-(4-(tert-butyl)-2-((3,4,4- | ¹H-NMR (400 MHz, DMSO-d₆) δ 3.66 (s, 3H), 3.32-3.49 |
| | trifluorobut-3-en-1-yl)thio)oxazol-5- | (m, 2H), 2.68-2.92 (m, 2H), 2.10 (s, 3H), 1.12 (s, 9H); |
| | yl)ethan-1-one O-methyl oxime | |
| 258 | (E)-1-(4-(tert-butyl)-2-((3,4,4- | ¹H-NMR (400 MHz, CDCl₃) 3.85 (s, 3H), 3.26-3.33 (m, |
| | trifluorobut-3-en-1-yl)thio)oxazol-5- | 2H), 2.79-2.90 (m, 2H), 2.57 (q, , J = 6.8 Hz, 2H), 1.31 (s, |
| | yl)propan-1-one O-methyl oxime | 9H), 0.96 (t, J = 6.8 Hz, 3H); |
| 259 | (E)-1-(4-(tert-butyl)-2-((3,4,4- | ¹H-NMR (400 MHz, CDCl₃) δ 4.19-4.30 (q, J = 7.2 Hz, |
| | trifluorobut-3-en-1-yl)thio)oxazol-5- | 2H), 3.30-3.39 (m, 2H), 2.79-2.91 (m, 2H), 2.14 (s, 3H), |
| | yl)ethan-1-one O-ethyl oxime | 1.30-1.37 (m, 12H); |
| 260 | (E)-4-methoxy-2-((3,4,4-trifluorobut-3- | ¹H-NMR (400 MHz, DMSO-d₆) δ 7.78 (s, 1H), 4.04 (s, |
| | en-1-yl)sulfinyl)thiazole-5-carbaldehyde O- | 3H), 4.00 (s, 3H), 3.56-3.63 (m, 1H), 3.34 (t, J = 7.0 Hz, |
| | methyl oxime | 1H), 2.78-2.88 (m, 1H), 2.62-2.71 (m, 1H); LCMS (M +1) = |
| | | 329.00; |
| 261 | (E)-4-methoxy-2-((3,4,4-trifluorobut-3- | ¹H-NMR (400 MHz, DMSO-d₆) δ 7.76 (s, 1H), 4.23 (q, J = |
| | en-1-yl)sulfinyl)thiazole-5- | 7.1 Hz, 2H), 4.01 (s, 3H), 3.53-3.60 (m, 1H), 3.32 (t, J = |
| | carbaldehyde O-ethyl oxime | 7.0 Hz, 1H), 2.75-2.83 (m, 1H), 2.62-2.70 (m, 1H), 1.23- |
| | | 1.30 (t, J = 7.1 Hz, 3H); LCMS (M + 1) = 343.00; |
| 262 | (E)-4-(tert-butyl)-2-((3,4,4-trifluorobut- | ¹H-NMR (400 MHz, CDCl₃) δ 8.25 (s, 1H), 4.01 (d, J = |
| | 3-en-1-yl)thio)oxazole-5-carbaldehyde | 7.0 Hz, 2H), 3.33-3.40 (m, 2H), 2.81-2.92 (m, 2H), 1.32 (s, |
| | O-cyclopropylmethyl oxime | 9H), 0.59-0.64 (m, 2H), 0.32-0.36 (m, 2H); |
| 263 | (E)-4-methyl-2-((3,4,4-trifluorobut-3- | ¹H-NMR (400 MHz, CDCl₃) δ 8.27 (s, 1H), 3.90 (d, J = |
| | en-1-yl)thio)oxazole-5-carbaldehyde O- | 6.9 Hz, 2H), 3.31-3.48 (m, 2H), 2.79-2.81 (m, 2H), 2.18 (s, |
| | cyclopropylmethyl oxime | 3H), 1.07-1.18 (m, 1H), 0.48-0.59 (m, 2H), 0.23-0.27 (m, |
| | | 2H); |
| 264 | 2-methyl-1 -(4-methyl-2-((3,4,4- | ¹H-NMR (400 MHz, DMSO-d₆) δ 3.86 (s, 3H), 3.83 (s, |
| | trifluorobut-3-en-1-yl)thio)oxazol-5- | 3H), 3.33-3.37 (m, 4H), 2.77-2.88 (m, 4H), 2.47 (d, J = 7.1 |
| | yl)propan-1-one O-methyl oxime | Hz, 1H), 2.32 (d, J = 7.3 Hz, 1H), 2.25 (s, 3H), 2.09 (s, |
| | | 3H),1.02 (d, J = 6.7 Hz, 6H), 0.81-0.91 (d, J = 6.7 Hz, 6H); |
| 265 | 3-methyl-1-(4-methyl-2-((3,4,4- | ¹H-NMR (400 MHz, DMSO-d₆) δ 3.85(s, 3H), 3.39 (t, J = |
| | trifluorobut-3-en-1-yl)thio)oxazol-5- | 6.7 Hz, 2H), 3.32 (s, 2H), 2.68-2.90 (m, 2H), 2.51 (s, 3H), |
| | yl)butan-1-one O-methyl oxime | 2.24 (m, 1H), 0.71-0.88 (m, 6H); |
| 266 | (E)-4-methyl-2-((3,4,4-trifluorobut-3- | ¹H-NMR (400 MHz, DMSO-d₆) δ 8.46 (s, 1H), 3.97-4.04 |
| | en-1-yl)sulfinyl)oxazole-5- | (m, 2H), 3.48-3.68 (m, 1H), 3.34 (s, 1H), 2.67-2.95 (m, |
| | carbaldehyde O-cyclopropylmethyl | 2H), 1.01-1.27 (m, 1H), 0.50-0.56 (m, 2H), 0.28-0.35 (m, |
| | oxime | 2H); |
| 267 | (E)-4-methyl-2-((3,4,4-trifluorobut-3- | ¹H-NMR (400 MHz, DMSO-d₆) δ 8.50 (s, 1H), 4.00-4.06 |
| | en-1-yl)sulfonyl)oxazole-5- | (m, 2H), 2.83-2.94 (m, 2H), 2.28 (m, 2H), 2.18 (s, 3H), |
| | carbaldehyde O-cyclopropylmethyl | 1.30 (m, 1H), 0.49-0.57 (m, 2H), 0.25-0.32 (m, 2H); |
| | oxime | |
| 268 | 5-(thiophen-2-yl)-3-(2-((3,4,4- | ¹H-NMR (400 MHz, DMSO-d₆) δ 8.80 (d, J = 4.4 Hz, 1H), |
| | trifluorobut-3-en-1-yl)sulfinyl)thiazol- | 8.14-8.19 (m, 2H), 7.40 (dd, J = 5.0, 3.8 Hz, 1H), 3.67- |
| | 5-yl)-1,2,4-oxadiazole | 3.74 (m, 1H), 3.41-3.48 (m, 1H), 2.64-2.94 (m, 2H); |
| | | LCMS (M + 1): 391.00; |
| 269 | 5-cyclobutyl-3-(2-((3,4,4-trifluorobut- | ¹H-NMR (400 MHz, DMSO-d₆) δ 8.37 (d, J = 1.5 Hz, 1H), |
| | 3-en-1-yl)thio)thiazol-5-yl)-1,2,4- | 3.87-3.94 (m, 1H), 3.55 (t, J = 6.8 Hz, 2H), 2.84-2.94 (m, |
| | oxadiazole | 2H), 2.40-2.46 (m, 4H), 2.07-2.17 (m, 2H); LCMS (M + 1): |
| | | 348.05; |
| 270 | 5-(thiophen-2-yl)-3-(2-((3,4,4- | ¹H-NMR (400 MHz, DMSO-d₆) δ 8.91-8.95 (m, 1H), 8.15- |
| | trifluorobut-3-en-1-yl)sulfonyl)thiazol- | 8.20 (m, 2H), 7.41 (dd, J = 5.0, 3.8 Hz, 1H), 4.03 (t, J = 7.0 |
| | 5-yl)-1,2,4-oxadiazole | Hz, 2H), 2.87-2.97 (m, 2H); LCMS (M + 1): 408.00; |
| 271 | 5-cyclopropyl-3-(2-((3,4,4-trifluorobut- | ¹H-NMR (400 MHz, DMSO-d₆) δ 8.33 (d, 1H), 3.54 (t, J = |
| | 3-en-1-yl)thio)thiazol-5-yl)-1,2,4- | 6.8 Hz, 2H), 2.82-2.93 (m, 2H), 2.38-2.44 (m, 1H), 1.09- |
| | oxadiazole | 1.33 (m, 4H); LCMS (M + 1): 334.05; |
| 272 | (E)-4-methyl-2-((3,4,4-trffluorobut-3- | ¹H-NMR (400 MHz, DMSO-d₆) δ 8.29 (s, 1H), 3.85-4.06 |
| | en-1-yl)thio)oxazole-5-carbaldehyde O- | (m, 2H), 3.41 (m, 2H), 2.81-2.91 (m, 2H), 2.18 (s, 3H), 2.0 |
| | isobutyl oxime | (m, 1H) 0.74-0.92 (m, 6H); |
| 273 | (E)-2-methyl-1-(4-methyl-2-((3,4,4- | ¹H-NMR (400 MHz, DMSO-d₆) δ 3.92 (s, 3H), 3.89 |
| | trifluorobut-3-en-1-yl)sulfinyl)oxazol- | (s, 3H), 3.60-3.67 (m, 2H), 3.40-3.55 (m, 2H), 2.75-2.89 |
| | 5-yl)propan-1-one O-methyl oxime | (m, 2H), 2.28 (s, 3H), 2.14 (s, 3H),1.15 (d, J = 9.6 Hz, 6H), |
| | | 1.08 (d, J = 9.6 Hz, 6H); |
| 274 | 3-methyl-1-(4-methyl-2-((3,4,4- | ¹H-NMR (400 MHz, DMSO-d₆) δ 3.94 (s, 3H), 3.61-3.68 |
| | trifluorobut-3-en-1-yl)sulfinyl)oxazol- | (m, 1H), 3.49-3.56 (m, 1H), 2.73-2.96 (m, 2H), 2.55-2.68 |
| | 5-yl)butan-1-one O-methyl oxime | (m, 2H), 2.35 (d, J = 10.0 Hz, 3H), 1.89-2.00 (m, 1H), |
| | | 0.85-0.94 (m, 6H); |
| 275 | (E)-2-((3,4,4-trifluorobut-3-en-1- | ¹H-NMR (400 MHz, DMSO-d₆) δ 8.14 (s, 1H), 7.97 (s, |
| | yl)thio)thiazole-5-carbaldehyde O- | 1H), 4.01 (d, J = 6.7 Hz, 2H), 3.48 (t, J = 6.7 Hz, 2H), |
| | cyclopropylmethyl oxime | 2.77-2.88 (m, 2H), 1.12-1.18 (m, 1H), 0.51-0.55 (m, 2H), |
| | | 0.30 (td, J = 5.2, 3.9 Hz, 2H); |
| 276 | (E)-2-((3,4,4-trifluorobut-3-en-1- | ¹H-NMR (400 MHz, DMSO-d₆) δ 8.51 (s, 1H), 8.24 (s, |
| | yl)sulfinyl)thiazole-5-carbaldehyde O- | 1H), 4.15 (d, J = 7.3 Hz, 2H), , 3.59-3.66 (m, 1H), 3.38 (m, |
| | cyclopropylmethyl oxime | 1H), 2.77-2.92 (m, 1H), 2.58-2.73 (m, 1H), 1.09-1.34 (m, |
| | | 1H), , 0.51-0.62 (m, 2H), 0.32-0.40 (m, 2H); |
| 277 | 5-cyclopropyl-3-(2-((3,4,4-trifluorobut- | ¹H-NMR (400 MHz, DMSO-d₆) δ 8.77-8.81 (m, 1H), 4.00 |
| | 3-en-1-yl)sulfonyl)thiazol-5-yl)-1,2,4- | (t, J = 7.1 Hz, 2H), 2.85-2.95 (m, 2H), 2.49 (d, J = 1.2 Hz, |
| | oxadiazole | 1H), 1.31-1.37 (m, 2H), 1.21-1.25 (m, 2H), 1.17-1.00 |
| | | (2H); LCMS (M + 1): 366.10; |
| 278 | 5-cyclobutyl-3-(2-((3,4,4-trifluorobut- | ¹H-NMR (400 MHz, DMSO-d₆) δ 8.86 (s, 1H), 3.98-4.03 |
| | 3-en-1-yl)sulfonyl)thiazol-5-yl)-1,2,4- | (m, 2H), 2.85-2.96 (m, 2H), 2.38-2.48 (m, 4H), 2.09-2.19 |
| | oxadiazole | (m, 1H), 1.95-2.04 (m, 1H); LCMS: 380.15 M + 1 |
| 279 | 3-(2-((3,4,4-trifluorobut-3-en-1- | ¹H-NMR (400 MHz, DMSO-d₆) δ 8.90 (d, J = 2.2 Hz, 1H), |
| | yl)sulfinyl)thiazol-5-yl)-5- | 3.69-3.76 (m, 1H), 3.41-3.49 (m, 1H), 2.82-2.96 (m, 1H), |
| | (trifluoromethyl)-1,2,4-oxadiazole | 2.63-2.75 (m, 1H); LCMS (M + 1): 377.95; |
| 280 | 3-(2-((3,4,4-trifluorobut-3-en-1- | ¹H-NMR (400 MHz, DMSO-d₆) δ 9.03 (t, J = 7.2 Hz, 1H), |
| | yl)sulfonyl)thiazol-5-yl)-5- | 4.05 (t, J = 7.0 Hz, 2H), 2.87-2.98 (m, 2H); |
| | (trifluoromethyl)-1,2,4-oxadiazole | |
| 281 | 4-cyclopropyl-2-((3,4,4-trifluorobut-3- | ¹H-NMR (400 MHz, DMSO-d₆) δ 8.16 (s, 1H), 3.95 (s, |
| | en-1-yl)thio)thiazole-5-carbaldehyde | 3H), 3.41 (t, J = 7.0 Hz, 2H), 2.77-2.85 (m, 2H), 2.44-2.48 |
| | O-methyl oxime | (m, 1H), 0.95-1.04 (m, 4H); LCMS (M + 1) = 322.90; |
| 282 | 4-cyclopropyl-2-((3,4,4-trifluorobut-3- | ¹H-NMR (400 MHz, DMSO-d₆) δ 8.16 (s, 1H), 4.22 (q, J = |
| | en-1-yl)thio)thiazole-5-carbaldehyde | 7.0 Hz, 2H), 3.41 (t, J = 6.8 Hz, 2H), 2.75-2.86 (m, 2H), |
| | O-ethyl oxime | 2.43-2.48 (m, 1H), 1.28 (t, J = 7.1 Hz, 3H), 0.95-1.04 (m, |
| | | 4H); LCMS (M + 1) = 337.10; |
| 283 | 4-cyclopropyl-2-((3,4,4-trifluorobut-3- | ¹H-NMR (400 MHz, DMSO-d₆) δ 8.33 (s, 1H), 4.03 (s, |
| | en-1-yl)sulfinyl)thiazole-5- | 3H), 3.51-3.57 (m, 1H), 3.26 (q, J = 6.9 Hz, 1H), 2.74-2.84 |
| | carbaldehyde O-methyl oxime | (m, 1H), 2.53-2.66 (m, 2H), 1.01-1.09 (m, 2H), 0.90-1.00 |
| | | (m, 2H); LCMS (M + 1) = 339.10; |
| 284 | 4-cyclopropyl-2-((3,4,4-trifluorobut-3- | ¹H-NMR (400 MHz, DMSO-d₆) δ 8.81 (s, 1H), 8.46 (s, |
| | en-1-yl)sulfonyl)thiazole-5- | 1H), 4.08 (s, 3H), 3.92 (s, 3H), 3.85 (m, 4H), 2.82 (m, 4H), |
| | carbaldehyde O-methyl oxime | 2.62 (s, 1H), 1.22 (s, 1H), 1.04-1.11 (m, 4H), 0.96 (m, 4H); |
| | | LCMS (M + 1) = 355.10; |
| 285 | 5-cyclopropyl-3-(2-((3,4,4-trifluorobut- | ¹H-NMR (400 MHz, DMSO-d₆) δ 8.66 (d, J = 7.3 Hz, 1H), |
| | 3-en-1-yl)sulfinyl)thiazol-5-yl)-1,2,4- | 3.64-3.71 (m, 1H), 3.38-3.45 (m, 1H), 2.80-2.93 (m, 1H), |
| | oxadiazole | 2.62-2.75 (m, 1H), 2.42-2.49 (m, 1H), 1.29-1.36 (m, 2H), |
| | | 1.20-1.24 (m, 2H); LCMS (M + 1): 350.05; |
| 286 | 2-((4,4-difluorobut-3-en-1- | ¹H-NMR (400 MHz, DMSO-d₆) δ 8.45 (s, 1H), 8.13 (d, J = |
| | yl)thio)thiazole-5-carbaldehyde O- | 7.6 Hz, 1H), 7.98 (s, 1H), 7.94-7.93 (0H), 4.60 (dtd, J = |
| | methyl oxime | 26.7, 7.9, 2.6 Hz, 1H), 3.97 (s, 3H), 3.85 (s, 1H), 3.34 (t, |
| | | J = 6.7 Hz, 5H), 3.33 (s, 0H), 3.29-3.29 (1H), 2.40-2.46 (m, |
| | | 3H); LCMS (M + 1) (M + 1): 265.05; |
| 287 | 4-cyclopropyl-2-((3,4,4-trifluorobut-3- | ¹H-NMR (400 MHz, DMSO-d₆) δ 8.75 (s, 1H), 8.48 (s, |
| | en-1-yl)sulfinyl)thiazole-5- | 1H), 4.37 (q, J = 7.1 Hz, 2H), 4.16 (q, J = 7.0 Hz, 2H), |
| | carbaldehyde O-ethyl oxime | 3.85 (t, J = 7.1 Hz, 2H), 3.51-3.58 (m, 1H), 3.28 (t, J = 7.0 |
| | | Hz, 1H), 2.77-2.89 (m, 2H), 2.60-2.71 (m, 3H), 2.42-2.47 |
| | | (m, 1H), 1.33 (t, J = 7.1 Hz, 3H), 1.25 (t, J = 7.0 Hz, 3H), |
| | | 1.10-1.15 (m, 2H), 0.98-1.04 (m, 4H), 0.91-0.95 (m, 2H); |
| | | LCMS (M + 1) = 353.10; |
| 288 | 4-cyclopropyl-2-((3,4,4-trifluorobut-3- | ¹H-NMR (400 MHz, DMSO-d₆) δ 8.82 (s, 1H), 8.48 (s, |
| | en-1-yl)sulfonyl)thiazole-5- | 1H), 4.37 (q, J = 7.0 Hz, 2H), 4.19 (q, J = 7.0 Hz, 2H), |
| | carbaldehyde O-ethyl oxime | 3.84-3.89 (m, 3H), 2.78-2.89 (m, 3H), 2.61-2.65 (m, 1H), |
| | | 1.33 (t, J = 7.1 Hz, 3H), 1.26 (t, J = 7.1 Hz, 3H), 1.10-1.15 |
| | | (m, 2H), 1.05-1.08 (m, 2H), 0.98-1.02 (m, 2H), 0.96 (m, |
| | | 2H); LCMS (M + 1) = 369.05; |
| 289 | (Z)-2-((4,4-difluorobut-3-en-1- | ¹H-NMR (400 MHz, DMSO-d₆) δ 7.78 (s,1H), 7.73 (s,1H), |
| | yl)thio)oxazole-5-carbaldehyde O- | 4.58 (dtd, J = 26.7, 7.9, 2.6 Hz, 1H), 4.03-4.18 (m, 1H), |
| | methyl oxime | 4.00 (s, 3H), 3.29 (d, J = 7.1 Hz, 2H) 2.41-2.46 (m, 2H); |
| | | LCMS (M + 1): 249.10 |
| 290 | (E)-2-((4,4-difluorobut-3-en-1- | ¹H-NMR (400 MHz, DMSO-d₆) δ 8.19 (s, 1H), 7.51 (s, |
| | yl)thio)oxazole-5-carbaldehyde O- | 1H), 4.58 (dtd, J = 26.6, 7.9, 2.6 Hz, 1H), 3.88 (s, 3H), |
| | methyl oxime | 3.29 (t, J = 7.0 Hz, 2H), 2.44 (ddt, J = 14.8, 6.9, 1.7 Hz, |
| | | 2H); LCMS (M + 1): 249.10; |
| 291 | (Z)-2-((4,4-difluorobut-3-en-1- | ¹H-NMR (400 MHz, DMSO-d₆) δ 7.83 (s,1H), 7.72(s, 1H) |
| | yl)thio)oxazole-5-carbaldehyde O- | 4.42-4.64 (m, 1H), 4.01-4.28 (m, 2H), 3.27-3.32 (m, 3H), |
| | ethyl oxime | 2.44 (ddt, J = 14.9, 6.8, 1.7 Hz, 2H), 1.23-1.32 (m, 3H); |
| | | LCMS (M + 1): 263.15 |
| 292 | (E)-2-((4,4-difluorobut-3-en-1- | ¹H-NMR (400 MHz, DMSO-d₆) δ 8.21 (s, 1H), 7.47 (s, |
| | yl)thio)oxazole-5-carbaldehyde O- | 1H), 4.58 (dtd, J = 26.7, 7.9, 2.6 Hz, 1H), 4.10-4.33 (m, |
| | ethyl oxime | 2H), 3.29 (t, J = 6.8 Hz, 2H), 2.44 (ddt, J = 14.9, 6.9, 1.7 |
| | | Hz, 2H), 1.05-1.41 (m, 3H); LCMS (M + 1): 263.15; |
| 293 | (E)-4-methyl-2-((3,4,4-trifluorobut-3- | ¹H-NMR (400 MHz, DMSO-d₆) δ 8.43 (s, 1H), 3.95 (s, |
| | en-1-yl)sulfinyl)oxazole-5- | 3H), 3.48-3.75 (m, 2H), 2.68-2.96 (m, 2H), 2.32 (s, 3H); |
| | carbaldehyde O-methyl oxime | |
| 294 | (E)-4-methyl-2-((3,4,4-trifluorobut-3- | ¹H-NMR (400 MHz, DMSO-d₆) δ 8.47 (s, 1H), 3.96 (s, |
| | en-1-yl)sulfonyl)oxazole-5- | 3H), 3.89-4.03 (m, 2H), 2.83-2.94 (m, 2H), 2.35 (s, 3H); |
| | carbaldehyde O-methyl oxime | |
| 295 | (E)-2-((3,4,4-trifluorobut-3-en-1- | ¹H-NMR (400 MHz, DMSO-d₆) δ 8.61 (s, 1H), 8.31 (s, |
| | yl)sulfonyl)thiazole-5-carbaldehyde O- | 1H), 4.18 (d, J = 7.3 Hz, 2H), 3.92 (t, J = 7.0 Hz, 2H), |
| | cyclopropylmethyl oxime | 2.81-2.92 (m, 2H), 1.20-1.25 (m, 1H), 0.56-0.60 (m, 2H), |
| | | 0.34-0.38 (m, 2H); |
| 296 | (E)-2-((4,4-difluorobut-3-en-1- | ¹H-NMR (400 MHz, DMSO-d₆) δ 8.13 (s, 1H), 7.97 (s, |
| | yl)thio)thiazole-5-carbaldehyde O- | 1H), 4.60 (dtd, J = 26.7, 7.9, 2.5 Hz, 1H), 4.44 (td, J = |
| | isopropyl oxime | 12.5, 6.4 Hz, 1H), 3.32-3.35 (m, 2H), 2.40-2.46 (m, 1H), |
| | | 1.13-1.43 (m, 6H); LCMS (M + 1): 292.05; |
| 297 | (E)-2-((4,4-difluorobut-3-en-1- | ¹H-NMR (400 MHz, DMSO-d₆) δ 8.47 (t, J = 7.2 Hz, 1H), |
| | yl)sulfinyl)thiazole-5-carbaldehyde O- | 8.15 (d, J = 5.9 Hz, 1H), 4.50-4.63 (m, 2H), 3.37-3.44 (m, |
| | isopropyl oxime | 1H), 3.18-3.25 (m, 1H), 2.50-2.49 (1H), 2.44 (ddt, J = |
| | | 14.8, 8.2, 1.7 Hz, 1H), 2.20-2.30 (m, 1H), 1.33 (d, J = 6.4 |
| | | Hz, 6H), LCMS (M + 1): 308.40; |
| 298 | 5-cyclobutyl-3-(2-((3,4,4-trifluorobut- | ¹H-NMR (400 MHz, DMSO-d₆) δ 8.72 (s,1H), 3.92-4.00 |
| | 3-en-1-yl)sulfinyl)thiazol-5-yl)-1,2,4- | (m, 1H), 3.65-3.72 (m, 1H), 2.80-2.95 (m, 1H), 2.61-2.76 |
| | oxadiazole | (m, 1H), 2.42-2.48 (m, 4H), 2.07-2.18 (m, 1H), 1.94-2.04 |
| | | (m, 1H), LCMS: 364.10 M + 1 |
| 299 | (E)-2-((4,4-difluorobut-3-en-1- | ¹H-NMR (400 MHz, DMSO-d₆) δ 8.14 (d, J = 2.0 Hz, 1H), |
| | yl)thio)thiazole-5-carbaldehyde O- | 7.98 (d, J = 3.2 Hz, 1H), 4.60 (dtd, J = 26.6, 7.9, 2.6 Hz, |
| | cyclopropylmethyl oxime | 1H), 4.03 (d, J = 7.1 Hz, 2H), 3.35 (d, J = 6.8 Hz, 2H), |
| | | 2.40-2.46 (m, 2H), 1.12-1.22 (m, 1H), 0.49-0.57 (m, 2H), |
| | | 0.26-0.33 (m, 2H); LCMS (M + 1) = 305.1; |
| 300 | 2-((4,4-difluorobut-3-en-1- | ¹H-NMR (400 MHz, DMSO-d₆) δ 8.62 (s, 1H), 8.60 (s, |
| | yl)sulfonyl)thiazole-5-carbaldehyde O- | 1H), 8.38 (d, J = 0.5 Hz, 1H), 8.31 (s, 1H), 4.45-4.57 (m, |
| | ethyl oxime | 1H), 4.45-4.57 (m, 1H), 4.38 (q, J = 7.0 Hz, 2H), 4.19 (t, |
| | | J = 7.0 Hz, 2H), 3.74 (dd, J = 7.2, 4.0 Hz, 2H), 3.74 (dd, J = |
| | | 7.2, 4.0 Hz, 2H), 2.41-2.45 (m, 2H), 2.41-2.45 (m, 2H), |
| | | 1.35 (d, J = 7.1 Hz, 3H), 1.23-1.27 (m, 3H); LCMS (M + 1) = |
| | | 311.1; |
| 301 | (E)-2-((4,4-difluorobut-3-en-1- | ¹H-NMR (400 MHz, DMSO-d₆) δ 8.36 (s, 1H), 7.77 (s, |
| | yl)sulfinyl)oxazole-5-carbaldehyde O- | 1H), 4.54-4.67 (m, 1H), 3.95 (s, 3H), 3.38-3.52 (m, 2H), |
| | methyl oxime | 2.34-2.47 (m, 2H), LCMS (M + 1): 265.10 |
| 302 | (E)-2-((4,4-difluorobut-3-en-1- | ¹H-NMR (400 MHz, DMSO-d₆) δ 8.13 (d, J = 3.2 Hz, 1H), |
| | yl)thio)thiazole-5-carbaldehyde O- | 7.97 (s, 1H), 4.59 (dtd, J = 26.7, 7.9, 2.6 Hz, 1H), 4.18 (d, |
| | cyclobutylmethyl oxime | J = 6.8 Hz, 2H), 3.32-3.34 (m, 2H), 2.65-2.72 (m, 1H), 2.40- |
| | | 2.44 (m, 2H), 2.00-2.07 (m, 2H), 1.78-1.95 (m, 4H); |
| | | LCMS (M + 1): 318.9; |
| 303 | (Z)-2-((4,4-difluorobut-3-en-1- | ¹H-NMR (400 MHz, DMSO-d₆) δ 8.44-8.57 (s, 1H), 7.92 |
| | yl)thio)thiazole-5-carbaldehyde O- | (s, 1H), 4.60 (dtd, J = 26.7, 7.9, 2.6 Hz, 1H), 3.85-3.90 (m, |
| | cyclopropylmethyl oxime | 2H), 2.40-2.46 (m, 2H), 1.06-1.16 (m, 1H), 0.46-0.58 (m, |
| | | 2H), 0.25-0.33 (m, 2H); |
| 304 | (E)-2-((4,4-difluorobut-3-en-1- | ¹H-NMR (400 MHz, DMSO-d₆) δ 8.46 (d, J = 7.3 Hz, 1H), |
| | yl)sulfinyl)thiazole-5-carbaldehyde O- | 8.16 (s, 1H), 4.56 (dtd, J = 26.4, 7.7, 2.5 Hz, 1H), 4.28 (d, |
| | cyclobutylmethyl oxime | J = 6.8 Hz, 2H), 3.38-3.45 (m, 1H), 3.18-3.24 (m, 1H), 2.68- |
| | | 2.75 (m, 1H), 2.44 (dd, J = 14.7, 8.1 Hz, 1H), 2.20-2.34 |
| | | (m, 1H), 2.00-2.08 (m, 2H), 1.79-1.98 (m, 3H); |
| | | LCMS (M + 1): 335.02; |
| 305 | (Z)-2-((4,4-difluorobut-3-en-1- | ¹H-NMR (400 MHz, DMSO-d₆) δ 8.58 (s, 1H), 8.25 (d, J = |
| | yl)sulfinyl)thiazole-5-carbaldehyde O- | 0.5 Hz, 1H), 4.58 (dtd, J = 26.4, 7.8, 2.4 Hz, 1H), 3.96 (d, |
| | cyclopropylmethyl oxime | J = 7.3 Hz, 2H), 3.36-3.43 (m, 1H), 3.18-3.25 (m, 1H), 2.43- |
| | | 2.47 (m, 1H), 2.44 (dd, J = 14.7, 8.1 Hz, 1H), 2.22-2.28 |
| | | (m, 1H), 1.09-1.18 (m, 1H), 0.51-0.56 (m, 2H), 0.28-0.32 |
| | | (m, 2H); LCMS (M + 1): 321.05; |
| 306 | (Z)-2-((4,4-difluorobut-3-en-1- | ¹H-NMR (400 MHz, DMSO-d₆) δ 8.64 (d, J = 0.4 Hz, 1H), |
| | yl)sulfonyl)thiazole-5-carbaldehyde O- | 8.37 (d, J = 0.5 Hz, 1H), 4.52 (dtd, J = 26.0, 7.8, 2.3 Hz, |
| | cyclopropylmethyl oxime | 1H), 3.99 (d, J = 7.3 Hz, 2H), 3.74 (t, J = 7.3 Hz, 2H), |
| | | 2.39-2.46 (m, 2H), 1.10-1.24 (m, 1H), 0.50-0.57 (m, 2H), |
| | | 0.29-0.33 (m, 2H); LCMS: 336.95; |
| 307 | (E)-2-((4,4-difluorobut-3-en-1- | ¹H-NMR (400 MHz, DMSO-d₆) δ 8.60 (s, 1H), 8.31 (s, |
| | yl)sulfonyl)thiazole-5-carbaldehyde O- | 1H), 4.51 (dtd, J = 26.2, 7.8, 2.4 Hz, 1H), 4.18 (d, J = 7.3 |
| | cyclopropylmethyl oxime | Hz, 2H), 3.74 (t, J = 7.2 Hz, 2H), 2.39-2.46 (m, 2H), 1.21- |
| | | 1.27 (m, 1H), 0.54-0.60 (m, 2H), 0.34-0.40 (m, 2H); |
| | | LCMS (M + 1) = 337.1; |
| 308 | (E)-2-((4,4-difluorobut-3-en-1- | ¹H-NMR (400 MHz, DMSO-d₆) δ 8.58-8.60 (m, 1H), 8.30 |
| | yl)sulfonyl)thiazole-5-carbaldehyde O- | (s, 1H), 4.50 (dtd, J = 26.1, 7.8, 2.4 Hz, 1H), 4.33 (d, J = |
| | cyclobutylmethyl oxime | 6.8 Hz, 2H), 3.78 (dt, J = 34.3, 7.2 Hz, 2H), 2.68-2.79 (m, |
| | | 1H), 2.33-2.45 (m, 2H), 1.80-2.10 (m, 6H); LCMS (M + 1): |
| | | 351.05; |
| 309 | 5-ethyl-3-(2-((3,4,4-trifluorobut-3-en- | ¹H-NMR (400 MHz, DMSO-d₆) δ 8.34-8.44 (m, 1H), 3.54- |
| | 1-yl)thio)thiazol-5-yl)-1,2,4-oxadiazole | 3.60 (q, 2H), 2.99-3.17 (m, 2H), 2.83-2.94 (m, 2H), 1.27- |
| | | 1.35 (t, 3H); LCMS (M + 1): 322.05; |
| 310 | 5-ethyl-3-(2-((3,4,4-trifluorobut-3-en- | ¹H-NMR (400 MHz, DMSO-d₆) δ 8.34 (s, 1H), 3.60- |
| | 1-yl)sulfinyl)thiazol-5-yl)-1,2,4- | 3.68 (m, 1H), 3.36-3.46 (m, 1H), 3.12 (q, 2H) 2.78-2.92 (m, |
| | oxadiazole | 1H), 2.60-2.74 (m, 1H) 1.35 (t, 3H); LCMS (M + 1): 338.05; |
| 311 | 5-ethyl-3-(2-((3,4,4-trifluorobut-3-en- | ¹H-NMR (400 MHz, DMSO-d₆) δ 8.34 (s, 1H), 3.60- |
| | 1-yl)sulfonyl)thiazol-5-yl)-1,2,4- | 3.68 (m, 1H), 3.36-3.46 (m, 1H), 3.12 (q, 2H) 2.78-2.92 (m, |
| | oxadiazole | 1H), 2.60-2.74 (m, 1H) 1.35 (t, 3H); |
| 312 | 5-(tert-butyl)-3-(2-((3,4,4-trifluorobut- | ¹H-NMR (400 MHz, DMSO-d₆) δ 8.34-8.38 (m, 1H), 3.55 |
| | 3-en-1-yl)thio)thiazol-5-yl)-1,2,4- | (t, J = 6.8 Hz, 2H), 2.67-2.93 (m, 2H), 1.41-1.46 (m, 9H); |
| | oxadiazole | LCMS (M + 1) (M + 1): 350.00; |
| 313 | 5-(3,4-difluorophenyl)-3-(2-((3,4,4- | ¹H-NMR (400 MHz, DMSO-d₆) δ 8.47-8.56 (m, 1H), 8.05- |
| | trifluorobut-3-en-1-yl)thio)thiazol-5- | 8.34 (m, 1H), 7.74-7.81 (m, 1H), 3.58 (t, J = 6.8 Hz, 2H), |
| | yl)-1,2,4-oxadiazole | 2.85-2.96 (m, 2H); LCMS (M + 1): 405.90; |
| 314 | (E)-4-methyl-2-((3,4,4-trifluorobut-3- | ¹H-NMR (400 MHz, DMSO-d₆) δ 8.27 (s, 1H), 4.07 (d, J = |
| | en-1-yl)thio)oxazole-5-carbaldehyde O- | 6.8 Hz, 2H), 3.41 (t, J = 6.7 Hz, 2H), 2.87-2.92 (m, 1H), |
| | cyclobutylmethyl oxime | 2.83 (dt, J = 6.8, 2.8 Hz, 1H), 2.64 (t, J = 7.3 Hz, 1H), 2.19 |
| | | (s, 3H), 1.98-2.05 (m, 2H), 1.82-1.90 (m, 2H), 1.74-1.81 |
| | | (m, 2H); |
| 315 | 5-(tert-butyl)-3-(2-((3,4,4-trifluorobut- | ¹H-NMR (400 MHz, DMSO-d₆) δ 8.70-8.73 (s, 1H), 3.66- |
| | 3-en-1-yl)sulfinyl)thiazol-5-yl)-1,2,4- | 3.73 (m, 1H), 3.38-3.46 (m, 1H), 2.80-2.95 (m, 1H), 2.59- |
| | oxadiazole | 2.74 (m, 1H), 1.46 (s, 9H); LCMS (M + 1): 365.85; |
| 316 | 5-(tert-butyl)-3-(2-((3,4,4-trifluorobut- | ¹H-NMR (400 MHz, DMSO-d₆) δ 8.85 (s, 1H), 4.01 (t, J = |
| | 3-en-1-yl)sulfonyl)thiazol-5-yl)-1,2,4- | 7.1 Hz, 2H), 2.85-2.95 (m, 2H), 1.30-1.62 (s, 9H); |
| | oxadiazole | LCMS (M + 1): 382.00; |
| 317 | 5-(3,4-difluorophenyl)-3-(2-((3,4,4- | ¹H-NMR (400 MHz, DMSO-d₆) δ 8.80-8.82 (m, 1H), 8.26- |
| | trifluorobut-3-en-1-yl)sulfinyl)thiazol- | 8.31 (m, 1H), 8.09-8.13 (m, 1H), 7.76-7.83 (m, 1H), 3.68- |
| | 5-yl)-1,2,4-oxadiazole | 3.75 (m, 1H), 3.41-3.49 (m, 1H), 2.84-2.97 (m, 1H), 2.65- |
| | | 2.78 (m, 1H); |
| 318 | 5-(2-chloro-6-fluorophenyl)-3-(2- | ¹H-NMR (400 MHz, DMSO-d₆) δ 10.35 (s, 1H), 8.48 (s, |
| | ((3,4,4-trifluorobut-3-en-1- | 1H), 7.64 (dd, J = 8.6, 8.1 Hz, 1H), 7.55 (dd, J = 8.6, 1.0 |
| | yl)thio)thiazol-5-yl)isoxazole | Hz, 1H), 7.34 (dd, J = 7.8, 0.7 Hz, 1H), 3.52-3.57 (m, 2H), |
| | | 2.66-2.93 (m, 2H); LCMS (M + 1): 420.95; |
| 319 | 4-chloro-2-((3,4,4-trifluorobut-3-en-1- | ¹H-NMR (400 MHz, DMSO-d₆) δ 8.25 (s, 1H), 7.85 (s, |
| | yl)thio)thiazole-5-carbaldehyde O- | 1H), 4.00 (d, J = 7.6 Hz, 3H), 3.87 (s, 1H), 3.48 (t, J = 6.9 |
| | methyl oxime | Hz, 2H), 2.78-2.89 (m, 2H), 1.98 (s, 1H), 1.17 (d, J = 7.0 |
| | | Hz, 1H); LCMS (M + 1): 316.90;; |
| 320 | 5-(2-bromo-6-fluorophenyl)-3-(2- | ¹H-NMR (400 MHz, DMSO-d₆) δ 10.34 (s, 1H), 8.48 (d, |
| | ((3,4,4-trifluorobut-3-en-1- | J = 8.6 Hz, 1H), 7.48-7.58 (m, 3H), 3.51 (t, J = 6.7 Hz, 2H), |
| | yl)thio)thiazol-5-yl)isoxazole | 2.81-2.92 (m, 2H); LCMS (M + 1): 466.70; |
| 321 | (E)-4-(methoxymethyl)-2-((3,4,4- | ¹H-NMR (400 MHz, DMSO-d₆) δ 8.09 (t, J = 5.6 Hz, 1H), |
| | trifluorobut-3-en-1-yl)thio)thiazole-5- | 4.60-4.64 (m, 2H), 3.96 (d, J = 14.9 Hz, 3H), 3.48 (t, J = |
| | carbaldehyde O-methyl oxime | 6.8 Hz, 2H), 3.29 (s, 3H), 2.79-2.90 (m, 2H); |
| | | LCMS (M + 1): 326.7; |
| 322 | (E)-4-(methoxymethyl)-2-((3,4,4- | ¹H-NMR (400 MHz, DMSO-d₆) δ 8.25 (dd, J = 6.5, 2.1 |
| | trifluorobut-3-en-1-yl)sulfinyl)thiazole- | Hz, 1H), 4.74 (d, J = 1.2 Hz, 2H), 4.06 (d, J = 12.2 Hz, |
| | 5-carbaldehyde O-methyl oxime | 3H), 3.58-3.65 (m, 1H), 3.36-3.39 (m, 1H), 3.30 (d, J = 4.2 |
| | | Hz, 3H), 2.78-2.90 (m, 1H), 2.57-2.72 (m, 1H); |
| | | LCMS (M + 1): 342.90; |
| 323 | (E)-4-(methoxymethyl)-2-((3,4,4- | ¹H-NMR (400 MHz, DMSO-d₆) δ 8.37 (d, J = 7.8 Hz, 1H), |
| | trifluorobut-3-en-1- | 4.79 (s, 2H), 4.13 (s, 3H), 3.90-3.95 (m, 2H), 3.33 (d, J = |
| | yl)sulfonyl)thiazole-5-carbaldehyde O- | 0.7 Hz, 3H), 2.81-2.91 (m, 2H); LCMS (M + 1) (M + 1): |
| | methyl oxime | 359.0; |
| 324 | 4-(methylamino)-2-((3,4,4-trifluorobut- | ¹H-NMR (400 MHz, CDCl₃) δ 7.28 (s, 1H),4.32 (d, J = 3.4 |
| | 3-en-1-yl)thio)thiazole-5-carbaldehyde | Hz, 1H), 3.93 (d, J = 11.9 Hz, 3H), 3.34-3.37 (m, 2H), 3.08 |
| | O-methyl oxime | (d, J = 4.9 Hz, 3H), 2.75-2.86 (m, 2H); LCMS (M + 1): |
| | | 311.75; |
| 325 | 5-(difluoromethyl)-2-((3,4,4- | ¹H-NMR (400 MHz, CDCl₃) δ 7.77 (t, J = 2.4 Hz, 1H), |
| | trifluorobut-3-en-1-yl)thio)thiazole | 6.83 (t, J = 55.1 Hz, 1H), 3.44 (t, J = 7.0 Hz, 2H), 2.76- |
| | | 2.86 (m, 2H); LCMS (M + 1): 275.95; |
| 326 | 5-(difluoromethyl)-2-((3,4,4- | ¹H-NMR (400 MHz, CDCl₃) δ 8.11 (t, J = 1.9 Hz, 1H), |
| | trifluorobut-3-en-1-yl)sulfinyl)thiazole | 6.94 (t, J = 55.0 Hz, 1H), 3.42-3.49 (m, 1H), 3.29-3.36 (m, |
| | | 1H), 2.88-2.99 (m, 1H), 2.58-2.68 (m, 1H); LCMS (M − 1): |
| | | 289.15; |
| 327 | 2-((3,4,4-trifluorobut-3-en-1- | ¹H-NMR (400 MHz, CDCl₃) δ 8.07 (s, 1H), 3.49 (t, J = |
| | yl)thio)thiazole-5-carbonitrile | 7.0 Hz, 2H), 2.77-2.88 (m, 2H); GCMS (M+): 250.0; |
| 328 | 2-((3,4,4-trifluorobut-3-en-1- | ¹H-NMR (400 MHz, CDCl₃) δ 8.38 (s, 1H), 3.46-3.53 (m, |
| | yl)sulfinyl)thiazole-5-carbonitrile | 1H), 3.31-3.39 (m, 1H), 2.88-3.01 (m, 1H), 2.58-2.70 (m, |
| | | 1H); LCMS (M + 1): 267.25; |
| 329 | 5-(difluoromethyl)-2-((3,4,4- | ¹H-NMR (400 MHz, CDCl₃) δ 8.19 (t, J = 1.6 Hz, 1H), |
| | trifluorobut-3-en-1-yl)sulfonyl)thiazole | 6.98 (t, J = 54.8 Hz, 1H), 3.65-3.69 (m, 2H), 2.88-2.98 (m, |
| | | 2H); LCMS (M − 1): 306.20; |
| 330 | 5-(difluoromethyl)-2-((3,4,4- | ¹H-NMR (400 MHz, CDCl₃) δ 7.66 (s, 1H), 7.20 (t, J = |
| | trifluorobut-3-en-1-yl)thio)oxazole | 52.4 Hz, 1H) 4.02 (t, J = 6.8 Hz, 2H), 2.79-2.60 (m, 2H); |
| | | LCMS (M + 1): 260.05; |
| 331 | 2-((3,4,4-trifluorobut-3-en-1- | ¹H-NMR (400 MHz, CDCl₃) δ 7.67 (s, 1H), 3.41 (t, J = |
| | yl)thio)oxazole-5-carbonitrile | 7.2 Hz, 2H), 2.79-2.89 (m, 2H); |
| 332 | 2-((3,4,4-trifluorobut-3-en-1- | ¹H-NMR (400 MHz, CDCl₃) δ 8.45 (s, 1H), 3.68-3.72 (m, |
| | yl)sulfonyl)thiazole-5-carbonitrile | 2H), 2.89-3.00 (m, 2H); LCMS (M − 1): 280.80; |
| 333 | 2-((3,4,4-trifluorobut-3-en-1- | ¹H-NMR (400 MHz, CDCl₃) δ 7.89 (s, 1H), 3.50-3.58 (m, |
| | yl)sulfinyl)oxazole-5-carbonitrile | 2H), 2.94-3.05 (s, 1H), 2.76-2.88 (s, 1H); |
| 334 | 5-(difluoromethyl)-2-((3,4,4- | ¹H-NMR (400 MHz, CDCl₃) δ 7.55 (s, 1H), 6.77 (t = 53.6 |
| | trifluorobut-3-en-1-yl)sulfinyl)oxazole | Hz, 1H) 3.58-3.45 (m, 2H), 3.00-2.92 (s, 1H), 2.88-2.79 (s, |
| | | 1H); |
| 335 | 5-(difluoromethyl)-4-methyl-2-((3,4,4- | ¹H-NMR (400 MHz, CDCl₃) δ 6.81 (t, J = 55 Hz, 1H), |
| | trifluorobut-3-en-1-yl)thio)thiazole | 3.40 (m, 2H), 2.74-2.84 (m, 2H), 2.42 (s, 3H); LCMS |
| | | (M + 1): 289.95; |
| 336 | 4-methyl-2-((3,4,4-trifluorobut-3-en-1- | ¹H-NMR (400 MHz, CDCl₃) δ 3.45 (t, J = 6.8 Hz, 2H), |
| | yl)thio)thiazole-5-carbonitrile | 2.75-2.86 (m, 2H), 2.55 (s, 3H); LCMS (M + 1): 264.90; |
| 337 | 5-(difluoromethyl)-4-methyl-2-((3,4,4- | ¹H-NMR (400 MHz, CDCl₃) δ 6.90 ( t, J = 55 Hz, 1H), |
| | trifluorobut-3-en-1-yl)sulfinyl)thiazole | 3.38-3.45 (m, 1H), 3.26-3.33 (m, 1H), 2.87-2.96 (m, 2H), |
| | | 2.54 (s, 3H); LCMS (M + 1): 305.90; |
| 338 | 4-methyl-2-((3,4,4-trifluorobut-3-en-1- | ¹H-NMR (400 MHz, CDCl₃) δ 3.43-3.50 (m, 1H), 3.30- |
| | yl)sulfinyl)thiazole-5-carbonitrile | 3.35 (m, 1H), 2.69 (s, 3H); |
| 339 | 4-methyl-2-((3,4,4-trifluorobut-3-en-1- | ¹H-NMR (400 MHz, CDCl₃) δ 3.64-3.68 (m, 2H), 2.88- |
| | yl)sulfonyl)thiazole-5-carbonitrile | 2.98 (m, 2H), 2.72 (s, 3H); |
| 340 | 5-(difluoromethyl)-4-methyl-2((3,4,4- | ¹H-NMR (400 MHz, CDCl₃) δ 6.93 (t, J = 55 Hz, 1H), |
| | trifluorobut-3-en-1-yl)sulfonyl)thiazole | 3.61-3.65 (m, 2H), 2.86-2.97 (m, 2H), 2.59 (s, 3H); |
| 341 | 5-(difluoromethyl)-4-methyl-2-((3,4,4- | ¹H-NMR (400 MHz, CDCl₃) δ 6.66 (t, J = 53.3 Hz, 1H), |
| | trifluorobut-3-en-1-yl)thio)oxazole | 3.37 (t, J = 7.0 Hz, 2H), 2.78-2.89 (m, 2H), 2.27 (t, J = 2.7 |
| | | Hz, 3H); LCMS (M + 1): 273.90; |
| 342 | 4-(tert-butyl)-5-(difluoromethyl)-2- | ¹H-NMR (400 MHz, CDCl₃) δ 6.84 (t, J = 52.6 Hz, 1H), |
| | ((3,4,4-trifluorobut-3-en-1- | 3.35 (t, J = 7.2 Hz, 2H), 2.81-2.92 (m, 2H), 1.32-1.34 (m, |
| | yl)thio)oxazole | 9H); LCMS (M + 1): 316.00; |
| 343 | 4-(tert-butyl)-5-(difluoromethyl)-2- | ¹H-NMR (400 MHz, DMSO-d₆) δ 7.06 (t, J = 51.2 Hz, |
| | ((3,4,4-trifluorobut-3-en-1- | 1H), 2.82 (t, J = 7.0 Hz, 2H), 2.87-2.60 (m, 2H), 1.25 (s, |
| | yl)sulfonyl)oxazole | 9H); LCMS (M − 1): 345.65; |
| 344 | 4-methyl-2-((3,4,4-trifluorobut-3-en-1- | ¹H-NMR (400 MHz, CDCl₃) δ 3.41 (t, J = 7.0 Hz, 2H), |
| | yl)thio)oxazole-5-carbonitrile | 2.80-2.90 (m, 2H), 2.35 (s, 3H); LCMS (M + 1): 249.05; |
| 345 | 4-(tert-butyl)-2-((3,4,4-trifluorobut-3- | ¹H-NMR (400 MHz, CDCl₃) δ 3.40 (t, J = 7.1 Hz, 2H), |
| | en-1-yl)thio)oxazole-5-carbonitrile | 2.81-2.91 (m, 2H), 1.34 (s, 9H); LCMS (M + 1): 291.05; |
| 346 | 4-methoxy-2-((3,4,4-trifluorobut-3-en- | ¹H-NMR (400 MHz, CDCl₃) δ 4.11 (s, 3H), 3.42 (t, J = |
| | 1-yl)thio)thiazole-5-carbonitrile | 6.8 Hz, 2H), 2.75-2.87 (m, 2H); LCMS (M +1): 280.55; |
| 347 | 4-methoxy-2-((3,4,4-trifluorobut-3-en- | ¹H-NMR (400 MHz, CDCl₃) δ 4.15 (s, 3H), 3.45-3.49 (m, |
| | 1-yl)sulfinyl)thiazole-5-carbonitrile | 1H), 3.27-3.33 (m, 1H), 2.88-2.99 (m, 1H), 2.61-2.72 (m, |
| | | 1H); LCMS: 297.0; |
| 348 | 4-methyl-2-((3,4,4-trifluorobut-3-en-1- | ¹H-NMR (400 MHz, CDCl₃) δ 3.54 (m, 2H), 2.94-3.09 |
| | yl)sulfinyl)oxazole-5-carbonitrile | (m, 1H), 2.77-2.91 (m, 1H), 2.46 (s, 3H); LCMS (M + 1): |
| | | 265.20; |
| 349 | 4-(tert-butyl)-2-((3,4,4-trifluorobut-3- | ¹H-NMR (400 MHz, CDCl₃) δ 3.51-3.58 (m, 2H), 2.85- |
| | en-1-yl)sulfonyl)oxazole-5-carbonitrile | 3.01 (m, 2H), 1.41 (s, 9H); LCMS (M + 1): 323.10 |
| 350 | 5-(difluoromethyl)-4-methyl-2-((3,4,4- | ¹H-NMR (400 MHz, CDCl₃) δ 6.78 (t, J = 53.2 Hz, 1H), |
| | trifluorobut-3-en-1-yl)sulfonyl)oxazole | 3.45-3.57 (m, 2H), 2.78-3.04 (m, 2H), 2.39 (s, 3H); LCMS |
| | | (M + 1): 306.55; |
| 351 | 4-cyclopropyl-5-(difluoromethyl)-2- | ¹H-NMR (400 MHz, CDCl₃) δ 6.99 (t, J = 55.1 Hz, 1H), |
| | ((3,4,4-trifluorobut-3-en-1- | 3.35 (t, J = 7.3 Hz, 2H), 2.66-2.84 (m, 2H), 1.90-1.97 (m, |
| | yl)thio)thiazole | 1H), 1.08-1.11 (m, 4H); LCMS (M + 1): 316.15; |
| 352 | 4-cyclopropyl-5-(difluoromethyl)-2- | ¹H-NMR (400 MHz, DMSO-d₆) δ 7.55-7.82 (t, J = 55.1 |
| | ((3,4,4-trifluorobut-3-en-1- | Hz, 1H), 3.56-3.63 (m, 1H), 3.29-3.32 (m, 1H), 2.63-2.86 |
| | yl)sulfinyl)thiazole | (m, 1H), 2.22-2.36 (m, 1H), 1.90-1.97 (m, 1H), 0.92-1.13 |
| | | (m, 4H); LCMS (M + 1): 332.15; |
| 353 | 4-cyclopropyl-5-(difluoromethyl)-2- | ¹H-NMR (400 MHz, DMSO-d₆) δ 7.74 (t, J = 53.6, 0.8 Hz, |
| | ((3,4,4-trifluorobut-3-en-1- | 1H), 3.93 (t, J = 7.0 Hz, 2H), 2.81-2.92 (m, 2H), 2.34-2.38 |
| | yl)sulfonyl)thiazole | (m, 1H), 1.11-1.15 (m, 2H), 0.97-1.01 (m, 2H); LCMS (M − |
| | | 1): 345.60; |
| 354 | 4-cyclopropyl-2-((3,4,4-trifluorobut-3- | ¹H-NMR (400 MHz, DMSO-d₆) δ 3.47 (t, J = 7.0 Hz, 2H), |
| | en-1-yl)thio)thiazole-5-carbonitrile | 2.78-2.87 (m, 2H), 2.16-2.23 (m, 1H), 1.14-1.19 (m, 2H), |
| | | 1.02-1.05 (m, 2H); LCMS (M + 1): 290.85; |
| 355 | 4-cyclopropyl-2-((3,4,4-trifluorobut-3- | ¹H-NMR (400 MHz, DMSO-d₆) δ 3.60-3.67 (m, 1H), 3.34- |
| | en-1-yl)sulfinyl)thiazole-5-carbonitrile | 3.38 (m, 1H), 2.77-2.88 (m, 1H), 2.61-2.74 (m, 1H), 2.27- |
| | | 2.34 (m, 1H), 1.19-1.27 (m, 2H), 1.01-1.10 (m, 2H); |
| | | LCMS (M + 1): 306.90; |
| 356 | 4-cyclopropyl-2-((3,4,4-trifluorobut-3- | ¹H-NMR (400 MHz, DMSO-d₆) δ 3.96 (t, J = 7.0 Hz, 2H), |
| | en-1-yl)sulfonyl)thiazole-5-carbonitrile | 2.82-2.93 (m, 2H), 2.32-2.38 (m, 1H), 1.24-1.29 (m, 2H), |
| | | 1.05-1.09 (m, 2H); LCMS (M − 1): 320.90; |
| 357 | 4-chloro-2-((3,4,4-trifluorobut-3-en-1- | ¹H-NMR (400 MHz, DMSO-d₆) δ 3.54 (t, J = 6.8 Hz, 2H), |
| | yl)thio)thiazole-5-carbonitrile | 2.81-2.92 (m, 2H); LCMS (M + 1): 285.69; |
| 358 | 4-cyclopropyl-2-((3,4,4-trifluorobut-3- | ¹H-NMR (400 MHz, DMSO-d₆) δ 3.54 (t, J = 6.8 Hz, 2H), |
| | en-1-yl)thio)thiazole | 2.81-2.92 (m, 2H); LCMS (M + 1): 285.69; |
| 359 | 4-cyclopropyl-2-((3,4,4-trifluorobut-3- | ¹H-NMR (400 MHz, DMSO-d₆) δ 7.72 (s, 1H), 3.48-3.55 |
| | en-1-yl)sulfinyl)thiazole | (m, 1H), 3.27 (q, J = 6.9 Hz, 1H), 2.73-2.86 (m, 1H), 2.57- |
| | | 2.67 (m, 1H), 2.13-2.22 (m, 1H), 0.94-0.98 (m, 2H), 0.79- |
| | | 0.85 (m, 2H); LCMS (M + 1): 281.8; |
| 360 | 4-cyclopropyl-2-((3,4,4-trifluorobut-3- | ¹H-NMR (400 MHz, DMSO-d₆) δ 7.92 (s, 1H), 3.83 (t, J = |
| | en-1-yl)sulfonyl)thiazole | 7.0 Hz, 2H), 2.78-2.88 (m, 2H), 2.19-2.24 (m, 1H), 0.97- |
| | | 1.03 (m, 2H), 0.83-0.87 (m, 2H); LCMS (M + 1): 297.8; |
| 361 | 4-methyl-2-((3,4,4-trifluorobut-3-en-1- | ¹H-NMR (400 MHz, DMSO-d₆) δ 3.68-3.73 (m, 1H), 3.53 |
| | yl)sulfonyl)oxazole-5-carbonitrile | (dd, J = 13.9, 7.6 Hz, 1H), 2.75-2.97 (m, 2H), 2.39 (s, 3H); |
| | | LCMS (M + 1): 281.1; |
| 362 | (E)-1-methyl-2-((3,4,4-trifluorobut-3- | ¹H-NMR (400 MHz, DMSO-d₆) δ 7.78 (s, 1H), 7.59 (s, |
| | en-1-yl)thio)-¹H-imidazole-5- | 1H), 4.21 (q, J = 7.0 Hz, 2H), 3.62 (s, 3H), 3.32 (t, J = 3.3 |
| | carbaldehyde O-ethyl oxime | Hz, 2H), 2.73-2.81 (m, 2H), 1.24-1.30 (m, 3H), |
| | | LCMS (M + 1): M + 1: 294.00; |
| 363 | (Z)-1-methyl-2-((3,4,4-trifluorobut-3- | ¹H-NMR (400 MHz, DMSO-d₆) δ 8.17 (s, 1H), 7.30 (s, |
| | en-1-yl)thio)-¹H-imidazole-5- | 1H), 4.11 (q, J = 7.0 Hz, 2H), 3.70-3.94 (s, 3H), 3.30-3.39 |
| | carbaldehyde O-ethyl oxime | (s, 3H), 2.72-2.82 (m, 2H), 1.22-1.41 (m, 3H); LCMS: |
| | | (M + 1): 294.00; |
| 364 | (E)-1-methyl-2-((3,4,4-trifluorobut-3- | ¹H-NMR (400 MHz, DMSO-d₆) δ 7.76 (s, 1H), 7.51 (s, |
| | en-1-yl)thio)-¹H-imidazole-5- | 1H), 3.99 (s, 3H), 3.62 (s, 3H), 3.30-3.35 (m, 2H), 2.71- |
| | carbaldehyde O-methyl oxime | 2.82 (m, 2H); LCMS (M + 1): 280.20; |
| 365 | (Z)-1-methyl-2-((3,4,4-trifluorobut-3- | ¹H-NMR (400 MHz, DMSO-d₆) 8.18 (s, 1H), 7.25-7.33 (s, |
| | en-1-yl)thio)-¹H-imidazole-5- | 1H), 3.78-4.05 (s, 3H), 3.68-3.75 (s, 3H), 3.31-3.39 (m, |
| | carbaldehyde O-methyl oxime | 2H), 2.68-2.83 (m, 2H); LCMS (M + 1): 279.85; |
| 366 | (Z)-1-methyl-2-((3,4,4-trifluorobut-3- | ¹H-NMR (400 MHz, DMSO-d₆) δ 8.34 (s, 1H), 7.50 (s, |
| | en-1-yl)sulfinyl)-¹H-imidazole-5- | 1H), 3.99 (s, 3H), 3.91 (s, 3H), 3.64 (q, J = 6.7 Hz, 1H), |
| | carbaldehyde O-methyl oxime | 3.49-3.56 (m, 1H), 2.80-2.88 (m, 2H); LCMS (M + 1): |
| | | 296.00; |
| 367 | 4-chloro-2-((3,4,4-trifluorobut-3-en-1- | ¹H-NMR (400 MHz, DMSO-d₆) δ 9.87 (s, 1H), 3.57 (d, J = |
| | yl)thio)thiazole-5-carbaldehyde | 6.8 Hz, 2H), 2.83-2.94 (m, 2H); LCMS (M + 1): 287.7; |
| 368 | (4-chloro-2-((3,4,4-trifluorobut-3-en-1- | ¹H-NMR (400 MHz, DMSO-d₆) δ 5.80 (t, J = 5.6 Hz, 1H), |
| | yl)thio)thiazol-5-yl)methanol | 4.58 (d, J = 5.6 Hz, 2H), 3.43 (t, J = 6.8 Hz, 2H), 2.77-2.85 |
| | | (m, 2H); LCMS (M + 1): 290.05; |

* Compound names generated using Chemdraw Professional 17.1

As described herein the compounds of formula (I) shows an extremely high nematicidal activity which is exerted with respect to nematodes which attack on important agricultural crops. The compounds of the present invention were also showing extremely high fungicidal activity which is exerted with respect to numerous phytopathogenic fungi which attack on important agricultural crops. The compounds of the present invention were assessed for their activity against one or more of the following nematodes and fungal diseases.

### BIOLOGICAL EXAMPLES

### Biological Test Examples for Plant Parasitic Nematodes

### Example 1: Meloidogyne incognita (Root-Knot Nematode): In Vitro Test

Meloidogyne Incognita:

The test compounds at a concentration of 300 ppm were introduced into 500 μL of distilled water containing 50 Meloidogyne incognita juveniles into 24-well plates. The suspension was lightly shaken for uniform mixing of compounds. The test plates were covered with lids, and were kept for incubation at 25° C. temperature and 90% relative humidity. Dead/inactive nematodes were counted at an interval of 48, 72 and 96 h under a microscope & the percent mortality was calculated. Compounds

| | | | | | | | | | | | |
|---|---|---|---|---|---|---|---|---|---|---|---|
| | | | 1 | 2 | 5 | 6 | 8 | 10 | 11 | 12 | 13 |
| 14 | 15 | 16 | 17 | 22 | 23 | 34 | 36 | 40 | 51 | 53 | 54 |
| 8 | 60 | 65 | 66 | 67 | 68 | 69 | 70 | 71 | 72 | 74 | 75 |
| 76 | 77 | 79 | 81 | 82 | 87 | 102 | 103 | 118 | 122 | 128 | 130 |
| 132 | 134 | 176 | 182 | 183 | 211 | 213 | 216 | 222 | 233 | 234 | 235 |
| 236 | 237 | 238 | 266 | 273 | 286 | 289 | 290 | 292 | 326 | 327 | 328 |
| 329 | 330 | 331 | 332 | 333 | 337 | 338 | 339 | 340 | 348 | 350 | at 300 ppm |

showed more than 90% mortality in the tests, where there was no mortality in untreated check.
Meloidogyne incognita: In Vivo Test

Cucumber plants were grown in seedling tray containing mixture of Sand:Soil:FYM:Cocopeat in ratio of 1:1:1:1. One mL of test compound at tested concentration was applied into the soil mixture with the help of micropipette when the cucumber seedlings ten days old. Approximately 2000 freshly hatched second-stage juveniles of Meloidogyne incognita were inoculated. The treated plants were allowed to grow at 27° C. temperature in greenhouse. Observation of gall rating was recorded after 15 days of application. Plants were carefully uprooted and roots were washed thoroughly. The gall rating was observed on 0-10 scale as described by Zeck (1971) as mentioned below:
0=no galls
1=very few small galls
2=numerous small galls
3=numerous small galls of which some are grown together
4=numerous small and some big galls
5=25% of roots severely galled
6=50% of roots severely galled
7=75% of roots severely galled
8=no healthy roots but plant is still green
9=roots rotting and plant dying
10=plant and roots dead

Compounds

| | | | | | | | | | | | |
|---|---|---|---|---|---|---|---|---|---|---|---|
| | 1 | 2 | 5 | 6 | 7 | 8 | 10 | 11 | 12 | 13 | 14 |
| 15 | 16 | 17 | 22 | 23 | 34 | 36 | 40 | 51 | 53 | 54 | 58 |
| 60 | 65 | 66 | 67 | 68 | 69 | 70 | 71 | 72 | 74 | 75 | 76 |
| 77 | 79 | 81 | 82 | 87 | 102 | 103 | 118 | 122 | 128 | 130 | 132 |
| 134 | 176 | 181 | 182 | 183 | 187 | 211 | 213 | 216 | 219 | 222 | 233 |
| 234 | 235 | 236 | 237 | 238 | 266 | 273 | 286 | 289 | 290 | 292 | 326 |
| 327 | 328 | 329 | 330 | 331 | 332 | 333 | 337 | 338 | 339 | 340 | 348 |
| 350 | at 300 ppm | | | | | | | | | | |

recorded less than 3 gall rating in the tests, where there was extensive galling (up to 8) in untreated check.

### Biological Test Examples for Fungal Pathogens

### Example 2: Pyricularia oryzae (Rice Blast)

Compounds were dissolved in 0.3% dimethyl sulfoxide & then added to Potato Dextrose Agar medium just prior to dispensing it into Petri dishes. 5 mL medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 25° C. temperature and 60% relative humidity for seven days and radial growth was measured. Compounds

| | | | | | | | | | | | |
|---|---|---|---|---|---|---|---|---|---|---|---|
| | | | | | 1 | 2 | 3 | 4 | 5 | 6 | 7 |
| 8 | 10 | 11 | 12 | 14 | 15 | 16 | 17 | 18 | 21 | 22 | 23 |
| 26 | 27 | 28 | 31 | 36 | 38 | 39 | 40 | 41 | 42 | 48 | 49 |
| 51 | 52 | 53 | 54 | 55 | 56 | 57 | 58 | 59 | 60 | 61 | 62 |
| 63 | 64 | 65 | 66 | 67 | 68 | 69 | 70 | 71 | 72 | 73 | 74 |
| 75 | 76 | 77 | 78 | 79 | 80 | 81 | 85 | 86 | 87 | 89 | 90 |
| 91 | 94 | 96 | 98 | 102 | 103 | 114 | 115 | 118 | 119 | 120 | 121 |
| 122 | 123 | 124 | 127 | 128 | 130 | 131 | 132 | 134 | 135 | 141 | 142 |
| 143 | 147 | 148 | 149 | 150 | 152 | 153 | 156 | 158 | 159 | 160 | 161 |
| 163 | 165 | 169 | 170 | 171 | 174 | 176 | 178 | 181 | 183 | 184 | 185 |
| 186 | 187 | 188 | 189 | 190 | 193 | 194 | 196 | 197 | 198 | 199 | 200 |
| 206 | 211 | 213 | 215 | 219 | 226 | 229 | 230 | 231 | 232 | 237 | 246 |
| 251 | 252 | 254 | 255 | 256 | 257 | 258 | 259 | 260 | 261 | 262 | 263 |
| 264 | 265 | 266 | 273 | 274 | 276 | 286 | 287 | 288 | 289 | 290 | 291 |
| 292 | 293 | 294 | 295 | 296 | 297 | 298 | 300 | 301 | 304 | 305 | 306 |
| 307 | 310 | 311 | 316 | 319 | 320 | 326 | 327 | 328 | 329 | 330 | 331 |
| 332 | 333 | 334 | 335 | 336 | 337 | 338 | 339 | 340 | 344 | 348 | 349 |
| 354 | 355 | 356 | 358 | 359 | 360 | 361 | at 300 ppm | | | | |

gave more than 70% control in these tests when compared to the untreated check which showed extensive disease development.

### Example 3: Rhizoctonia solani (Rice Sheath Blight/Potato Black Scurf)

Compounds were dissolved in 0.3% dimethyl sulfoxide & then added to Potato Dextrose Agar medium just prior to dispensing it into Petri dishes. 5 mL medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 25° C. temperature and 60% relative humidity for seven days and radial growth was measured. Compounds

| | | | | | | | | | | | |
|---|---|---|---|---|---|---|---|---|---|---|---|
| | | | | | 1 | 3 | 4 | 5 | 6 | 7 | 8 |
| 11 | 16 | 17 | 18 | 22 | 23 | 31 | 36 | 40 | 48 | 49 | 50 |
| 51 | 52 | 53 | 62 | 71 | 73 | 77 | 78 | 79 | 80 | 81 | 82 |
| 83 | 87 | 90 | 103 | 123 | 124 | 125 | 127 | 128 | 130 | 131 | 134 |
| 148 | 153 | 156 | 158 | 160 | 161 | 163 | 165 | 167 | 174 | 178 | 181 |
| 184 | 185 | 211 | 213 | 219 | 230 | 232 | 237 | 249 | 266 | 267 | 276 |
| 286 | 289 | 290 | 291 | 292 | 293 | 294 | 298 | 300 | 301 | 305 | 307 |
| 308 | 310 | 311 | 326 | 327 | 328 | 329 | 331 | 332 | 333 | 334 | 335 |
| 336 | 338 | 339 | 340 | 348 | 349 | 355 | 356 | 358 | 359 | 360 | 361 |
| at 300 ppm | | | | | | | | | | | |

gave more than 70% control in these tests when compared to the untreated check which showed extensive disease development.

### Example 4: Botrytis cinerea (Gray Mold)

Compounds were dissolved in 0.3% dimethyl sulfoxide & then added to Potato Dextrose Agar medium just prior to dispensing it into Petri dishes. 5 mL medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 22° C. temperature and 90% relative humidity for seven days and radial growth was measured. Compounds

| | | | | | | | | | | | |
|---|---|---|---|---|---|---|---|---|---|---|---|
| | | | | | 1 | 3 | 4 | 5 | 6 | 7 | 8 |
| 11 | 17 | 18 | 23 | 26 | 27 | 31 | 38 | 39 | 40 | 41 | 42 |
| 48 | 49 | 50 | 51 | 52 | 62 | 63 | 68 | 71 | 72 | 73 | 76 |
| 77 | 78 | 79 | 80 | 83 | 85 | 86 | 87 | 91 | 101 | 103 | 124 |
| 125 | 127 | 128 | 130 | 131 | 132 | 134 | 135 | 141 | 153 | 158 | 160 |
| 178 | 198 | 199 | 211 | 213 | 219 | 226 | 230 | 232 | 263 | 264 | 266 |
| 267 | 276 | 279 | 280 | 285 | 286 | 289 | 290 | 291 | 292 | 293 | 294 |
| 300 | 301 | 305 | 310 | 311 | 317 | 327 | 328 | 329 | 332 | 333 | 334 |
| 336 | 337 | 338 | 339 | 340 | 348 | 349 | 351 | 352 | 355 | 358 | 360 |
| at 300 ppm | | | | | | | | | | | |

gave more than 70% in these tests when compared to the untreated check which showed extensive disease development.

### Example 5: Alternaria solani (Early Blight of Tomato/Potato)

Compounds were dissolved in 0.3% dimethyl sulfoxide & then added to Potato Dextrose Agar medium just prior to dispensing it into Petri dishes. 5 mL medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 25° C. temperature and 60% relative humidity for seven days and radial growth was measured. Compounds

| | | | | | | | | | | | |
|---|---|---|---|---|---|---|---|---|---|---|---|
| | | | | | 4 | 5 | 7 | 8 | 11 | 18 | 23 |
| 31 | 36 | 39 | 40 | 48 | 49 | 50 | 51 | 53 | 57 | 60 | 62 |
| 63 | 68 | 69 | 70 | 71 | 72 | 73 | 77 | 78 | 79 | 80 | 81 |
| 83 | 84 | 85 | 87 | 90 | 101 | 103 | 116 | 124 | 125 | 130 | 131 |
| 134 | 154 | 156 | 163 | 165 | 171 | 174 | 178 | 185 | 196 | 211 | 213 |
| 215 | 219 | 230 | 231 | 232 | 237 | 246 | 247 | 249 | 252 | 260 | 261 |
| 262 | 263 | 264 | 265 | 266 | 267 | 268 | 271 | 277 | 278 | 281 | 285 |
| 287 | 288 | 289 | 290 | 292 | 293 | 294 | 296 | 298 | 300 | 301 | 310 |
| 311 | 313 | 328 | 332 | 333 | 334 | 336 | 338 | 339 | 340 | 348 | 349 |
| 350 | 351 | 352 | 353 | 355 | 356 | 358 | 360 | 361 | at 300 ppm | | |

gave more than 70% control in these tests when compared to the untreated check which showed extensive disease development.

### Example 6: Colletotrichum capsici (Anthracnose)

Compounds were dissolved in 0.3% dimethyl sulfoxide & then added to Potato Dextrose Agar medium just prior to dispensing it into Petri dishes. 5 mL medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 25° C. temperature and 60% relative humidity for seven days and radial growth was measured. Compounds

| | | | | | | | | | | | |
|---|---|---|---|---|---|---|---|---|---|---|---|
| | | | | | 3 | 6 | 7 | 8 | 17 | 18 | 20 |
| 21 | 26 | 27 | 28 | 31 | 36 | 38 | 41 | 42 | 48 | 49 | 51 |
| 52 | 57 | 60 | 62 | 63 | 68 | 71 | 72 | 73 | 76 | 77 | 78 |
| 79 | 80 | 83 | 87 | 91 | 103 | 116 | 124 | 125 | 128 | 130 | 131 |
| 132 | 134 | 141 | 148 | 158 | 161 | 163 | 165 | 169 | 171 | 174 | 178 |
| 181 | 185 | 195 | 211 | 213 | 219 | 226 | 230 | 231 | 232 | 246 | 249 |
| 251 | 254 | 255 | 260 | 261 | 263 | 266 | 267 | 272 | 279 | 285 | 286 |
| 289 | 290 | 291 | 292 | 293 | 294 | 301 | 311 | 314 | 328 | 329 | 332 |
| 333 | 334 | 336 | 338 | 339 | 348 | 349 | 351 | 355 | 356 | 358 | 361 |
| at 300 ppm | | | | | | | | | | | |

gave more than 70% control in these tests when compared to the untreated check which showed extensive disease development.

### Example 7: Septoria lycopersici (Leaf Spot of Tomato)

Compounds were dissolved in 0.3% dimethyl sulfoxide & then added to Potato Dextrose Agar medium just prior to dispensing it into Petri dishes. 5 mL medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 25° C. temperature and 70% relative humidity for seven days and radial growth was measured. Compounds

| | | | | | | | | | | | |
|---|---|---|---|---|---|---|---|---|---|---|---|
| | | | | | 1 | 3 | 6 | 7 | 8 | 11 | 17 |
| 18 | 22 | 23 | 26 | 27 | 31 | 36 | 38 | 41 | 48 | 49 | 51 |
| 52 | 53 | 54 | 55 | 58 | 60 | 62 | 63 | 65 | 67 | 68 | 70 |
| 71 | 72 | 73 | 75 | 76 | 77 | 78 | 79 | 83 | 86 | 87 | 90 |
| 91 | 103 | 122 | 123 | 124 | 125 | 127 | 128 | 132 | 134 | 141 | 148 |
| 149 | 150 | 158 | 160 | 161 | 163 | 165 | 174 | 178 | 181 | 211 | 213 |
| 215 | 219 | 226 | 230 | 231 | 232 | 246 | 249 | 254 | 263 | 266 | 276 |
| 279 | 281 | 282 | 286 | 289 | 290 | 291 | 292 | 293 | 294 | 314 | 328 |
| 332 | 333 | 334 | 336 | 337 | 338 | 339 | 340 | 348 | 349 | 351 | 352 |
| 355 | 356 | 359 | 360 | 361 | at 300 ppm | | | | | | |

gave more than 70% control in these tests when compared to the untreated check which showed extensive disease development.

### Example 8: Fusarium culmorum (Foot Rot of Cereals)

Compounds were dissolved in 0.3% dimethyl sulfoxide & then added to Potato Dextrose Agar medium just prior to dispensing it into petri dishes. 5 mL medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 25° C. temperature and 60% relative humidity for seven days and radial growth was measured. Compounds

| | | | | | | | | | | | |
|---|---|---|---|---|---|---|---|---|---|---|---|
| | | | | | 4 | 7 | 22 | 23 | 31 | 40 | 48 |
| 69 | 71 | 73 | 75 | 103 | 165 | 172 | 174 | 177 | 178 | 211 | 213 |
| 219 | 232 | 249 | 268 | 289 | 294 | 301 | 327 | 328 | 333 | 334 | 335 |
| 336 | 338 | 339 | 348 | 355 | at 300 ppm | | | | | | |

gave 70% control in these tests when compared to the untreated check which showed extensive disease development.

Having described the invention with reference to certain preferred aspects, other aspects will become apparent to one skilled in the art from consideration of the specification. It will be apparent to those skilled in the art that many modifications, both to materials and methods, may be practiced without departing from the scope of the invention.

1. A compound of general formula (I); 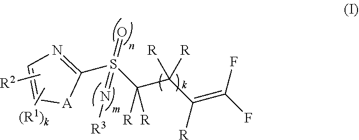
wherein;
A represent O, NR⁴ or S;
n, m and k represent integers, wherein n=0-2, m=0-1 and k=0-2;
R is selected from the group consisting of hydrogen, halogen and C₁-C₃-alkyl;
R¹ is selected from the group consisting of R^{1a}, SCN, SF₅, NO₂, C₁-C₈-alkyl-S(O)₀₋₂R⁴, C₁-C₈-alkyl-(C═O)—R⁴, C(R^{4a})═NR⁴, S(O)₀₋₂C₅-C₁₂-aryl, S(O)₀₋₂C₇-C₁₉-aralkyl, C₃-C₁₀-halocycloalkyl, C₄-C₁₀-cycloalkenyl, C₅-C₁₀-cycloalkynyl, C₁-C₈-alkyloxy-C₃-C₁₀-cycloalkyl, C₇-C₁₉-aralkyl, C₅-C₁₂-bicycloalkyl and C₇-C₁₂-bicycloalkenyl; wherein one or more carbon atoms of the cyclic ring system may be replaced by heteroatoms selected from the group consisting of N, O, S and optionally including 1 to 3 ring members selected from the group consisting of C(═O), C(═S), S(O)₀₋₂ and Si(R⁴)₂; R¹ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
wherein R^{1a} is selected from the group consisting of hydrogen, X, CN, (C═O)—R⁴, OR⁴, N(R⁴)₂, S(O)₀₋₂R⁴, Si(R⁴)₃, C₂-C₁₂-alkenyl, C₂-C₁₂-alkynyl, haloalkyl, C₂-C₁₂-haloalkenyl, C₂-C₁₂-haloalkynyl and C₃-C₁₀-cycloalkyl; R^{1a} may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R² represent following fragment G 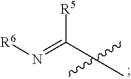
R⁵ is selected from the group consisting of hydrogen, X, CN, C₁-C₁₂-alkyl, C₂-C₁₂-alkenyl, C₂-C₁₂-alkynyl, C₁-C₁₂-haloalkyl, C₂-C₁₂-haloalkenyl, C₂-C₁₂-haloalkynyl, C₃-C₁₀-cycloalkyl, C₃-C₁₀-halocycloalkyl, C₄-C₁₀-cycloalkenyl, C₅-C₁₀-cycloalkynyl, C₄-C₁₀-halocycloalkylalkyl, C₃-C₁₀-aryl and C₇-C₁₉-aralkyl; R⁵ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R⁶ is selected from the group consisting of hydrogen, OR⁴, N(R⁴)₂, Si(R⁴)₃, (C═O)—R⁴, S(O)₀₋₂R⁴, C₁-C₈-alkyl-S(O)₀₋₂R⁴, C₁-C₈-alkyl-(C═O)—R⁴, S(O)₀₋₂C₅-C₁₉-aryl, S(O)₀₋₂C₇-C₁₉-aralkyl, C₁-C₁₂-alkyl, C₂-C₁₂-alkenyl, C₂-C₁₂-alkynyl, C₁-C₁₂-haloalkyl, C₂-C₁₂-haloalkenyl, C₂-C₁₂-haloalkynyl, C₃-C₁₀-cycloalkyl, C₃-C₁₀-halocycloalkyl C₄-C₁₀-cycloalkenyl, C₅-C₁₀-cycloalkynyl, C₃-C₁₀-cycloalkyloxy, C₃-C₁₀-cycloalkylthio, C₆-C₁₀-aryl, C₇-C₁₉-aralkyl, C₅-C₁₂-bicycloalkyl, C₇-C₁₂-bicycloalkenyl and C₃-C₁₀-heterocyclyl; or
R⁵ and R⁶ together with the atom to which they are attached or together with further atoms selected from the group consisting of C, N, O, S and optionally including 1 to 3 ring members selected from the group consisting of C(═O), C(═S), S(O)₀₋₂ and Si(R⁴)₂, may form a four to seven membered ring, which for its part may be substituted by one or more R⁷;
R⁷ is selected from the group consisting of hydrogen, X, CN, SCN, SF₅, R⁴, OR⁴, NO₂, N(R⁴)₂, Si(R⁴)₃, (C═O)—R⁴, S(O)₀₋₂R⁴, C₁-C₈-alkyl-S(O)₀₋₂R⁴, C₁-C₈-alkyl-(C═O)—R⁴, C₂-C₆-alkenyl, C₂-C₆-alkynyl, C₂-C₆-haloalkenyl, C₂-C₆-haloalkynyl, C₃-C₁₀-cycloalkyl, C₃-C₁₀-halocycloalkyl, C₄-C₁₀-cycloalkenyl, C₅-C₁₀-cycloalkynyl, C₇-C₁₀-aralkyl and C₃-C₁₀-heterocyclyl; R⁶ and R⁷ are may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R³ is selected from the group consisting of hydrogen, CN, (C═O)—R⁴, OR⁴, N(R⁴)₂, S(O)₀₋₂R⁴, Si(R⁴)₃, C₂-C₁₂-alkenyl, C₂-C₁₂-alkynyl, C₂-C₁₂-haloalkenyl, C₂-C₁₂-haloalkynyl, C₃-C₁₀-cycloalkyl, C₄-C₁₀-cycloalkenyl and C₅-C₁₀-cycloalkynyl; R³ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R⁴ is selected from the group consisting of hydrogen, OR^{4a}, N(R^{4a})₂, C₁-C₆-alkyl, C₁-C₆-alkenyl, C₁-C₆-alkynyl, C₁-C₆-haloalkyl, C₂-C₆-haloalkenyl, C₂-C₆-haloalkynyl, C₃-C₁₂-cycloalkyl, C₃-C₁₀-halocycloalkyl, C₄-C₁₂-cycloalkenyl, C₅-C₁₂-cycloalkynyl, C₆-C₁₀-aryl, C₇-C₁₉-aralkyl and C₃-C₁₂-heterocyclyl; R⁴ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R^{4a} is selected from the group consisting of hydrogen, C₁-C₆-alkyl and C₃-C₆-cycloalkyl;
when m=1 then R² may or may not be present;
X represents halogen;
and/or stereoisomer or agriculturally acceptable salts or tautomers or N-oxides thereof.

2. The compound as claimed in claim 1, wherein said compound of formula (I) is represented by formula 1a; 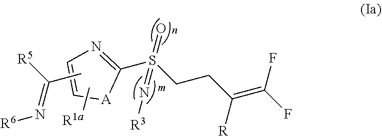
wherein;
A represent 0 or S;
n and m represent integers wherein n=0-2 and m=0-1;
R^{1a} is selected from the group consisting of hydrogen, X, CN, OR⁴, N(R⁴)₂, (C═O)—R⁴, S(O)₀₋₂R⁴, C(R^{4a})═NR⁴, C₂-C₁₂-alkenyl, C₂-C₁₂-alkynyl, haloalkyl, C₂-C₁₂-haloalkenyl, C₂-C₁₂-haloalkynyl, C₃-C₁₀-cycloalkyl, C₃-C₁₀-halocycloalkyl, C₄-C₁₀-cycloalkenyl and C₇-C₁₉-aralkyl; wherein one or more carbon atoms of the cyclic ring system may be replaced by heteroatoms selected from the group consisting of N, O, S and optionally including 1 to 3 ring members selected from the group consisting of C(═O), C(═S), S(O)₀₋₂ and Si(R⁴)₂; R^{1a} may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R³ is selected from the group consisting of hydrogen, CN, (C═O)—R⁴, S(O)₀₋₂R⁴, Si(R⁴)₃, C₁-C₁₂-alkyl, C₂-C₁₂-alkenyl, C₁-C₁₂-haloalkyl, C₂-C₁₂-haloalkenyl, C₃-C₁₀-cycloalkyl and C₄-C₁₀-cycloalkylalkyl; R³ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R⁴ is selected from the group consisting of hydrogen, OR^{4a}, N(R^{4a})₂, C₂-C₆-haloalkenyl, C₃-C₁₂-cycloalkyl, C₃-C₁₀-halocycloalkyl, C₄-C₁₂-cycloalkenyl, C₆-C₁₀-aryl, C₇-C₁₉-aralkyl and C₃-C₁₂-heterocyclyl; R⁴ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂,
R^{4a} is selected from the group consisting of hydrogen, C₁-C₆-alkyl and C₃-C₆-cycloalkyl;
R⁵ is selected from the group consisting of hydrogen, X, CN, C₂-C₁₂-alkenyl, C₂-C₁₂-alkynyl, C₂-C₁₂-haloalkenyl, C₃-C₁₀-cycloalkyl, C₃-C₁₀-halocycloalkyl, C₄-C₁₀-cycloalkylalkyl, C₆-C₁₀-aryl and C₇-C₁₀-aralkyl; R⁵ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R⁶ is selected from the group consisting of hydrogen, OR⁴, N(R⁴)₂, Si(R⁴)₃, (C═O)—R⁴, S(O)₀₋₂R⁴, C₁-C₈-alkyl-S(O)₀₋₂R⁴, C₁-C₈-alkyl-(C═O)—R⁴, S(O)₀₋₂C₅-C₁₉-aryl, S(O)₀₋₂C₇-C₁₉-aralkyl, C₂-C₁₂-alkenyl, C₂-C₁₂-alkynyl, C₁-C₁₂-haloalkyl, C₂-C₁₂-haloalkenyl, C₂-C₁₂-haloalkynyl, C₃-C₁₀-cycloalkyl, C₃-C₁₀-halocycloalkyl C₄-C₁₀-cycloalkenyl, C₆-C₁₀-aryl, C₇-C₁₉-aralkyl, C₅-C₁₂-bicycloalkyl, C₇-C₁₂-bicycloalkenyl and C₃-C₁₀-heterocyclyl; or
R⁵ and R⁶ together with the atom to which they are attached or together with further atoms selected from the group consisting of C, N, O, S and optionally including 1 to 3 ring members selected from the group consisting of C(═O), C(═S), S(O)₀₋₂ and Si(R⁴)₂, may form a four to seven membered ring, which for its part may be substituted by one or more R⁷;
R⁷ is selected from the group consisting of hydrogen, X, CN, SCN, SF₅, R⁴, OR⁴, NO₂, N(R⁴)₂, Si(R⁴)₃, (C═O)—R⁴, S(O)₀₋₂R⁴, C₁-C₈-alkyl-S(O)₀₋₂R⁴, C₁-C₈-alkyl-(C═O)—R⁴, C₂-C₆-alkenyl, C₂-C₆-alkynyl, C₂-C₆-haloalkenyl, C₂-C₆-haloalkynyl, C₃-C₁₀-cycloalkyl, C₃-C₁₀-halocycloalkyl, C₄-C₁₀-cycloalkenyl, C₅-C₁₀-cycloalkynyl, C₆-C₁₀-aryl, C₇-C₁₉-aralkyl and C₃-C₁₀-heterocyclyl; R⁶ and R⁷ are may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
X represents halogen;
and/or stereoisomer or agriculturally acceptable salts or tautomers or N-oxides thereof.

3. The compound as claimed in claim 1, wherein said compound of formula I is represented by formula Ib; 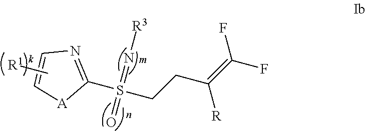
wherein;
A represent O, NR⁴ or S;
n, m and k represent integers wherein n=0-1, m=1 and k=0-2;
R¹ is selected from the group consisting of R^{1a}, SCN, SF₅, NO₂, C₁-C₈-alkyl-S(O)₀₋₂R⁴, C₁-C₆-alkyl-OR⁴, C₁-C₈-alkyl-(C═O)—R⁴, C(R^{4a})═NR⁴, S(O)₀₋₂C₅-C₁₂-aryl, S(O)₀₋₂C₇-C₁₉-aralkyl, C₅-C₁₀-halocycloalkyl, C₄-C₁₀-cycloalkenyl, C₅-C₁₀-cycloalkynyl, C₁-C₈-alkyloxy-C₅-C₁₀-cycloalkyl, C₁-C₈-alkylthio-C₅-C₁₀-cycloalkyl, C₆-C₁₀-aryl, C₇-C₁₉-aralkyl, C₈-C₁₂-bicycloalkyl and C₇-C₁₂-bicycloalkenyl; wherein one or more carbon atoms of the cyclic ring system may be replaced by heteroatoms selected from the group consisting of N, O, S and optionally including 1 to 3 ring members selected from the group consisting of C(═O), C(═S), S(O)₀₋₂ and Si(R⁴)₂; R¹ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R^{1a} is selected from the group consisting of hydrogen, X, CN, (C═O)—R⁴, OR⁴, N(R⁴)₂, S(O)₀₋₂R⁴, Si(R⁴)₃, C₁-C₆-alkyl, C₂-C₆-alkenyl, C₂-C₆-alkynyl, C₁-C₆-haloalkyl, C₂-C₆-haloalkenyl, C₂-C₆-haloalkynyl and C₃-C₆-cycloalkyl; R^{1a} may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
X represents halogen;
R³ is selected from the group consisting of hydrogen, CN, (C═O)—R⁴, OR⁴, N(R⁴)₂, S(O)₀₋₂R⁴, Si(R⁴)₃, C₁-C₁₂-alkyl, C₂-C₁₂-alkenyl, C₂-C₁₂-alkynyl, C₁-C₁₂-haloalkyl, C₂-C₁₂-haloalkenyl, C₂-C₁₂-haloalkynyl, C₅-C₁₀-cycloalkyl and C₄-C₁₀-cycloalkenyl; R³ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R⁴ is selected from the group consisting of hydrogen, OR^{4a}, N(R^{4a})₂, C₁-C₆-alkyl, C₁-C₆-alkenyl, C₁-C₆-alkynyl, C₁-C₆-haloalkyl, C₂-C₆-haloalkenyl, C₂-C₆-haloalkynyl, C₃-C₁₂-cycloalkyl, C₅-C₁₀-halocycloalkyl, C₄-C₆-cycloalkenyl, C₆-C₁₀-aryl, C₇-C₁₉-aralkyl and C₃-C₆-heterocyclyl; R⁴ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R^{4a} is selected from the group consisting of hydrogen, C₁-C₆-alkyl and C₃-C₆-cycloalkyl;
and/or stereoisomer or agriculturally acceptable salts or tautomers or N-oxides thereof.

4. The compound as claimed in claim 1, wherein said compound of formula I is represented by formula Ic; 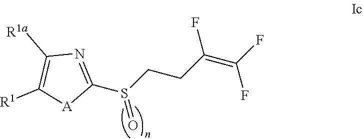
wherein;
A represent O, NR⁴ or S;
n represents integers of 0-2;
R¹ is selected from the group consisting of CN, C₂-C₁₂-alkynyl, C₁-C₆-haloalkyl, C₂-C₆-haloalkenyl, C₂-C₆-haloalkynyl, C₃-C₆-cycloalkyl, C₃-C₆-halocycloalkyl, C₄-C₆-cycloalkenyl, C₁-C₆-haloalkyloxy, C₁-C₆-haloalkylthio, C₁-C₆-haloalkylamino, C₁-C₆-dihaloalkylamino, C₁-C₆-haloalkyloxy-C₁-C₆-alkyl, C₁-C₆-haloalkylamino-C₁-C₆-alkyl, C₁-C₆-dihaloalkylamino-C₁-C₆-alkyl and C₁-C₆-haloalkylthio-C₁-C₆-alkyl; wherein one or more carbon atoms of the cyclic ring system may be replaced by heteroatoms selected from the group consisting of N, O, S and optionally including 1 to 3 ring members selected from the group consisting of C(═O), C(═S), S(O)₀₋₂ and Si(R⁴)₂; R¹ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R^{1a} is selected from the group consisting of hydrogen, X, CN, (C═O)—R⁴, OR⁴, N(R⁴)₂, S(O))₀₋₂R⁴, Si(R⁴)₃, C₁-C₆-alkyl, C₂-C₆-alkenyl, C₂-C₆-alkynyl, C₁-C₆-haloalkyl, C₂-C₆-haloalkenyl, C₂-C₆-haloalkynyl and C₃-C₆-cycloalkyl; R^{1a} may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
X represents halogen;
R⁴ is selected from the group consisting of hydrogen; N(R^{4a})₂, C₁-C₆-alkyl, C₁-C₆-alkenyl, C₁-C₆-alkynyl, C₁-C₆-haloalkyl, C₂-C₆-haloalkenyl, C₂-C₆-haloalkynyl, C₃-C₁₂-cycloalkyl, C₅-C₁₀-halocycloalkyl, C₄-C₆-cycloalkenyl, C₆-C₁₀-aryl, C₇-C₁₉-aralkyl and C₃-C₆-heterocyclyl; R⁴ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂,
R^{4a} is selected from the group consisting of hydrogen, C₁-C₆-alkyl and C₃-C₆-cycloalkyl;
and/or stereoisomer or agriculturally acceptable salts or tautomers or N-oxides thereof.

5. The compound as claimed in claim 1, wherein said compound of formula (I) is selected from (E)-2-((3,4,4-trifluorobut-3-en-1-yl)thio)thiazole-5-carbaldehyde O-methyl oxime, (E)-2-((3,4,4-trifluorobut-3-en-1-yl)thio)thiazole-5-carbaldehyde O-methyl oxime, (Z)-2-((3,4,4-trifluorobut-3-en-1-yl)thio)oxazole-5-carbaldehyde O-methyl oxime, (E)-2-((3,4,4-trifluorobut-3-en-1-yl)sulfinyl)thiazole-5-carbaldehyde O-methyl oxime, (Z)-2-((3,4,4-trifluorobut-3-en-1-yl)sulfinyl)thiazole-5-carbaldehyde O-methyl oxime, (E)-2-((3,4,4-trifluorobut-3-en-1-yl)thio)oxazole-5-carbaldehyde O-methyl oxime, (E)-2-((3,4,4-trifluorobut-3-en-1-yl)sulfinyl)oxazole-5-carbaldehyde O-methyl oxime, (Z)-2-((3,4,4-trifluorobut-3-en-1-yl)sulfinyl)oxazole-5-carbaldehyde O-methyl oxime, (E)-2-((3,4,4-trifluorobut-3-en-1-yl)sulfonyl)oxazole-5-carbaldehyde O-methyl oxime, 2-((3,4,4-trifluorobut-3-en-1-yl)thio)thiazole-5-carbaldehyde O-ethyl oxime, (Z)-2-((3,4,4-trifluorobut-3-en-1-yl)sulfonyl)thiazole-5-carbaldehyde O-methyl oxime, (E)-2-((3,4,4-trifluorobut-3-en-1-yl)thio)thiazole-5-carbaldehyde O-ethyl oxime, (Z)-2-((3,4,4-trifluorobut-3-en-1-yl)thio)oxazole-5-carbaldehyde O-ethyl oxime, (E)-2-((((2-((3,4,4-trifluorobut-3-en-1-yl)thio)thiazol-5-yl)methylene)amino)oxy)acetonitrile, (E)-2-((3,4,4-trifluorobut-3-en-1-yl)thio)thiazole-5-carbaldehyde O-isopropyl oxime, (E)-2-((3,4,4-trifluorobut-3-en-1-yl)thio)thiazole-5-carbaldehyde O-cyclopropylmethyl oxime, (E)-2-((3,4,4-trifluorobut-3-en-1-yl)thio)oxazole-5-carbaldehyde O-ethyl oxime, (Z)-2-((3,4,4-trifluorobut-3-en-1-yl)sulfinyl)oxazole-5-carbaldehyde O-ethyl oxime, (Z)-4-phenyl-2-((3,4,4-trifluorobut-3-en-1-yl)thio)thiazole-5-carbaldehyde O-methyl oxime, (E)-4-phenyl-2-((3,4,4-trifluorobut-3-en-1-yl)thio)thiazole-5-carbaldehyde O-methyl oxime, (Z)-2-((3,4,4-trifluorobut-3-en-1-yl)thio)thiazole-5-carbaldehyde O-ethyl oxime, (E)-2-((3,4,4-trifluorobut-3-en-1-yl)sulfinyl)thiazole-5-carbaldehyde O-ethyl oxime, (E)-2-((3,4,4-trifluorobut-3-en-1-yl)sulfonyl)thiazole-5-carbaldehyde O-ethyl oxime, (Z)-4-phenyl-2-((3,4,4-trifluorobut-3-en-1-yl)thio)thiazole-5-carbaldehyde O-ethyl oxime, (E)-4-phenyl-2-((3,4,4-trifluorobut-3-en-1-yl)thio)thiazole-5-carbaldehyde O-ethyl oxime, (E)-4-methyl-2-((3,4,4-trifluorobut-3-en-1-yl)thio)thiazole-5-carbaldehyde O-methyl oxime, (Z)-4-methyl-2-((3,4,4-trifluorobut-3-en-1-yl)thio)thiazole-5-carbaldehyde O-methyl oxime, (Z)-4-phenyl-2-((3,4,4-trifluorobut-3-en-1-yl)sulfinyl)thiazole-5-carbaldehyde O-methyl oxime, (E)-4-phenyl-2-((3,4,4-trifluorobut-3-en-1-yl)sulfonyl)thiazole-5-carbaldehyde O-methyl oxime, (E)-4-phenyl-2-((3,4,4-trifluorobut-3-en-1-yl)sulfinyl)thiazole-5-carbaldehyde O-methyl oxime, (Z)-2-((3,4,4-trifluorobut-3-en-1-yl)sulfonyl)oxazole-5-carbaldehyde O-ethyl oxime, (E)-2-((3,4,4-trifluorobut-3-en-1-yl)sulfonyl)oxazole-5-carbaldehyde O-ethyl oxime, (E)-4-(tert-butyl)-2-((3,4,4-trifluorobut-3-en-1-yl)thio)thiazole-5-carbaldehyde O-methyl oxime, (E)-2-((3,4,4-trifluorobut-3-en-1-yl)thio)thiazole-5-carbaldehyde O-isopentyl oxime, (E)-2-((3,4,4-trifluorobut-3-en-1-yl)thio)thiazole-5-carbaldehyde O-cyclohexyl oxime, (E)-2-((3,4,4-trifluorobut-3-en-1-yl)sulfinyl)oxazole-5-carbaldehyde O-ethyl oxime, (E)-2-((3,4,4-trifluorobut-3-en-1-yl)sulfonyl)thiazole-5-carbaldehyde O-cyclohexyl oxime, (E)-1-(2-((3,4,4-trifluorobut-3-en-1-yl)thio)thiazol-5-yl)ethan-1-one O-methyl oxime, (E)-4-methyl-2-((3,4,4-trifluorobut-3-en-1-yl)sulfinyl)thiazole-5-carbaldehyde O-methyl oxime, (E)-4-methyl-2-((3,4,4-trifluorobut-3-en-1-yl)sulfonyl)thiazole-5-carbaldehyde O-methyl oxime, (E)-4-methyl-2-((3,4,4-trifluorobut-3-en-1-yl)thio)thiazole-5-carbaldehyde O-ethyl oxime, (Z)-4-methyl-2-((3,4,4-trifluorobut-3-en-1-yl)thio)thiazole-5-carbaldehyde O-ethyl oxime, (Z)-2-((3,4,4-trifluorobut-3-en-1-yl)sulfonyl)thiazole-5-carbaldehyde O-cyclobutylmethyl oxime, (Z)-2-((3,4,4-trifluorobut-3-en-1-yl)thio)thiazole-5-carbaldehyde O-cyclobutylmethyl oxime, (E)-2-((3,4,4-trifluorobut-3-en-1-yl)thio)thiazole-5-carbaldehyde O-cyclobutylmethyl oxime, (E)-2-((3,4,4-trifluorobut-3-en-1-yl)thio)oxazole-5-carbaldehyde O-isopentyl oxime, (Z)-2-((3,4,4-trifluorobut-3-en-1-yl)thio)oxazole-5-carbaldehyde O-isopentyl oxime, (Z)-2-((3,4,4-trifluorobut-3-en-1-yl)sulfinyl)oxazole-5-carbaldehyde O-isopentyl oxime, (Z)-2-((3,4,4-trifluorobut-3-en-1-yl)sulfonyl)oxazole-5-carbaldehyde O-isopentyl oxime, (E)-2-((3,4,4-trifluorobut-3-en-1-yl)sulfonyl)oxazole-5-carbaldehyde O-isopentyl oxime, (E)-2-((3,4,4-trifluorobut-3-en-1-yl)sulfinyl)oxazole-5-carbaldehyde O-isopentyl oxime, (E)-2-((3,4,4-trifluorobut-3-en-1-yl)thio)thiazole-4-carbaldehyde O-methyl oxime, (E)-1-(2-((3,4,4-trifluorobut-3-en-1-yl)sulfonyl)thiazol-5-yl)ethan-1-one O-methyl oxime, (E)-1-(2-((3,4,4-trifluorobut-3-en-1-yl)sulfinyl)thiazol-5-yl)ethan-1-one O-methyl oxime, (Z)-1-(2-((3,4,4-trifluorobut-3-en-1-yl)thio)thiazol-5-yl)ethan-1-one O-methyl oxime, (E)-1-(2-((3,4,4-trifluorobut-3-en-1-yl)thio)thiazol-5-yl)ethan-1-one O-ethyl oxime, (Z)-1-(2-((3,4,4-trifluorobut-3-en-1-yl)thio)thiazol-5-yl)ethan-1-one O-ethyl oxime, (E)-1-(2-((3,4,4-trifluorobut-3-en-1-yl)sulfinyl)thiazol-5-yl)ethan-1-one O-ethyl oxime, (E)-1-(2-((3,4,4-trifluorobut-3-en-1-yl)sulfonyl)thiazol-5-yl)ethan-1-one O-ethyl oxime, (Z)-1-(2-((3,4,4-trifluorobut-3-en-1-yl)sulfinyl)thiazol-5-yl)ethan-1-one O-ethyl oxime, (Z)-1-(2-((3,4,4-trifluorobut-3-en-1-yl)sulfonyl)thiazol-5-yl)ethan-1-one O-ethyl oxime, (Z)-2-((3,4,4-trifluorobut-3-en-1-yl)thio)thiazole-4-carbaldehyde O-methyl oxime, (Z)-2-((3,4,4-trifluorobut-3-en-1-yl)thio)thiazole-4-carbaldehyde O-ethyl oxime, (E)-2-((3,4,4-trifluorobut-3-en-1-yl)sulfinyl)thiazole-4-carbaldehyde O-methyl oxime, (E)-2-((3,4,4-trifluorobut-3-en-1-yl)sulfinyl)thiazole-4-carbaldehyde O-ethyl oxime, (E)-2-((3,4,4-trifluorobut-3-en-1-yl)sulfonyl)thiazole-4-carbaldehyde O-methyl oxime, (E)-2-((3,4,4-trifluorobut-3-en-1-yl)sulfonyl)thiazole-4-carbaldehyde O-ethyl oxime, (E)-5-chloro-2-((3,4,4-trifluorobut-3-en-1-yl)thio)thiazole-4-carbaldehyde O-methyl oxime, (E)-5-chloro-2-((3,4,4-trifluorobut-3-en-1-yl)sulfonyl)thiazole-4-carbaldehyde O-methyl oxime, (E)-2-((3,4,4-trifluorobut-3-en-1-yl)sulfonyl)thiazole-5-carbaldehyde O-methyl oxime, (Z)-2-((3,4,4-trifluorobut-3-en-1-yl)sulfonyl)oxazole-5-carbaldehyde O-methyl oxime, (E)-2-((3,4,4-trifluorobut-3-en-1-yl)thio)oxazole-5-carbaldehyde O-cyclopropylmethyl oxime, N-((5-((Z)-(methoxyimino)methyl)thiazol-2-yl)(3,4,4-trifluorobut-3-en-1-yl)-λ⁴ sulfaneylidene)cyanamide, (E)-4-methyl-2-((3,4,4-trifluorobut-3-en-1-yl)sulfonyl)thiazole-5-carbaldehyde O-ethyl oxime, (E)-4-methyl-2-((3,4,4-trifluorobut-3-en-1-yl)sulfinyl)thiazole-5-carbaldehyde O-ethyl oxime, (E)-2-((3,4,4-trifluorobut-3-en-1-yl)thio)oxazole-5-carbaldehyde O-isopropyl oxime, (E)-2-((3,4,4-trifluorobut-3-en-1-yl)sulfinyl)oxazole-5-carbaldehyde O-isopropyl oxime, (E)-2-((3,4,4-trifluorobut-3-en-1-yl)sulfonyl)oxazole-5-carbaldehyde O-isopropyl oxime, (E)-2-((3,4,4-trifluorobut-3-en-1-yl)sulfinyl)oxazole-5-carbaldehyde O-cyclopropylmethyl oxime, (E)-2-((3,4,4-trifluorobut-3-en-1-yl)sulfonyl)oxazole-5-carbaldehyde O-cyclopropylmethyl oxime, (Z)-2-((3,4,4-trifluorobut-3-en-1-yl)sulfonyl)thiazole-5-carbaldehyde O-ethyl oxime, (Z)-2-((3,4,4-trifluorobut-3-en-1-yl)thio)oxazole-5-carbaldehyde O-cyclopropylmethyl oxime, (Z)-2-((3,4,4-trifluorobut-3-en-1-yl)sulfonyl)oxazole-5-carbaldehyde O-cyclopropylmethyl oxime, (Z)-1-(2-((3,4,4-trifluorobut-3-en-1-yl)thio)thiazol-5-yl)ethan-1-one O-cyclopropylmethyl oxime, (Z)-1-(2-((3,4,4-trifluorobut-3-en-1-yl)sulfonyl)thiazol-5-yl)ethan-1-one O-cyclopropylmethyl oxime, (Z)-1-(2-((3,4,4-trifluorobut-3-en-1-yl)sulfinyl)thiazol-5-yl)ethan-1-one O-cyclopropylmethyl oxime, (Z)-2-((3,4,4-trifluorobut-3-en-1-yl)sulfinyl)thiazole-5-carbaldehyde O-ethyl oxime, (Z)-2-((3,4,4-trifluorobut-3-en-1-yl)thio)thiazole-5-carbaldehyde O-isopropyl oxime, (Z)-2-((3,4,4-trifluorobut-3-en-1-yl)sulfonyl)thiazole-5-carbaldehyde O-isopropyl oxime, (E)-2-((3,4,4-trifluorobut-3-en-1-yl)sulfinyl)thiazole-5-carbaldehyde O-isopropyl oxime, N-((E)-(4-((E)-(methoxyimino)methyl)thiazol-2-yl)(3,4,4-trifluorobut-3-en-1-yl)-l4-sulfaneylidene)cyanamide, (E)-1-(2-((3,4,4-trifluorobut-3-en-1-yl)thio)thiazol-5-yl)ethan-1-one O-cyclopropylmethyl oxime, (E)-phenyl(2-((3,4,4-trifluorobut-3-en-1-yl)thio)thiazol-5-yl)methanone O-methyl oxime, (E)-phenyl(2-((3,4,4-trifluorobut-3-en-1-yl)sulfinyl)thiazol-5-yl)methanone O-methyl oxime, (E)-phenyl(2-((3,4,4-trifluorobut-3-en-1-yl)thio)thiazol-5-yl)methanone O-ethyl oxime, (E)-phenyl(2-((3,4,4-trifluorobut-3-en-1-yl)sulfonyl)thiazol-5-yl)methanone O-methyl oxime, (E)-phenyl(2-((3,4,4-trifluorobut-3-en-1-yl)thio)thiazol-5-yl)methanone oxime, (E)-phenyl(2-((3,4,4-trifluorobut-3-en-1-yl)sulfinyl)thiazol-5-yl)methanone O-ethyl oxime, (E)-phenyl(2-((3,4,4-trifluorobut-3-en-1-yl)sulfonyl)thiazol-5-yl)methanone O-ethyl oxime, (E)-phenyl(2-((3,4,4-trifluorobut-3-en-1-yl)thio)thiazol-5-yl)methanone O-cyclopropylmethyl oxime, (E)-2-((3,4,4-trifluorobut-3-en-1-yl)thio)oxazole-5-carbaldehyde O-cyclobutylmethyl oxime, (Z)-2-((3,4,4-trifluorobut-3-en-1-yl)thio)oxazole-5-carbaldehyde O-isopropyl oxime, (Z)-2-((3,4,4-trifluorobut-3-en-1-yl)sulfinyl)oxazole-5-carbaldehyde O-cyclopropylmethyl oxime, (E)-phenyl(2-((3,4,4-trifluorobut-3-en-1-yl)sulfinyl)thiazol-5-yl)methanone O-cyclopropylmethyl oxime, (E)-phenyl(2-((3,4,4-trifluorobut-3-en-1-yl)sulfinyl)thiazol-5-yl)methanone oxime, (E)-phenyl(2-((3,4,4-trifluorobut-3-en-1-yl)sulfonyl)thiazol-5-yl)methanone O-cyclopropylmethyl oxime, (E)-phenyl(2-((3,4,4-trifluorobut-3-en-1-yl)thio)thiazol-5-yl)methanone O-propyl oxime, (E)-phenyl(2-((3,4,4-trifluorobut-3-en-1-yl)thio)thiazol-5-yl)methanone O-isopentyl oxime, (E)-phenyl(2-((3,4,4-trifluorobut-3-en-1-yl)sulfonyl)thiazol-5-yl)methanone oxime, (E)-phenyl(2-((3,4,4-trifluorobut-3-en-1-yl)sulfinyl)thiazol-5-yl)methanone O-propyl oxime, (E)-phenyl(2-((3,4,4-trifluorobut-3-en-1-yl)sulfonyl)thiazol-5-yl)methanone O-propyl oxime, (E)-phenyl(2-((3,4,4-trifluorobut-3-en-1-yl)sulfinyl)thiazol-5-yl)methanone O-isopentyl oxime, (E)-phenyl(2-((3,4,4-trifluorobut-3-en-1-yl)sulfonyl)thiazol-5-yl)methanone O-isopentyl oxime, (E)-1-(2-((3,4,4-trifluorobut-3-en-1-yl)sulfinyl)thiazol-5-yl)ethan-1-one O-cyclopropylmethyl oxime, (E)-1-(2-((3,4,4-trifluorobut-3-en-1-yl)sulfonyl)thiazol-5-yl)ethan-1-one O-cyclopropylmethyl oxime, (Z)-1-(2-((3,4,4-trifluorobut-3-en-1-yl)thio)thiazol-5-yl)ethan-1-one O-isopropyl oxime, (E)-1-(2-((3,4,4-trifluorobut-3-en-1-yl)thio)thiazol-5-yl)ethan-1-one O-isopropyl oxime, (Z)-1-(2-((3,4,4-trifluorobut-3-en-1-yl)sulfinyl)thiazol-5-yl)ethan-1-one O-isopropyl oxime, (Z)-1-(2-((3,4,4-trifluorobut-3-en-1-yl)sulfonyl)thiazol-5-yl)ethan-1-one O-isopropyl oxime, (E)-1-(2-((3,4,4-trifluorobut-3-en-1-yl)sulfinyl)thiazol-5-yl)ethan-1-one O-isopropyl oxime, (E)-1-(2-((3,4,4-trifluorobut-3-en-1-yl)sulfonyl)thiazol-5-yl)ethan-1-one O-isopropyl oxime, (Z)-1-(2-((3,4,4-trifluorobut-3-en-1-yl)sulfinyl)thiazol-5-yl)ethan-1-one O-methyl oxime, (Z)-1-(2-((3,4,4-trifluorobut-3-en-1-yl)sulfonyl)thiazol-5-yl)ethan-1-one O-methyl oxime, (Z)-2-((3,4,4-trifluorobut-3-en-1-yl)sulfinyl)oxazole-5-carbaldehyde O-isopropyl oxime, (E)-2-((3,4,4-trifluorobut-3-en-1-yl)sulfinyl)oxazole-5-carbaldehyde O-cyclobutylmethyl oxime, (E)-2-((3,4,4-trifluorobut-3-en-1-yl)sulfonyl)oxazole-5-carbaldehyde O-cyclobutylmethyl oxime, (Z)-1-(2-((3,4,4-trifluorobut-3-en-1-yl)thio)oxazol-5-yl)ethan-1-one O-methyl oxime, (E)-1-(2-((3,4,4-trifluorobut-3-en-1-yl)thio)oxazol-5-yl)ethan-1-one O-methyl oxime, (E)-1-(2-((3,4,4-trifluorobut-3-en-1-yl)sulfonyl)oxazol-5-yl)ethan-1-one O-methyl oxime, (E)-1-(2-((3,4,4-trifluorobut-3-en-1-yl)sulfinyl)oxazol-5-yl)ethan-1-one O-methyl oxime, (Z)-2-((3,4,4-trifluorobut-3-en-1-yl)sulfonyl)oxazole-5-carbaldehyde O-isopropyl oxime, (E)-1-(2-((3,4,4-trifluorobut-3-en-1-yl)thio)oxazol-5-yl)ethan-1-one O-ethyl oxime, (E)-1-(2-((3,4,4-trifluorobut-3-en-1-yl)sulfonyl)oxazol-5-yl)ethan-1-one O-ethyl oxime, (Z)-1-(2-((3,4,4-trifluorobut-3-en-1-yl)sulfonyl)oxazol-5-yl)ethan-1-one O-methyl oxime, (Z)-1-(2-((3,4,4-trifluorobut-3-en-1-yl)thio)oxazol-5-yl)ethan-1-one O-ethyl oxime, (E)-2-((3,4,4-trifluorobut-3-en-1-yl)sulfinyl)thiazole-5-carbaldehyde O-isopentyl oxime, (E)-2-((3,4,4-trifluorobut-3-en-1-yl)sulfonyl)thiazole-5-carbaldehyde O-isopentyl oxime, (E)-1-(2-((3,4,4-trifluorobut-3-en-1-yl)thio)thiazol-5-yl)ethan-1-one O-isopentyl oxime, (E)-1-(2-((3,4,4-trifluorobut-3-en-1-yl)sulfinyl)thiazol-5-yl)ethan-1-one O-isopentyl oxime, (E)-1-(2-((3,4,4-trifluorobut-3-en-1-yl)sulfonyl)thiazol-5-yl)ethan-1-one O-isopentyl oxime, (E)-1-(2-((3,4,4-trifluorobut-3-en-1-yl)thio)thiazol-4-yl)ethan-1-one O-ethyl oxime, (E)-1-(2-((3,4,4-trifluorobut-3-en-1-yl)sulfinyl)thiazol-4-yl)ethan-1-one O-ethyl oxime, (E)-1-(2-((3,4,4-trifluorobut-3-en-1-yl)sulfonyl)thiazol-4-yl)ethan-1-one O-ethyl oxime, (Z)-1-(2-((3,4,4-trifluorobut-3-en-1-yl)thio)thiazol-5-yl)ethan-1-one O-isopentyl oxime, (Z)-1-(2-((3,4,4-trifluorobut-3-en-1-yl)sulfinyl)thiazol-5-yl)ethan-1-one O-isopentyl oxime, (Z)-1-(2-((3,4,4-trifluorobut-3-en-1-yl)sulfonyl)thiazol-5-yl)ethan-1-one O-isopentyl oxime, (E)-4-methyl-2-((3,4,4-trifluorobut-3-en-1-yl)thio)thiazole-5-carbaldehyde O-isopropyl oxime, (E)-1-(2-((3,4,4-trifluorobut-3-en-1-yl)thio)thiazol-4-yl)ethan-1-one O-methyl oxime, (E)-1-(2-((3,4,4-trifluorobut-3-en-1-yl)sulfinyl)thiazol-4-yl)ethan-1-one O-methyl oxime, (E)-1-(2-((3,4,4-trifluorobut-3-en-1-yl)sulfonyl)thiazol-4-yl)ethan-1-one O-methyl oxime, (E)-4-methyl-2-((3,4,4-trifluorobut-3-en-1-yl)thio)thiazole-5-carbaldehyde O-cyclopropylmethyl oxime, (E)-2-((3,4,4-trifluorobut-3-en-1-yl)thio)-4-(trifluoromethyl)thiazole-5-carbaldehyde O-methyl oxime, (E)-2-((3,4,4-trifluorobut-3-en-1-yl)sulfinyl)-4-(trifluoromethyl)thiazole-5-carbaldehyde O-methyl oxime, (E)-2-((3,4,4-trifluorobut-3-en-1-yl)sulfonyl)-4-(trifluoromethyl)thiazole-5-carbaldehyde O-methyl oxime, (E)-2-((3,4,4-trifluorobut-3-en-1-yl)thio)-4-(trifluoromethyl)thiazole-5-carbaldehyde O-ethyl oxime, (E)-2-((3,4,4-trifluorobut-3-en-1-yl)sulfinyl)-4-(trifluoromethyl)thiazole-5-carbaldehyde O-ethyl oxime, (E)-2-((3,4,4-trifluorobut-3-en-1-yl)sulfonyl)-4-(trifluoromethyl)thiazole-5-carbaldehyde O-ethyl oxime, (E)-4-methyl-2-((3,4,4-trifluorobut-3-en-1-yl)sulfinyl)thiazole-5-carbaldehyde O-cyclopropylmethyl oxime, (E)-4-methyl-2-((3,4,4-trifluorobut-3-en-1-yl)sulfonyl)thiazole-5-carbaldehyde O-cyclopropylmethyl oxime, (E)-4-methyl-2-((3,4,4-trifluorobut-3-en-1-yl)sulfinyl)thiazole-5-carbaldehyde O-isopropyl oxime, N-((5-chlorothiazol-2-yl)(3,4,4-trifluorobut-3-en-1-yl)-λ⁴-sulfaneylidene)cyanamide, N-(thiazol-2-yl(3,4,4-trifluorobut-3-en-1-yl)-λ⁴-sulfaneylidene)cyanamide, N-((5-chlorothiazol-2-yl)(oxo)(3,4,4-trifluorobut-3-en-1-yl)-λ⁶-sulfaneylidene)cyanamide, N-(oxo(thiazol-2-yl)(3,4,4-trifluorobut-3-en-1-yl)-λ⁶-sulfaneylidene)cyanamide, N-((5-bromothiazol-2-yl)(oxo)(3,4,4-trifluorobut-3-en-1-yl)-λ⁶-sulfaneylidene)cyanamide, imino(thiazol-2-yl)(3,4,4-trifluorobut-3-en-1-yl)-λ⁶-sulfanone, (ethylimino)(thiazol-2-yl)(3,4,4-trifluorobut-3-en-1-yl)-λ⁶-sulfanone, 2,2,2-trifluoro-N-(oxo(thiazol-2-yl)(3,4,4-trifluorobut-3-en-1-yl)-λ⁶-sulfaneylidene)acetamide, (5-chlorothiazol-2-yl)(imino)(3,4,4-trifluorobut-3-en-1-yl)-λ⁶-sulfanone, (5-chlorothiazol-2-yl)(ethylimino)(3,4,4-trifluorobut-3-en-1-yl)-λ⁶-sulfanone, (methylimino)(thiazol-2-yl)(3,4,4-trifluorobut-3-en-1-yl)-λ⁶-sulfanone, (propylimino)(thiazol-2-yl)(3,4,4-trifluorobut-3-en-1-yl)-λ⁶-sulfanone, ((cyclopropylmethyl)imino)(thiazol-2-yl)(3,4,4-trifluorobut-3-en-1-yl)-λ⁶-sulfanone, N-((5-bromothiazol-2-yl)(3,4,4-trifluorobut-3-en-1-yl)-λ⁴-sulfaneylidene)cyanamide, (5-bromothiazol-2-yl)(imino)(3,4,4-trifluorobut-3-en-1-yl)-λ⁶-sulfanone, (5-bromothiazol-2-yl)(ethylimino)(3,4,4-trifluorobut-3-en-1-yl)-λ⁶-sulfanone, (5-(difluoromethyl)thiazol-2-yl)(imino)(3,4,4-trifluorobut-3-en-1-yl)-λ⁶-sulfanone, 2-(3,4,4-trifluorobut-3-en-1-ylsulfonimidoyl)thiazole-5-carbonitrile, (5-bromothiazol-2-yl)(methylimino)(3,4,4-trifluorobut-3-en-1-yl)-λ⁶-sulfanone, (5-chlorothiazol-2-yl)(methylimino)(3,4,4-trifluorobut-3-en-1-yl)-λ⁶-sulfanone, N-((5-(difluoromethyl)thiazol-2-yl)(3,4,4-trifluorobut-3-en-1-yl)-λ⁴-sulfaneylidene)cyanamide, N-((4-methylthiazol-2-yl)(3,4,4-trifluorobut-3-en-1-yl)-λ⁴-sulfaneylidene)cyanamide, N-((4-methylthiazol-2-yl)(oxo)(3,4,4-trifluorobut-3-en-1-yl)-λ⁶-sulfaneylidene)cyanamide, N-((5-chloro-4-methylthiazol-2-yl)(3,4,4-trifluorobut-3-en-1-yl)-λ⁴-sulfaneylidene)cyanamide, N-((5-chloro-4-methylthiazol-2-yl)(oxo)(3,4,4-trifluorobut-3-en-1-yl)-λ⁶-sulfaneylidene)cyanamide, (5-chloro-4-methylthiazol-2-yl)(imino)(3,4,4-trifluorobut-3-en-1-yl)-λ⁶-sulfanone, N-((4-(tert-butyl)thiazol-2-yl)(3,4,4-trifluorobut-3-en-1-yl)-λ⁴-sulfaneylidene)cyanamide, (4-(tert-butyl)thiazol-2-yl)(imino)(3,4,4-trifluorobut-3-en-1-yl)-λ⁶-sulfanone, (4-(tert-butyl)thiazol-2-yl)(methylimino)(3,4,4-trifluorobut-3-en-1-yl)-λ⁶-sulfanone, imino(4-phenylthiazol-2-yl)(3,4,4-trifluorobut-3-en-1-yl)-λ⁶-sulfanone, (5-bromo-4-phenylthiazol-2-yl)(imino)(3,4,4-trifluorobut-3-en-1-yl)-λ⁶-sulfanone, (5-chloro-4-phenylthiazol-2-yl)(imino)(3,4,4-trifluorobut-3-en-1-yl)-λ⁶-sulfanone, N-((4-(tert-butyl)-5-chlorothiazol-2-yl)(3,4,4-trifluorobut-3-en-1-yl)-λ⁴-sulfaneylidene)cyanamide, (4-(tert-butyl)-5-chlorothiazol-2-yl)(imino)(3,4,4-trifluorobut-3-en-1-yl)-λ⁶-sulfanone, (4-(tert-butyl)-5-chlorothiazol-2-yl)(methylimino)(3,4,4-trifluorobut-3-en-1-yl)-λ⁶-sulfanone, (4-(tert-butyl)-5-chlorothiazol-2-yl)(ethylimino)(3,4,4-trifluorobut-3-en-1-yl)-λ⁶-sulfanone, N-((5-bromo-4-(tert-butyl)thiazol-2-yl)(3,4,4-trifluorobut-3-en-1-yl)-λ⁴-sulfaneylidene)cyanamide, (5-bromo-4-(tert-butyl)thiazol-2-yl)(imino)(3,4,4-trifluorobut-3-en-1-yl)-λ⁶-sulfanone, (5-bromo-4-(tert-butyl)thiazol-2-yl)(methylimino)(3,4,4-trifluorobut-3-en-1-yl)-λ⁶-sulfanone, (5-bromo-4-(tert-butyl)thiazol-2-yl)(ethylimino)(3,4,4-trifluorobut-3-en-1-yl)-λ⁶-sulfanone, (4,5-dimethylthiazol-2-yl)(imino)(3,4,4-trifluorobut-3-en-1-yl)-λ⁶-sulfanone, imino(4-methylthiazol-2-yl)(3,4,4-trifluorobut-3-en-1-yl)-λ⁶-sulfanone, (methylimino)(4-methylthiazol-2-yl)(3,4,4-trifluorobut-3-en-1-yl)-λ⁶-sulfanone, (ethylimino)(4-methylthiazol-2-yl)(3,4,4-trifluorobut-3-en-1-yl)-λ⁶-sulfanone, (5-bromo-4-phenylthiazol-2-yl)(methylimino)(3,4,4-trifluorobut-3-en-1-yl)-λ⁶-sulfanone, N-((4,5-dimethylthiazol-2-yl)(3,4,4-trifluorobut-3-en-1-yl)-λ⁴-sulfaneylidene)cyanamide, 5-(2-chlorophenyl)-3-(2-((3,4,4-trifluorobut-3-en-1-yl)thio)thiazol-5-yl)-4,5-dihydroisoxazole, 5-phenyl-3-(2-((3,4,4-trifluorobut-3-en-1-yl)thio)thiazol-5-yl)-4,5-dihydroisoxazole, 5-(2-chlorophenyl)-3-(2-((3,4,4-trifluorobut-3-en-1-yl)sulfinyl)thiazol-5-yl)-4,5-dihydroisoxazole, 5-(2-chlorophenyl)-3-(2-((3,4,4-trifluorobut-3-en-1-yl)sulfonyl)thiazol-5-yl)-4,5-dihydroisoxazole, 3-(2-((3,4,4-trifluorobut-3-en-1-yl)thio)thiazol-5-yl)-1,2,4-oxadiazole, 5-phenyl-3-(2-((3,4,4-trifluorobut-3-en-1-yl)sulfonyl)thiazol-5-yl)-4,5-dihydroisoxazole, 3-(2-((3,4,4-trifluorobut-3-en-1-yl)sulfinyl)thiazol-5-yl)-1,2,4-oxadiazole, 5-(4-chlorophenyl)-3-(2-((3,4,4-trifluorobut-3-en-1-yl)thio)thiazol-5-yl)isoxazole, 5-methyl-3-(2-((3,4,4-trifluorobut-3-en-1-yl)thio)thiazol-5-yl)-1,2,4-oxadiazole, 5-methyl-3-(2-((3,4,4-trifluorobut-3-en-1-yl)sulfinyl)thiazol-5-yl)-1,2,4-oxadiazole, 5-(2H-tetrazol-5-yl)-2-((3,4,4-trifluorobut-3-en-1-yl)thio)thiazole, 5-(4-chlorophenyl)-3-(2-((3,4,4-trifluorobut-3-en-1-yl)sulfonyl)thiazol-5-yl)isoxazole, 5-methyl-3-(2-((3,4,4-trifluorobut-3-en-1-yl)sulfonyl)thiazol-5-yl)-1,2,4-oxadiazole, 5-(2H-tetrazol-5-yl)-2-((3,4,4-trifluorobut-3-en-1-yl)sulfonyl)thiazole, 3-(2-((3,4,4-trifluorobut-3-en-1-yl)thio)thiazol-5-yl)-1,2,4-oxadiazol-5(4H)-one, 3-(2-((3,4,4-trifluorobut-3-en-1-yl)sulfinyl)thiazol-5-yl)-1,2,4-oxadiazol-5(4H)-one, 5-(2H-tetrazol-5-yl)-2-((3,4,4-trifluorobut-3-en-1-yl)sulfinyl)thiazole, 3-(2-((3,4,4-trifluorobut-3-en-1-yl)sulfonyl)thiazol-5-yl)-1,2,4-oxadiazol-5(4H)-one, 5-(4-chlorophenyl)-3-(2-((3,4,4-trifluorobut-3-en-1-yl)thio)thiazol-4-yl)isoxazole, 3-(2-((3,4,4-trifluorobut-3-en-1-yl)thio)thiazol-4-yl)-5-(trifluoromethyl)-1,2,4-oxadiazole, 3-(2-((3,4,4-trifluorobut-3-en-1-yl)sulfinyl)thiazol-4-yl)-5-(trifluoromethyl)-1,2,4-oxadiazole, 3-(2-((3,4,4-trifluorobut-3-en-1-yl)sulfonyl)thiazol-4-yl)-5-(trifluoromethyl)-1,2,4-oxadiazole, 3-(2-((3,4,4-trifluorobut-3-en-1-yl)thio)thiazol-5-yl)-5-(trifluoromethyl)-1,2,4-oxadiazole, 5-methyl-3-(2-((3,4,4-trifluorobut-3-en-1-yl)thio)thiazol-4-yl)-1,2,4-oxadiazole, N-((4-(5-methyl-1,2,4-oxadiazol-3-yl)thiazol-2-yl)(3,4,4-trifluorobut-3-en-1-yl)-λ⁴-sulfaneylidene)cyanamide, N-((3,4,4-trifluorobut-3-en-1-yl)(5-(5-(trifluoromethyl)-1,2,4-oxadiazol-3-yl)thiazol-2-yl)-λ⁴-sulfaneylidene)cyanamide, (Z)-2-((4,4-difluorobut-3-en-1-yl)sulfonyl)thiazole-5-carbaldehyde O-ethyl oxime, (E)-2-((4,4-difluorobut-3-en-1-yl)thio)thiazole-5-carbaldehyde O-methyl oxime, (Z)-2-((4,4-difluorobut-3-en-1-yl)thio)thiazole-5-carbaldehyde O-methyl oxime, (Z)-2-((4,4-difluorobut-3-en-1-yl)sulfonyl)thiazole-5-carbaldehyde O-methyl oxime, (E)-2-((4,4-difluorobut-3-en-1-yl)sulfonyl)thiazole-5-carbaldehyde O-ethyl oxime, (E)-4-methyl-2-((3,4,4-trifluorobut-3-en-1-yl)thio)oxazole-5-carbaldehyde O-methyl oxime, (E)-4-methyl-2-((3,4,4-trifluorobut-3-en-1-yl)thio)oxazole-5-carbaldehyde O-ethyl oxime, (E)-4-(tert-butyl)-2-((3,4,4-trifluorobut-3-en-1-yl)thio)oxazole-5-carbaldehyde O-methyl oxime, (E)-4-(tert-butyl)-2-((3,4,4-trifluorobut-3-en-1-yl)thio)oxazole-5-carbaldehyde O-ethyl oxime, (E)-5-chloro-2-((3,4,4-trifluorobut-3-en-1-yl)thio)thiazole-4-carbaldehyde O-ethyl oxime, (E)-5-chloro-2-((3,4,4-trifluorobut-3-en-1-yl)sulfinyl)thiazole-4-carbaldehyde O-ethyl oxime, (E)-5-chloro-2-((3,4,4-trifluorobut-3-en-1-yl)sulfinyl)thiazole-4-carbaldehyde O-methyl oxime, 4-methoxy-2-((3,4,4-trifluorobut-3-en-1-yl)sulfonyl)thiazole-5-carbaldehyde O-ethyl oxime, (E)-5-((2-phenylhydrazineylidene)methyl)-2-((3,4,4-trifluorobut-3-en-1-yl)thio)thiazole, (E)-5-((2-methylhydrazineylidene)methyl)-2-((3,4,4-trifluorobut-3-en-1-yl)thio)thiazole, (E)-2-((3,4,4-trifluorobut-3-en-1-yl)thio)thiazole-5-carbaldehyde O-benzyl oxime, (E)-5-((2-phenylhydrazineylidene)methyl)-2-((3,4,4-trifluorobut-3-en-1-yl)thio)oxazole, (E)-2-((3,4,4-trifluorobut-3-en-1-yl)sulfinyl)thiazole-5-carbaldehyde O-benzyl oxime, 4-methoxy-2-((3,4,4-trifluorobut-3-en-1-yl)thio)thiazole-5-carbaldehyde O-methyl oxime, 4-methoxy-2-((3,4,4-trifluorobut-3-en-1-yl)sulfonyl)thiazole-5-carbaldehyde O-methyl oxime, (E)-2-((3,4,4-trifluorobut-3-en-1-yl)sulfonyl)thiazole-5-carbaldehyde O-benzyl oxime, (E)-1-(4-methyl-2-((3,4,4-trifluorobut-3-en-1-yl)thio)oxazol-5-yl)ethan-1-one O-methyl oxime, (E)-1-(4-methyl-2-((3,4,4-trifluorobut-3-en-1-yl)thio)oxazol-5-yl)ethan-1-one O-ethyl oxime, (E)-1-(4-methyl-2-((3,4,4-trifluorobut-3-en-1-yl)thio)oxazol-5-yl)propan-1-one O-methyl oxime, (E)-1-(4-(tert-butyl)-2-((3,4,4-trifluorobut-3-en-1-yl)thio)oxazol-5-yl)ethan-1-one O-methyl oxime, (E)-1-(4-(tert-butyl)-2-((3,4,4-trifluorobut-3-en-1-yl)thio)oxazol-5-yl)propan-1-one O-methyl oxime, (E)-1-(4-(tert-butyl)-2-((3,4,4-trifluorobut-3-en-1-yl)thio)oxazol-5-yl)ethan-1-one O-ethyl oxime, (E)-4-methoxy-2-((3,4,4-trifluorobut-3-en-1-yl)sulfinyl)thiazole-5-carbaldehyde O-methyl oxime, (E)-4-methoxy-2-((3,4,4-trifluorobut-3-en-1-yl)sulfinyl)thiazole-5-carbaldehyde O-ethyl oxime, (E)-4-(tert-butyl)-2-((3,4,4-trifluorobut-3-en-1-yl)thio)oxazole-5-carbaldehyde O-cyclopropylmethyl oxime, (E)-4-methyl-2-((3,4,4-trifluorobut-3-en-1-yl)thio)oxazole-5-carbaldehyde O-cyclopropylmethyl oxime, 2-methyl-1-(4-methyl-2-((3,4,4-trifluorobut-3-en-1-yl)thio)oxazol-5-yl)propan-1-one O-methyl oxime, 3-methyl-1-(4-methyl-2-((3,4,4-trifluorobut-3-en-1-yl)thio)oxazol-5-yl)butan-1-one O-methyl oxime, (E)-4-methyl-2-((3,4,4-trifluorobut-3-en-1-yl)sulfinyl)oxazole-5-carbaldehyde O-cyclopropylmethyl oxime, (E)-4-methyl-2-((3,4,4-trifluorobut-3-en-1-yl)sulfonyl)oxazole-5-carbaldehyde O-cyclopropylmethyl oxime, 5-(thiophen-2-yl)-3-(2-((3,4,4-trifluorobut-3-en-1-yl)sulfinyl)thiazol-5-yl)-1,2,4-oxadiazole, 5-cyclobutyl-3-(2-((3,4,4-trifluorobut-3-en-1-yl)thio)thiazol-5-yl)-1,2,4-oxadiazole, 5-(thiophen-2-yl)-3-(2-((3,4,4-trifluorobut-3-en-1-yl)sulfonyl)thiazol-5-yl)-1,2,4-oxadiazole, 5-cyclopropyl-3-(2-((3,4,4-trifluorobut-3-en-1-yl)thio)thiazol-5-yl)-1,2,4-oxadiazole, (E)-4-methyl-2-((3,4,4-trifluorobut-3-en-1-yl)thio)oxazole-5-carbaldehyde O-isobutyl oxime, (E)-2-methyl-1-(4-methyl-2-((3,4,4-trifluorobut-3-en-1-yl)sulfinyl)oxazol-5-yl)propan-1-one O-methyl oxime, 3-methyl-1-(4-methyl-2-((3,4,4-trifluorobut-3-en-1-yl)sulfinyl)oxazol-5-yl)butan-1-one O-methyl oxime, (E)-2-((3,4,4-trifluorobut-3-en-1-yl)thio)thiazole-5-carbaldehyde O-cyclopropylmethyl oxime, (E)-2-((3,4,4-trifluorobut-3-en-1-yl)sulfinyl)thiazole-5-carbaldehyde O-cyclopropylmethyl oxime, 5-cyclopropyl-3-(2-((3,4,4-trifluorobut-3-en-1-yl)sulfonyl)thiazol-5-yl)-1,2,4-oxadiazole, 5-cyclobutyl-3-(2-((3,4,4-trifluorobut-3-en-1-yl)sulfonyl)thiazol-5-yl)-1,2,4-oxadiazole, 3-(2-((3,4,4-trifluorobut-3-en-1-yl)sulfinyl)thiazol-5-yl)-5-(trifluoromethyl)-1,2,4-oxadiazole, 3-(2-((3,4,4-trifluorobut-3-en-1-yl)sulfonyl)thiazol-5-yl)-5-(trifluoromethyl)-1,2,4-oxadiazole, 4-cyclopropyl-2-((3,4,4-trifluorobut-3-en-1-yl)thio)thiazole-5-carbaldehyde O-methyl oxime, 4-cyclopropyl-2-((3,4,4-trifluorobut-3-en-1-yl)thio)thiazole-5-carbaldehyde O-ethyl oxime, 4-cyclopropyl-2-((3,4,4-trifluorobut-3-en-1-yl)sulfinyl)thiazole-5-carbaldehyde O-methyl oxime, 4-cyclopropyl-2-((3,4,4-trifluorobut-3-en-1-yl)sulfonyl)thiazole-5-carbaldehyde O-methyl oxime, 5-cyclopropyl-3-(2-((3,4,4-trifluorobut-3-en-1-yl)sulfinyl)thiazol-5-yl)-1,2,4-oxadiazole, 2-((4,4-difluorobut-3-en-1-yl)thio)thiazole-5-carbaldehyde O-methyl oxime, 4-cyclopropyl-2-((3,4,4-trifluorobut-3-en-1-yl)sulfinyl)thiazole-5-carbaldehyde O-ethyl oxime, 4-cyclopropyl-2-((3,4,4-trifluorobut-3-en-1-yl)sulfonyl)thiazole-5-carbaldehyde O-ethyl oxime, (Z)-2-((4,4-difluorobut-3-en-1-yl)thio)oxazole-5-carbaldehyde O-methyl oxime, (E)-2-((4,4-difluorobut-3-en-1-yl)thio)oxazole-5-carbaldehyde O-methyl oxime, (Z)-2-((4,4-difluorobut-3-en-1-yl)thio)oxazole-5-carbaldehyde O-ethyl oxime, (E)-2-((4,4-difluorobut-3-en-1-yl)thio)oxazole-5-carbaldehyde O-ethyl oxime, (E)-4-methyl-2-((3,4,4-trifluorobut-3-en-1-yl)sulfinyl)oxazole-5-carbaldehyde O-methyl oxime, (E)-4-methyl-2-((3,4,4-trifluorobut-3-en-1-yl)sulfonyl)oxazole-5-carbaldehyde O-methyl oxime, (E)-2-((3,4,4-trifluorobut-3-en-1-yl)sulfonyl)thiazole-5-carbaldehyde O-cyclopropylmethyl oxime, (E)-2-((4,4-difluorobut-3-en-1-yl)thio)thiazole-5-carbaldehyde O-isopropyl oxime, (E)-2-((4,4-difluorobut-3-en-1-yl)sulfinyl)thiazole-5-carbaldehyde O-isopropyl oxime, 5-cyclobutyl-3-(2-((3,4,4-trifluorobut-3-en-1-yl)sulfinyl)thiazol-5-yl)-1,2,4-oxadiazole, (E)-2-((4,4-difluorobut-3-en-1-yl)thio)thiazole-5-carbaldehyde O-cyclopropylmethyl oxime, 2-((4,4-difluorobut-3-en-1-yl)sulfonyl)thiazole-5-carbaldehyde O-ethyl oxime, (E)-2-((4,4-difluorobut-3-en-1-yl)sulfinyl)oxazole-5-carbaldehyde O-methyl oxime, (E)-2-((4,4-difluorobut-3-en-1-yl)thio)thiazole-5-carbaldehyde O-cyclobutylmethyl oxime, (Z)-2-((4,4-difluorobut-3-en-1-yl)thio)thiazole-5-carbaldehyde O-cyclopropylmethyl oxime, (E)-2-((4,4-difluorobut-3-en-1-yl)sulfinyl)thiazole-5-carbaldehyde O-cyclobutylmethyl oxime, (Z)-2-((4,4-difluorobut-3-en-1-yl)sulfinyl)thiazole-5-carbaldehyde O-cyclopropylmethyl oxime, (Z)-2-((4,4-difluorobut-3-en-1-yl)sulfonyl)thiazole-5-carbaldehyde O-cyclopropylmethyl oxime, (E)-2-((4,4-difluorobut-3-en-1-yl)sulfonyl)thiazole-5-carbaldehyde O-cyclopropylmethyl oxime, (E)-2-((4,4-difluorobut-3-en-1-yl)sulfonyl)thiazole-5-carbaldehyde O-cyclobutylmethyl oxime, 5-ethyl-3-(2-((3,4,4-trifluorobut-3-en-1-yl)thio)thiazol-5-yl)-1,2,4-oxadiazole, 5-ethyl-3-(2-((3,4,4-trifluorobut-3-en-1-yl)sulfinyl)thiazol-5-yl)-1,2,4-oxadiazole, 5-ethyl-3-(2-((3,4,4-trifluorobut-3-en-1-yl)sulfonyl)thiazol-5-yl)-1,2,4-oxadiazole, 5-(tert-butyl)-3-(2-((3,4,4-trifluorobut-3-en-1-yl)thio)thiazol-5-yl)-1,2,4-oxadiazole, 5-(3,4-difluorophenyl)-3-(2-((3,4,4-trifluorobut-3-en-1-yl)thio)thiazol-5-yl)-1,2,4-oxadiazole, (E)-4-methyl-2-((3,4,4-trifluorobut-3-en-1-yl)thio)oxazole-5-carbaldehyde O-cyclobutylmethyl oxime, 5-(tert-butyl)-3-(2-((3,4,4-trifluorobut-3-en-1-yl)sulfinyl)thiazol-5-yl)-1,2,4-oxadiazole, 5-(tert-butyl)-3-(2-((3,4,4-trifluorobut-3-en-1-yl)sulfonyl)thiazol-5-yl)-1,2,4-oxadiazole, 5-(3,4-difluorophenyl)-3-(2-((3,4,4-trifluorobut-3-en-1-yl)sulfinyl)thiazol-5-yl)-1,2,4-oxadiazole, 5-(2-chloro-6-fluorophenyl)-3-(2-((3,4,4-trifluorobut-3-en-1-yl)thio)thiazol-5-yl)isoxazole, 4-chloro-2-((3,4,4-trifluorobut-3-en-1-yl)thio)thiazole-5-carbaldehyde O-methyl oxime, 5-(2-bromo-6-fluorophenyl)-3-(2-((3,4,4-trifluorobut-3-en-1-yl)thio)thiazol-5-yl)isoxazole, (E)-4-(methoxymethyl)-2-((3,4,4-trifluorobut-3-en-1-yl)thio)thiazole-5-carbaldehyde O-methyl oxime, (E)-4-(methoxymethyl)-2-((3,4,4-trifluorobut-3-en-1-yl)sulfinyl)thiazole-5-carbaldehyde O-methyl oxime, (E)-4-(methoxymethyl)-2-((3,4,4-trifluorobut-3-en-1-yl)sulfonyl)thiazole-5-carbaldehyde O-methyl oxime, 4-(methylamino)-2-((3,4,4-trifluorobut-3-en-1-yl)thio)thiazole-5-carbaldehyde O-methyl oxime, 5-(difluoromethyl)-2-((3,4,4-trifluorobut-3-en-1-yl)thio)thiazole, 5-(difluoromethyl)-2-((3,4,4-trifluorobut-3-en-1-yl)sulfinyl)thiazole, 2-((3,4,4-trifluorobut-3-en-1-yl)thio)thiazole-5-carbonitrile, 2-((3,4,4-trifluorobut-3-en-1-yl)sulfinyl)thiazole-5-carbonitrile, 5-(difluoromethyl)-2-((3,4,4-trifluorobut-3-en-1-yl)sulfonyl)thiazole, 5-(difluoromethyl)-2-((3,4,4-trifluorobut-3-en-1-yl)thio)oxazole, 2-((3,4,4-trifluorobut-3-en-1-yl)thio)oxazole-5-carbonitrile, 2-((3,4,4-trifluorobut-3-en-1-yl)sulfonyl)thiazole-5-carbonitrile, 2-((3,4,4-trifluorobut-3-en-1-yl)sulfinyl)oxazole-5-carbonitrile, 5-(difluoromethyl)-2-((3,4,4-trifluorobut-3-en-1-yl)sulfinyl)oxazole, 5-(difluoromethyl)-4-methyl-2-((3,4,4-trifluorobut-3-en-1-yl)thio)thiazole, 4-methyl-2-((3,4,4-trifluorobut-3-en-1-yl)thio)thiazole-5-carbonitrile, 5-(difluoromethyl)-4-methyl-2-((3,4,4-trifluorobut-3-en-1-yl)sulfinyl)thiazole, 4-methyl-2-((3,4,4-trifluorobut-3-en-1-yl)sulfinyl)thiazole-5-carbonitrile, 4-methyl-2-((3,4,4-trifluorobut-3-en-1-yl)sulfonyl)thiazole-5-carbonitrile, 5-(difluoromethyl)-4-methyl-2-((3,4,4-trifluorobut-3-en-1-yl)sulfonyl)thiazole, 5-(difluoromethyl)-4-methyl-2-((3,4,4-trifluorobut-3-en-1-yl)thio)oxazole, 4-(tert-butyl)-5-(difluoromethyl)-2-((3,4,4-trifluorobut-3-en-1-yl)thio)oxazole, 4-(tert-butyl)-5-(difluoromethyl)-2-((3,4,4-trifluorobut-3-en-1-yl)sulfonyl)oxazole, 4-methyl-2-((3,4,4-trifluorobut-3-en-1-yl)thio)oxazole-5-carbonitrile, 4-(tert-butyl)-2-((3,4,4-trifluorobut-3-en-1-yl)thio)oxazole-5-carbonitrile, 4-methoxy-2-((3,4,4-trifluorobut-3-en-1-yl)thio)thiazole-5-carbonitrile, 4-methoxy-2-((3,4,4-trifluorobut-3-en-1-yl)sulfinyl)thiazole-5-carbonitrile, 4-methyl-2-((3,4,4-trifluorobut-3-en-1-yl)sulfinyl)oxazole-5-carbonitrile, 4-(tert-butyl)-2-((3,4,4-trifluorobut-3-en-1-yl)sulfonyl)oxazole-5-carbonitrile, 5-(difluoromethyl)-4-methyl-2-((3,4,4-trifluorobut-3-en-1-yl)sulfonyl)oxazole, 4-cyclopropyl-5-(difluoromethyl)-2-((3,4,4-trifluorobut-3-en-1-yl)thio)thiazole, 4-cyclopropyl-5-(difluoromethyl)-2-((3,4,4-trifluorobut-3-en-1-yl)sulfinyl)thiazole, 4-cyclopropyl-5-(difluoromethyl)-2-((3,4,4-trifluorobut-3-en-1-yl)sulfonyl)thiazole, 4-cyclopropyl-2-((3,4,4-trifluorobut-3-en-1-yl)thio)thiazole-5-carbonitrile, 4-cyclopropyl-2-((3,4,4-trifluorobut-3-en-1-yl)sulfinyl)thiazole-5-carbonitrile, 4-cyclopropyl-2-((3,4,4-trifluorobut-3-en-1-yl)sulfonyl)thiazole-5-carbonitrile, 4-chloro-2-((3,4,4-trifluorobut-3-en-1-yl)thio)thiazole-5-carbonitrile, 4-cyclopropyl-2-((3,4,4-trifluorobut-3-en-1-yl)thio)thiazole, 4-cyclopropyl-2-((3,4,4-trifluorobut-3-en-1-yl)sulfinyl)thiazole, 4-cyclopropyl-2-((3,4,4-trifluorobut-3-en-1-yl)sulfonyl)thiazole, 4-methyl-2-((3,4,4-trifluorobut-3-en-1-yl)sulfonyl)oxazole-5-carbonitrile, (E)-1-methyl-2-((3,4,4-trifluorobut-3-en-1-yl)thio)-1H-imidazole-5-carbaldehyde O-ethyl oxime, (Z)-1-methyl-2-((3,4,4-trifluorobut-3-en-1-yl)thio)-1H-imidazole-5-carbaldehyde O-ethyl oxime, (E)-1-methyl-2-((3,4,4-trifluorobut-3-en-1-yl)thio)-1H-imidazole-5-carbaldehyde O-methyl oxime, (Z)-1-methyl-2-((3,4,4-trifluorobut-3-en-1-yl)thio)-1H-imidazole-5-carbaldehyde O-methyl oxime, (Z)-1-methyl-2-((3,4,4-trifluorobut-3-en-1-yl)sulfinyl)-1H-imidazole-5-carbaldehyde O-methyl oxime, 4-chloro-2-((3,4,4-trifluorobut-3-en-1-yl)thio)thiazole-5-carbaldehyde and (4-chloro-2-((3,4,4-trifluorobut-3-en-1-yl)thio)thiazol-5-yl)methanol.

6. A process for preparing a compound of formula (I) and/or a salt thereof as claimed in claim 1, wherein said process comprises at least one of the following steps (a) to (p):
a) converting substituted thioether compound of formula (4) to afford compound of formula (2) followed by alkylating or acylating the compound of formula (2) to afford compound of formula (1) according to reaction scheme as depicted below: 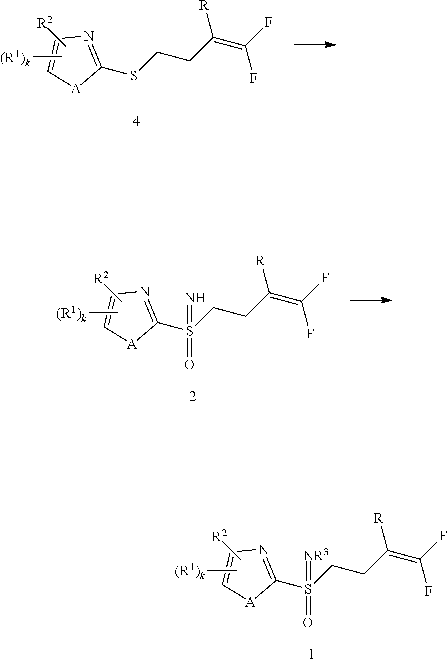
b) converting sulfoxide compound of formula (5) to sulfoximine compound of formula (2) followed by alkylating or acylating the compound of formula (2) to afford compound of formula (1) according to reaction scheme as depicted below: 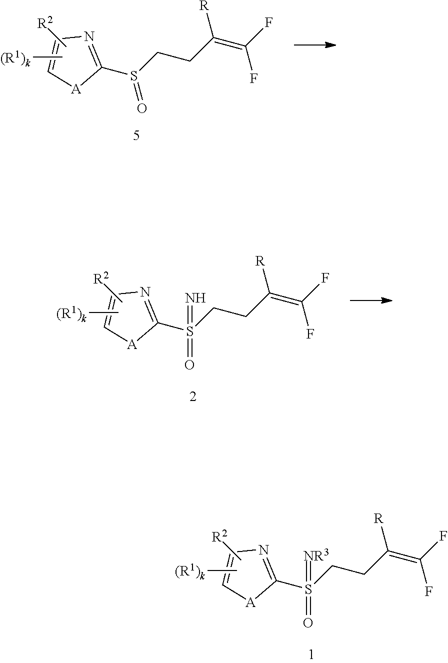
c) reacting substituted thioether compound of formula (4) with cyanamide compound to afford compound of formula (3) followed by oxidizing the compound of formula (3) to afford compound of formula (1) according to reaction scheme as depicted below: 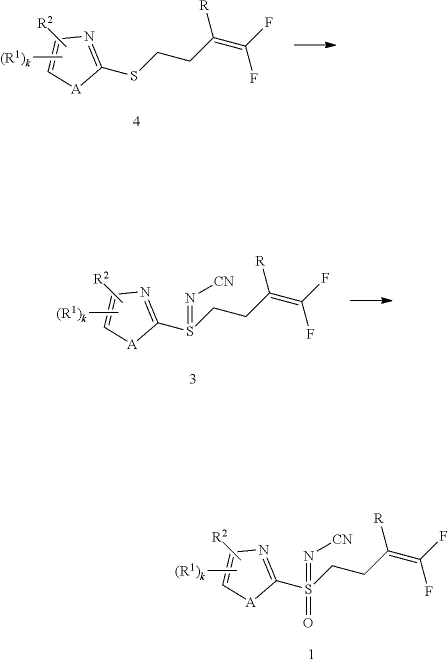
d) oxidizing substituted thioether of formula (4) to afford compound of formula (5) or (6) according to reaction scheme as depicted below: 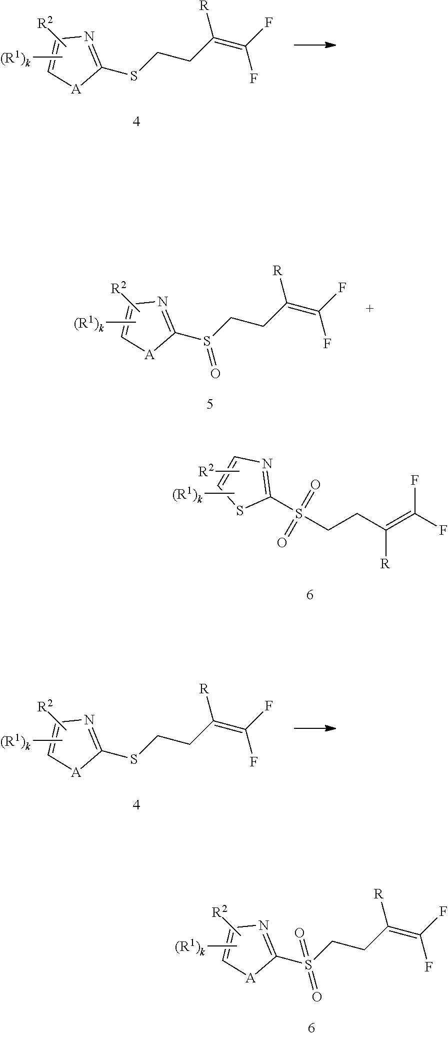
e) reducing ester compound of formula (15) to alcohol compound of formula (16) followed by oxidizing the compound of formula (16) to afford corresponding aldehyde compound of formula (4a or 4b) according to reaction scheme as depicted below: 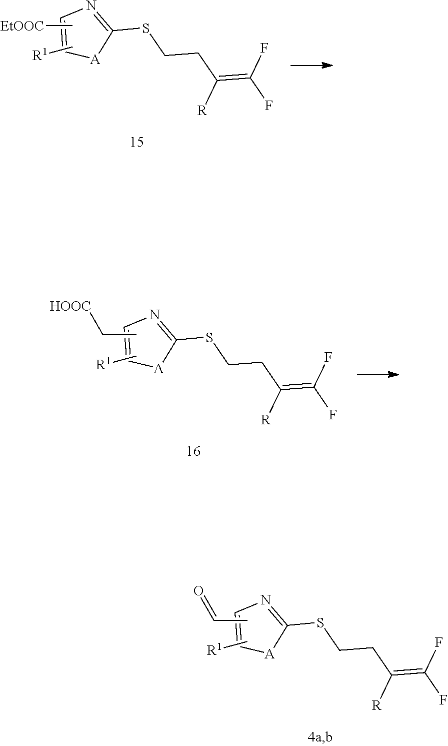
f) formylating thio compound of formula (18) to afford aldehyde compound of formula (4b) according to reaction scheme as depicted below: 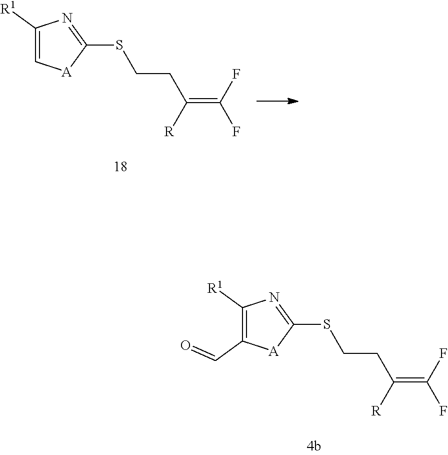
g) converting halide compound of formula (21) to thio compound of formula (22) followed by alkylation with compound of formula (14) to afford compound of formula (4a or 4b) according to reaction scheme as depicted below: 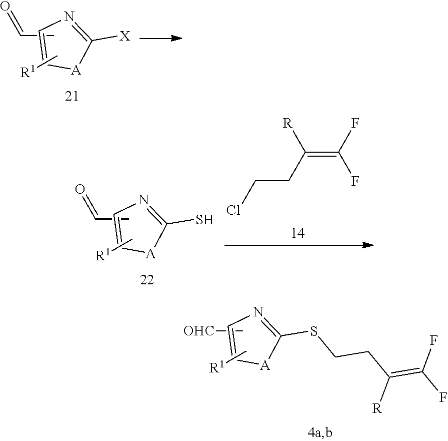
h) reacting aldehyde compound of formula (4a or 4b) with Grignard reagent to afford compound of formula (9) followed by oxidizing the compound of formula (9) to afford corresponding ketone compound of formula (10) according to reaction scheme as depicted below: 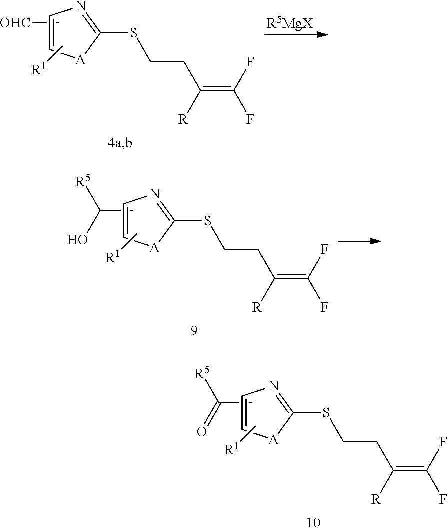
i) reacting aldehyde compound of formula (4a or 4b) with (un)substituted hydroxyl amine optionally followed by reaction with alkyl halide to afford compound of formula (8) which is then converted to compound of formula 1a following step (a) according to reaction scheme as depicted below: 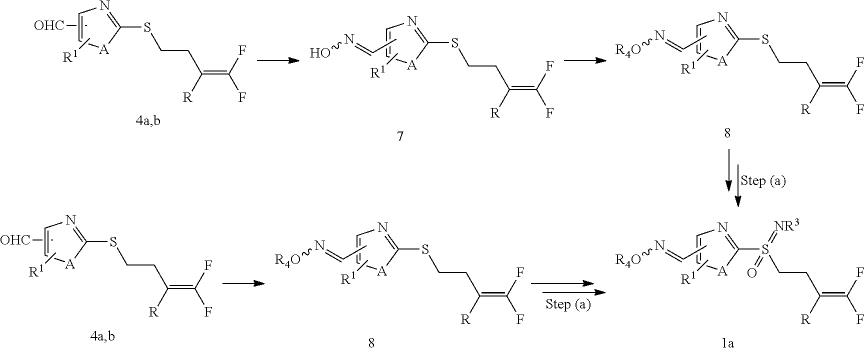
j) reacting ketone compound of formula (10) with substituted hydroxyl amine to afford compound of formula (11) followed by reaction with alkyl halide to afford compound of formula (12) which is then converted to compound of formula 1a following step (a) according to reaction scheme as depicted below: 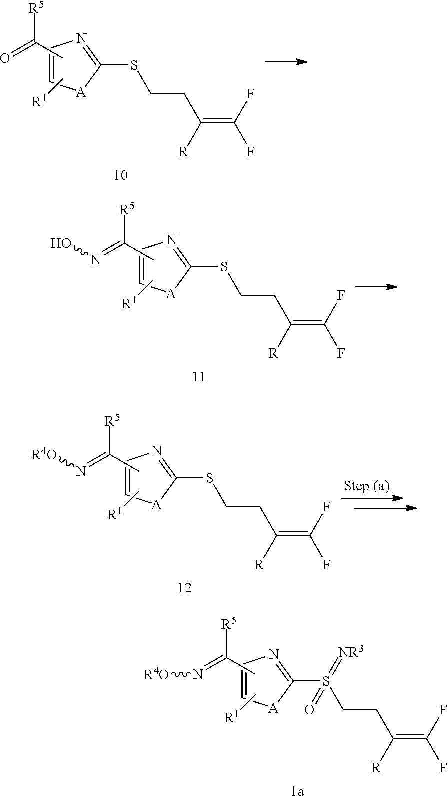
k) reacting substituted thioether compound of formula (18) with cyanamide compound to afford compound of formula (21) followed by oxidizing the compound of formula (21) to afford compound of formula (1a) according to reaction scheme as depicted below: 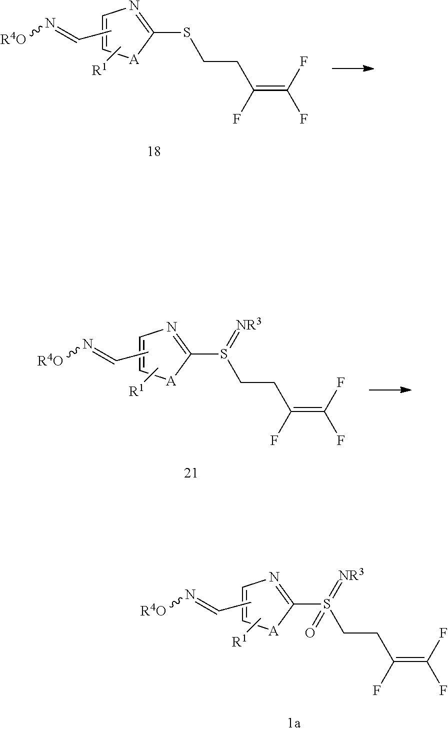
l) reacting compound of formula (4a or 4b) with suitable fluorinating agent to afford compound of formula (32) which is then converted to compound of formula (1b) following step (a) according to reaction scheme as depicted below: 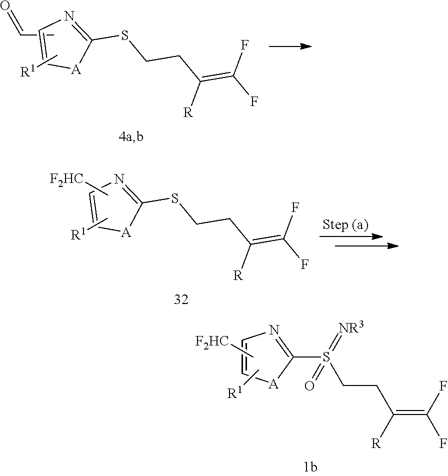
m) reacting compound of formula (7) with substituted styrene of formula (23) to afford compound of formula (24) which is then converted to compound of formula (1a) following step (a) respectively according to reaction schemes as depicted below: 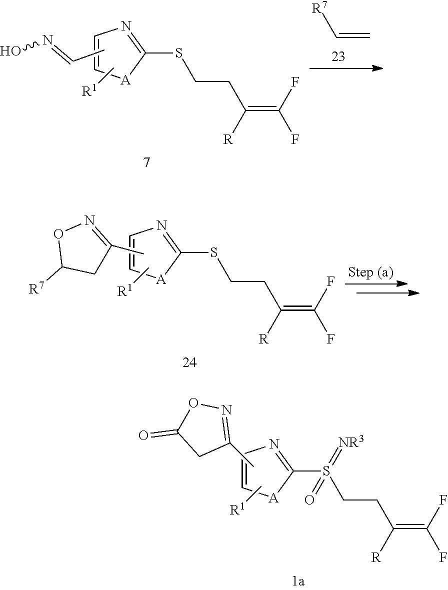
n) reacting compound of formula (7) with substituted alkyne of formula (25) to afford compound of formula (26) which is then converted to compound of formula (1a) following step (a) according to reaction scheme as depicted below: 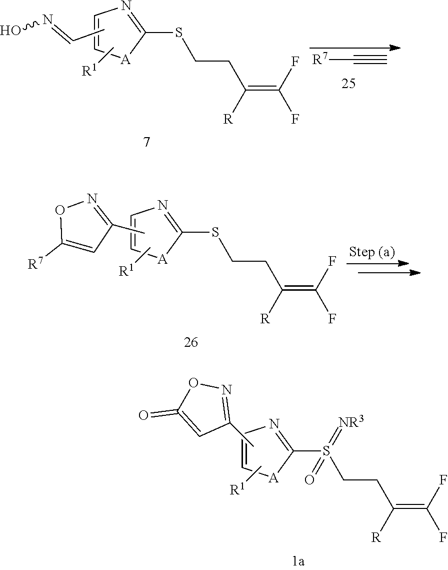
o) reacting oxime compound of formula (7) with phosphonic anhydride compound and further reacting with hydroxyl amine to afford compound of formula (28) followed by reaction with acid chloride/acid anhydride (29 or 29a) to afford compound of formula (30) which is then converted to compound of formula (1a) using step (a) according to reaction schemes as depicted below: 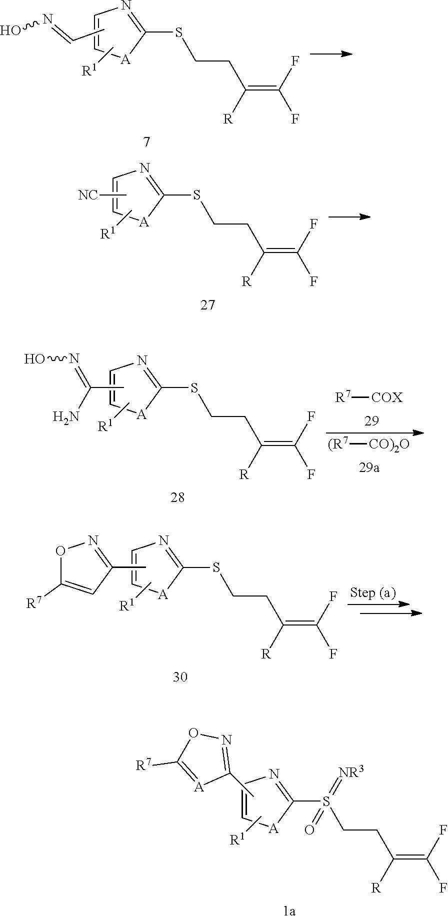
p) reacting compound of formula (28) with carbonylating agent to afford compound of formula (31) followed by converting the compound of formula (31) to compound of formula (1a) following step (a) according to reaction schemes as depicted below:

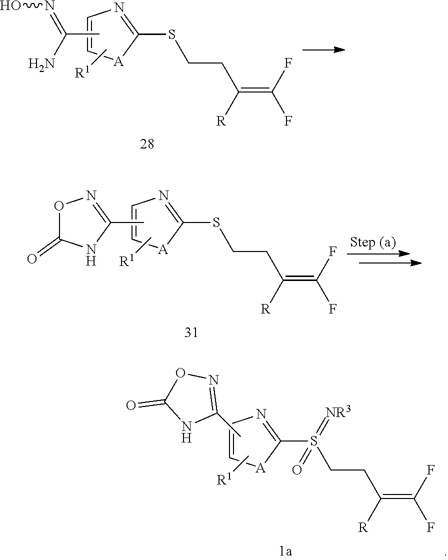

7. A composition for controlling or preventing phytopathogenic microorganisms comprising a compound of general formula (I), stereoisomer, agriculturally acceptable salts, tautomers or N-oxides thereof as claimed in claim 1 and one or more inert carriers.

8. The composition as claimed in claim 7, wherein said composition may additionally comprises one or more active compatible compound selected from fungicides, insecticides, nematicides, acaricides, biopesticides, herbicides, plant growth regulators, antibiotics, nutrients or fertilizers.

9. The composition as claimed in claim 7, wherein the concentration of the compound of general formula (I) ranges from 1 to 90% by weight with respect to the total weight of the composition, preferably from 5 to 50% by weight with respect to the total weight of the composition.

10. A combination comprising the compound of general formula (I), stereoisomer, agriculturally acceptable salts, tautomers or N-oxides thereof as claimed in claim 1 and one or more active compatible compound selected from fungicides, insecticides, nematicides, acaricides, biopesticides, herbicides, plant growth regulators, antibiotics, nutrients and fertilizers.

11. Use of compound of general formula (I), stereoisomers, agriculturally acceptable salts, tuatomers or N-oxides thereof or composition or combination thereof as claimed in claim 1, for controlling or preventing agricultural crops and/or horticultural crops against phytopathogenic fungi, bacteria, insects, nematodes, mites.

12. The use of compound of general formula (I) as claimed in claim 11, for controlling or preventing agricultural crops and/or horticultural crops against nematodes and phytopathogenic fungi.

13. The use of the compounds of general formula (I) as claimed in claim 11, wherein said agricultural crops are selected from cereals, corn, rice, soybean and other leguminous plants, fruits and fruit trees, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers, other vegetables and ornamentals.

14. A seed comprising compound of formula (I) and/or stereoisomers or agriculturally acceptable salts or tuatomers or N-oxides thereof or composition or combination thereof as claimed in claim 1, wherein the amount of the compound of the formula (I) or an N-oxide or an agriculturally acceptable salt thereof is ranging from 0.1 g to 10 kg per 100 kg of seed.

15. A method of controlling or preventing infestation of useful plants by phytopathogenic microorganisms in agricultural crops and/or horticultural crops, wherein the compound of general formula (I) and/or stereoisomers or agriculturally acceptable salts or tuatomers or N-oxides thereof or composition or combination thereof as claimed in claim 1, is applied to the plants, to parts thereof or a locus thereof.

16. A method of controlling or preventing infestation of useful plants by phytopathogenic microorganisms in agricultural crops and/or horticultural crops, wherein the compound of general formula (I) and/or stereoisomers or agriculturally acceptable salts or tuatomers or N-oxides thereof or composition or combination thereof as claimed in claim 1 is applied to the seeds of plants.

17. A method of controlling or preventing phytopathogenic microorganisms in agricultural crops and or horticultural crops using the compound of general formula (I) and/or stereoisomers or agriculturally acceptable salts or tuatomers or N-oxides thereof or composition or combination thereof as claimed in claim 1 comprises a step of applying an effective dosage of the compound or the composition or the combination, in amounts ranging from 1 g to 5 kg per hectare of agricultural and/or horticultural crops.

18. A compound of general formula (II), 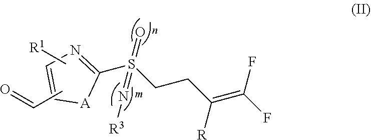
wherein;
A represent O, NR⁴ or S;
n, m, and k represents integers wherein n=0-2, m=0-1 and k=0-2;
R is selected from the group consisting of hydrogen, halogen and C₁-C₆-alkyl;
R¹ is selected from the group consisting of hydrogen, X, CN, SCN, SF₅, OR⁴, NO₂, N(R⁴)₂, Si(R⁴⁾₃, (C═O)—R⁴, S(O)₀₋₂R⁴, C₁-C₈-alkyl-S(O)₀₋₂R⁴, C₁-C₆-alkyl-OR⁴, C₁-C₈-alkyl-(C═O)—R⁴, C(R^{4a})═NR⁴, S(O)₀₋₂C₅-C₁₂-aryl, S(O)₀₋₂C₇-C₁₉-aralkyl, C₁-C₁₂-alkyl, C₂-C₁₂-alkenyl, C₂-C₁₂-alkynyl, C₁-C₁₂-haloalkyl, C₂-C₁₂-haloalkenyl, C₂-C₁₂-haloalkynyl, C₅-C₁₀-cycloalkyl, C₅-C₁₀-halocycloalkyl, C₄-C₁₀-cycloalkenyl, C₅-C₁₀-cycloalkynyl, C₁-C₈-alkyloxy-C₅-C₁₀-cycloalkyl, C₁-C₈-alkylthio-C₅-C₁₀-cycloalkyl, C₇-C₁₉-aralkyl, C₅-C₁₂-bicycloalkyl and C₇-C₁₂-bicycloalkenyl; wherein the cyclic ring system one or more carbon atoms may be replaced by heteroatoms selected from the group consisting of N, O, S and optionally including 1 to 3 ring members selected from the group consisting of C(═O), C(═S), S(O)₀₋₂ and Si(R⁴)₂; R¹ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂;
R⁴ is selected from the group consisting of hydrogen, N(R^{4a})₂, C₁-C₆-alkyl, C₁-C₆-alkenyl, C₁-C₆-alkynyl, C₁-C₆-haloalkyl, C₂-C₆-haloalkenyl, C₂-C₆-haloalkynyl, C₃-C₁₂-cycloalkyl, C₅-C₁₀-halocycloalkyl, C₄-C₁₂-cycloalkenyl, C₅-C₁₂-cycloalkynyl and C₃-C₁₂-heterocyclyl; R⁴ may be optionally substituted by one or more groups selected from the group consisting of X, R^{4a}, OR^{4a}, SR^{4a}, N(R^{4a})₂, Si(R^{4a})₃, COOR^{4a}, CN and CON(R^{4a})₂,
R^{4a} is selected from the group consisting of hydrogen, C₁-C₆-alkyl and C₃-C₆-cycloalkyl;
X represents halogen.
